(19) United States
(12) Patent Application Publication
(10) Pub. No.: US 2024/0254239 A1
(43) Pub. Date: Aug. 1, 2024

(54) DEPLETION OF ACTIVATED HEPATIC STELLATE CELLS (HSCS) AND USES THEREOF

(71) Applicant: LAEKNA THERAPEUTICS SHANGHAI CO., LTD., Shanghai (CN)

(72) Inventors: Chris Xiangyang LU, Shanghai (CN); Xiang-Ju Justin GU, Shanghai (CN); Minhua ZHANG, Shanghai (CN); Ruipeng ZHANG, Shanghai (CN)

(73) Assignee: LAEKNA THERAPEUTICS SHANGHAI CO., LTD., Shanghai (CN)

(21) Appl. No.: 18/574,243

(22) PCT Filed: Jun. 30, 2022
(86) PCT No.: PCT/CN2022/102692

§ 371 Date: Dec. 26, 2023

(30) Foreign Application Priority Data
Jul. 2, 2021 (WO) PCT/CN2021/104201

(51) Int. Cl. C07K 16/28; A61K 39/00; A61P 1/16; C07K 16/22; C07K 16/40

(52) U.S. Cl. CPC C07K 16/2851; A61P 1/16; C07K 16/22; C07K 16/2803; C07K 16/2863; C07K 16/2896; C07K 16/40; A61K 2039/505; C07K 2317/24; C07K 2317/52; C07K 2317/565; C07K 2317/72; C07K 2317/732

(57) ABSTRACT

A binding molecule comprising a binding domain that binds to an antigen expressed on an activated hepatic stellate cells (HSC), and a functional domain that is capable of enhancing an antibody effector function such as antibody-dependent cell-mediated cytotoxicity (ADCC) toward activated HSCs. Uses of such binding molecule are also provided herein.

### CROSS-REFERENCE TO RELATED APPLICATIONS

This application claims benefit of priority of International Patent Application No. PCT/CN2021/104201 filed on Jul. 2, 2021, the disclosure of which is incorporated by reference herein in its entirety.

### SEQUENCE LISTING

This application incorporates by reference a Sequence Listing submitted with this application as a text file entitled “14668-006-228_SEQ_LISTING.txt,” which was created on Jun. 27, 2022, and is 568, 110 bytes in size.

### 1. FIELD

Provided herein are molecules capable of binding to activated hepatic stellate cells (HSCs), pharmaceutical compositions comprising same, and uses thereof.

### 2. BACKGROUND

Activated hepatic stellate cells (HSC) are the key type of cells mediating fibrogenesis upon liver injury. Therapeutic approach targeting to activated HSCs is highly needed to slow or prevent fibrosis formation and improve quality of life in the patients with liver fibrosis or cirrhosis. The present disclosure addresses this and other needs in the art. 3. SUMMARY

In one aspect, provided herein is a binding molecule comprising one or more binding domain(s) that bind(s) to one or more antigen(s) expressed on an activated hepatic stellate cells (HSC), and a functional domain that is capable of enhancing an antibody effector function toward activated HSCs. In some embodiments, the antibody effector function is antibody-dependent cell-mediated cytotoxicity (ADCC). In some embodiments, the functional domain is an Fc region comprising one or more mutation(s) that enhances ADCC. In other embodiments, the functional domain is a domain that activates an immune cell. In some embodiments, the immune cell is a NK cell.

In some embodiments, the functional domain is the Fc region comprising one or more mutation(s) that enhances ADCC. In some embodiments, the one or more mutation(s) of the Fc region is at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In other embodiments, the functional domain is the domain that activates the immune cell, and wherein the functional domain promotes immune cell activation signaling or suppresses immune cell inhibitory signaling.

In some embodiments, the functional domain binds to and/or modulates a receptor on an immune cell. In some embodiments, the functional domain modulates a receptor that activates the immune cell. In other embodiments, the functional domain modulates a receptor that inhibits the immune cell. In some embodiments, the receptor is selected from a group consisting of NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some embodiments, the functional domain binds to NKG2D. In some embodiments, the functional domain is derived from a NKG2D ligand. In some embodiments, the functional domain is the extracellular domain of MICA, MICB, ULBP1, or ULBP2 or a fragment or a variant thereof, and wherein optionally the functional domain comprises an amino acid sequence of any one of SEQ ID NO: 90-93 or a fragment thereof. In some embodiments, the functional domain comprises an amino acid sequence of SEQ ID NO: 90 or a fragment thereof. In some embodiments, the functional domain comprises an amino acid sequence of SEQ ID NO: 91 or a fragment thereof. In some embodiments, the functional domain comprises an amino acid sequence of SEQ ID NO: 92 or a fragment thereof. In some embodiments, the functional domain comprises an amino acid sequence of SEQ ID NO: 93 or a fragment thereof.

In some embodiments, the functional domain binds to NKp46. In some embodiments, the functional domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the functional domain binds to TGFb. In some embodiments, the functional domain comprises TGFb receptor II extracellular domain (TRII) or a fragment or a variant thereof, and wherein optionally the functional domain comprises the amino acid sequence of SEQ ID NO: 94 or a fragment thereof.

In some embodiments, the functional domain binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the functional domain binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some embodiments, in addition to the functional domain that activates the immune cell described above, the binding molecule further comprises an Fc region comprising one or more mutation(s) that enhances ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antigen expressed on the HSC is selected from a group consisting of 5HT1B, 5HTIF, 5HT2A, 5-HT2B, 5-HT7, A2a, A2b, a1b1 integrin, a2bi integrin, a5b1 integrin, a6b4 integrin, a8b1 integrin, avb1 integrin, avb3 integrin, ACVR2A, ACVR2B, AdipoR1, AdipoR2, ADRA1A, ADRA1B, ANTXR1, AT1, AT2, BAMB1, BMPR2, C5aR, CB1, CB2, CCR1, CCR2, CCR5, CCR7, CD105, CD112, CD14, CD146, CD155, CD248, CD36, CD38, CD40, CD44, CD49e, CD62e, CD73, CD95, c-MET, CNTFR, CXCR3, CXCR4, DDR1, DDR2, EGFR, ETA, ETB, FAP, FGFR2, FN, gp130, GPC3, GPR91, ICAM-1, IGF-1R, IGF-2R, IL-10R2, IL-11RA, IL-17RA, IL-20R1, IL-20R2, IL-22R1, IL-6R, KCNE4, ITGA8, LRP, MICA, MICB, NCAM, NGFR, NPR-B, NPR3, OB-Ra, OB-Rb, OPRD1, P2X4, P2X7, P2Y6, p75NTR, PAFR, PAR1, PAR2, PAR4, PDGFRA, PDGFRB, PD-L1, PD-L2, Ptc, PTH-1R, RAGE, SIRPA, CD47, SYP, TGFBR1, TGFBR2, TGFBR3, TLR2, TLR3, TLR4, TLR7, TLR9, TNFR1, TRKB, TRKC, ULBP1, ULPB2, uPAR, VACM-1, VEGFR-1, and VEGFR-2.

In some embodiments, the antigen is PDGFRb. In some embodiments, the binding domain comprises an HCDR1, an HCDR2, and an HCDR3 as set forth in SEQ ID NO: 67, and a LCDR1, a LCDR2, and a LCDR3 as set forth in SEQ ID NO: 68. In some embodiments, the HCDR1 comprises the amino acid sequence of SEQ ID NO: 1, the HCDR2 comprises the amino acid sequence of SEQ ID NO: 2, the HCDR3 comprises the amino acid sequence of SEQ ID NO: 3, the LCDR1 comprises the amino acid sequence of SEQ ID NO: 4, the LCDR2 comprises the amino acid sequence of SEQ ID NO: 5, and the LCDR3 comprises the amino acid sequence of SEQ ID NO: 6. In some embodiments, the binding domain comprises a VH comprising the amino acid sequence of SEQ ID NO: 67 and a VL comrpsiing the amino acid sequence of SEQ ID NO: 68.

In some embodiments, the antigen is SIRPA. In some embodiments, the binding domain comprises (i) an HCDR1, an HCDR2, and an HCDR3 as set forth in SEQ ID NO: 69, and a LCDR1, a LCDR2, and a LCDR3 as set forth in SEQ ID NO: 70; (ii) an HCDR1, an HCDR2, and an HCDR3 as set forth in SEQ ID NO: 71, and a LCDR1, a LCDR2, and a LCDR3 as set forth in SEQ ID NO: 72; or (iii) a HCDR1, an HCDR2, and an HCDR3 as set forth in SEQ ID NO: 73, and a LCDR1, a LCDR2, and a LCDR3 as set forth in SEQ ID NO: 74. In some embodiments, (i) the HCDR1 comprises the amino acid sequence of SEQ ID NO: 7, the HCDR2 comprises the amino acid sequence of SEQ ID NO: 8, the HCDR3 comprises the amino acid sequence of SEQ ID NO: 9, the LCDR1 comprises the amino acid sequence of SEQ ID NO: 10, the LCDR2 comprises the amino acid sequence of SEQ ID NO: 11, and the LCDR3 comprises the amino acid sequence of SEQ ID NO: 12; (ii) the HCDR1 comprises the amino acid sequence of SEQ ID NO: 13, the HCDR2 comprises the amino acid sequence of SEQ ID NO: 14, the HCDR3 comprises the amino acid sequence of SEQ ID NO: 15, the LCDR1 comprises the amino acid sequence of SEQ ID NO: 16, the LCDR2 comprises the amino acid sequence of SEQ ID NO: 17, and the LCDR3 comprises the amino acid sequence of SEQ ID NO: 18; or (iii) the HCDR1 comprises the amino acid sequence of SEQ ID NO: 19, the HCDR2 comprises the amino acid sequence of SEQ ID NO: 20, the HCDR3 comprises the amino acid sequence of SEQ ID NO: 21, the LCDR1 comprises the amino acid sequence of SEQ ID NO: 22, the LCDR2 comprises the amino acid sequence of SEQ ID NO: 23, and the LCDR3 comprises the amino acid sequence of SEQ ID NO: 24. In some embodiments, the binding domain comprises: (i) a VH comprising the amino acid sequence of SEQ ID NO: 69 and a VL comrpsiing the amino acid sequence of SEQ ID NO: 70; (ii) a VH comprising the amino acid sequence of SEQ ID NO: 71 and a VL comrpsiing the amino acid sequence of SEQ ID NO: 72; or (iii) a VH comprising the amino acid sequence of SEQ ID NO: 73 and a VL comrpsiing the amino acid sequence of SEQ ID NO: 74.

In some embodiments, the antigen is FAPα. In some embodiments, the binding domain comprises (i) an HCDR1, an HCDR2, and an HCDR3 as set forth in SEQ ID NO: 75, and a LCDR1, a LCDR2, and a LCDR3 as set forth in SEQ ID NO: 76; or (ii) an HCDR1, an HCDR2, and an HCDR3 as set forth in SEQ ID NO: 77, and a LCDR1, a LCDR2, and a LCDR3 as set forth in SEQ ID NO: 78. In some embodiments, (i) the HCDR1 comprises the amino acid sequence of SEQ ID NO: 25, the HCDR2 comprises the amino acid sequence of SEQ ID NO: 26, the HCDR3 comprises the amino acid sequence of SEQ ID NO: 27, the LCDR1 comprises the amino acid sequence of SEQ ID NO: 28, the LCDR2 comprises the amino acid sequence of SEQ ID NO: 29, and the LCDR3 comprises the amino acid sequence of SEQ ID NO: 30; or (ii) the HCDR1 comprises the amino acid sequence of SEQ ID NO: 31, the HCDR2 comprises the amino acid sequence of SEQ ID NO: 32, the HCDR3 comprises the amino acid sequence of SEQ ID NO: 33, the LCDR1 comprises the amino acid sequence of SEQ ID NO: 34, the LCDR2 comprises the amino acid sequence of SEQ ID NO: 35, and the LCDR3 comprises the amino acid sequence of SEQ ID NO: 36. In some embodiments, the binding domain comprises: (i) a VH comprising the amino acid sequence of SEQ ID NO: 75 and a VL comrpsiing the amino acid sequence of SEQ ID NO: 76; or (ii) a VH comprising the amino acid sequence of SEQ ID NO: 77 and a VL comrpsiing the amino acid sequence of SEQ ID NO: 78.

In some embodiments, the antigen is PD-L1. In some embodiments, the binding domain comprises an HCDR1, an HCDR2, and an HCDR3 as set forth in SEQ ID NO: 79, and a LCDR1, a LCDR2, and a LCDR3 as set forth in SEQ ID NO: 80. In some embodiments, the HCDR1 comprises the amino acid sequence of SEQ ID NO: 37, the HCDR2 comprises the amino acid sequence of SEQ ID NO: 38, the HCDR3 comprises the amino acid sequence of SEQ ID NO: 39, the LCDR1 comprises the amino acid sequence of SEQ ID NO: 40, the LCDR2 comprises the amino acid sequence of SEQ ID NO: 41, and the LCDR3 comprises the amino acid sequence of SEQ ID NO: 42. In some embodiments, the binding domain comprises a VH comprising the amino acid sequence of SEQ ID NO: 79 and a VL comrpsiing the amino acid sequence of SEQ ID NO: 80.

In some embodiments, the antigen is uPAR. In some embodiments, the binding domain comprises an HCDR1, an HCDR2, and an HCDR3 as set forth in SEQ ID NO: 81, and a LCDR1, a LCDR2, and a LCDR3 as set forth in SEQ ID NO: 82. In some embodiments, the HCDR1 comprises the amino acid sequence of SEQ ID NO: 43, the HCDR2 comprises the amino acid sequence of SEQ ID NO: 44, the HCDR3 comprises the amino acid sequence of SEQ ID NO: 45, the LCDR1 comprises the amino acid sequence of SEQ ID NO: 46, the LCDR2 comprises the amino acid sequence of SEQ ID NO: 47, and the LCDR3 comprises the amino acid sequence of SEQ ID NO: 48. In some embodiments, the binding domain comprises a VH comprising the amino acid sequence of SEQ ID NO: 81 and a VL comrpsiing the amino acid sequence of SEQ ID NO: 82.

In some embodiments, the antigen is IGF-IR. In some embodiments, the binding domain comprises: (i) an HCDR1, an HCDR2, and an HCDR3 as set forth in SEQ ID NO: 83, and a LCDR1, a LCDR2, and a LCDR3 as set forth in SEQ ID NO: 84; or (ii) an HCDR1, an HCDR2, and an HCDR3 as set forth in SEQ ID NO: 85, and a LCDR1, a LCDR2, and a LCDR3 as set forth in SEQ ID NO: 86. In some embodiments, (i) the HCDR1 comprises the amino acid sequence of SEQ ID NO: 49, the HCDR2 comprises the amino acid sequence of SEQ ID NO: 50, the HCDR3 comprises the amino acid sequence of SEQ ID NO: 51, the LCDR1 comprises the amino acid sequence of SEQ ID NO: 52, the LCDR2 comprises the amino acid sequence of SEQ ID NO: 53, and the LCDR3 comprises the amino acid sequence of SEQ ID NO: 54; or (ii) the HCDR1 comprises the amino acid sequence of SEQ ID NO: 55, the HCDR2 comprises the amino acid sequence of SEQ ID NO: 56, the HCDR3 comprises the amino acid sequence of SEQ ID NO: 57, the LCDR1 comprises the amino acid sequence of SEQ ID NO: 58, the LCDR2 comprises the amino acid sequence of SEQ ID NO: 59, and the LCDR3 comprises the amino acid sequence of SEQ ID NO: 60. In some embodiments, the binding domain comprises: (i) a VH comprising the amino acid sequence of SEQ ID NO: 83 and a VL comrpsiing the amino acid sequence of SEQ ID NO: 84; or (ii) a VH comprising the amino acid sequence of SEQ ID NO: 85 and a VL comrpsiing the amino acid sequence of SEQ ID NO: 86.

In some embodiments, the antigen is ANTXR1. In some embodiments, the binding domain comprises: (i) an HCDR1, an HCDR2, and an HCDR3 as set forth in SEQ ID NO: 225, and a LCDR1, a LCDR2, and a LCDR3 as set forth in SEQ ID NO: 226; or (ii) an HCDR1, an HCDR2, and an HCDR3 as set forth in SEQ ID NO: 233, and a LCDR1, a LCDR2, and a LCDR3 as set forth in SEQ ID NO: 234. In some embodiments, (i) the HCDR1 comprises the amino acid sequence of SEQ ID NO:227, the HCDR2 comprises the amino acid sequence of SEQ ID NO: 228, the HCDR3 comprises the amino acid sequence of SEQ ID NO: 229, the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 232; or (ii) the HCDR1 comprises the amino acid sequence of SEQ ID NO: 235, the HCDR2 comprises the amino acid sequence of SEQ ID NO: 236, the HCDR3 comprises the amino acid sequence of SEQ ID NO: 237, the LCDR1 comprises the amino acid sequence of SEQ ID NO: 238, the LCDR2 comprises the amino acid sequence of SEQ ID NO: 239, and the LCDR3 comprises the amino acid sequence of SEQ ID NO: 240. In some embodiments, the binding domain comprises: (i) a VH comprising the amino acid sequence of SEQ ID NO: 225 and a VL comrpsiing the amino acid sequence of SEQ ID NO: 226; or (ii) a VH comprising the amino acid sequence of SEQ ID NO: 233 and a VL comrpsiing the amino acid sequence of SEQ ID NO: 234.

In some embodiments, the antigen is CD248. In some embodiments, the binding domain comprises: (i) an HCDR1, an HCDR2, and an HCDR3 as set forth in SEQ ID NO: 241, and a LCDR1, a LCDR2, and a LCDR3 as set forth in SEQ ID NO: 242; or (ii) an HCDR1, an HCDR2, and an HCDR3 as set forth in SEQ ID NO: 249, and a LCDR1, a LCDR2, and a LCDR3 as set forth in SEQ ID NO: 250. In some embodiments, (i) the HCDR1 comprises the amino acid sequence of SEQ ID NO:243, the HCDR2 comprises the amino acid sequence of SEQ ID NO: 244, the HCDR3 comprises the amino acid sequence of SEQ ID NO: 245, the LCDR1 comprises the amino acid sequence of SEQ ID NO: 246, the LCDR2 comprises the amino acid sequence of SEQ ID NO: 247, and the LCDR3 comprises the amino acid sequence of SEQ ID NO: 248; or (ii) the HCDR1 comprises the amino acid sequence of SEQ ID NO: 251, the HCDR2 comprises the amino acid sequence of SEQ ID NO: 252, the HCDR3 comprises the amino acid sequence of SEQ ID NO: 253, the LCDR1 comprises the amino acid sequence of SEQ ID NO: 254, the LCDR2 comprises the amino acid sequence of SEQ ID NO: 255, and the LCDR3 comprises the amino acid sequence of SEQ ID NO: 256. In some embodiments, the binding domain comprises: (i) a VH comprising the amino acid sequence of SEQ ID NO: 241 and a VL comrpsiing the amino acid sequence of SEQ ID NO: 242; or (ii) a VH comprising the amino acid sequence of SEQ ID NO: 249 and a VL comrpsiing the amino acid sequence of SEQ ID NO: 250.

In some embodiments, the antigen is GPC3. In some embodiments, the binding domain comprises an HCDR1, an HCDR2, and an HCDR3 as set forth in SEQ ID NO: 257, and a LCDR1, a LCDR2, and a LCDR3 as set forth in SEQ ID NO: 258. In some embodiments, the HCDR1 comprises the amino acid sequence of SEQ ID NO: 259, the HCDR2 comprises the amino acid sequence of SEQ ID NO: 260, the HCDR3 comprises the amino acid sequence of SEQ ID NO: 261, the LCDR1 comprises the amino acid sequence of SEQ ID NO: 262, the LCDR2 comprises the amino acid sequence of SEQ ID NO: 263, and the LCDR3 comprises the amino acid sequence of SEQ ID NO: 264. In some embodiments, the binding domain comprises a VH comprising the amino acid sequence of SEQ ID NO: 257 and a VL comrpsiing the amino acid sequence of SEQ ID NO: 258.

In some embodiments, the antigen is a NKG2D ligand, such as MICA, MICB, ULBP1, or ULBP2. In some embodiments, the binding domain comprises the NKG2D extracellular domain or a fragment or a variant thereof, and wherein optionally the binding domain comprises the amino acid sequence of SEQ ID NO: 89 or a fragment thereof.

In some embodiments, the binding molecule is an IgG antibody or a fusion protein comprising the IgG antibody. In some embodiments, the antibody is a humanized antibody.

In another aspect, provided herein is a nucleic acid molecule encoding the binding molecule provided herein or a fragment thereof.

In another aspect, provided herein is a vector comprising the nucleic acid molecule provided herein.

In another aspect, provided herein is a host cell transformed with the vector provided herein.

In another aspect, provided herein is a composition comprising a therapeutically effective amount of the binding molecule, the nucleic acid molecule, or the vector provided herein, and a pharmaceutically acceptable excipient.

In yet another aspect, provided herein is a method of treating a disease or disorder in a subject, comprising administering to the subject the composition provided herein. In some embodiments, the disease or disorder is associated with activiated HSCs. In some embodiments, the disease or disorder is liver fibrosis.

In yet another aspect, provided herein is a method of depleting activated HSCs in a subject, comprising administering to the subject the composition provided herein. In some embodiments, the subject has a disease or disorder associated with activiated HSCs. In some embodiments, the disease or disorder is liver fibrosis.

### 4. BRIEF DESCRIPTION OF THE FIGURES

FIGS. 1A-1J show dose dependent binding of antibodies or recombinant fusion proteins to the respective antigens measured by ELISA. (FIG. 1A) α-PDGFRb 1 S239D/1332E binding to Human PDGFRb ECD; (FIG. 1B) NKG2D-hIgG1 Fc S239D/1332E binding to human ULBP1 ECD; (FIG. 1C) α-SIRPA 1 S239D/1332E, α-SIRPA 2 S239D/1332E, and α-SIRPA 3 S239D/1332E binding to human SIRPA ECD; (FIG. 1D) α-FAPa 1 S239D/1332E binding to human FAPa ECD; (FIG. 1E) α-uPAR 1 S239D/1332E binding to human uPAR ECD; (FIG. 1F) α-IGF1R 1 S239D/1332E and α-IGF1R 2 S239D/1332E binding to human IGF-IR ECD; (FIG. 1G) α-PDL1 1 S239D/1332E binding to human PD-L1 ECD. (FIG. 1H) α-GPC3 1 mIgG2a S239D/1332E binding to human GPC3 ECD. (FIG. 11) α-ANTXR1 1 mIgG2a S239D/1332E and α-ANTXR1 2 mIgG2a S239D/1332E binding to human ANTXR1 ECD. (FIG. 1J) α-CD248 1 mIgG2a S239D/1332E and α-CD248 2 mIgG2a S239D/1332E binding to human CD248 ECD.

FIGS. 2A-2F show surface expression levels of the antigens on human TGFb1-activated primary human HSC cells. (FIG. 2A) Mean fluorescence intensity of 10 ug/ml α-PDGFRb 1 S239D/1332E, α-SIRPA 2 S239D/1332E and α-FAPa 2 WT FACS binding; (FIG. 2B) Mean fluorescence intensity of 10 ug/ml α-PDGFRb 1 S239D/1332E, α-uPAR 1 S239D/1332E, α-IGF1R 1 S239D/1332E and α-IGF1R 2 S239D/1332E FACS binding; (FIG. 2C) Mean fluorescence intensity of 10 ug/ml α-PDGFRb 1 S239D/1332E, α-PDL1 1 S239D/1332E FACS binding; (FIG. 2D) Mean fluorescence intensity of 10 ug/ml NKG2D hIgG1 Fc S239D/1332E FACS binding. (FIG. 2E) Mean fluorescence intensity of 10 ug/ml α-GPC3 1 mIgG2a S239D/1332E, α-CD248 1 mIgG2a S239D/1332E and α-CD248 2 mIgG2a S239D/1332E FACS binding. (FIG. 2F) Mean fluorescence intensity of 10 ug/ml α-ANTXR1 1 mIgG2a S239D/1332E and α-ANTXR1 2 mIgG2a S239D/1332E FACS binding.

FIGS. 3A-3G show testing antibodies or proteins increased cytotoxic activity of primary NK cells or PBMC cells against TGFb1-activated primary human HSC cells. Data were presented as Mean+SD (n=3/group) (FIG. 3A) Dose dependent cytotoxicity induced by α-PDGFRb 1 S239D/1332E (primary NK: HSC cells=4:1); (FIG. 3B) Cytotoxicity induced by 10 ug/ml NKG2D hIgG1 Fc S239D/1332E and 10 ug/ml α-PDGFRb 1 S239D/1332E (primary NK: HSC cells=4:1); (FIG. 3C) Dose dependent cytotoxicity induced by α-SIRPA 1 S239D/1332E, α-SIRPA 2 S239D/1332E and α-SIRPA 3 S239D/1332E (primary NK: HSC cells=4:1); (FIG. 3D) Dose dependent cytotoxicity induced by α-FAPa 1 S239D/1332E (primary NK: HSC cells=4:1); (FIG. 3E) Dose dependent cytotoxicity induced by α-uPAR 1 S239D/1332E, α-IGF1R 1 S239D/1332E, and α-IGF1R 2 S239D/1332E (primary PBMC: HSC cells=50:1). 10 ug/ml α-PDGFRb 1 S239D/1332E was used as the positive control of the assay; (FIG. 3F) Dose dependent cytotoxicity induced by α-PD-L1 1 S239D/1332E (primary NK: HSC cells=4:1). 10 ug/ml α-PDGFRb 1 S239D/1332E was used as the positive control of the assay. (FIG. 3G) Testing antibodies increased cytotoxic activity of primary human PBMC cells against TGFb1-activated primary human HSC cells (primary PBMC: HSC cells=25:1). α-SIRPA 2 mIgG2a S239D/1332E was used as positive control. Mouse IgG2a isotype control was used as negative control. Data were presented as Mean+SD (n=2/group).

FIGS. 4A-4C show the effector function of Fc region was critical for antibodies or proteins to exert ADCC cytotoxicity against TGFb1-activated human primary HSC cells. Data were presented as Mean+SD (n=2-3/group) (FIG. 4A) Dose dependent cytotoxicity induced by α-SIRPA 2 with different Fc mutation (primary PBMC: HSC cells=50:1). Effector function enhancing mutations (S239D/1332E, S239D/A330L/1332E, L235V/F243L/R292P/Y300L/P396L, F243L/R292P/Y300L) increased ADCC cytotoxicity, while effector function silencing mutation (L234A/L235A) abolished ADCC cytotoxicity. WT: wild type; (FIG. 4B) Dose dependent cytotoxicity induced by α-PDGFRb 1 with different Fc mutations (primary NK: HSC cells=4:1). Antibody with S239D/1332E mutation dose dependently induced cytotoxicity against primary HSC cells in the NK/HSC co-culture. N297S mutation completely abolished ADCC effect. (FIG. 4C) 10 ug/ml NKG2D hIgG1 Fc S239D/1332E induced more cytotoxicity than 10 ug/ml NKG2D hIgG1 Fc L234A/L235A (primary PBMC: HSC cells=30:1).

FIG. 5 shows the reduction of total PDGFRb levels in the liver after single dose of 1B3-TRII in CCL4-treated C57BL/6J mice was dependent on Fc effector function. 100 ug total protein of liver lysate was loaded onto the gel. Total PDGFRb was blotted using anti-PDGFRb antibody (abcam Ab32570). Alpha-tubulin was blotted as loading control (Beyotime, AF0001). 1B3-TRII S239D/1332E treatment reduced total PDGFRb protein levels while 1B3-TRII L234A/L235A treatment did not. 1B3-TRII: anti-mouse PDGFRb antibody 1B3 with human TGFb receptor II ECD linked to heavy chain C-terminus;

FIG. 6 shows comparable antibody concentrations in the liver after single dose of 1B3-TRII S239D/1332E or 1B3-TRII L234A/L235A in CCL4-treated C57BL/6J mice. Antibody concentration was determined by ELISA. Data were presented as Mean±SD (n=3/group). 1B3-TRII: anti-mouse PDGFRb antibody 1B3 with human TGFb receptor II ECD linked to heavy chain C-terminus;

FIG. 7 shows comparable antibody concentrations in the serum after multiple doses of 1B3-TRII S239D/1332E and 1B3-TRII L234A/L235A in CCL4-treated C57BL/6J mice. Serum antibody concentrations was measured at day 9 and day 27 after the start of the dosing. Antibody concentration was determined by direct ELISA. Data were presented as Mean±SD (n=8-10/group).

FIGS. 8A-8C show bispecific antibody α-PDGFRb 1-NKG2D KIH S239D/1332E further increased cytotoxicity against TGFb1-activated HSC cells comparing to α-PDGFRb 1 S239D/1332E or NKG2D hIgG1 Fc S239D/1332E. (FIG. 8A) α-PDGFRb 1 S239D/1332E and α-PDGFRb 1-NKG2D KIH S239D/1332E dose dependently bind to Human PDGFRb ECD measured by ELISA; (FIG. 8B) NKG2D hIgG1 Fc S239D/1332E and α-PDGFRb 1-NKG2D KIH S239D/1332E dose dependently bind to human MICA ECD measured by ELISA. (FIG. 8C) Cytotoxicity induced dose dependently by α—PDGFRb 1 S239D/1332E, NKG2D hIgG1 Fc S239D/1332E or α-PDGFRb 1-NKG2D KIH S239D/1332E treatment (primary PBMC: aHSC cells =25:1). α-PDGFRb 1-NKG2D KIH S239D/1332E induced more maximum cytotoxicity comparing to either α—PDGFRb 1 S239D/1332E or NKG2D hIgG1 Fc S239D/1332E. Data were presented as Mean±SD (n=2/group).

FIG. 9 shows fusion of ULBP2 to N-term of α-PDGFRb 1 heavy chain increased cytotoxic activity of primary PBMC cells against TGFb1-activated human primary HSC cells. co-culture of primary PBMC and activated HSC cells (primary PBMC: HSC cells=30:1) were treated with 10 ug/ml ULBP2-HC α-PDGFRb 1 L234A/L235A or α-PDGFRb 1 L234A/L235A. hIgG1 isotype control was used as negative control. Cytotoxicity to HSC cells were determined using LDH release. Data were presented as Mean±SD (n=3/group). Data were analyzed using one way ANOVA followed by multiple comparison to α-PDGFRb 1 L234A/L235A group (* p<0.05).

FIG. 10 shows fusion of α-NKp46 1 to α-PDGFRb 1 further increased cytotoxic activity of primary NK cells against TGFb1-activated HSC cells. co-culture of primary NK and activated HSC cells (primary NK: HSC cells=4:1) were treated with various concentrations of α-PDGFRb 1-α-NKp46 1 S239D/1332E, α-PDGFRb 1-α-NKp46 1 N297S, α-PDGFRb 1-NA S239D/1332E, α-PDGFRb 1-NA N297S. Cytotoxicity to HSC cells were determined using LDH release. Data were presented as Mean±SD (n=2/group).

FIGS. 11A-11D shows α-TIGIT1 IgG1 further enhanced α—PDGFRb 1 S239D/1332E-induced ADCC cytotoxicity of primary NK cells against TGFb1-activated HSC cells. (FIG. 11A) Mean fluorescence intensity of anti-CD155-BV421 (Biolegend, 337631) and isotype control-BV421 (BD Biosciences, 562438) FACS binding to HSC cells treated with or without 2 ng/ml TGFb1 treatment. (FIG. 11B) Mean fluorescence intensity of anti-CD112-APC (Biolegend, 337412) and isotype control-APC (BD pharmingen, 555751) FACS binding to HSC cells treated with or without 2 ng/ml TGFb1 treatment. (FIG. 11C) Cytotoxicity against activated HSCs induced by 3 ug/ml α-TIGIT IgG1 or α-PVRIG IgG4 treatment comparing to human IgG4 isotype control (primary NK: HSC cells =8:1). (FIG. 11D) 3 ug/ml α-TIGIT IgG1 further increased cytotoxicity induced by α-PDGFRb 1 S239D/1332E against activated HSC cells (primary PBMC: HSC cells =25:1). Data were presented as Mean±SD (n=2/group).

FIGS. 12A-12C show blocking TGFb signaling by TGFb receptor II ECD (TRII) enhanced anti-PDGFRb antibody-induced cytotoxicity against activated HSC cells. (FIG. 12A) α-PDGFRb 1 TRII S239D/1332E had similar ADCC effect comparing to α-PDGFRb 1 S239D/1332E alone. Co-culture of primary NK and activated HSC cells (primary NK: HSC cells=4:1) were treated with various concentrations of α-PDGFRb 1 TRII S239D/1332E or α-PDGFRb 1 S239D/1332E. Cytotoxicity to HSC cells were determined using LDH release. Data were presented as Mean±SD (n=2/group). (FIG. 12B) Serum antibody concentrations after multiple doses of 1B3-TRII S239D/1332E, 1B3 S239D/1332E or 1B3 WT in CCL4-treated C57BL/6J mice. Antibody concentration was determined by direct ELISA. Data were presented as Mean±SD (n=8-10/group). (FIG. 12C) 1B3 TRII S239D/1332E treatment caused more Liver total PDGFRb protein reduction than 1B3 S239D/1332E treatment. Liver total PDGFRb protein levels were determined by sandwich ELISA. Data were presented as Mean±SD (n=5-10/group). Data were analyzed using one-way ANOVA followed by multiple comparisons to isotype control group (* p<0.05; *** p<0.001; **** p<0.0001). Data for 1B3-TRII SI group and 1B3-SI group was analyzed by un-paired t-test (* p<0.05). 1B3: anti-mouse PDGFRb antibody; 1B3-TRII: anti-mouse PDGFRb antibody 1B3 with human TRII linked to heavy chain C-terminus; ISO-TRII: isotype control antibody with human TRII linked to heavy chain C-terminus

FIGS. 13A-13F shows single dose treatment of 1B3 S239D/1332E reduced HSC marker expression in CCL4-lesioned C57BL/6J mice. (FIG. 13A) Liver PDGFRb protein levels were reduced after single dose of 1B3 S239D/1332E treatment. (FIG. 13B) Liver aSMA protein levels were reduced after single dose of 1B3 S239D/1332E treatment. (FIG. 13C) Liver aSMA mRNA levels were reduced 4 hours after single dose of 1B3 S239D/1332E treatment. (FIG. 13D) Liver GFAP mRNA levels were not changed significantly after single dose of 1B3 S239D/1332E treatment. (FIG. 13E) Liver Desmin mRNA levels were reduced 4 hours after single dose of 1B3 S239D/1332E treatment. (FIG. 13F) Liver LRAT mRNA levels were not changed significantly after single dose of 1B3 S239D/1332E treatment. Data were presented as Mean±SD (n=3/group). Data were analyzed using two way ANOVA followed by multiple comparison to PBS group (*p<0.05, ** p<0.01, *** p<0.001, **** p<0.0001).

FIG. 14A-14F shows single dose treatment of 1B3 S239D/1332E increased NK/immune cell activation marker expression in CCL4-lesioned C57BL/6J mice, which correlated with the level of apoptotic cells in the liver (FIG. 14A) Liver NKG2D mRNA levels were increased 48 hours after single dose of 1B3 S239D/1332E treatment. (FIG. 14B) Liver NKp46 mRNA levels were increased 48 hours after single dose of 1B3 S239D/1332E treatment. (FIG. 14C) Liver granzyme B mRNA levels were increased 48 hours after single dose of 1B3 S239D/1332E treatment. (FIG. 14D) % Liver granzyme B strong positive cells were increased after single dose of 1B3 S239D/1332E treatment. (FIG. 14E) % cleaved caspase 3 positive cells in the liver were reduced at 4 hours and increased at 48 hours after single dose of 1B3 S239D/1332E treatment. Data were presented as Mean±SD (n=3/group). Data were analyzed using two way ANOVA followed by multiple comparison to PBS group (*p<0.05, ** p<0.01, *** p<0.001, **** p<0.0001). (FIG. 14F) Significant correlation between % cleaved caspase 3 positive cells in the liver and % liver granzyme B strong positive cells in the liver after 1B3 S239D/1332E treatment. Data were generated using linear regression (p=0.0016).

FIGS. 15A-15B shows liver fibrosis as measured by Sirius Red staining was reduced by 1B3-TRII S239D/1332E and 1B3-S239D/1332E treatment in CCL4-lesioned mice. (FIG. 15A) Liver sections were stained with Sirius Red. The percentage of Sirius Red staining positive area was quantified by software HALO. Data were presented as Mean±SD (n=5-10/group). Data were analyzed using one-way ANOVA followed by multiple comparisons to isotype control group (* p<0.05; ** p<0.01). 1B3: anti-mouse PDGFRb antibody; 1B3-TRII: anti-mouse PDGFRb antibody 1B3 with human TRII linked to heavy chain C-terminus; ISO-TRII: isotype control antibody with human TRII linked to heavy chain C-terminus. (FIG. 15B) Positive correlation between total PDGFRb protein levels (OD450 by ELISA) and the percentage of Sirius Red staining positive area. Data were generated using linear regression (p<0.0001).

FIGS. 16A-16B shows antibody concentrations in the serum was higher after multiple doses of 1B3 WT comparing to that of 1B3 S239D/1332E in CDAA diet-treated C57BL/6J mice. Serum antibody concentrations was measured at day 23 (FIG. 16A) and day 42 (FIG. 16B) after the start of the dosing. Antibody concentration was determined by direct ELISA. Data were presented as Mean±SD (n=8/group).

FIGS. 17A-17E shows treatment of 1B3 WT and 1B3 S239D/1332E reduced liver total PDGFRb levels (FIG. 17A), CDAA diet-induced ALT increase (FIG. 17B), AST increase (FIG. 17C), and % lipid droplet increase in the liver (FIG. 17D) in C57BL/6J mice. Only treatment of 1B3 S239D/1332E reduced CDAA diet-induced liver fibrosis measured as % Sirius red positive area in the liver (FIG. 17E). Data were presented as Mean±SD (n=8/group). Data were analyzed using one way ANOVA followed by multiple comparison to PBS group (** p<0.01, *** p<0.001).

### 5. DETAILED DESCRIPTION

The present disclosure is based in part on the surprising finding that antibodies engineered with a moiety of ADCC enhancing property demonstrate superior effects for treating an activated HSC associated disease or disorder such as liver fibrosis.

### 5.1. Definitions

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010). Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments thereof (e.g., domain antibodies), as described below. An antibody can be human, humanized, chimeric and/or affinity matured, as well as an antibody from other species, for example, mouse, rabbit, llama, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). Antibodies also include, but are not limited to, synthetic antibodies, recombinantly produced antibodies, antibodies including from Camelidae species (e.g., llama or alpaca) or their humanized variants, intrabodies, anti-idiotypic (anti-Id) antibodies, and functional fragments (e.g., antigen binding fragments) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be agonistic antibodies or antagonistic antibodies. Antibodies may be neither agonistic nor antagonistic.

An “antigen” is a structure to which an antibody can selectively bind. A target antigen may be a polypeptide, carbohydrate, nucleic acid, lipid, hapten, or other naturally occurring or synthetic compound. In some embodiments, the target antigen is a polypeptide. In certain embodiments, an antigen is associated with a cell, for example, is present on or in a cell.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as an antigen, is the affinity of the antibody or functional fragment for that epitope. The ratio of dissociation rate (k_{off}) to association rate (kₒₙ) of a binding molecule (e.g., an antibody) to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent antigen, come in contact with antibodies containing multiple binding sites, the interaction of antibody with antigen at one site will increase the probability of a reaction at a second site. The strength of such multiple interactions between a multivalent antibody and antigen is called the avidity.

In connection with the binding molecules described herein terms such as “bind to,” “that specifically bind to,” and analogous terms are also used interchangeably herein and refer to binding molecules of antigen binding domains that specifically bind to an antigen, such as a polypeptide. A binding molecule or antigen binding domain that binds to or specifically binds to an antigen can be identified, for example, by immunoassays, Octet®, Biacore®, or other techniques known to those of skill in the art. In some embodiments, a binding molecule or antigen binding domain binds to or specifically binds to an antigen when it binds to an antigen with higher affinity than to any cross-reactive antigen as determined using experimental techniques, such as radioimmunoassay (RIA) and enzyme linked immunosorbent assay (ELISA). Typically, a specific or selective reaction will be at least twice background signal or noise and may be more than 10 times background. See, e.g., Fundamental Immunology 332-36 (Paul ed., 2d ed. 1989) for a discussion regarding binding specificity. In certain embodiments, the extent of binding of a binding molecule or antigen binding domain to a “non-target” protein is less than about 10% of the binding of the binding molecule or antigen binding domain to its particular target antigen, for example, as determined by fluorescence activated cell sorting (FACS) analysis or RIA. A binding molecule or antigen binding domain that binds to an antigen includes one that is capable of binding the antigen with sufficient affinity such that the binding molecule is useful, for example, as a therapeutic and/or diagnostic agent in targeting the antigen. In certain embodiments, a binding molecule or antigen binding domain that binds to an antigen has a dissociation constant (K_{D}) of less than or equal to 1 μM, 800 nM, 600 nM, 550 nM, 500 nM, 300 nM, 250 nM, 100 nM, 50 nM, 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, a binding molecule or antigen binding domain binds to an epitope of an antigen that is conserved among the antigen from different species.

In certain embodiments, the binding molecules or antigen binding domains can comprise “chimeric” sequences in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55). Chimeric sequences may include humanized sequences.

In certain embodiments, the binding molecules or antigen binding domains can comprise portions of “humanized” forms of nonhuman (e.g., camelid, murine, non-human primate) antibodies that include sequences from human immunoglobulins (e.g., recipient antibody) in which the native CDR residues are replaced by residues from the corresponding CDR of a nonhuman species (e.g., donor antibody) such as camelid, mouse, rat, rabbit, or nonhuman primate having the desired specificity, affinity, and capacity. In some instances, one or more FR region residues of the human immunoglobulin sequences are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., Nature 321:522-25 (1986); Riechmann et al., Nature 332:323-29 (1988); Presta, Curr. Op. Struct. Biol. 2:593-96 (1992); Carter et al., Proc. Natl. Acad. Sci. USA 89:4285-89 (1992); U.S. Pat. Nos. 6,800,738; 6,719,971; 6,639,055; 6,407,213; and 6,054,297.

In certain embodiments, the binding molecules or antigen binding domains can comprise portions of a “fully human antibody” or “human antibody,” wherein the terms are used interchangeably herein and refer to an antibody that comprises a human variable region and, for example, a human constant region. The binding molecules may comprise an antibody sequence. In specific embodiments, the terms refer to an antibody that comprises a variable region and constant region of human origin. “Fully human” antibodies, in certain embodiments, can also encompass antibodies which bind polypeptides and are encoded by nucleic acid sequences which are naturally occurring somatic variants of human germline immunoglobulin nucleic acid sequence. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). A “human antibody” is one that possesses an amino acid sequence which corresponds to that of an antibody produced by a human and/or has been made using any of the techniques for making human antibodies. This definition of a human antibody specifically excludes a humanized antibody comprising non-human antigen-binding residues. Human antibodies can be produced using various techniques known in the art, including phage-display libraries (Hoogenboom and Winter, J. Mol. Biol. 227:381 (1991); Marks et al., J. Mol. Biol. 222:581 (1991)) and yeast display libraries (Chao et al., Nature Protocols 1: 755-68 (2006)). Also available for the preparation of human monoclonal antibodies are methods described in Cole et al., Monoclonal Antibodies and Cancer Therapy 77 (1985); Boerner et al., J. Immunol. 147(1):86-95 (1991); and van Dijk and van de Winkel, Curr. Opin. Pharmacol. 5: 368-74 (2001). Human antibodies can be prepared by administering the antigen to a transgenic animal that has been modified to produce such antibodies in response to antigenic challenge, but whose endogenous loci have been disabled, e.g., mice (see, e.g., Jakobovits, Curr. Opin. Biotechnol. 6(5):561-66 (1995); Brüggemann and Taussing, Curr. Opin. Biotechnol. 8(4):455-58 (1997); and U.S. Pat. Nos. 6,075,181 and 6,150,584 regarding XENOMOUSE™ technology). See also, for example, Li et al., Proc. Natl. Acad. Sci. USA 103:3557-62 (2006) regarding human antibodies generated via a human B-cell hybridoma technology.

In certain embodiments, the binding molecules or antigen binding domains can comprise portions of a “recombinant human antibody,” wherein the phrase includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al., Nucl. Acids Res. 20:6287-6295 (1992)) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and a VL regions of the recombinant antibodies are sequences that, while derived from and related to human germline VH and a VL sequences, may not naturally exist within the human antibody germline repertoire in vivo.

In certain embodiments, the binding molecules or antigen binding domains can comprise a portion of a “monoclonal antibody,” wherein the term as used herein refers to an antibody obtained from a population of substantially homogeneous antibodies, e.g., the individual antibodies comprising the population are identical except for possible naturally occurring mutations that may be present in minor amounts or well-known post-translational modifications such as amino acid iomerizatio or deamidation, methionine oxidation or asparagine or glutamine deamidation, each monoclonal antibody will typically recognize a single epitope on the antigen. In specific embodiments, a “monoclonal antibody,” as used herein, is an antibody produced by a single hybridoma or other cell. The term “monoclonal” is not limited to any particular method for making the antibody. For example, the monoclonal antibodies useful in the present disclosure may be prepared by the hybridoma methodology first described by Kohler et al., Nature 256:495 (1975), or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352:624-28 (1991) and Marks et al., J. Mol. Biol. 222:581-97 (1991), for example. Other methods for the preparation of clonal cell lines and of monoclonal antibodies expressed thereby are well known in the art. See, e.g., Short Protocols in Molecular Biology (Ausubel et al. eds., 5th ed. 2002).

A typical 4-chain antibody unit is a heterotetrameric glycoprotein composed of two identical light (L) chains and two identical heavy (H) chains. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for u and & isotypes. Each L chain has at the N-terminus, a variable domain (VL) followed by a constant domain (CL) at its other end. The VL is aligned with the VH, and the CL is aligned with the first constant domain of the heavy chain (CH1). Particular amino acid residues are believed to form an interface between the light chain and heavy chain variable domains. The pairing of a VH and a VL together forms a single antigen-binding site. For the structure and properties of the different classes of antibodies, see, for example, Basic and Clinical Immunology 71 (Stites et al. eds., 8th ed. 1994); and Immunobiology (Janeway et al. eds., 5^{th} ed. 2001).

The term “Fab” or “Fab region” refers to an antibody region that binds to antigens. A conventional IgG usually comprises two Fab regions, each residing on one of the two arms of the Y-shaped IgG structure. Each Fab region is typically composed of one variable region and one constant region of each of the heavy and the light chain. More specifically, the variable region and the constant region of the heavy chain in a Fab region are VH and CHI regions, and the variable region and the constant region of the light chain in a Fab region are VL and CL regions. The VH, CHI, VL, and CL in a Fab region can be arranged in various ways to confer an antigen binding capability according to the present disclosure. For example, VH and CHI regions can be on one polypeptide, and a VL and CL regions can be on a separate polypeptide, similarly to a Fab region of a conventional IgG. Alternatively, VH, CHI, VL and CL regions can all be on the same polypeptide and oriented in different orders as described in more detail the sections below.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5^{th} ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering according to Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while u and & contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (2) based on the amino acid sequence of the constant domains.

As used herein, the terms “hypervariable region,” “HVR,” “Complementarity Determining Region,” and “CDR” are used interchangeably. A “CDR” refers to one of three hypervariable regions (H1, H2 or H3) within the non-framework region of the immunoglobulin (Ig or antibody) VH B-sheet framework, or one of three hypervariable regions (L1, L2 or L3) within the non-framework region of the antibody VL B-sheet framework. CDR1, CDR2 and CDR3 in VH domain are also referred to as HCDR1, HCDR2 and HCDR3, respectively. CDR1, CDR2 and CDR3 in VL domain are also referred to as LCDR1, LCDR2 and LCDR3, respectively. Accordingly, CDRs are variable region sequences interspersed within the framework region sequences.

CDR regions are well known to those skilled in the art and have been defined by well-known numbering systems. For example, the Kabat Complementarity Determining Regions (CDRs) are based on sequence variability and are the most commonly used (see, e.g., Kabat et al., supra; Nick Deschacht et al., J Immunol 2010; 184:5696-5704). Chothia refers instead to the location of the structural loops (see, e.g., Chothia and Lesk, J. Mol. Biol. 196:901-17 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dübel eds., 2d ed. 2010)). The “contact” hypervariable regions are based on an analysis of the available complex crystal structures. Another universal numbering system that has been developed and widely adopted is ImMunoGeneTics (IMGT) Information SystemR (Lafranc et al., Dev. Comp. Immunol. 27(1):55-77 (2003)). IMGT is an integrated information system specializing in immunoglobulins (IG), T-cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-70 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). The residues from each of these hypervariable regions or CDRs are exemplified in the below.

Exemplary CDRs According to Various Numbering Systems

| Loop | Kabat | AbM | Chothia | Contact | IMGT |
|---|---|---|---|---|---|
| CDR L1 | L24--L34 | L24--L34 | L26--L32 or | L30--L36 | L27--L38 |
| | | | L24--L34 | | |
| CDR L2 | L50--L56 | L50--L56 | L50--L52 or | L46--L55 | L56--L65 |
| | | | L50--L56 | | |
| CDR L3 | L89--L97 | L89--L97 | L91--L96 or | L89--L96 | L105-L117 |
| | | | L89--L97 | | |
| CDR H1 | H31--H35B | H26--H35B | H26-- | H30--H35B | H27--H38 |
| | (Kabat | | H32 . . . 34 | | |
| | Numbering) | | | | |
| CDR H1 | H31--H35 | H26--H35 | H26--H32 | H30--H35 | |
| | (Chothia | | | | |
| | Numbering) | | | | |
| CDR H2 | H50--H65 | H50--H58 | H53--H55 or | H47--H58 | H56--H65 |
| | | | H52--H56 | | |
| CDR H3 | H95--H102 | H95--H102 | H96--H101 | H93--H101 | H105-H117 |
| | | | or H95-- | | |
| | | | H102 | | |

The boundaries of a given CDR may vary depending on the scheme used for identification. Thus, unless otherwise specified, the terms “CDR” and “complementary determining region” of a given antibody or region thereof, such as a variable region, as well as individual CDRs (e.g., CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, Chothia, or Contact method. In other cases, the particular amino acid sequence of a CDR is given. It should be noted CDR regions may also be defined by a combination of various numbering systems, e.g., a combination of Kabat and Chothia numbering systems, or a combination of Kabat and IMGT numbering systems. Therefore, the term such as “a CDR1 as set forth in a specific VH” includes any CDR1 as defined by the exemplary CDR numbering systems described above, but is not limited thereby. Once a variable region (e.g., a VH or VL) is given, those skilled in the art would understand that CDRs within the region can be defined by different numbering systems or combinations thereof.

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH.

The term “constant region” or “constant domain” refers to a carboxy terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CHI, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A functional Fc region possesses an effector function of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

As used herein, an “epitope” is a term in the art and refers to a localized region of an antigen to which a binding molecule (e.g., an antibody) can specifically bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope). It will be appreciated by one of skill in the art that, in general, a linear epitope may or may not be dependent on secondary, tertiary, or quaternary structure. For example, in some embodiments, a binding molecule binds to a group of amino acids regardless of whether they are folded in a natural three dimensional protein structure. In other embodiments, a binding molecule requires amino acid residues making up the epitope to exhibit a particular conformation (e.g., bend, twist, turn or fold) in order to recognize and bind the epitope.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The term “specificity” refers to selective recognition of an antigen binding protein for a particular epitope of an antigen. Natural antibodies, for example, are monospecific. The term “multispecific” as used herein denotes that an antigen binding protein has two or more antigen-binding sites of which at least two bind different antigens. “Bispecific” as used herein denotes that an antigen binding protein has two different antigen-binding specificities. The term “monospecific” antibody as used herein denotes an antigen binding protein that has one or more binding sites each of which bind the same antigen.

The term “valent” as used herein denotes the presence of a specified number of binding sites in an antigen binding protein. A natural antibody for example or a full length antibody has two binding sites and is bivalent. As such, the terms “trivalent”, “tetravalent”, “pentavalent” and “hexavalent” denote the presence of two binding site, three binding sites, four binding sites, five binding sites, and six binding sites, respectively, in an antigen binding protein.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogs of an amino acid, including but not limited to, unnatural amino acids, as well as other modifications known in the art. It is understood that, because the polypeptides of this disclosure may be based upon antibodies or other members of the immunoglobulin superfamily, in certain embodiments, a “polypeptide” can occur as a single chain or as two or more associated chains.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces a binding molecule of the present disclosure may include a parent hybridoma cell, as well as bacterial and eukaryotic host cells into which nucleic acids encoding the antibodies have been introduced. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule. Specifically, an “isolated” nucleic acid molecule encoding an antibody described herein is a nucleic acid molecule that is identified and separated from at least one contaminant nucleic acid molecule with which it is ordinarily associated in the environment in which it was produced.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operatively linked,” and similar phrases (e.g., genetically fused), when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

The term “vector” refers to a substance that is used to carry or include a nucleic acid sequence, including for example, a nucleic acid sequence encoding a binding molecule (e.g., an antibody) as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and a VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18^{th} ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Lippincott Williams & Wilkins: Philadelphia, P A, 2005; Handbook of Pharmaceutical Excipients, 6^{th} ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009. In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical compounds, may contain a binding molecule (e.g., an antibody), for example, in isolated or purified form, together with a suitable amount of excipients.

The term “effective amount” or “therapeutically effective amount” as used herein refers to the amount of an antibody or a therapeutic molecule comprising an agent and the antibody or pharmaceutical composition provided herein which is sufficient to result in the desired outcome.

The terms “subject” and “patient” may be used interchangeably. As used herein, in certain embodiments, a subject is a mammal, such as a non-primate or a primate (e.g., human). In specific embodiments, the subject is a human. In one embodiment, the subject is a mammal, e.g., a human, diagnosed with a disease or disorder. In another embodiment, the subject is a mammal, e.g., a human, at risk of developing a disease or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat,” “treatment” and “treating” refer to the reduction or amelioration of the progression, severity, and/or duration of a disease or disorder resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s) (e.g., diabetes or a cancer).

As used herein, “delaying” the development of a disease means to defer, hinder, slow, retard, stabilize, and/or postpone development of the disease. This delay can be of varying lengths of time, depending on the history of the disease and/or individual being treated. As is evident to one skilled in the art, a sufficient or significant delay can, in effect, encompass prevention, in that the individual does not develop the disease. A method that “delays” development of a disease is a method that reduces probability of disease development in a given time frame and/or reduces the extent of the disease in a given time frame, when compared to not using the method. Such comparisons are typically based on clinical studies, using a statistically significant number of individuals.

The term “activated hepatic stellate cell HSC associated disease or disorder” or similar terms as used herein refers to a disease or disorder comprising tissues with activated HSCs including those diseases or disorders at least partically caused by activated HSCs. In some embodiments, the disease or disorder is characterized by an abnormal amount (e.g., higher than normal amount) of activated HSCs. Such diseases or disorders include liver fibrosis for example.

The terms “about” and “approximately” mean within 20%, within 15%, within 10%, within 9%, within 8%, within 7%, within 6%, within 5%, within 4%, within 3%, within 2%, within 1%, or less of a given value or range.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2. Binding Molecules

As shown in Section 7 below, a binding molecule targeting activated hepatic stellate cells (HSCs) when engineered to possess enhanced ADCC (e.g., by Fc mutations) is significantly more effective in depleting HSCs. Surprisingly, such a binding molecule with enhanced ADCC performs dramatically different from a comparable binding molecule without such enhanced ADCC property in in vivo studies for therapeutic effects, and thus enhanced ADCC is critical for such activated HSCs binding molecules to exert therapeutic effects and for use in treating activated HSCs related disease or disorder such as liver fibrosis. Without being bound to any theory, such dramatic difference in therapeutic effect conferred by enhanced ADCC appears unique to HSCs and may be due to characteristics of this specific type of cells. Therefore, among other advantages provided herein, the present disclosure offers new therapeutic strategies (including compositions and methods) for HSCs associated disease or disorder.

Thus, among other things, the present disclosure provides HSC binding molecules comprising means for enhancing an effector function (such as ADCC) and uses thereof. In one aspect, provided herein is a binding molecule comprising a binding domain that binds to an antigen expressed on an activated hepatic stellate cells (HSC), and a functional domain that is capable of enhancing an antibody effector function such as antibody-dependent cell-mediated cytotoxicity (ADCC). In some embodiments, the binding molecule is an Fc containing molecule. In some embodiments, the binding molecule is an antibody (including antigen binding fragment of an intact antibody). In other embodiments, the binding molecule comprises a binding domain fused to an Fc region. As described in more details below, the means for enhancing ADCC includes but not limited to incorporating ADCC enhancing Fc mutations and additional binding domains that activate immune cells.

In certain embodiments, the binding molecule provided herein increases depletion of HSCs by at least 10% as compared with a comparable binding molecule without the functional domain that enhances ADCC. In certain embodiments, the binding molecule provided herein increases depletion of HSCs by at least 20% as compared with a comparable binding molecule without the functional domain that enhances ADCC. In certain embodiments, the binding molecule provided herein increases depletion of HSCs by at least 30% as compared with a comparable binding molecule without the functional domain that enhances ADCC. In certain embodiments, the binding molecule provided herein increases depletion of HSCs by at least 40% as compared with a comparable binding molecule without the functional domain that enhances ADCC. In certain embodiments, the binding molecule provided herein increases depletion of HSCs by at least 50% as compared with a comparable binding molecule without the functional domain that enhances ADCC. In certain embodiments, the binding molecule provided herein increases depletion of HSCs by at least 60% as compared with a comparable binding molecule without the functional domain that enhances ADCC. In certain embodiments, the binding molecule provided herein increases depletion of HSCs by at least 70% as compared with a comparable binding molecule without the functional domain that enhances ADCC. In certain embodiments, the binding molecule provided herein increases depletion of HSCs by at least 80% as compared with a comparable binding molecule without the functional domain that enhances ADCC. In certain embodiments, the binding molecule provided herein increases depletion of HSCs by at least 90% as compared with a comparable binding molecule without the functional domain that enhances ADCC. In certain embodiments, the binding molecule provided herein increases depletion of HSCs by at least 10%-90% as compared with a comparable binding molecule without the functional domain that enhances ADCC. In certain embodiments, the binding molecule provided herein increases depletion of HSCs by at least 20%-90% as compared with a comparable binding molecule without the functional domain that enhances ADCC. In certain embodiments, the binding molecule provided herein increases depletion of HSCs by at least 30%-90% as compared with a comparable binding molecule without the functional domain that enhances ADCC. In certain embodiments, the binding molecule provided herein increases depletion of HSCs by at least 40%-90% as compared with a comparable binding molecule without the functional domain that enhances ADCC. In certain embodiments, the binding molecule provided herein increases depletion of HSCs by at least 50%-90% as compared with a comparable binding molecule without the functional domain that enhances ADCC. In certain embodiments, the binding molecule provided herein increases depletion of HSCs by at least 60%-90% as compared with a comparable binding molecule without the functional domain that enhances ADCC. In certain embodiments, the binding molecule provided herein increases depletion of HSCs by at least 70%-90% as compared with a comparable binding molecule without the functional domain that enhances ADCC. In certain embodiments, the binding molecule provided herein increases depletion of HSCs by at least 70%-90% as compared with a comparable binding molecule without the functional domain that enhances ADCC.

In certain embodiments, the binding molecule provided herein increases depletion of HSCs by at least 2 folds as compared with a comparable binding molecule without the functional domain that enhances ADCC. In certain embodiments, the binding molecule provided herein increases depletion of HSCs by at least 3 folds as compared with a comparable binding molecule without the functional domain that enhances ADCC. In certain embodiments, the binding molecule provided herein increases depletion of HSCs by at least 4 folds as compared with a comparable binding molecule without the functional domain that enhances ADCC. In certain embodiments, the binding molecule provided herein increases depletion of HSCs by at least 5 folds as compared with a comparable binding molecule without the functional domain that enhances ADCC. In certain embodiments, the binding molecule provided herein increases depletion of HSCs by at least 6 folds as compared with a comparable binding molecule without the functional domain that enhances ADCC. In certain embodiments, the binding molecule provided herein increases depletion of HSCs by at least 7 folds as compared with a comparable binding molecule without the functional domain that enhances ADCC. In certain embodiments, the binding molecule provided herein increases depletion of HSCs by at least 8 folds as compared with a comparable binding molecule without the functional domain that enhances ADCC. In certain embodiments, the binding molecule provided herein increases depletion of HSCs by at least 9 folds as compared with a comparable binding molecule without the functional domain that enhances ADCC. In certain embodiments, the binding molecule provided herein increases depletion of HSCs by at least 10 folds as compared with a comparable binding molecule without the functional domain that enhances ADCC. In certain embodiments, the binding molecule provided herein increases depletion of HSCs by at least 2-10 folds as compared with a comparable binding molecule without the functional domain that enhances ADCC. In certain embodiments, the binding molecule provided herein increases depletion of HSCs by at least 3-10 folds as compared with a comparable binding molecule without the functional domain that enhances ADCC. In certain embodiments, the binding molecule provided herein increases depletion of HSCs by at least 4-10 folds as compared with a comparable binding molecule without the functional domain that enhances ADCC. In certain embodiments, the binding molecule provided herein increases depletion of HSCs by at least 5-10 folds as compared with a comparable binding molecule without the functional domain that enhances ADCC. In certain embodiments, the binding molecule provided herein increases depletion of HSCs by at least 6-10 folds as compared with a comparable binding molecule without the functional domain that enhances ADCC. In certain embodiments, the binding molecule provided herein increases depletion of HSCs by at least 7-10 folds as compared with a comparable binding molecule without the functional domain that enhances ADCC. In certain embodiments, the binding molecule provided herein increases depletion of HSCs by at least 8-10 folds as compared with a comparable binding molecule without the functional domain that enhances ADCC. In certain embodiments, the binding molecule provided herein increases depletion of HSCs by at least 9-10 folds as compared with a comparable binding molecule without the functional domain that enhances ADCC.

### Multispecific Binding Molecules

The multispecific binding molecule provided herein comprises one or more HSC binding domain(s) capable of binding to one or more antigen(s) expressed on HSCs, including for example, 5HT1B, 5HTIF, 5HT2A, 5-HT2B, 5-HT7, A2a, A2b, a1b1 integrin, a2bi integrin, a5b1 integrin, a6b4 integrin, a8b1 integrin, avb1 integrin, avb3 integrin, ACVR2A, ACVR2B, AdipoR1, AdipoR2, ADRA1A, ADRA1B, ANTXR1, AT1, AT2, BAMB1, BMPR2, C5aR, CB1, CB2, CCR1, CCR2, CCR5, CCR7, CD105, CD112, CD14, CD146, CD155, CD248, CD36, CD38, CD40, CD44, CD49e, CD62e, CD73, CD95, c-MET, CNTFR, CXCR3, CXCR4, DDR1, DDR2, EGFR, ETA, ETB, FAP, FGFR2, FN, gp130, GPC3, GPR91, ICAM-1, IGF-1R, IGF-2R, IL-10R2, IL-11RA, IL-17RA, IL-20R1, IL-20R2, IL-22R1, IL-6R, KCNE4, ITGA8, LRP, MICA, MICB, NCAM, NGFR, NPR-B, NPR3, OB-Ra, OB-Rb, OPRD1, P2X4, P2X7, P2Y6, p75NTR, PAFR, PARI PAR2 PAR4, PDGFRA, PDGFRB, PD-L1, PD-L2, Ptc, PTH-1R, RAGE, SIRPA, CD47, SYP, TGFBR1, TGFBR2, TGFBR3, TLR2, TLR3, TLR4, TLR7, TLR9, TNFR1, TRKB, TRKC, ULBP1, ULPB2, uPAR, VACM-1, VEGFR-1, and VEGFR-2. In some embodiments, the HSC binding domain is as described or derived from the antibodies described in section HSC Binding Domain below.

In some embodiments, the multispecific binding molecule provides herein comprises a first binding domain capable of binding to a first antigen expressed on HSCs, and a second binding domain capable of binding to a second antigen expressed on HSCs, the first and the second antigens are selected from a group consisting of for example, 5HT1B, 5HTIF, 5HT2A, 5-HT2B, 5-HT7, A2a, A2b, a1b1 integrin, a2bi integrin, a5b1 integrin, a6b4 integrin, a8b1 integrin, avb1 integrin, avb3 integrin, ACVR2A, ACVR2B, AdipoR1, AdipoR2, ADRA1A, ADRA1B, ANTXR1, AT1, AT2, BAMB1, BMPR2, C5aR, CB1, CB2, CCR1, CCR2, CCR5, CCR7, CD105, CD112, CD14, CD146, CD155, CD248, CD36, CD38, CD40, CD44, CD49e, CD62e, CD73, CD95, c-MET, CNTFR, CXCR3, CXCR4, DDR1, DDR2, EGFR, ETA, ETB, FAP, FGFR2, FN, gp130, GPC3, GPR91, ICAM-1, IGF-1R, IGF-2R, IL-10R2, IL-11RA, IL-17RA, IL-20R1, IL-20R2, IL-22R1, IL-6R, KCNE4, ITGA8, LRP, MICA, MICB, NCAM, NGFR, NPR-B, NPR3, OB-Ra, OB-Rb, OPRD1, P2X4, P2X7, P2Y6, p75NTR, PAFR, PARI PAR2 PAR4, PDGFRA, PDGFRB, PD-L1, PD-L2, Ptc, PTH-1R, RAGE, SIRPA, CD47, SYP, TGFBR1, TGFBR2, TGFBR3, TLR2, TLR3, TLR4, TLR7, TLR9, TNFR1, TRKB, TRKC, ULBP1, ULPB2, uPAR, VACM-1, VEGFR-1, and VEGFR-2. In some embodiments, the first and second HSC binding domains are as described or derived from the antibodies described in section HSC Binding Domain below.

In some embodiments, the multispecific binding molecule provided herein further comprises a functional domain that is capable of enhancing an antibody effector function toward activated HSCs. In some embodiments, the antibody effector function is antibody-dependent cell-mediated cytotoxicity (ADCC). In some embodiments, the functional domain is an Fc region comprising one or more mutation(s) that enhances ADCC. In other embodiments, the functional domain is a domain that activates an immune cell. In some embodiments, the immune cell is a NK cell.

In some embodiments, the functional domain is the Fc region comprising one or more mutation(s) that enhances ADCC. In some embodiments, the multispecific molecule provided herein comprising a variant Fc region as described in section Binding Molecules Comprising A Variant Fc Region below.

In some embodiments, the functional domain binds to and/or modulates a receptor on an immune cell. In some embodiments, the functional domain modulates a receptor that activates the immune cell. In other embodiments, the functional domain modulates a receptor that inhibits the immune cell. In some embodiments, the receptor is selected from a group consisting of NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, TIGIT, PVRIG, and A2a. In some embodiments, the functional domain is as described or derived from the antibodies described in section Binding Molecules Comprising An Immune Cell Activating Domain below.

In some embodiments, the multispecific molecule provided herein is a multispecific antibody. The antibodies provided herein include, but are not limited to, synthetic antibodies, monoclonal antibodies, recombinantly produced antibodies, human antibodies, humanized antibodies, chimeric antibodies, etc.

In particular, the antibodies provided herein include immunoglobulin molecules and immunologically active portions of immunoglobulin molecules, i.e., molecules that contain an antigen binding site that immunospecifically binds to an antigen. The immunoglobulin molecules provided herein can be of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody provided herein is an IgG antibody, such as an IgG1 antibody, IgG2 antibody or IgG4 antibody (e.g., IgG4 nullbody and variants of IgG4 antibodies). In a specific embodiment, the IgG antibody is an IgG1 antibody.

Any multispecific antibody platform or formats known in the art can be used in the present disclosure, including any known bispecific antibody formats in the art.

In some embodiments, the multispecific antibodies include IgG-like molecules with complementary CH3 domains that promote heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus) and the DuoBody (Genmab A/S).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (ImClone/Eli Lilly), Ts2Ab (MedImmune/AZ) and BsAb (Zymogenetics), HERCULES (Biogen Idec) and TvAb (Roche).

In some embodiments, Fc fusion molecules can include ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine—China).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnoland Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length bispecific antibodies provided herein can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each binding a distinct epitope. Other methods of making multispecific antibodies are known and contemplated.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.”

Other strategies such as promoting heavy chain heterodimerization using electrostatic interactions by substituting positively charged residues at one CH3 surface and negatively charged residues at a second CH3 surface can be used, as described in US Pat. Publ. No. US2010/0015133; US Pat. Publ. No. US2009/0182127; US Pat. Publ. No. US2010/028637; or US Pat. Publ. No. US2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M T394W/F405A_Y407V, T366L K392M_T394W/F405A_Y407V, L351Y_Y407A/T366A_K409F, L351Y_Y407A/T366V K409F Y407A/T366A_K409F, or T350V L351Y_F405A_Y407V/T350V T366L K392L T394W as described in U.S. Pat. Publ. No. US2012/0149876 or U.S. Pat. Publ. No. US2013/0195849.

In addition to methods described above, bispecific antibodies provided herein can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 can be used.

### Binding Molecules Comprising A Variant Fc Region

In some embodiments, the functional domain that confers the enhanced effector function is an Fc variant comprising one or more mutation(s) that enhances ADCC. Any known Fc mutations for enhancing ADCC may be used in the present binding molecules, including but not limited to those described in, e.g., Sondermann et al., Nature, 406: 267-273 (2000); U.S. Pat. No. 8,951,517B2; U.S. Pat. No. 9,714,282B2; and US20090208500A1, each of which is incorporated herein by reference in its entirety. For example, the one or more mutation(s) of the Fc region can be at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, there is only one amino acid mutation in the Fc region. In other embodiments, there are multiple amino acid mutations, such as 2, 3, 4, 5, 6, 7, 8, 9, or more amino acid mutations, in the Fc region in the present binding molecules.

In some embodiments, the present Fc region comprises an amino acid mutation at the position of 233. In some embodiments, the present Fc region comprises an amino acid mutation at the position of 234. In some embodiments, the present Fc region comprises an amino acid mutation at the position of 235. In some embodiments, the present Fc region comprises an amino acid mutation at the position of 236. In some embodiments, the present Fc region comprises an amino acid mutation at the position of 237. In some embodiments, the present Fc region comprises an amino acid mutation at the position of 238. In some embodiments, the present Fc region comprises an amino acid mutation at the position of 239. In some embodiments, the present Fc region comprises an amino acid mutation at the position of 240. In some embodiments, the present Fc region comprises an amino acid mutation at the position of 241. In some embodiments, the present Fc region comprises an amino acid mutation at the position of 243. In some embodiments, the present Fc region comprises an amino acid mutation at the position of 244. In some embodiments, the present Fc region comprises an amino acid mutation at the position of 245. In some embodiments, the present Fc region comprises an amino acid mutation at the position of 246. In some embodiments, the present Fc region comprises an amino acid mutation at the position of 247. In some embodiments, the present Fc region comprises an amino acid mutation at the position of 248. In some embodiments, the present Fc region comprises an amino acid mutation at the position of 250. In some embodiments, the present Fc region comprises an amino acid mutation at the position of 253. In some embodiments, the present Fc region comprises an amino acid mutation at the position of 254. In some embodiments, the present Fc region comprises an amino acid mutation at the position of 255. In some embodiments, the present Fc region comprises an amino acid mutation at the position of 256. In some embodiments, the present Fc region comprises an amino acid mutation at the position of 258. In some embodiments, the present Fc region comprises an amino acid mutation at the position of 260. In some embodiments, the present Fc region comprises an amino acid mutation at the position of 262. In some embodiments, the present Fc region comprises an amino acid mutation at the position of 263. In some embodiments, the present Fc region comprises an amino acid mutation at the position of 264. In some embodiments, the present Fc region comprises an amino acid mutation at the position of 265. In some embodiments, the present Fc region comprises an amino acid mutation at the position of 266. In some embodiments, the present Fc region comprises an amino acid mutation at the position of 267. In some embodiments, the present Fc region comprises an amino acid mutation at the position of 268. In some embodiments, the present Fc region comprises an amino acid mutation at the position of 268. In some embodiments, the present Fc region comprises an amino acid mutation at the position of 269. In some embodiments, the present Fc region comprises an amino acid mutation at the position of 270. In some embodiments, the present Fc region comprises an amino acid mutation at the position of 272. In some embodiments, the present Fc region comprises an amino acid mutation at the position of 274. In some embodiments, the present Fc region comprises an amino acid mutation at the position of 276. In some embodiments, the present Fc region comprises an amino acid mutation at the position of 280. In some embodiments, the present Fc region comprises an amino acid mutation at the position of 282. In some embodiments, the present Fc region comprises an amino acid mutation at the position of 283. In some embodiments, the present Fc region comprises an amino acid mutation at the position of 285. In some embodiments, the present Fc region comprises an amino acid mutation at the position of 286. In some embodiments, the present Fc region comprises an amino acid mutation at the position of 288. In some embodiments, the present Fc region comprises an amino acid mutation at the position of 290. In some embodiments, the present Fc region comprises an amino acid mutation at the position of 292. In some embodiments, the present Fc region comprises an amino acid mutation at the position of 293. In some embodiments, the present Fc region comprises an amino acid mutation at the position of 294. In some embodiments, the present Fc region comprises an amino acid mutation at the position of 295. In some embodiments, the present Fc region comprises an amino acid mutation at the position of 296. In some embodiments, the present Fc region comprises an amino acid mutation at the position of 297. In some embodiments, the present Fc region comprises an amino acid mutation at the position of 298. In some embodiments, the present Fc region comprises an amino acid mutation at the position of 299. In some embodiments, the present Fc region comprises an amino acid mutation at the position of 300. In some embodiments, the present Fc region comprises an amino acid mutation at the position of 301. In some embodiments, the present Fc region comprises an amino acid mutation at the position of 303. In some embodiments, the present Fc region comprises an amino acid mutation at the position of 307. In some embodiments, the present Fc region comprises an amino acid mutation at the position of 311. In some embodiments, the present Fc region comprises an amino acid mutation at the position of 312. In some embodiments, the present Fc region comprises an amino acid mutation at the position of 313. In some embodiments, the present Fc region comprises an amino acid mutation at the position of 315. In some embodiments, the present Fc region comprises an amino acid mutation at the position of 317. In some embodiments, the present Fc region comprises an amino acid mutation at the position of 318. In some embodiments, the present Fc region comprises an amino acid mutation at the position of 320. In some embodiments, the present Fc region comprises an amino acid mutation at the position of 322. In some embodiments, the present Fc region comprises an amino acid mutation at the position of 325. In some embodiments, the present Fc region comprises an amino acid mutation at the position of 326. In some embodiments, the present Fc region comprises an amino acid mutation at the position of 327. In some embodiments, the present Fc region comprises an amino acid mutation at the position of 328. In some embodiments, the present Fc region comprises an amino acid mutation at the position of 329. In some embodiments, the present Fc region comprises an amino acid mutation at the position of 330. In some embodiments, the present Fc region comprises an amino acid mutation at the position of 331. In some embodiments, the present Fc region comprises an amino acid mutation at the position of 332. In some embodiments, the present Fc region comprises an amino acid mutation at the position of 333. In some embodiments, the present Fc region comprises an amino acid mutation at the position of 334. In some embodiments, the present Fc region comprises an amino acid mutation at the position of 335. In some embodiments, the present Fc region comprises an amino acid mutation at the position of 337. In some embodiments, the present Fc region comprises an amino acid mutation at the position of 338. In some embodiments, the present Fc region comprises an amino acid mutation at the position of 339. In some embodiments, the present Fc region comprises an amino acid mutation at the position of 340. In some embodiments, the present Fc region comprises an amino acid mutation at the position of 342. In some embodiments, the present Fc region comprises an amino acid mutation at the position of 344. In some embodiments, the present Fc region comprises an amino acid mutation at the position of 345. In some embodiments, the present Fc region comprises an amino acid mutation at the position of 347. In some embodiments, the present Fc region comprises an amino acid mutation at the position of 355. In some embodiments, the present Fc region comprises an amino acid mutation at the position of 356. In some embodiments, the present Fc region comprises an amino acid mutation at the position of 358. In some embodiments, the present Fc region comprises an amino acid mutation at the position of 359. In some embodiments, the present Fc region comprises an amino acid mutation at the position of 360. In some embodiments, the present Fc region comprises an amino acid mutation at the position of 361. In some embodiments, the present Fc region comprises an amino acid mutation at the position of 362. In some embodiments, the present Fc region comprises an amino acid mutation at the position of 373. In some embodiments, the present Fc region comprises an amino acid mutation at the position of 375. In some embodiments, the present Fc region comprises an amino acid mutation at the position of 376. In some embodiments, the present Fc region comprises an amino acid mutation at the position of 378. In some embodiments, the present Fc region comprises an amino acid mutation at the position of 380. In some embodiments, the present Fc region comprises an amino acid mutation at the position of 382. In some embodiments, the present Fc region comprises an amino acid mutation at the position of 383. In some embodiments, the present Fc region comprises an amino acid mutation at the position of 384. In some embodiments, the present Fc region comprises an amino acid mutation at the position of 386. In some embodiments, the present Fc region comprises an amino acid mutation at the position of 388. In some embodiments, the present Fc region comprises an amino acid mutation at the position of 389. In some embodiments, the present Fc region comprises an amino acid mutation at the position of 390. In some embodiments, the present Fc region comprises an amino acid mutation at the position of 391. In some embodiments, the present Fc region comprises an amino acid mutation at the position of 392. In some embodiments, the present Fc region comprises an amino acid mutation at the position of 396. In some embodiments, the present Fc region comprises an amino acid mutation at the position of 398. In some embodiments, the present Fc region comprises an amino acid mutation at the position of 400. In some embodiments, the present Fc region comprises an amino acid mutation at the position of 401. In some embodiments, the present Fc region comprises an amino acid mutation at the position of 413. In some embodiments, the present Fc region comprises an amino acid mutation at the position of 414. In some embodiments, the present Fc region comprises an amino acid mutation at the position of 415. In some embodiments, the present Fc region comprises an amino acid mutation at the position of 416. In some embodiments, the present Fc region comprises an amino acid mutation at the position of 418. In some embodiments, the present Fc region comprises an amino acid mutation at the position of 419. In some embodiments, the present Fc region comprises an amino acid mutation at the position of 421. In some embodiments, the present Fc region comprises an amino acid mutation at the position of 422. In some embodiments, the present Fc region comprises an amino acid mutation at the position of 424. In some embodiments, the present Fc region comprises an amino acid mutation at the position of 428. In some embodiments, the present Fc region comprises an amino acid mutation at the position of 430. In some embodiments, the present Fc region comprises an amino acid mutation at the position of 433. In some embodiments, the present Fc region comprises an amino acid mutation at the position of 434. In some embodiments, the present Fc region comprises an amino acid mutation at the position of 435. In some embodiments, the present Fc region comprises an amino acid mutation at the position of 436. In some embodiments, the present Fc region comprises an amino acid mutation at the position of 437. In some embodiments, the present Fc region comprises an amino acid mutation at the position of 438. In some embodiments, the present Fc region comprises an amino acid mutation at the position of 439. In some embodiments, the present Fc region comprises an amino acid mutation at the position of 440. In some embodiments, the present Fc region comprises an amino acid mutation at the position of 442. In some embodiments, the present Fc region comprises an amino acid mutation at the position of 444. In some embodiments, the present Fc region comprises an amino acid mutation at the position of 447. All above mentioned amino acid position in a Fc region are according to EU numbering.

In some specific embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

### Binding Molecules Comprising An Immune Cell Activating Domain

In other embodiments, the functional domain is a domain that activates an immune cell. In some embodiments, the functional domain also comprises a binding domain so that the binding molecule is a multi-specific (e.g., bi-specific) binding molecule. In some embodiments, the immune cell is a NK cell. In other embodiments, the functional domain is the domain that activates the immune cell by promoting immune cell activation signaling or suppresses immune cell inhibitory signaling.

In some embodiments, the functional domain binds to and/or modulates a receptor on an immune cell. In some embodiments, the functional domain modulates a receptor that activates the immune cell. In other embodiments, the functional domain modulates a receptor that inhibits the immune cell. In some embodiments, the receptor is selected from a group consisting of NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a. In some embodiments, the functional domain binds to and/or modulates NKp46. In some embodiments, the functional domain binds to and/or modulates NKp30. In some embodiments, the functional domain binds to and/or modulates NKp44. In some embodiments, the functional domain binds to and/or modulates NKG2C. In some embodiments, the functional domain binds to and/or modulates CD94. In some embodiments, the functional domain binds to and/or modulates KIR2DS1. In some embodiments, the functional domain binds to and/or modulates KIR2DS4. In some embodiments, the functional domain binds to and/or modulates KIR2DS2. In some embodiments, the functional domain binds to and/or modulates KIR2DL4. In some embodiments, the functional domain binds to and/or modulates KIR3DS1. In some embodiments, the functional domain binds to and/or modulates CD160. In some embodiments, the functional domain binds to and/or modulates NKG2D. In some embodiments, the functional domain binds to and/or modulates NKp80. In some embodiments, the functional domain binds to and/or modulates DNAM1. In some embodiments, the functional domain binds to and/or modulates 2B4. In some embodiments, the functional domain binds to and/or modulates NTB-A. In some embodiments, the functional domain binds to and/or modulates CRACC. In some embodiments, the functional domain binds to and/or modulates 4-1BB. In some embodiments, the functional domain binds to and/or modulates OX40. In some embodiments, the functional domain binds to and/or modulates CRTAM. In some embodiments, the functional domain binds to and/or modulates CD16. In some embodiments, the functional domain binds to and/or modulates LFA1. In some embodiments, the functional domain binds to and/or modulates NKG2A. In some embodiments, the functional domain binds to and/or modulates KIR2DL1. In some embodiments, the functional domain binds to and/or modulates KIR2DL2. In some embodiments, the functional domain binds to and/or modulates KIR2DL3. In some embodiments, the functional domain binds to and/or modulates KIR3DL1. In some embodiments, the functional domain binds to and/or modulates KIR3DL2. In some embodiments, the functional domain binds to and/or modulates LILRB1. In some embodiments, the functional domain binds to and/or modulates KIR2DL5. In some embodiments, the functional domain binds to and/or modulates KLRG1. In some embodiments, the functional domain binds to and/or modulates CD161. In some embodiments, the functional domain binds to and/or modulates SIGLEC7. In some embodiments, the functional domain binds to and/or modulates SIGLEC9. In some embodiments, the functional domain binds to and/or modulates LAIR1. In some embodiments, the functional domain binds to and/or modulates TIGIT. In some embodiments, the functional domain binds to and/or modulates CD96. In some embodiments, the functional domain binds to and/or modulates PD-1. In some embodiments, the functional domain binds to and/or modulates PVRIG. In some embodiments, the functional domain binds to and/or modulates CD122. In some embodiments, the functional domain binds to and/or modulates CD132. In some embodiments, the functional domain binds to and/or modulates CD218a. In some embodiments, the functional domain binds to and/or modulates CD218b. In some embodiments, the functional domain binds to and/or modulates IL12Rb1. In some embodiments, the functional domain binds to and/or modulates IL 12Rb2. In some embodiments, the functional domain binds to and/or modulates IL21R. In some embodiments, the functional domain binds to and/or modulates TGFBR1. In some embodiments, the functional domain binds to and/or modulates TGFBR2. In some embodiments, the functional domain binds to and/or modulates TGFBR3. In some embodiments, the functional domain binds to and/or modulates ACVR2A. In some embodiments, the functional domain binds to and/or modulates ACVR2B. In some embodiments, the functional domain binds to and/or modulates ALK4. In some embodiments, the functional domain binds to and/or modulates A2a.

In some specific embodiments, the functional domain binds to NKG2D. In some embodiments, the functional domain is derived from a NKG2D ligand. In some embodiments, the functional domain is the extracellular domain of MICA, MICB, ULBP1, or ULBP2 or a fragment or a variant thereof. In some embodiments, the functional domain is the extracellular domain of MICA or a fragment or a variant thereof. In some embodiments, the functional domain is the extracellular domain of MICB or a fragment or a variant thereof. In some embodiments, the functional domain is the extracellular domain of ULBP1 or a fragment or a variant thereof. In some embodiments, the functional domain is the extracellular domain of ULBP2 or a fragment or a variant thereof.

In some embodiments, the functional domain comprises an amino acid sequence of SEQ ID NO: 90 or a fragment thereof. In some embodiments, the functional domain comprises an amino acid sequence at least 75%, 80%, 85%, 90%, 91%, 92%, 93%, 94%, 95%, 96%, 97%, 98%, or 99% identical to SEQ ID NO: 90.

In some embodiments, the functional domain comprises an amino acid sequence of SEQ ID NO: 91 or a fragment thereof. In some embodiments, the functional domain comprises an amino acid sequence at least 75%, 80%, 85%, 90%, 91%, 92%, 93%, 94%, 95%, 96%, 97%, 98%, or 99% identical to SEQ ID NO: 91.

In some embodiments, the functional domain comprises an amino acid sequence of SEQ ID NO: 92 or a fragment thereof. In some embodiments, the functional domain comprises an amino acid sequence at least 75%, 80%, 85%, 90%, 91%, 92%, 93%, 94%, 95%, 96%, 97%, 98%, or 99% identical to SEQ ID NO: 92.

In some embodiments, the functional domain comprises an amino acid sequence of SEQ ID NO: 93 or a fragment thereof. In some embodiments, the functional domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the functional domain binds to NKp46. In some embodiments, the functional domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the functional domain binds to TGFb. In some embodiments, the functional domain comprises TGFb receptor II extracellular domain (TRII) or a fragment or a variant thereof. In some embodiments, the functional domain comprises the amino acid sequence of SEQ ID NO: 94 or a fragment thereof. In some embodiments, the functional domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the functional domain binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the functional domain binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

### Binding Molecules Comprising A Variant Fc Region and An Immune Cell Activating Domain

In some embodiments, the binding molecules provided herein comprise two or more means for enhancing an effector function such as ADCC. In some embodiments, the binding molecules provided herein comprises an ADCC enhancing Fc variant comprising one or more mutations and a domain for activating immune cells, each of which is described in more details above and briefly below.

In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

### HSC Binding Domain

The present binding molecules comprise one or more binding domain(s) capable of binding to one or more antigen(s) expressed on HSCs, including for example, 5HT1B, 5HTIF, 5HT2A, 5-HT2B, 5-HT7, A2a, A2b, a1b1 integrin, a2bi integrin, a5b1 integrin, a6b4 integrin, a8b1 integrin, avb1 integrin, avb3 integrin, ACVR2A, ACVR2B, AdipoR1, AdipoR2, ADRA1A, ADRA1B, ANTXR1, AT1, AT2, BAMB1, BMPR2, C5aR, CB1, CB2, CCR1, CCR2, CCR5, CCR7, CD105, CD112, CD14, CD146, CD155, CD248, CD36, CD38, CD40, CD44, CD49e, CD62e, CD73, CD95, c-MET, CNTFR, CXCR3, CXCR4, DDR1, DDR2, EGFR, ETA, ETB, FAP, FGFR2, FN, gp130, GPC3, GPR91, ICAM-1, IGF-1R, IGF-2R, IL-10R2, IL-11RA, IL-17RA, IL-20R1, IL-20R2, IL-22R1, IL-6R, KCNE4, ITGA8, LRP, MICA, MICB, NCAM, NGFR, NPR-B, NPR3, OB-Ra, OB-Rb, OPRD1, P2X4, P2X7, P2Y6, p75NTR, PAFR, PARI PAR2 PAR4, PDGFRA, PDGFRB, PD-L1, PD-L2, Ptc, PTH-IR, RAGE, SIRPA, CD47, SYP, TGFBR1, TGFBR2, TGFBR3, TLR2, TLR3, TLR4, TLR7, TLR9, TNFR1, TRKB, TRKC, ULBP1, ULPB2, uPAR, VACM-1, VEGFR-1, and VEGFR-2.

In some embodiments, the binding domain binds to 5HT1B. In some embodiments, the binding domain binds to 5HTIF. In some embodiments, the binding domain binds to 5HT2A. In some embodiments, the binding domain binds to 5-HT2B. In some embodiments, the binding domain binds to 5-HT7. In some embodiments, the binding domain binds to A2a. In some embodiments, the binding domain binds to A2b. In some embodiments, the binding domain binds to a1b1 integrin. In some embodiments, the binding domain binds to a2bi integrin. In some embodiments, the binding domain binds to a5b 1 integrin. In some embodiments, the binding domain binds to a6b4 integrin. In some embodiments, the binding domain binds to a8b1 integrin. In some embodiments, the binding domain binds to avb 1 integrin. In some embodiments, the binding domain binds to avb3 integrin. In some embodiments, the binding domain binds to ACVR2A. In some embodiments, the binding domain binds to ACVR2B. In some embodiments, the binding domain binds to AdipoR1. In some embodiments, the binding domain binds to AdipoR2. In some embodiments, the binding domain binds to ADRA1A. In some embodiments, the binding domain binds to ADRA1B. In some embodiments, the binding domain binds to AT1. In some embodiments, the binding domain binds to AT2. In some embodiments, the binding domain binds to BAMBI. In some embodiments, the binding domain binds to BMPR2. In some embodiments, the binding domain binds to C5aR. In some embodiments, the binding domain binds to CB1. In some embodiments, the binding domain binds to CB2. In some embodiments, the binding domain binds to CCR1. In some embodiments, the binding domain binds to CCR2. In some embodiments, the binding domain binds to CCR5. In some embodiments, the binding domain binds to CCR7. In some embodiments, the binding domain binds to CD105. In some embodiments, the binding domain binds to CD112. In some embodiments, the binding domain binds to CD14. In some embodiments, the binding domain binds to CD146. In some embodiments, the binding domain binds to CD155. In some embodiments, the binding domain binds to CD36. In some embodiments, the binding domain binds to CD38. In some embodiments, the binding domain binds to CD40. In some embodiments, the binding domain binds to CD44. In some embodiments, the binding domain binds to CD49e. In some embodiments, the binding domain binds to CD62e. In some embodiments, the binding domain binds to CD73. In some embodiments, the binding domain binds to CD95. In some embodiments, the binding domain binds to c-MET. In some embodiments, the binding domain binds to CXCR3. In some embodiments, the binding domain binds to CXCR4. In some embodiments, the binding domain binds to DDR1. In some embodiments, the binding domain binds to DDR2. In some embodiments, the binding domain binds to EGFR. In some embodiments, the binding domain binds to ETA. In some embodiments, the binding domain binds to ETB. In some embodiments, the binding domain binds to FAP. In some embodiments, the binding domain binds to FGFR2. In some embodiments, the binding domain binds to FN. In some embodiments, the binding domain binds to gp130. In some embodiments, the binding domain binds to GPR91. In some embodiments, the binding domain binds to ICAM-1. In some embodiments, the binding domain binds to IGF-1R. In some embodiments, the binding domain binds to IGF-2R. In some embodiments, the binding domain binds to IL-10R2. In some embodiments, the binding domain binds to IL-11RA. In some embodiments, the binding domain binds to IL-17RA. In some embodiments, the binding domain binds to IL-20R1. In some embodiments, the binding domain binds to IL-20R2. In some embodiments, the binding domain binds to IL-22R1. In some embodiments, the binding domain binds to IL-6R. In some embodiments, the binding domain binds to ITGA8. In some embodiments, the binding domain binds to LRP. In some embodiments, the binding domain binds to MICA. In some embodiments, the binding domain binds to MICB. In some embodiments, the binding domain binds to NCAM. In some embodiments, the binding domain binds to NPR-B. In some embodiments, the binding domain binds to OB-Ra. In some embodiments, the binding domain binds to OB-Rb. In some embodiments, the binding domain binds to OPRD1. In some embodiments, the binding domain binds to P2X4. In some embodiments, the binding domain binds to P2X7. In some embodiments, the binding domain binds to P2Y6. In some embodiments, the binding domain binds to p75NTR. In some embodiments, the binding domain binds to PAFR. In some embodiments, the binding domain binds to PAR1. In some embodiments, the binding domain binds to PAR2. In some embodiments, the binding domain binds to PAR4. In some embodiments, the binding domain binds to PDGFRA. In some embodiments, the binding domain binds to PDGFRB. In some embodiments, the binding domain binds to PD-L1. In some embodiments, the binding domain binds to PD-L2. In some embodiments, the binding domain binds to Ptc. In some embodiments, the binding domain binds to RAGE. In some embodiments, the binding domain binds to SIRPA. In some embodiments, the binding domain binds to CD47. In some embodiments, the binding domain binds to SYP. In some embodiments, the binding domain binds to TGFBR1. In some embodiments, the binding domain binds to TGFBR2. In some embodiments, the binding domain binds to TGFBR3. In some embodiments, the binding domain binds to TLR2. In some embodiments, the binding domain binds to TLR3. In some embodiments, the binding domain binds to TLR4. In some embodiments, the binding domain binds to TLR7. In some embodiments, the binding domain binds to TLR9. In some embodiments, the binding domain binds to TNFR1. In some embodiments, the binding domain binds to TRKB. In some embodiments, the binding domain binds to TRKC. In some embodiments, the binding domain binds to ULBP1. In some embodiments, the binding domain binds to ULPB2. In some embodiments, the binding domain binds to uPAR. In some embodiments, the binding domain binds to VACM-1. In some embodiments, the binding domain binds to VEGFR-1. In some embodiments, the binding domain binds to VEGFR-2. In some embodiments, the binding domain binds to ANTXR1. In some embodiments, the binding domain binds to CD248. In some embodiments, the binding domain binds to CNTFR. In some embodiments, the binding domain binds to GPC3. In some embodiments, the binding domain binds to KCNE4. In some embodiments, the binding domain binds to NGFR. In some embodiments, the binding domain binds to NPR3. In some embodiments, the binding domain binds to PTH-1R.

In certain embodiments, the HSC binding domain provided herein is derived from an antibody including antigen binding fragment thereof. In some embodiments, the antibody or antigen-binding fragment thereof comprises a Fab, a Fab′, a F(ab′)₂, a Fd, a single chain Fv or scFv, a disulfide linked Fv, a V-NAR domain, an IgNar, an intrabody, an IgGACH2, a minibody, a F(ab′)₃, a tetrabody, a triabody, a diabody, a single-domain antibody, DVD-Ig, Fcab, mAb², a (scFv)₂, a scFv-Fc, or a synthetic HSC binding module. The term “antibody,” “immunoglobulin,” or “Ig” is used interchangeably herein, and is used in the broadest sense and specifically covers, for example, monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, and multispecific antibodies (e.g., bispecific antibodies so long as they exhibit the desired biological activity), formed from at least two intact antibodies. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck, ed., 2d ed. 1995); and Kuby, Immunology (3d ed. 1997). In specific embodiments, the specific molecular antigen can be bound by an antibody provided herein, including a polypeptide or an epitope. Antibodies also include, but are not limited to, synthetic antibodies, recombinantly produced antibodies, camelized antibodies or their humanized variants, intrabodies, and anti-idiotypic (anti-Id) antibodies. The term “antibody” as used herein also comprises any binding molecule having a Fc region and a functional fragment (e.g., an antigen-binding fragment) of any of the above, which refers to a portion of an antibody heavy or light chain polypeptide that retains some or all of the binding activity of the antibody from which the fragment was derived. Non-limiting examples of functional fragments (e.g., antigen binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers, ed., 1995); Huston, et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. An antibody provided herein can have a domain that specifically binds to an antigen. An antibody provided herein can comprise a kappa light chain. An antibody provided herein can comprise a lambda light chain. Antibodies may be agonistic antibodies or antagonistic antibodies. Exemplary constant regions are listed in Table 5 (see SEQ ID NOs: 143 to 154).

In other embodiments, the binding domain that binds to an antigen expressed on HSCs is not derived from an antibody. In some embodiments, the binding domain that binds to an antigen expressed on HSCs is derived from a natural peptide (e.g., a natural ligand or natural receptor) that binds to an antigen expressed on HSCs.

### Peptide Linkers

In some embodiments, a binding molecule provided herein further comprises one or more linkers between the above described various domains. The various domains described herein may be fused to each other via peptide linkers. In some embodiments, certain domains are directly fused to each other without any peptide linkers. The peptide linkers connecting different domains may be the same or different. In some embodiments, a polypeptide provided herein comprises a peptide linker between certain domains, but not other domains therein.

Each peptide linker in a polypeptide provided herein may have the same or different length and/or sequence. Each peptide linker may be selected and optimized independently. The length, the degree of flexibility and/or other properties of the peptide linker(s) used in the present fusion proteins may have some influence on properties, including but not limited to the affinity, specificity or avidity for one or more particular target molecules. In some embodiment, a peptide linker comprises flexible residues (such as glycine and serine) so that the adjacent domains are free to move relative to each other. For example, a glycine-serine doublet can be a suitable peptide linker.

The peptide linker can be of any suitable length. In some embodiments, the peptide linker is at least about any of 1, 2, 3, 4, 5, 6, 7, 8, 9, 10, 11, 12, 13, 14, 15, 16, 17, 18, 19, 20, 25, 30, 35, 40, 50, or more amino acids long. In some embodiments, the peptide linker is no more than about any of 100, 75, 50, 40, 35, 30, 25, 20, 19, 18, 17, 16, 15, 14, 13, 12, 11, 10, 9, 8, 7, 6, 5 or fewer amino acids long. In some embodiments, the length of the peptide linker is any of about 1 amino acid to about 10 amino acids, about 1 amino acids to about 20 amino acids, about 1 amino acid to about 30 amino acids, about 5 amino acids to about 15 amino acids, about 10 amino acids to about 25 amino acids, about 5 amino acids to about 30 amino acids, about 10 amino acids to about 30 amino acids long, or about 30 amino acids to about 50 amino acids.

The peptide linker may have a naturally occurring sequence, or a non-naturally occurring sequence. For example, a sequence derived from the hinge region of heavy chain only antibodies may be used as the linker. See, for example, WO1996/34103. In some embodiments, the peptide linker is a flexible linker. In some embodiments, a peptide linker provided herein is a (GxS)n linker, wherein x and n, independently can be an integer between 3 and 12, including 3, 4, 5, 6, 7, 8, 9, 10, 11, 12, or more. Exemplary flexible linkers include but not limited to glycine polymers (G)n, glycine-serine polymers (including, for example, (GS), (GSGGS)n, (GGGS)n, and (GGGGS)n, where n is an integer of at least one), glycine-alanine polymers, alanine-serine polymers, and other flexible linkers known in the art. Exemplary peptide linkers are listed in Table 3 below. In some embodiments, the peptide linker provided herein comprises the amino acid sequence of SEQ ID NO: 95. In some embodiments, the peptide linker provided herein comprises the amino acid sequence of SEQ ID NO: 96. In some embodiments, the peptide linker provided herein comprises the amino acid sequence of SEQ ID NO: 97. In some embodiments, the peptide linker provided herein comprises the amino acid sequence of SEQ ID NO: 98. In some embodiments, the peptide linker provided herein comprises the amino acid sequence of SEQ ID NO: 99. In some embodiments, the peptide linker provided herein comprises the amino acid sequence of SEQ ID NO: 100. In some embodiments, the peptide linker provided herein comprises the amino acid sequence of SEQ ID NO: 101. In some embodiments, the peptide linker provided herein comprises the amino acid sequence of SEQ ID NO: 102. In some embodiments, the peptide linker provided herein comprises the amino acid sequence of SEQ ID NO: 103. In some embodiments, the peptide linker provided herein comprises the amino acid sequence of SEQ ID NO: 104. In some embodiments, the peptide linker provided herein comprises the amino acid sequence of SEQ ID NO: 105.

The fusion protein of the present disclosure may comprise a hinge domain that is located between domains described above. A hinge domain is an amino acid segment that is generally found between two domains of a protein and may allow for flexibility of the protein and movement of one or both of the domains relative to one another.

The hinge domain may contain about 10⁻¹⁰⁰ amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length.

In some embodiments, the hinge domain is a hinge domain of a naturally occurring protein. In some embodiments, the hinge domain is at least a portion of a hinge domain of a naturally occurring protein. Hinge domains of antibodies, such as an IgG, IgA, IgM, IgE, or IgD antibodies, are also compatible for use in the fusion proteins described herein. In some embodiments, the hinge domain is the hinge domain that joins the constant domains CHI and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or lgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Non-naturally occurring peptides may also be used as hinge domains for the fusion protein described herein.

Other linkers known in the art, for example, as described in WO2016014789, WO2015158671, WO2016102965, US20150299317, WO2018067992, U.S. Pat. No. 7,741,465, Colcher et al., J. Nat. Cancer Inst. 82:1191-1197 (1990), and Bird et al., Science 242:423-426 (1988) may also be included in the fusion proteins provided herein, the disclosure of each of which is incorporated herein by reference.

### Exemplary Binding Molecules That Bind PDGFRb

PDGFRb is an exemplary antigen expressed on HSCs. In some embodiments, the binding domain targeting HSCs in the present binding molecule comprises an anti-PDGFRb antibody described below or a variant thereof.

In some embodiments, the anti-PDGFRb antibody provided herein binds to PDGFRb (e.g., human PDGFRb) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-PDGFRb antibodies provide herein are those described in Section 7 below. Thus, in some embodiments, the antibody provided herein comprises one or more CDR sequences of SEQ ID NO: 67 or SEQ ID NO: 68. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-PDGFRb antibody is humanized. In some embodiments, the anti-PDGFRb antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 67, and a LCDR1, a LCDR2, and a LCDR3 as set forth in SEQ ID NO: 68. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to PDGFRb comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 1; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 2, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 3; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 4; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 5; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 6. In some embodiments, the anti-PDGFRb antibody is humanized. In some embodiments, the anti-PDGFRb antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 1, the HCDR2 comprises the amino acid sequence of SEQ ID NO: 2, the HCDR3 comprises the amino acid sequence of SEQ ID NO: 3, the LCDR1 comprises the amino acid sequence of SEQ ID NO: 4, the LCDR2 comprises the amino acid sequence of SEQ ID NO: 5, and the LCDR3 comprises the amino acid sequence of SEQ ID NO: 6.

In some embodiments, the antibody further comprises one or more framework regions of SEQ ID NO: 67 or SEQ ID NO: 68. In some embodiments, the antibody provided herein is a humanized antibody. Framework regions described herein are determined based upon the boundaries of the CDR numbering system. In other words, if the CDRs are determined by, e.g., Kabat, IMGT, or Chothia, then the framework regions are the amino acid residues surrounding the CDRs in the variable region in the format, from the N-terminus to C-terminus: FR1-CDR1-FR2-CDR2-FR3-CDR3-FR4. For example, FRI is defined as the amino acid residues N-terminal to the CDR1 amino acid residues as defined by, e.g., the Kabat numbering system, the IMGT numbering system, or the Chothia numbering system, FR2 is defined as the amino acid residues between CDR1 and CDR2 amino acid residues as defined by, e.g., the Kabat numbering system, the IMGT numbering system, or the Chothia numbering system, FR3 is defined as the amino acid residues between CDR2 and CDR3 amino acid residues as defined by, e.g., the Kabat numbering system, the IMGT numbering system, or the Chothia numbering system, and FR4 is defined as the amino acid residues C-terminal to the CDR3 amino acid residues as defined by, e.g., the Kabat numbering system, the IMGT numbering system, or the Chothia numbering system.

In some embodiments, the antibody or antigen binding fragment provided herein comprises a VH comprising the amino acid sequence of SEQ ID NO: 67, and a VL comprising the amino acid sequence of SEQ ID NO: 68.

In certain embodiments, an antibody described herein or an antigen binding fragment thereof comprises amino acid sequences with certain percent identity relative to any antibody provided herein, for example, those described in Section 7 below.

The determination of percent identity between two sequences (e.g., amino acid sequences or nucleic acid sequences) can be accomplished using a mathematical algorithm. A non-limiting example of a mathematical algorithm utilized for the comparison of two sequences is the algorithm of Karlin and Altschul, Proc. Natl. Acad. Sci. U.S.A. 87:2264 2268 (1990), modified as in Karlin and Altschul, Proc. Natl. Acad. Sci. U.S.A. 90:5873 5877 (1993). Such an algorithm is incorporated into the NBLAST and XBLAST programs of Altschul et al., J. Mol. Biol. 215:403 (1990). BLAST nucleotide searches can be performed with the NBLAST nucleotide program parameters set, e.g., for score=100, word length=12 to obtain nucleotide sequences homologous to a nucleic acid molecules described herein. BLAST protein searches can be performed with the XBLAST program parameters set, e.g., to score 50, word length=3 to obtain amino acid sequences homologous to a protein molecule described herein. To obtain gapped alignments for comparison purposes, Gapped BLAST can be utilized as described in Altschul et al., Nucleic Acids Res. 25:3389 3402 (1997). Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another non-limiting example of a mathematical algorithm utilized for the comparison of sequences is the algorithm of Myers and Miller, CABIOS 4:11-17 (1998). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

In some embodiments, the antibody provide herein contains substitutions (e.g., conservative substitutions), insertions, or deletions relative to the reference sequence, but the anti-PDGFRb antibody comprising that sequence retains the ability to bind to PDGFRb. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-PDGFRb antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 67, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 68, and the antibody or antigen binding fragment binds to PDGFRb.

In some embodiments, functional epitopes can be mapped, e.g., by combinatorial alanine scanning, to identify amino acids in the PDGFRb protein that are necessary for interaction with anti-PDGFRb antibodies provided herein. In some embodiments, conformational and crystal structure of anti-PDGFRb antibody bound to PDGFRb may be employed to identify the epitopes. In some embodiments, the present disclosure provides an antibody that specifically binds to the same epitope as any of the anti-PDGFRb antibodies provided herein. For example, in some embodiments, the antibody or antigen binding fragment provided herein binds to the same epitope as an anti-PDGFRb antibody comprising a VH comprising the amino acid sequence of SEQ ID NO: 67, and a VL comprising the amino acid sequence of SEQ ID NO: 68.

In some embodiments, provided herein is an anti-PDGFRb antibody, or antigen binding fragment thereof, that specifically binds to PDGFRb competitively with any one of the anti-PDGFRb antibodies described herein. In some embodiments, the antibody or antigen binding fragment provided herein specifically binds to PDGFRb competitively with an anti-PDGFRb antibody comprising a VH comprising the amino acid sequence of SEQ ID NO: 67, and a VL comprising the amino acid sequence of SEQ ID NO: 68.

In some embodiments, provided herein is a PDGFRb binding protein comprising any one of the anti-PDGFRb antibodies described above. In some embodiments, the PDGFRb binding protein is a monoclonal antibody, including a mouse, chimeric, humanized or human antibody. In some embodiments, the anti-PDGFRb antibody is an antibody fragment, e.g., a scFv. In some embodiments, the PDGFRb binding protein is a fusion protein comprising the anti-PDGFRb antibody provided herein. In other embodiments, the PDGFRb binding protein is a multispecific antibody comprising the anti-PDGFRb antibody provided herein. Other exemplary PDGFRb binding molecules are described in more detail in the following sections.

In some embodiments, the anti-PDGFRb antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRG1, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4, Table 6 and Table 7.

In a specific embodiment, provided herein is a binding molecule comprising a first chain comprising the amino acid sequence of SEQ ID NO: 106 and a second chain comprising the amino acid sequence of SEQ ID NO: 107.

In a specific embodiment, provided herein is a binding molecule comprising a first chain comprising the amino acid sequence of SEQ ID NO: 126 and a second chain comprising the amino acid sequence of SEQ ID NO: 127.

In a specific embodiment, provided herein is a binding molecule comprising a first chain comprising the amino acid sequence of SEQ ID NO: 155 and a second chain comprising the amino acid sequence of SEQ ID NO: 156.

In a specific embodiment, provided herein is a binding molecule comprising a first chain comprising the amino acid sequence of SEQ ID NO: 157 and a second chain comprising the amino acid sequence of SEQ ID NO: 158.

In a specific embodiment, provided herein is a binding molecule comprising a first chain comprising the amino acid sequence of SEQ ID NO: 159 and a second chain comprising the amino acid sequence of SEQ ID NO: 160.

In a specific embodiment, provided herein is a binding molecule comprising a first chain comprising the amino acid sequence of SEQ ID NO: 161 and a second chain comprising the amino acid sequence of SEQ ID NO: 162.

In a specific embodiment, provided herein is a binding molecule comprising a first chain comprising the amino acid sequence of SEQ ID NO: 163 and a second chain comprising the amino acid sequence of SEQ ID NO: 164.

In a specific embodiment, provided herein is a binding molecule comprising a first chain comprising the amino acid sequence of SEQ ID NO: 165 and a second chain comprising the amino acid sequence of SEQ ID NO: 166.

In a specific embodiment, provided herein is a binding molecule comprising a first chain comprising the amino acid sequence of SEQ ID NO: 167 and a second chain comprising the amino acid sequence of SEQ ID NO: 168.

In a specific embodiment, provided herein is a binding molecule comprising a first chain comprising the amino acid sequence of SEQ ID NO: 169 and a second chain comprising the amino acid sequence of SEQ ID NO: 170.

In a specific embodiment, provided herein is a binding molecule comprising a first chain comprising the amino acid sequence of SEQ ID NO: 171 and a second chain comprising the amino acid sequence of SEQ ID NO: 172.

In a specific embodiment, provided herein is a binding molecule comprising a first chain comprising the amino acid sequence of SEQ ID NO: 173 and a second chain comprising the amino acid sequence of SEQ ID NO: 174.

In a specific embodiment, provided herein is a binding molecule comprising a first chain comprising the amino acid sequence of SEQ ID NO: 175 and a second chain comprising the amino acid sequence of SEQ ID NO: 176.

In a specific embodiment, provided herein is a binding molecule comprising a first chain comprising the amino acid sequence of SEQ ID NO: 177 and a second chain comprising the amino acid sequence of SEQ ID NO: 178.

In a specific embodiment, provided herein is a binding molecule comprising a first chain comprising the amino acid sequence of SEQ ID NO: 179 and a second chain comprising the amino acid sequence of SEQ ID NO: 180.

In a specific embodiment, provided herein is a binding molecule comprising a first chain comprising the amino acid sequence of SEQ ID NO: 181 and a second chain comprising the amino acid sequence of SEQ ID NO: 182.

In a specific embodiment, provided herein is a binding molecule comprising a first chain comprising the amino acid sequence of SEQ ID NO: 183 and a second chain comprising the amino acid sequence of SEQ ID NO: 184.

In a specific embodiment, provided herein is a binding molecule comprising a first chain comprising the amino acid sequence of SEQ ID NO: 185 and a second chain comprising the amino acid sequence of SEQ ID NO: 186.

In a specific embodiment, provided herein is a binding molecule comprising a first chain comprising the amino acid sequence of SEQ ID NO: 187 and a second chain comprising the amino acid sequence of SEQ ID NO: 188.

In a specific embodiment, provided herein is a binding molecule comprising a first chain comprising the amino acid sequence of SEQ ID NO: 189 and a second chain comprising the amino acid sequence of SEQ ID NO: 190.

In a specific embodiment, provided herein is a binding molecule comprising a first chain comprising the amino acid sequence of SEQ ID NO: 191 and a second chain comprising the amino acid sequence of SEQ ID NO: 192.

In a specific embodiment, provided herein is a binding molecule comprising a first chain comprising the amino acid sequence of SEQ ID NO: 193 and a second chain comprising the amino acid sequence of SEQ ID NO: 194.

In a specific embodiment, provided herein is a binding molecule comprising a first chain comprising the amino acid sequence of SEQ ID NO: 195 and a second chain comprising the amino acid sequence of SEQ ID NO: 196.

In a specific embodiment, provided herein is a binding molecule comprising a first chain comprising the amino acid sequence of SEQ ID NO: 197 and a second chain comprising the amino acid sequence of SEQ ID NO: 198.

In a specific embodiment, provided herein is a binding molecule comprising a first chain comprising the amino acid sequence of SEQ ID NO: 199 and a second chain comprising the amino acid sequence of SEQ ID NO: 200.

In a specific embodiment, provided herein is a binding molecule comprising a first chain comprising the amino acid sequence of SEQ ID NO: 201 and a second chain comprising the amino acid sequence of SEQ ID NO: 202.

In a specific embodiment, provided herein is a binding molecule comprising a first chain comprising the amino acid sequence of SEQ ID NO: 203 and a second chain comprising the amino acid sequence of SEQ ID NO: 204.

In a specific embodiment, provided herein is a binding molecule comprising a first chain comprising the amino acid sequence of SEQ ID NO: 205 and a second chain comprising the amino acid sequence of SEQ ID NO: 206.

In a specific embodiment, provided herein is a binding molecule comprising a first chain comprising the amino acid sequence of SEQ ID NO: 207 and a second chain comprising the amino acid sequence of SEQ ID NO: 208.

In a specific embodiment, provided herein is a binding molecule comprising a first chain comprising the amino acid sequence of SEQ ID NO: 209 and a second chain comprising the amino acid sequence of SEQ ID NO: 210.

In a specific embodiment, provided herein is a binding molecule comprising a first chain comprising the amino acid sequence of SEQ ID NO: 211 and a second chain comprising the amino acid sequence of SEQ ID NO: 212.

In a specific embodiment, provided herein is a binding molecule comprising a first chain comprising the amino acid sequence of SEQ ID NO: 213 and a second chain comprising the amino acid sequence of SEQ ID NO: 214.

In a specific embodiment, provided herein is a binding molecule comprising a first chain comprising the amino acid sequence of SEQ ID NO: 215, a second chain comprising the amino acid sequence of SEQ ID NO: 216, and a third chain comprising the amino acid sequence of SEQ ID NO: 217.

In a specific embodiment, provided herein is a binding molecule comprising a first chain comprising the amino acid sequence of SEQ ID NO: 218, a second chain comprising the amino acid sequence of SEQ ID NO: 219, and a third chain comprising the amino acid sequence of SEQ ID NO: 220.

In a specific embodiment, provided herein is a binding molecule comprising a first chain comprising the amino acid sequence of SEQ ID NO: 221 and a second chain comprising the amino acid sequence of SEQ ID NO: 222.

In a specific embodiment, provided herein is a binding molecule comprising a first chain comprising the amino acid sequence of SEQ ID NO: 223 and a second chain comprising the amino acid sequence of SEQ ID NO: 224.

In a specific embodiment, provided herein is a binding molecule comprising a first chain comprising the amino acid sequence of SEQ ID NO: 297, a second chain comprising the amino acid sequence of SEQ ID NO: 298, and a third chain comprising the amino acid sequence of SEQ ID NO: 299.

### Exemplary Binding Molecules That Bind SIRPA

SIRPA is another exemplary antigen expressed on HSCs. In some embodiments, the binding domain targeting HSCs in the present binding molecules comprises an anti-SIRPA antibody described below or a variant thereof.

In some embodiments, the anti-SIRPA antibody provided herein binds to SIRPA (e.g., human SIRPA) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-SIRPA antibodies provide herein are those described in Section 7 below. Thus, in some embodiments, the antibody provided herein comprises one or more CDR sequences of SEQ ID NOs: 69 to 74. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-SIRPA antibody is humanized. In some embodiments, the anti-SIRPA antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 69, and a LCDR1, a LCDR2, and a LCDR3 as set forth in SEQ ID NO: 70. In some embodiments, the antibody or antigen binding fragment provided herein comprises an HCDR1, an HCDR2, and an HCDR3 as set forth in SEQ ID NO: 71, and a LCDR1, a LCDR2, and a LCDR3 as set forth in SEQ ID NO: 72. In some embodiments, the antibody or antigen binding fragment provided herein comprises a HCDR1, an HCDR2, and an HCDR3 as set forth in SEQ ID NO: 73, and a LCDR1, a LCDR2, and a LCDR3 as set forth in SEQ ID NO: 74. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to SIRPA comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 7, 13, and 19; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 8, 14 and 20, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 9, 15, and 21; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 10, 16, and 22; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to any one of SEQ ID NOs: 11, 17, and 23; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to any one of SEQ ID NOs: 12, 18, and 24. In some embodiments, the anti-SIRPA antibody is humanized. In some embodiments, the anti-SIRPA antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 7, the HCDR2 comprises the amino acid sequence of SEQ ID NO: 8, the HCDR3 comprises the amino acid sequence of SEQ ID NO: 9, the LCDR1 comprises the amino acid sequence of SEQ ID NO: 10, the LCDR2 comprises the amino acid sequence of SEQ ID NO: 11, and the LCDR3 comprises the amino acid sequence of SEQ ID NO: 12. In some specific embodiments, in the antibody or antigen binding fragment provided herein, the HCDR1 comprises the amino acid sequence of SEQ ID NO: 13, the HCDR2 comprises the amino acid sequence of SEQ ID NO: 14, the HCDR3 comprises the amino acid sequence of SEQ ID NO: 15, the LCDR1 comprises the amino acid sequence of SEQ ID NO: 16, the LCDR2 comprises the amino acid sequence of SEQ ID NO: 17, and the LCDR3 comprises the amino acid sequence of SEQ ID NO: 18. In some specific embodiments, in the antibody or antigen binding fragment provided herein, the HCDR1 comprises the amino acid sequence of SEQ ID NO: 19, the HCDR2 comprises the amino acid sequence of SEQ ID NO: 20, the HCDR3 comprises the amino acid sequence of SEQ ID NO: 21, the LCDR1 comprises the amino acid sequence of SEQ ID NO: 22, the LCDR2 comprises the amino acid sequence of SEQ ID NO: 23, and the LCDR3 comprises the amino acid sequence of SEQ ID NO: 24.

In some embodiments, the antibody further comprises one or more framework regions of SEQ ID NOs: 69 to 74. In some embodiments, the antibody provided herein is a humanized antibody.

In some embodiments, the antibody or antigen binding fragment provided herein comprises a VH comprising the amino acid sequence of SEQ ID NO: 69, and a VL comprising the amino acid sequence of SEQ ID NO: 70.

In some embodiments, the antibody or antigen binding fragment provided herein comprises a VH comprising the amino acid sequence of SEQ ID NO: 71, and a VL comprising the amino acid sequence of SEQ ID NO: 72.

In some embodiments, the antibody or antigen binding fragment provided herein comprises a VH comprising the amino acid sequence of SEQ ID NO: 73, and a VL comprising the amino acid sequence of SEQ ID NO: 74.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-SIRPA antibody comprising that sequence retains the ability to bind to SIRPA. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-SIRPA antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 69, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 70, and the antibody or antigen binding fragment binds to SIRPA.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 71, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 72, and the antibody or antigen binding fragment binds to SIRPA.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 73, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 74, and the antibody or antigen binding fragment binds to SIRPA.

In some embodiments, functional epitopes can be mapped, e.g., by combinatorial alanine scanning, to identify amino acids in the SIRPA protein that are necessary for interaction with anti-SIRPA antibodies provided herein. In some embodiments, conformational and crystal structure of anti-SIRPA antibody bound to SIRPA may be employed to identify the epitopes. In some embodiments, the present disclosure provides an antibody that specifically binds to the same epitope as any of the anti-SIRPA antibodies provided herein. For example, in some embodiments, the antibody or antigen binding fragment provided herein binds to the same epitope as an anti-SIRPA antibody comprising a VH comprising the amino acid sequence of SEQ ID NO: 69, and a VL comprising the amino acid sequence of SEQ ID NO: 70. In some embodiments, the antibody or antigen binding fragment provided herein binds to the same epitope as an anti-SIRPA antibody comprising a VH comprising the amino acid sequence of SEQ ID NO: 71, and a VL comprising the amino acid sequence of SEQ ID NO: 72. In some embodiments, the antibody or antigen binding fragment provided herein binds to the same epitope as an anti-SIRPA antibody comprising a VH comprising the amino acid sequence of SEQ ID NO: 73, and a VL comprising the amino acid sequence of SEQ ID NO: 74.

In some embodiments, provided herein is an anti-SIRPA antibody, or antigen binding fragment thereof, that specifically binds to SIRPA competitively with any one of the anti-SIRPA antibodies described herein. In some embodiments, the antibody or antigen binding fragment provided herein specifically binds to SIRPA competitively with an anti-SIRPA antibody comprising a VH comprising the amino acid sequence of SEQ ID NO: 69, and a VL comprising the amino acid sequence of SEQ ID NO: 70. In some embodiments, the antibody or antigen binding fragment provided herein specifically binds to SIRPA competitively with an anti-SIRPA antibody comprising a VH comprising the amino acid sequence of SEQ ID NO: 71, and a VL comprising the amino acid sequence of SEQ ID NO: 72. In some embodiments, the antibody or antigen binding fragment provided herein specifically binds to SIRPA competitively with an anti-SIRPA antibody comprising a VH comprising the amino acid sequence of SEQ ID NO: 73, and a VL comprising the amino acid sequence of SEQ ID NO: 74.

In some embodiments, provided herein is a SIRPA binding protein comprising any one of the anti-SIRPA antibodies described above. In some embodiments, the SIRPA binding protein is a monoclonal antibody, including a mouse, chimeric, humanized or human antibody. In some embodiments, the anti-SIRPA antibody is an antibody fragment, e.g., a scFv. In some embodiments, the SIRPA binding protein is a fusion protein comprising the anti-SIRPA antibody provided herein. In other embodiments, the SIRPA binding protein is a multispecific antibody comprising the anti-SIRPA antibody provided herein. Other exemplary SIRPA binding molecules are described in more detail in the following sections.

In some embodiments, the anti-SIRPA antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRG1, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 108 and a second chain comprising the amino acid sequence of SEQ ID NO: 109.

In a specific embodiment, provided herein is a binding molecule comprising a first chain comprising the amino acid sequence of SEQ ID NO: 110 and a second chain comprising the amino acid sequence of SEQ ID NO: 111.

In a specific embodiment, provided herein is a binding molecule comprising a first chain comprising the amino acid sequence of SEQ ID NO: 112 and a second chain comprising the amino acid sequence of SEQ ID NO: 113.

In a specific embodiment, provided herein is a binding molecule comprising a first chain comprising the amino acid sequence of SEQ ID NO: 128 and a second chain comprising the amino acid sequence of SEQ ID NO: 129.

In a specific embodiment, provided herein is a binding molecule comprising a first chain comprising the amino acid sequence of SEQ ID NO: 130 and a second chain comprising the amino acid sequence of SEQ ID NO: 131.

In a specific embodiment, provided herein is a binding molecule comprising a first chain comprising the amino acid sequence of SEQ ID NO: 132 and a second chain comprising the amino acid sequence of SEQ ID NO: 133.

In a specific embodiment, provided herein is a binding molecule comprising a first chain comprising the amino acid sequence of SEQ ID NO: 134 and a second chain comprising the amino acid sequence of SEQ ID NO: 135.

In a specific embodiment, provided herein is a binding molecule comprising a first chain comprising the amino acid sequence of SEQ ID NO: 136 and a second chain comprising the amino acid sequence of SEQ ID NO: 137.

In a specific embodiment, provided herein is a binding molecule comprising a first chain comprising the amino acid sequence of SEQ ID NO: 291 and a second chain comprising the amino acid sequence of SEQ ID NO: 292.

### Exemplary Binding Molecules That Bind FAPa

FAPa is yet another exemplary antigen expressed on HSCs. In some embodiments, the binding domain targeting HSCs in the present binding molecules comprises an anti-FAPa antibody described below or a variant thereof.

In some embodiments, the anti-FAPa antibody provided herein binds to FAPa (e.g., human FAPα) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-FAPa antibodies provide herein are those described in Section 7 below. Thus, in some embodiments, the antibody provided herein comprises one or more CDR sequences of SEQ ID NOs: 75 to 78. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-FAPa antibody is humanized. In some embodiments, the anti-FAPa antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 75, and a LCDR1, a LCDR2, and a LCDR3 as set forth in SEQ ID NO: 76. In some embodiments, the antibody or antigen binding fragment provided herein comprises an HCDR1, an HCDR2, and an HCDR3 as set forth in SEQ ID NO: 77, and a LCDR1, a LCDR2, and a LCDR3 as set forth in SEQ ID NO: 78. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to FAPa comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 25 or 31; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 26 or 32, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 27 or 33; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 28 or 34; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 29 or 35; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 30 or 36. In some embodiments, the anti-FAPa antibody is humanized. In some embodiments, the anti-FAPa antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 25, the HCDR2 comprises the amino acid sequence of SEQ ID NO: 26, the HCDR3 comprises the amino acid sequence of SEQ ID NO: 27, the LCDR1 comprises the amino acid sequence of SEQ ID NO: 28, the LCDR2 comprises the amino acid sequence of SEQ ID NO: 29, and the LCDR3 comprises the amino acid sequence of SEQ ID NO: 30. In some specific embodiments, in the antibody or antigen binding fragment provided herein, the HCDR1 comprises the amino acid sequence of SEQ ID NO: 31, the HCDR2 comprises the amino acid sequence of SEQ ID NO: 32, the HCDR3 comprises the amino acid sequence of SEQ ID NO: 33, the LCDR1 comprises the amino acid sequence of SEQ ID NO: 34, the LCDR2 comprises the amino acid sequence of SEQ ID NO: 35, and the LCDR3 comprises the amino acid sequence of SEQ ID NO: 36.

In some embodiments, the antibody further comprises one or more framework regions of SEQ ID NOs: 75 to 78. In some embodiments, the antibody provided herein is a humanized antibody.

In some embodiments, the antibody or antigen binding fragment provided herein comprises a VH comprising the amino acid sequence of SEQ ID NO: 75, and a VL comprising the amino acid sequence of SEQ ID NO: 76.

In some embodiments, the antibody or antigen binding fragment provided herein comprises a VH comprising the amino acid sequence of SEQ ID NO: 77, and a VL comprising the amino acid sequence of SEQ ID NO: 78.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-FAPa antibody comprising that sequence retains the ability to bind to FAPα. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-FAPa antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 75, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 76, and the antibody or antigen binding fragment binds to FAPα.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 77, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 78, and the antibody or antigen binding fragment binds to FAPα.

In some embodiments, functional epitopes can be mapped, e.g., by combinatorial alanine scanning, to identify amino acids in the FAPa protein that are necessary for interaction with anti-FAPa antibodies provided herein. In some embodiments, conformational and crystal structure of anti-FAPa antibody bound to FAPa may be employed to identify the epitopes. In some embodiments, the present disclosure provides an antibody that specifically binds to the same epitope as any of the anti-FAPa antibodies provided herein. For example, in some embodiments, the antibody or antigen binding fragment provided herein binds to the same epitope as an anti-FAPa antibody comprising a VH comprising the amino acid sequence of SEQ ID NO: 75, and a VL comprising the amino acid sequence of SEQ ID NO: 76. In some embodiments, the antibody or antigen binding fragment provided herein binds to the same epitope as an anti-FAPa antibody comprising a VH comprising the amino acid sequence of SEQ ID NO: 77, and a VL comprising the amino acid sequence of SEQ ID NO: 78.

In some embodiments, provided herein is an anti-FAPa antibody, or antigen binding fragment thereof, that specifically binds to FAPa competitively with any one of the anti-FAPa antibodies described herein. In some embodiments, the antibody or antigen binding fragment provided herein specifically binds to FAPa competitively with an anti-FAPa antibody comprising a VH comprising the amino acid sequence of SEQ ID NO: 75, and a VL comprising the amino acid sequence of SEQ ID NO: 76. In some embodiments, the antibody or antigen binding fragment provided herein specifically binds to FAPa competitively with an anti-FAPa antibody comprising a VH comprising the amino acid sequence of SEQ ID NO: 77, and a VL comprising the amino acid sequence of SEQ ID NO: 78.

In some embodiments, provided herein is a FAPa binding protein comprising any one of the anti-FAPa antibodies described above. In some embodiments, the FAPa binding protein is a monoclonal antibody, including a mouse, chimeric, humanized or human antibody. In some embodiments, the anti-FAPa antibody is an antibody fragment, e.g., a scFv. In some embodiments, the FAPa binding protein is a fusion protein comprising the anti-FAPa antibody provided herein. In other embodiments, the FAPa binding protein is a multispecific antibody comprising the anti-FAPa antibody provided herein. Other exemplary FAPa binding molecules are described in more detail in the following sections.

In some embodiments, the anti-FAPa antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL 12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 114 and a second chain comprising the amino acid sequence of SEQ ID NO: 115.

In a specific embodiment, provided herein is a binding molecule comprising a first chain comprising the amino acid sequence of SEQ ID NO: 116 and a second chain comprising the amino acid sequence of SEQ ID NO: 117.

### Exemplary Binding Molecules That Bind PD-L1

PD-LI is yet another exemplary antigen expressed on HSCs. In some embodiments, the binding domain targeting HSCs in the present binding molecules comprises an anti-PD-L1 antibody described below or a variant thereof.

In some embodiments, the anti-PD-L1 antibody provided herein binds to PD-L1 (e.g., human PD-L1) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-PD-L1 antibodies provide herein are those described in Section 7 below. Thus, in some embodiments, the antibody provided herein comprises one or more CDR sequences of SEQ ID NOs: 79 and 80. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-PD-L1 antibody is humanized. In some embodiments, the anti-PD-L1 antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 79, and a LCDR1, a LCDR2, and a LCDR3 as set forth in SEQ ID NO: 80. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to PD-L1 comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 37; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 38, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 39; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 40; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 41; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 42. In some embodiments, the anti-PD-L1 antibody is humanized. In some embodiments, the anti-PD-L1 antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 37, the HCDR2 comprises the amino acid sequence of SEQ ID NO: 38, the HCDR3 comprises the amino acid sequence of SEQ ID NO: 39, the LCDR1 comprises the amino acid sequence of SEQ ID NO: 40, the LCDR2 comprises the amino acid sequence of SEQ ID NO: 41, and the LCDR3 comprises the amino acid sequence of SEQ ID NO: 42.

In some embodiments, the antibody further comprises one or more framework regions of SEQ ID NOs: 79 and 80. In some embodiments, the antibody provided herein is a humanized antibody.

In some embodiments, the antibody or antigen binding fragment provided herein comprises a VH comprising the amino acid sequence of SEQ ID NO: 79, and a VL comprising the amino acid sequence of SEQ ID NO: 80.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-PD-L1 antibody comprising that sequence retains the ability to bind to PD-L1. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-PD-L1 antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 79, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 80, and the antibody or antigen binding fragment binds to PD-L1.

In some embodiments, functional epitopes can be mapped, e.g., by combinatorial alanine scanning, to identify amino acids in the PD-L1 protein that are necessary for interaction with anti-PD-L1 antibodies provided herein. In some embodiments, conformational and crystal structure of anti-PD-L1 antibody bound to PD-LI may be employed to identify the epitopes. In some embodiments, the present disclosure provides an antibody that specifically binds to the same epitope as any of the anti-PD-L1 antibodies provided herein. For example, in some embodiments, the antibody or antigen binding fragment provided herein binds to the same epitope as an anti-PD-L1 antibody comprising a VH comprising the amino acid sequence of SEQ ID NO: 79, and a VL comprising the amino acid sequence of SEQ ID NO: 80.

In some embodiments, provided herein is an anti-PD-L1 antibody, or antigen binding fragment thereof, that specifically binds to PD-L1 competitively with any one of the anti-PD-L1 antibodies described herein. In some embodiments, the antibody or antigen binding fragment provided herein specifically binds to PD-L1 competitively with an anti-PD-LI antibody comprising a VH comprising the amino acid sequence of SEQ ID NO: 79, and a VL comprising the amino acid sequence of SEQ ID NO: 80.

In some embodiments, provided herein is a PD-L1 binding protein comprising any one of the anti-PD-L1 antibodies described above. In some embodiments, the PD-L1 binding protein is a monoclonal antibody, including a mouse, chimeric, humanized or human antibody. In some embodiments, the anti-PD-L1 antibody is an antibody fragment, e.g., a scFv. In some embodiments, the PD-L1 binding protein is a fusion protein comprising the anti-PD-L1 antibody provided herein. In other embodiments, the PD-L1 binding protein is a multispecific antibody comprising the anti-PD-L1 antibody provided herein. Other exemplary PD-L1 binding molecules are described in more detail in the following sections.

In some embodiments, the anti-PD-L1 antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 118 and a second chain comprising the amino acid sequence of SEQ ID NO: 119.

### Exemplary Binding Molecules That Bind uPAR

uPAR is yet another exemplary antigen expressed on HSCs. In some embodiments, the binding domain targeting HSCs in the present binding molecules comprises an anti-uPAR antibody described below or a variant thereof.

In some embodiments, the anti-uPAR antibody provided herein binds to uPAR (e.g., human uPAR) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-uPAR antibodies provide herein are those described in Section 7 below. Thus, in some embodiments, the antibody provided herein comprises one or more CDR sequences of SEQ ID NOs: 81 and 82. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-uPAR antibody is humanized. In some embodiments, the anti-uPAR antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 81, and a LCDR1, a LCDR2, and a LCDR3 as set forth in SEQ ID NO: 82. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to uPAR comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 43; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 44, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 45; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 46; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 47; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 48. In some embodiments, the anti-uPAR antibody is humanized. In some embodiments, the anti-uPAR antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 43, the HCDR2 comprises the amino acid sequence of SEQ ID NO: 44, the HCDR3 comprises the amino acid sequence of SEQ ID NO: 45, the LCDR1 comprises the amino acid sequence of SEQ ID NO: 46, the LCDR2 comprises the amino acid sequence of SEQ ID NO: 47, and the LCDR3 comprises the amino acid sequence of SEQ ID NO: 48.

In some embodiments, the antibody further comprises one or more framework regions of SEQ ID NOs: 81 and 82. In some embodiments, the antibody provided herein is a humanized antibody.

In some embodiments, the antibody or antigen binding fragment provided herein comprises a VH comprising the amino acid sequence of SEQ ID NO: 81, and a VL comprising the amino acid sequence of SEQ ID NO: 82.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-uPAR antibody comprising that sequence retains the ability to bind to uPAR. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-uPAR antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 81, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 82, and the antibody or antigen binding fragment binds to uPAR.

In some embodiments, functional epitopes can be mapped, e.g., by combinatorial alanine scanning, to identify amino acids in the uPAR protein that are necessary for interaction with anti-uPAR antibodies provided herein. In some embodiments, conformational and crystal structure of anti-uPAR antibody bound to uPAR may be employed to identify the epitopes. In some embodiments, the present disclosure provides an antibody that specifically binds to the same epitope as any of the anti-uPAR antibodies provided herein. For example, in some embodiments, the antibody or antigen binding fragment provided herein binds to the same epitope as an anti-uPAR antibody comprising a VH comprising the amino acid sequence of SEQ ID NO: 81, and a VL comprising the amino acid sequence of SEQ ID NO: 82.

In some embodiments, provided herein is an anti-uPAR antibody, or antigen binding fragment thereof, that specifically binds to uPAR competitively with any one of the anti-uPAR antibodies described herein. In some embodiments, the antibody or antigen binding fragment provided herein specifically binds to uPAR competitively with an anti-uPAR antibody comprising a VH comprising the amino acid sequence of SEQ ID NO: 81, and a VL comprising the amino acid sequence of SEQ ID NO: 82.

In some embodiments, provided herein is a uPAR binding protein comprising any one of the anti-uPAR antibodies described above. In some embodiments, the uPAR binding protein is a monoclonal antibody, including a mouse, chimeric, humanized or human antibody. In some embodiments, the anti-uPAR antibody is an antibody fragment, e.g., a scFv. In some embodiments, the uPAR binding protein is a fusion protein comprising the anti-uPAR antibody provided herein. In other embodiments, the uPAR binding protein is a multispecific antibody comprising the anti-uPAR antibody provided herein. Other exemplary uPAR binding molecules are described in more detail in the following sections.

In some embodiments, the anti-uPAR antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 120 and a second chain comprising the amino acid sequence of SEQ ID NO: 121.

### Exemplary Binding Molecules That Bind IGF-1R

IGF-IR is yet another exemplary antigen expressed on HSCs. In some embodiments, the binding domain targeting HSCs in the present binding molecules comprises an anti-IGF-IR antibody described below or a variant thereof.

In some embodiments, the anti-IGF-IR antibody provided herein binds to IGF-1R (e.g., human IGF-1R) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-IGF-IR antibodies provide herein are those described in Section 7 below. Thus, in some embodiments, the antibody provided herein comprises one or more CDR sequences of SEQ ID NOs: 83 to 86. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-IGF-IR antibody is humanized. In some embodiments, the anti-IGF-IR antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 83, and a LCDR1, a LCDR2, and a LCDR3 as set forth in SEQ ID NO: 84. In some embodiments, the antibody or antigen binding fragment provided herein comprises an HCDR1, an HCDR2, and an HCDR3 as set forth in SEQ ID NO: 85, and a LCDR1, a LCDR2, and a LCDR3 as set forth in SEQ ID NO: 86. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to IGF-1R comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 49 or 55; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 50 or 56, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 51 or 57; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 52 or 58; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 53 or 59; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 54 or 60. In some embodiments, the anti-IGF-1R antibody is humanized. In some embodiments, the anti-IGF-1R antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 49, the HCDR2 comprises the amino acid sequence of SEQ ID NO: 50, the HCDR3 comprises the amino acid sequence of SEQ ID NO: 51, the LCDR1 comprises the amino acid sequence of SEQ ID NO: 52, the LCDR2 comprises the amino acid sequence of SEQ ID NO: 53, and the LCDR3 comprises the amino acid sequence of SEQ ID NO: 54.

In some specific embodiments, in the antibody or antigen binding fragment provided herein, the HCDR1 comprises the amino acid sequence of SEQ ID NO: 55, the HCDR2 comprises the amino acid sequence of SEQ ID NO: 56, the HCDR3 comprises the amino acid sequence of SEQ ID NO: 57, the LCDR1 comprises the amino acid sequence of SEQ ID NO: 58, the LCDR2 comprises the amino acid sequence of SEQ ID NO: 59, and the LCDR3 comprises the amino acid sequence of SEQ ID NO: 60.

In some embodiments, the antibody further comprises one or more framework regions of SEQ ID NOs: 83 to 86. In some embodiments, the antibody provided herein is a humanized antibody.

In some embodiments, the antibody or antigen binding fragment provided herein comprises a VH comprising the amino acid sequence of SEQ ID NO: 83, and a VL comprising the amino acid sequence of SEQ ID NO: 84. In some embodiments, the antibody or antigen binding fragment provided herein comprises a VH comprising the amino acid sequence of SEQ ID NO: 85, and a VL comprising the amino acid sequence of SEQ ID NO: 86.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-IGF-1R antibody comprising that sequence retains the ability to bind to IGF-IR. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-IGF-IR antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 83, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 84, and the antibody or antigen binding fragment binds to IGF-1R.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 85, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 86, and the antibody or antigen binding fragment binds to IGF-1R.

In some embodiments, functional epitopes can be mapped, e.g., by combinatorial alanine scanning, to identify amino acids in the IGF-IR protein that are necessary for interaction with anti-IGF-IR antibodies provided herein. In some embodiments, conformational and crystal structure of anti-IGF-IR antibody bound to IGF-1R may be employed to identify the epitopes. In some embodiments, the present disclosure provides an antibody that specifically binds to the same epitope as any of the anti-IGF-IR antibodies provided herein. For example, in some embodiments, the antibody or antigen binding fragment provided herein binds to the same epitope as an anti-IGF-IR antibody comprising a VH comprising the amino acid sequence of SEQ ID NO: 83, and a VL comprising the amino acid sequence of SEQ ID NO: 84. In some embodiments, the antibody or antigen binding fragment provided herein binds to the same epitope as an anti-IGF-IR antibody comprising a VH comprising the amino acid sequence of SEQ ID NO: 85, and a VL comprising the amino acid sequence of SEQ ID NO: 86.

In some embodiments, provided herein is an anti-IGF-IR antibody, or antigen binding fragment thereof, that specifically binds to IGF-IR competitively with any one of the anti-IGF-1R antibodies described herein. In some embodiments, the antibody or antigen binding fragment provided herein specifically binds to IGF-IR competitively with an anti-IGF-1R antibody comprising a VH comprising the amino acid sequence of SEQ ID NO: 83, and a VL comprising the amino acid sequence of SEQ ID NO: 84. In some embodiments, the antibody or antigen binding fragment provided herein specifically binds to IGF-1R competitively with an anti-IGF-IR antibody comprising a VH comprising the amino acid sequence of SEQ ID NO: 85, and a VL comprising the amino acid sequence of SEQ ID NO: 86.

In some embodiments, provided herein is a IGF-IR binding protein comprising any one of the anti-IGF-IR antibodies described above. In some embodiments, the IGF-1R binding protein is a monoclonal antibody, including a mouse, chimeric, humanized or human antibody. In some embodiments, the anti-IGF-1R antibody is an antibody fragment, e.g., a scFv. In some embodiments, the IGF-1R binding protein is a fusion protein comprising the anti-IGF-1R antibody provided herein. In other embodiments, the IGF-1R binding protein is a multispecific antibody comprising the anti-IGF-1R antibody provided herein. Other exemplary IGF-1R binding molecules are described in more detail in the following sections.

In some embodiments, the anti-IGF-1R antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 122 and a second chain comprising the amino acid sequence of SEQ ID NO: 123.

In a specific embodiment, provided herein is a binding molecule comprising a first chain comprising the amino acid sequence of SEQ ID NO: 124 and a second chain comprising the amino acid sequence of SEQ ID NO: 125.

### Exemplary Binding Molecules That Bind ANTXR1

ANTXR1 is yet another exemplary antigen expressed on HSCs. In some embodiments, the binding domain targeting HSCs in the present binding molecules comprises an anti-ANTXR1 antibody described below or a variant thereof.

In some embodiments, the anti-ANTXR1 antibody provided herein binds to ANTXR1 (e.g., human ANTXR1) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-ANTXR1 antibodies provide herein are those described in Section 7 below. Thus, in some embodiments, the antibody provided herein comprises one or more CDR sequences of SEQ ID NOs: 225, 226, 233, and 234. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-ANTXR1 antibody is humanized. In some embodiments, the anti-ANTXR1 antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 225, and a LCDR1, a LCDR2, and a LCDR3 as set forth in SEQ ID NO: 226. In some embodiments, the antibody or antigen binding fragment provided herein comprises an HCDR1, an HCDR2, and an HCDR3 as set forth in SEQ ID NO: 233, and a LCDR1, a LCDR2, and a LCDR3 as set forth in SEQ ID NO: 234. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to ANTXR1 comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 227 or 235; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 228 or 236, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 229 or 237; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 230 or 238; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 231 or 239; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 232 or 240. In some embodiments, the anti-ANTXR1 antibody is humanized. In some embodiments, the anti-ANTXR1 antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 227, the HCDR2 comprises the amino acid sequence of SEQ ID NO: 228, the HCDR3 comprises the amino acid sequence of SEQ ID NO: 229, the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 232. In some specific embodiments, in the antibody or antigen binding fragment provided herein, the HCDR1 comprises the amino acid sequence of SEQ ID NO: 235, the HCDR2 comprises the amino acid sequence of SEQ ID NO: 236, the HCDR3 comprises the amino acid sequence of SEQ ID NO: 237, the LCDR1 comprises the amino acid sequence of SEQ ID NO: 238, the LCDR2 comprises the amino acid sequence of SEQ ID NO: 239, and the LCDR3 comprises the amino acid sequence of SEQ ID NO: 240.

In some embodiments, the antibody further comprises one or more framework regions of SEQ ID NOs: 225, 226, 233, and 234. In some embodiments, the antibody provided herein is a humanized antibody.

In some embodiments, the antibody or antigen binding fragment provided herein comprises a VH comprising the amino acid sequence of SEQ ID NO: 225, and a VL comprising the amino acid sequence of SEQ ID NO: 226.

In some embodiments, the antibody or antigen binding fragment provided herein comprises a VH comprising the amino acid sequence of SEQ ID NO: 233, and a VL comprising the amino acid sequence of SEQ ID NO: 234.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-ANTXR1 antibody comprising that sequence retains the ability to bind to ANTXR1. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-ANTXR1 antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 225, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 226, and the antibody or antigen binding fragment binds to ANTXR1.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 233, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 234, and the antibody or antigen binding fragment binds to ANTXR1.

In some embodiments, functional epitopes can be mapped, e.g., by combinatorial alanine scanning, to identify amino acids in the ANTXR1 protein that are necessary for interaction with anti-ANTXR1 antibodies provided herein. In some embodiments, conformational and crystal structure of anti-ANTXR1 antibody bound to ANTXR1 may be employed to identify the epitopes. In some embodiments, the present disclosure provides an antibody that specifically binds to the same epitope as any of the anti-ANTXR1 antibodies provided herein. For example, in some embodiments, the antibody or antigen binding fragment provided herein binds to the same epitope as an anti-ANTXR1 antibody comprising a VH comprising the amino acid sequence of SEQ ID NO: 225, and a VL comprising the amino acid sequence of SEQ ID NO: 226. In some embodiments, the antibody or antigen binding fragment provided herein binds to the same epitope as an anti-ANTXR1 antibody comprising a VH comprising the amino acid sequence of SEQ ID NO: 233, and a VL comprising the amino acid sequence of SEQ ID NO: 234.

In some embodiments, provided herein is an anti-ANTXR1 antibody, or antigen binding fragment thereof, that specifically binds to ANTXR1 competitively with any one of the anti-ANTXR1 antibodies described herein. In some embodiments, the antibody or antigen binding fragment provided herein specifically binds to ANTXR1 competitively with an anti-ANTXR1 antibody comprising a VH comprising the amino acid sequence of SEQ ID NO: 225, and a VL comprising the amino acid sequence of SEQ ID NO: 226. In some embodiments, the antibody or antigen binding fragment provided herein specifically binds to ANTXR1 competitively with an anti-ANTXR1 antibody comprising a VH comprising the amino acid sequence of SEQ ID NO: 233, and a VL comprising the amino acid sequence of SEQ ID NO: 234.

In some embodiments, provided herein is a ANTXR1 binding protein comprising any one of the anti-ANTXR1 antibodies described above. In some embodiments, the ANTXR1 binding protein is a monoclonal antibody, including a mouse, chimeric, humanized or human antibody. In some embodiments, the anti-ANTXR1 antibody is an antibody fragment, e.g., a scFv. In some embodiments, the ANTXR1 binding protein is a fusion protein comprising the anti-ANTXR1 antibody provided herein. In other embodiments, the ANTXR1 binding protein is a multispecific antibody comprising the anti-ANTXR1 antibody provided herein. Other exemplary ANTXR1 binding molecules are described in more detail in the following sections.

In some embodiments, the anti-ANTXR1 antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 281 and a second chain comprising the amino acid sequence of SEQ ID NO: 282.

In a specific embodiment, provided herein is a binding molecule comprising a first chain comprising the amino acid sequence of SEQ ID NO: 283 and a second chain comprising the amino acid sequence of SEQ ID NO: 284.

### Exemplary Binding Molecules That Bind CD248

CD248 is yet another exemplary antigen expressed on HSCs. In some embodiments, the binding domain targeting HSCs in the present binding molecules comprises an anti-CD248 antibody described below or a variant thereof.

In some embodiments, the anti-CD248 antibody provided herein binds to CD248 (e.g., human CD248) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-CD248 antibodies provide herein are those described in Section 7 below. Thus, in some embodiments, the antibody provided herein comprises one or more CDR sequences of SEQ ID NOs: 241, 242, 249, and 250. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-CD248 antibody is humanized. In some embodiments, the anti-CD248 antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 241, and a LCDR1, a LCDR2, and a LCDR3 as set forth in SEQ ID NO: 242. In some embodiments, the antibody or antigen binding fragment provided herein comprises an HCDR1, an HCDR2, and an HCDR3 as set forth in SEQ ID NO: 249, and a LCDR1, a LCDR2, and a LCDR3 as set forth in SEQ ID NO: 250. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to CD248 comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 243 or 251; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 244 or 252, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 245 or 253; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 246 or 254; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 247 or 255; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 248 or 256. In some embodiments, the anti-CD248 antibody is humanized. In some embodiments, the anti-CD248 antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 243, the HCDR2 comprises the amino acid sequence of SEQ ID NO: 244, the HCDR3 comprises the amino acid sequence of SEQ ID NO: 245, the LCDR1 comprises the amino acid sequence of SEQ ID NO: 246, the LCDR2 comprises the amino acid sequence of SEQ ID NO: 247, and the LCDR3 comprises the amino acid sequence of SEQ ID NO: 248. In some specific embodiments, in the antibody or antigen binding fragment provided herein, the HCDR1 comprises the amino acid sequence of SEQ ID NO: 251, the HCDR2 comprises the amino acid sequence of SEQ ID NO: 252, the HCDR3 comprises the amino acid sequence of SEQ ID NO: 253, the LCDR1 comprises the amino acid sequence of SEQ ID NO: 254, the LCDR2 comprises the amino acid sequence of SEQ ID NO: 255, and the LCDR3 comprises the amino acid sequence of SEQ ID NO: 256.

In some embodiments, the antibody further comprises one or more framework regions of SEQ ID NOs: 241, 242, 249, and 250. In some embodiments, the antibody provided herein is a humanized antibody.

In some embodiments, the antibody or antigen binding fragment provided herein comprises a VH comprising the amino acid sequence of SEQ ID NO: 241, and a VL comprising the amino acid sequence of SEQ ID NO: 242.

In some embodiments, the antibody or antigen binding fragment provided herein comprises a VH comprising the amino acid sequence of SEQ ID NO: 249, and a VL comprising the amino acid sequence of SEQ ID NO: 250.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-CD248 antibody comprising that sequence retains the ability to bind to CD248. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-CD248 antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 241, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 242, and the antibody or antigen binding fragment binds to CD248.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 249, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 250, and the antibody or antigen binding fragment binds to CD248.

In some embodiments, functional epitopes can be mapped, e.g., by combinatorial alanine scanning, to identify amino acids in the CD248 protein that are necessary for interaction with anti-CD248 antibodies provided herein. In some embodiments, conformational and crystal structure of anti-CD248 antibody bound to CD248 may be employed to identify the epitopes. In some embodiments, the present disclosure provides an antibody that specifically binds to the same epitope as any of the anti-CD248 antibodies provided herein. For example, in some embodiments, the antibody or antigen binding fragment provided herein binds to the same epitope as an anti-CD248 antibody comprising a VH comprising the amino acid sequence of SEQ ID NO: 241, and a VL comprising the amino acid sequence of SEQ ID NO: 242. In some embodiments, the antibody or antigen binding fragment provided herein binds to the same epitope as an anti-CD248 antibody comprising a VH comprising the amino acid sequence of SEQ ID NO: 249, and a VL comprising the amino acid sequence of SEQ ID NO: 250.

In some embodiments, provided herein is an anti-CD248 antibody, or antigen binding fragment thereof, that specifically binds to CD248 competitively with any one of the anti-CD248 antibodies described herein. In some embodiments, the antibody or antigen binding fragment provided herein specifically binds to CD248 competitively with an anti-CD248 antibody comprising a VH comprising the amino acid sequence of SEQ ID NO: 241, and a VL comprising the amino acid sequence of SEQ ID NO: 242. In some embodiments, the antibody or antigen binding fragment provided herein specifically binds to CD248 competitively with an anti-CD248 antibody comprising a VH comprising the amino acid sequence of SEQ ID NO: 249, and a VL comprising the amino acid sequence of SEQ ID NO: 250.

In some embodiments, provided herein is a CD248 binding protein comprising any one of the anti-CD248 antibodies described above. In some embodiments, the CD248 binding protein is a monoclonal antibody, including a mouse, chimeric, humanized or human antibody. In some embodiments, the anti-CD248 antibody is an antibody fragment, e.g., a scFv. In some embodiments, the CD248 binding protein is a fusion protein comprising the anti-CD248 antibody provided herein. In other embodiments, the CD248 binding protein is a multispecific antibody comprising the anti-CD248 antibody provided herein. Other exemplary CD248 binding molecules are described in more detail in the following sections.

In some embodiments, the anti-CD248 antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 285 and a second chain comprising the amino acid sequence of SEQ ID NO: 286.

In a specific embodiment, provided herein is a binding molecule comprising a first chain comprising the amino acid sequence of SEQ ID NO: 287 and a second chain comprising the amino acid sequence of SEQ ID NO: 288.

### Exemplary Binding Molecules That Bind GPC3

GPC3 is yet another exemplary antigen expressed on HSCs. In some embodiments, the binding domain targeting HSCs in the present binding molecules comprises an anti-GPC3 antibody described below or a variant thereof.

In some embodiments, the anti-GPC3 antibody provided herein binds to GPC3 (e.g., human GPC3) with a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). A variety of methods of measuring binding affinity are known in the art, any of which can be used for purposes of the present disclosure, including by RIA, for example, performed with the Fab version of an antibody of interest and its antigen (Chen et al., 1999, J. Mol Biol 293:865-81); by biolayer interferometry (BLI) or surface plasmon resonance (SPR) assays by Octet®, using, for example, an Octet®Red96 system, or by Biacore®, using, for example, a Biacore®TM-2000 or a Biacore®TM-3000. An “on-rate” or “rate of association” or “association rate” or “kₒₙ” may also be determined with the same biolayer interferometry (BLI) or surface plasmon resonance (SPR) techniques described above using, for example, the Octet®Red96, the Biacore®TM-2000, or the Biacore®TM-3000 system.

In some embodiments, the anti-GPC3 antibodies provide herein are those described in Section 7 below. Thus, in some embodiments, the antibody provided herein comprises one or more CDR sequences of SEQ ID NOs: 257 and 258. CDR sequences can be determined according to well-known numbering systems. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the anti-GPC3 antibody is humanized. In some embodiments, the anti-GPC3 antibody comprises an acceptor human framework, e.g., a human immunoglobulin framework or a human consensus framework.

In some embodiments, the antibody or antigen binding fragment provided herein comprises an HCDR1, an HCDR2, and an HCDR3 as set forth in SEQ ID NO: 257, and a LCDR1, a LCDR2, and a LCDR3 as set forth in SEQ ID NO: 258. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering.

In other embodiments, provided herein is an antibody that binds to GPC3 comprising an HCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 259; (ii) an HCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 260, (iii) an HCDR3 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 261; (iv) a LCDR1 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 262; (v) a LCDR2 comprising an amino acid sequence having at least 75%, 80%, 85%, 86%, 87%, 88%, 89%, 90%, 91%, 92%, 93%, 94%, 95%, 96%, 97%, 98%, 99%, or 100% sequence identity to SEQ ID NO: 263; and/or (vi) a LCDR3 comprising an amino acid sequence having at least 75%, 80%, 85%, 86%, 87%, 88%, 89%, 90%, 91%, 92%, 93%, 94%, 95%, 96%, 97%, 98%, 99%, 100% sequence identity to SEQ ID NO: 264. In some embodiments, the anti-GPC3 antibody is humanized. In some embodiments, the anti-GPC3 antibody comprises an acceptor human framework, e.g., a human immunoglobulin framework or a human consensus framework.

In some specific embodiments, in the antibody or antigen binding fragment provided herein, the HCDR1 comprises the amino acid sequence of SEQ ID NO: 259, the HCDR2 comprises the amino acid sequence of SEQ ID NO: 260, the HCDR3 comprises the amino acid sequence of SEQ ID NO: 261, the LCDR1 comprises the amino acid sequence of SEQ ID NO: 262, the LCDR2 comprises the amino acid sequence of SEQ ID NO: 263, and the LCDR3 comprises the amino acid sequence of SEQ ID NO: 264.

In some embodiments, the antibody further comprises one or more framework regions of SEQ ID NOs: 257 and 258. In some embodiments, the antibody provided herein is a humanized antibody.

In some embodiments, the antibody or antigen binding fragment provided herein comprises a VH comprising the amino acid sequence of SEQ ID NO: 257, and a VL comprising the amino acid sequence of SEQ ID NO: 258.

In certain embodiments, an antibody described herein or an antigen binding fragment thereof comprises amino acid sequences with certain percent identity relative to any antibody provided herein, for example, those described in Section 7 below.

In some embodiments, the antibody provide herein contains substitutions (e.g., conservative substitutions), insertions, or deletions relative to the reference sequence, but the anti-GPC3 antibody comprising that sequence retains the ability to bind to GPC3. In some embodiments, a total of 1 to 10 amino acids have been substituted, inserted and/or deleted in a reference amino acid sequence. In some embodiments, substitutions, insertions, or deletions occur in regions outside the CDRs (i.e., in the FRs). Optionally, the anti-GPC3 antibody provided herein includes post-translational modifications of a reference sequence.

In some embodiments, the antibody or antigen binding fragment provided herein comprises a VH domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 257, and a VL domain having at least 75%, at least 80%, at least 85%, at least 90%, at least 91%, at least 92%, at least 93%, at least 94%, at least 95%, at least 96%, at least 97%, at least 98%, or at least 99% sequence identity to the amino acid sequence of SEQ ID NO: 258, and the antibody or antigen binding fragment binds to GPC3.

In some embodiments, functional epitopes can be mapped, e.g., by combinatorial alanine scanning, to identify amino acids in the GPC3 protein that are necessary for interaction with anti-GPC3 antibodies provided herein. In some embodiments, conformational and crystal structure of anti-GPC3 antibody bound to GPC3 may be employed to identify the epitopes. In some embodiments, the present disclosure provides an antibody that specifically binds to the same epitope as any of the anti-GPC3 antibodies provided herein. For example, in some embodiments, the antibody or antigen binding fragment provided herein binds to the same epitope as an anti-GPC3 antibody comprising a VH comprising the amino acid sequence of SEQ ID NO: 257, and a VL comprising the amino acid sequence of SEQ ID NO: 258.

In some embodiments, provided herein is an anti-GPC3 antibody, or antigen binding fragment thereof, that specifically binds to GPC3 competitively with any one of the anti-GPC3 antibodies described herein. In some embodiments, the antibody or antigen binding fragment provided herein specifically binds to GPC3 competitively with an anti-GPC3 antibody comprising a VH comprising the amino acid sequence of SEQ ID NO: 257, and a VL comprising the amino acid sequence of SEQ ID NO: 258.

In some embodiments, provided herein is a GPC3 binding protein comprising any one of the anti-GPC3 antibodies described above. In some embodiments, the GPC3 binding protein is a monoclonal antibody, including a mouse, chimeric, humanized or human antibody. In some embodiments, the anti-GPC3 antibody is an antibody fragment, e.g., a scFv. In some embodiments, the GPC3 binding protein is a fusion protein comprising the anti-GPC3 antibody provided herein. In other embodiments, the GPC3 binding protein is a multispecific antibody comprising the anti-GPC3 antibody provided herein. Other exemplary GPC3 binding molecules are described in more detail in the following sections.

In some embodiments, the anti-GPC3 antibody or antigen binding protein according to any of the above embodiments may incorporate any of the features, singly or in combination, as described below.

In some embodiments, the antibody described above comprises an Fc variant with enhanced ADCC. In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the antibody described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL 12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising a first chain comprising the amino acid sequence of SEQ ID NO: 289 and a second chain comprising the amino acid sequence of SEQ ID NO: 290.

### Exemplary Binding Molecules That Bind A NKG2D Ligand

In certain embodiments, the binding domain that binds to an HSC antigen itself is not an antibody or derived from an antibody. In some embodiments, the binding domain that binds to an HSC antigen is derived from a natural ligand or receptor that binds to a cell surface antigen on HSCs. For example, in some embodiments, the antigen expressed on HSCs is a NKG2D ligand, such as MICA, MICB, ULBP1, or ULBP2. In some embodiments, the binding domain in the present binding molecules comprises the NKG2D extracellular domain or a fragment or a variant thereof. In some embodiments, the binding domain comprises the amino acid sequence of SEQ ID NO: 89 or a fragment thereof. In some embodiments, the binding domain comprises an amino acid sequence at least 75%, 80%, 85%, 90%, 91%, 92%, 93%, 94%, 95%, 96%, 97%, 98%, or 99% identical to SEQ ID NO: 89.

In some embodiments, the binding domain is fused to an Fc variant with enhanced ADCC (directly or via a peptide linker). In some embodiments, the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering. In some embodiments, the Fc region comprises S239D/1332E, S239D/A330L/1332E, L235V/F243L/R292P/Y300L/P396L, or F243L/R292P/Y300L mutations. In some embodiments, the Fc region comprises S239D/1332E mutations. In some embodiments, the Fc region comprises S239D/A330L/1332E mutations. In some embodiments, the Fc region comprises L235V/F243L/R292P/Y300L/P396L mutations. In some embodiments, the Fc region comprises F243L/R292P/Y300L mutations.

In some embodiments, the binding molecule described above comprises an immune cell activating domain can activate NK cells. The immune activating domain can activate the immune cell by promoting immune cell activation signaling or suppresses immune cell inhibitory signaling.

The immune cell activating domain may bind to and/or modulate a receptor on an immune cell. Exemplary receptors include NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, and A2a.

In some specific embodiments, the immune cell activating domain binds to NKG2D. In some embodiments, the immune cell activating domain is derived from a NKG2D ligand. In some embodiments, the immune cell activating domain is the extracellular domain of MICA, MICB, ULBP1, or ULBP2 or a fragment or a variant thereof. In some embodiments, the immune cell activating domain is the extracellular domain of MICA or a fragment or a variant thereof. In some embodiments, the immune cell activating domain is the extracellular domain of MICB or a fragment or a variant thereof. In some embodiments, the immune cell activating domain is the extracellular domain of ULBP1 or a fragment or a variant thereof. In some embodiments, the immune cell activating domain is the extracellular domain of ULBP2 or a fragment or a variant thereof.

In some embodiments, the immune cell activating domain comprises an amino acid sequence of SEQ ID NO: 90 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 90. In some embodiments, the immune cell activating domain comprises an amino acid sequence of SEQ ID NO: 91 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 91. In some embodiments, the immune cell activating domain comprises an amino acid sequence of SEQ ID NO: 92 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 92. In some embodiments, the immune cell activating domain comprises an amino acid sequence of SEQ ID NO: 93 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 93.

In some embodiments, the immune cell activating domain binds to NKp46. In some embodiments, the immune cell activating domain comprises an antibody (incuding an antigen binding fragment) that binds to NKp46. In some embodiments, the antibody comprises an HCDR1, an HCDR2, and an HCDR3 as set forth in SEQ ID NO: 87, and a LCDR1, a LCDR2, and a LCDR3 as set forth in SEQ ID NO: 88. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66. In some embodiments, the antibody comprises a VH comprising the amino acid sequence of SEQ ID NO: 87 and a VL comrpsiing the amino acid sequence of SEQ ID NO: 88.

In some embodiments, the immune cell activating domain binds to TGFb. In some embodiments, the immune cell activating domain comprises TGFb receptor II extracellular domain (TRII) or a fragment or a variant thereof. In some embodiments, the immune cell activating domain comprises the amino acid sequence of SEQ ID NO: 94 or a fragment thereof. In some embodiments, the immune cell activating domain comprises an amino acid sequence at least 75%, 80%, 85%, 90%, 91%, 92%, 93%, 94%, 95%, 96%, 97%, 98%, or 99% identical to SEQ ID NO: 94.

In some embodiments, the immune cell activating domain binds to TIGIT. In some embodiments, the immune cell activating domain comprises an antibody (incuding an antigen binding fragment) that binds to TIGIT. In some embodiments, the antibody comprises an HCDR1, an HCDR2, and an HCDR3 as set forth in SEQ ID NO: 265, and a LCDR1, a LCDR2, and a LCDR3 as set forth in SEQ ID NO: 266. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272. In some embodiments, the antibody comprises a VH comprising the amino acid sequence of SEQ ID NO: 265 and a VL comrpsiing the amino acid sequence of SEQ ID NO: 266.

In some embodiments, the immune cell activating domain binds to PVRIG. In some embodiments, the immune cell activating domain comprises an antibody (incuding an antigen binding fragment) that binds to PVRIG. In some embodiments, the antibody comprises an HCDR1, an HCDR2, and an HCDR3 as set forth in SEQ ID NO: 273, and a LCDR1, a LCDR2, and a LCDR3 as set forth in SEQ ID NO: 274. CDR sequences can be determined according to well-known numbering systems or a combination thereof. In some embodiments, the CDRs are according to IMGT numbering. In some embodiments, the CDRs are according to Kabat numbering. In some embodiments, the CDRs are according to AbM numbering. In other embodiments, the CDRs are according to Chothia numbering. In other embodiments, the CDRs are according to Contact numbering. In some embodiments, the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280. In some embodiments, the antibody comprises a VH comprising the amino acid sequence of SEQ ID NO: 273 and a VL comrpsiing the amino acid sequence of SEQ ID NO: 274.

In some specific embodiments, the binding molecules provided herein are as shown in Table 4.

In a specific embodiment, provided herein is a binding molecule comprising the amino acid sequence of SEQ ID NO: 138. In a specific embodiment, provided herein is a binding molecule comprising the amino acid sequence of SEQ ID NO: 139. In a specific embodiment, provided herein is a binding molecule comprising the amino acid sequence of SEQ ID NO: 140. In a specific embodiment, provided herein is a binding molecule comprising the amino acid sequence of SEQ ID NO: 141. In a specific embodiment, provided herein is a binding molecule comprising the amino acid sequence of SEQ ID NO: 142.

In a specific embodiment, provided herein is a binding molecule comprising a first chain comprising the amino acid sequence of SEQ ID NO: 297, a second chain comprising the amino acid sequence of SEQ ID NO: 298, and a third chain comprising the amino acid sequence of SEQ ID NO: 299.

### Humanized Antibodies

The antibodies described herein include humanized antibodies. Humanized antibodies, such as the humanized antibodies disclosed herein can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, Molecular Immunology 28(4/5):489-498 (1991); Studnicka et al., Protein Engineering 7(6):805-814 (1994); and Roguska et al., PNAS 91:969-973 (1994)), chain shuffling (U.S. Pat. No. 5,565,332), and techniques disclosed in, e.g., U.S. Pat. Nos. 6,407,213, 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8): 1717-22 (1995), Sandhu JS, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., Nature 321:522-25 (1986); Riechmann et al., Nature 332:323-27 (1988); and Verhoeyen et al., Science 239:1534-36 (1988)), by substituting hypervariable region sequences for the corresponding sequences of a human antibody. In a specific embodiment, humanization of the antibody provided herein is performed as described in Section 6 below.

In some cases, the humanized antibodies are constructed by CDR grafting, in which the amino acid sequences of the CDRs of the parent non-human antibody are grafted onto a human antibody framework. For example, Padlan et al. determined that only about one third of the residues in the CDRs actually contact the antigen, and termed these the “specificity determining residues,” or SDRs (Padlan et al., FASEB J. 9:133-39 (1995)). In the technique of SDR grafting, only the SDR residues are grafted onto the human antibody framework (see, e.g., Kashmiri et al., Methods 36:25-34 (2005)).

The choice of human variable domains to be used in making the humanized antibodies can be important to reduce antigenicity. For example, according to the so-called “best-fit” method, the sequence of the variable domain of a non-human antibody is screened against the entire library of known human variable-domain sequences. The human sequence that is closest to that of the non-human antibody may be selected as the human framework for the humanized antibody (Sims et al., J. Immunol. 151:2296-308 (1993); and Chothia et al., J. Mol. Biol. 196:901-17 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA 89:4285-89 (1992); and Presta et al., J. Immunol. 151:2623-32 (1993)). In some cases, the framework is derived from the consensus sequences of the most abundant human subclasses, V_{L}6 subgroup I (V_{L}6I) and VH subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169:1119-25 (2002)).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13:819-24 (2002)), Modeller (Sali and Blundell, J. Mol. Biol. 234:779-815 (1993)), and Swiss PDB Viewer (Guex and Peitsch, Electrophoresis 18:2714-23 (1997)). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., Mol. Immunol. 44:1986-98 (2007)).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, Nat. Biotechnol. 23:1105-16 (2005); Dufner et al., Trends Biotechnol. 24:523-29 (2006); Feldhaus et al., Nat. Biotechnol. 21:163-70 (2003); and Schlapschy et al., Protein Eng. Des. Sel. 17:847-60 (2004)).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, J. Mol. Biol. 224:487-99 (1992)), or from the more limited set of target residues identified by Baca et al. J. Biol. Chem. 272: 10678-84 (1997).

In FR shuffling, whole FRs are combined with the non-human CDRs instead of creating combinatorial libraries of selected residue variants (see, e.g., Dall'Acqua et al., Methods 36:43-60 (2005)). A one-step FR shuffling process may be used. Such a process has been shown to be efficient, as the resulting antibodies exhibited improved biochemical and physicochemical properties including enhanced expression, increased affinity, and thermal stability (see, e.g., Damschroder et al., Mol. Immunol. 44:3049-60 (2007)).

The “humaneering” method is based on experimental identification of essential minimum specificity determinants (MSDs) and is based on sequential replacement of non-human fragments into libraries of human FRs and assessment of binding. This methodology typically results in epitope retention and identification of antibodies from multiple subclasses with distinct human V-segment CDRs.

The “human engineering” method involves altering a non-human antibody or antibody fragment by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., Protein Engineering 7:805-14 (1994); U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody.

A deimmunized antibody is an antibody in which T-cell epitopes have been removed. Methods for making deimmunized antibodies have been described. See, e.g., Jones et al., Methods Mol Biol. 525:405-23 (2009), xiv, and De Groot et al., Cell. Immunol. 244:148-153(2006)). Deimmunized antibodies comprise T-cell epitope-depleted variable regions and human constant regions. Briefly, variable regions of an antibody are cloned and T-cell epitopes are subsequently identified by testing overlapping peptides derived from the variable regions of the antibody in a T cell proliferation assay. T cell epitopes are identified via in silico methods to identify peptide binding to human MHC class II. Mutations are introduced in the variable regions to abrogate binding to human MHC class II. Mutated variable regions are then utilized to generate the deimmunized antibody.

### Antibody Variants

In some embodiments, amino acid sequence modification(s) of the antibodies described herein are contemplated. For example, it may be desirable to optimize the binding affinity and/or other biological properties of the antibody, including but not limited to specificity, thermostability, expression level, glycosylation, reduced immunogenicity, or solubility. Thus, in addition to the antibodies described herein, it is contemplated that variants of the antibodies described herein can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody.

In some embodiments, the antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, or conjugation to one or more immunoglobulin domains (e.g., Fc or a portion of an Fc). Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

When the antibody provided herein is fused to an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the binding molecules provided herein may be made in order to create variants with certain improved properties.

In other embodiments, when the antibody provided herein is fused to an Fc region, antibody variants provided herein may have a carbohydrate structure that lacks fucose attached (directly or indirectly) to said Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 and US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108; and WO 2004/056312, and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

The binding molecules comprising an antibody provided herein are further provided with bisected oligosaccharides, e.g., in which a biantennary oligosaccharide attached to the Fc region is bisected by GlcNAc. Such variants may have reduced fucosylation and/or improved ADCC function. Examples of such variants are described, e.g., in WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such variants may have improved CDC function. Such variants are described, e.g., in WO 1997/30087; WO 1998/58964; and WO 1999/22764.

Binding molecules with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260; 5,624,821;
and WO 94/29351 concerning other examples of Fc region variants.

In some embodiments, it may be desirable to create cysteine engineered antibodies, in which one or more residues of an antibody are substituted with cysteine residues. In some embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein.

Variations may be a substitution, deletion, or insertion of one or more codons encoding the antibody or polypeptide that results in a change in the amino acid sequence as compared with the original antibody or polypeptide. Sites of interest for substitutional mutagenesis include the CDRs and FRs.

Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the parental antibodies.

Amino acid sequence insertions include amino- and/or carboxyl-terminal fusions ranging in length from one residue to polypeptides containing multiple residues, as well as intrasequence insertions of single or multiple amino acid residues. Examples of terminal insertions include an antibody with an N-terminal methionyl residue.

Antibodies generated by conservative amino acid substitutions are included in the present disclosure. In a conservative amino acid substitution, an amino acid residue is replaced with an amino acid residue having a side chain with a similar charge. As described above, families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined. Conservative (e.g., within an amino acid group with similar properties and/or side chains) substitutions may be made, so as to maintain or not significantly change the properties. Exemplary substitutions are shown in the table below.

| Original | Exemplary | Original | Exemplary |
|---|---|---|---|
| Residue | Substitutions | Residue | Substitutions |
| Ala (A) | Val; Leu; Ile | Leu (L) | Norleucine; Ile; Val; |
| | | | Met; Ala; Phe |
| Arg (R) | Lys; Gln; Asn | Lys (K) | Arg; Gln; Asn |
| Asn (N) | Gln; His; Asp, Lys; | Met (M) | Leu; Phe; Ile |
| | Arg | | |
| Asp (D) | Glu; Asn | Phe (F) | Trp; Leu; Val; Ile; Ala; |
| | | | Tyr |
| Cys (C) | Ser; Ala | Pro (P) | Ala |
| Gln (Q) | Asn; Glu | Ser (S) | Thr |
| Glu (E) | Asp; Gln | Thr (T) | Val; Ser |
| Gly (G) | Ala | Trp (W) | Tyr; Phe |
| His (H) | Asn; Gln; Lys; Arg | Tyr (Y) | Trp; Phe; Thr; Ser |
| Ile (I) | Leu; Val; Met; Ala; | Val (V) | Ile; Leu; Met; Phe; Ala; |
| | Phe; Norleucine | | Norleucine |

Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. For example, any cysteine residue not involved in maintaining the proper conformation of the antibody also may be substituted, for example, with another amino acid, such as alanine or serine, to improve the oxidative stability of the molecule and to prevent aberrant crosslinking. Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant antibody or fragment thereof being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. More detailed description regarding affinity maturation is provided in the section below.

In some embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some embodiments of the variant antibody sequences provided herein, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, Science, 244:1081-1085 (1989). In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

The variations can be made using methods known in the art such as oligonucleotide-mediated (site-directed) mutagenesis, alanine scanning, and PCR mutagenesis. Site-directed mutagenesis (see, e.g., Carter, Biochem J. 237:1-7 (1986); and Zoller et al., Nucl. Acids Res. 10:6487-500 (1982)), cassette mutagenesis (see, e.g., Wells et al., Gene 34:315-23 (1985)), or other known techniques can be performed on the cloned DNA to produce the antibody variant DNA.

In vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, Methods. Mol. Biol. 178:1-37 (2002); and Bradbury and Marks, J. Immunol. Methods 290:29-49 (2004).

In a yeast display system (see, e.g., Boder et al., Nat. Biotech. 15:553-57 (1997); and Chao et al., Nat. Protocols 1:755-68 (2006)), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the single chain antibody (e.g., scFv). Expression has been shown to correlate with the stability of the displayed protein, and thus antibodies can be selected for improved stability as well as affinity (see, e.g., Shusta et al., J. Mol. Biol. 292:949-56 (1999)). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 1014 in size (see, e.g., U.S. Pat. Publication 2003/0186374; and Blaise et al., Gene 342:211-18 (2004)).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., Nucleic Acids Res. 34:e127 (2006)). In mRNA display, a covalent bond between antibody and mRNA is established using puromycin as an adaptor molecule (Wilson et al., Proc. Natl. Acad. Sci. USA 98:3750-55 (2001)).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In some embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., J. Biol. Chem. 280:607-17 (2005)) or residues suspected of affecting affinity on experimental basis or structural reasons. Diversity may also be introduced by replacement of regions that are naturally diverse via DNA shuffling or similar techniques (see, e.g., Lu et al., J. Biol. Chem. 278:43496-507 (2003); U.S. Pat. Nos. 5,565,332 and 6,989,250). Alternative techniques target hypervariable loops extending into framework-region residues (see, e.g., Bond et al., J. Mol. Biol. 348:699-709 (2005)) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, antibodies can be immobilized onto solid supports, columns, pins, or cellulose/poly (vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, Nature Biotechnology 23:1105-16 (2005); Quiroz and Sinclair, Revista Ingeneria Biomedia 4:39-51 (2010); and references therein.

### Modifications of Antibodies

Covalent modifications of antibodies are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide as described above (see, e.g., Beck et al., Curr. Pharm. Biotechnol. 9:482-501 (2008); and Walsh, Drug Discov. Today 15:773-80 (2010)), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301, 144; 4,670,417; 4,791,192; or 4,179,337. The antibody of the disclosure may also be genetically fused or conjugated to one or more immunoglobulin constant regions or portions thereof (e.g., Fc) to extend half-life and/or to impart known Fc-mediated effector functions.

The antibody of the present disclosure may also be modified to form chimeric molecules comprising the antibody fused to another, heterologous polypeptide or amino acid sequence, for example, an epitope tag (see, e.g., Terpe, Appl. Microbiol. Biotechnol. 60:523-33 (2003)) or the Fc region of an IgG molecule (see, e.g., Aruffo, Antibody Fusion Proteins 221-42 (Chamow and Ashkenazi eds., 1999)).

Also provided herein are fusion proteins comprising the antibody of the disclosure and a heterologous polypeptide. In some embodiments, the heterologous polypeptide to which the antibody is genetically fused or chemically conjugated is useful for targeting the antibody to cells having cell surface-expressed the antigen.

### Other Binding Molecules Comprising the Antibodies

In another aspect, provided herein is a binding molecule comprising an antibody provided herein genetically fused or chemically conjugated to another agent. Exemplary binding molecules of the present disclosure are described herein.

### Fusion Protein

In various embodiments, the antibody provided herein can be genetically fused or chemically conjugated to another agent, for example, protein-based entities. The antibody may be chemically-conjugated to the agent, or otherwise non-covalently conjugated to the agent. The agent can be a peptide or antibody (or a fragment thereof).

Thus, in some embodiments, provided herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, about 100, about 150, about 200, about 250, about 300, about 350, about 400, about 450 or about 500 amino acids, or over 500 amino acids) to generate fusion proteins, as well as uses thereof. In particular, provided herein are fusion proteins comprising an antigen binding fragment of the antibody provided herein (e.g., CDR1, CDR2, and/or CDR3) and a heterologous protein, polypeptide, or peptide.

Moreover, antibodies provided herein can be fused to marker or “tag” sequences, such as a peptide, to facilitate purification. In specific embodiments, the marker or tag amino acid sequence is a hexa-histidine peptide, hemagglutinin (“HA”) tag, and “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., Immunol. Rev. 62:119-58 (1982); U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-39 (1991); Traunecker et al., Nature, 331:84-86 (1988); Zheng et al., J. Immunol. 154:5590-600 (1995); and Vil et al., Proc. Natl. Acad. Sci. USA 89:11337-41 (1992)).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., Curr. Opinion Biotechnol. 8:724-33 (1997); Harayama, Trends Biotechnol. 16(2):76-82 (1998); Hansson et al., J. Mol. Biol. 287:265-76 (1999); and Lorenzo and Blasco, Biotechniques 24(2):308-13 (1998)). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

In some embodiments, an antibody provided herein is conjugated to a second antibody to form an antibody heteroconjugate.

In various embodiments, the antibody is genetically fused to the agent. Genetic fusion may be accomplished by placing a linker (e.g., a polypeptide) between the antibody and the agent. The linker may be a flexible linker.

In various embodiments, the antibody is genetically conjugated to a therapeutic molecule, with a hinge region linking the antibody to the therapeutic molecule.

Also provided herein are methods for making the various fusion proteins provided herein. The various methods described in Section 5.4 may also be utilized to make the fusion proteins provided herein.

In a specific embodiment, the fusion protein provided herein is recombinantly expressed. Recombinant expression of a fusion protein provided herein may require construction of an expression vector containing a polynucleotide that encodes the protein or a fragment thereof. Once a polynucleotide encoding a protein provided herein or a fragment thereof has been obtained, the vector for the production of the molecule may be produced by recombinant DNA technology using techniques well-known in the art. Thus, methods for preparing a protein by expressing a polynucleotide containing an encoding nucleotide sequence are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding a fusion protein provided herein, or a fragment thereof, or a CDR, operably linked to a promoter.

The expression vector can be transferred to a host cell by conventional techniques and the transfected cells are then cultured by conventional techniques to produce a fusion protein provided herein. Thus, also provided herein are host cells containing a polynucleotide encoding a fusion protein provided herein or fragments thereof operably linked to a heterologous promoter.

A variety of host-expression vector systems may be utilized to express the fusion protein provided herein. Such host-expression systems represent vehicles by which the coding sequences of interest may be produced and subsequently purified, but also represent cells which may, when transformed or transfected with the appropriate nucleotide coding sequences, express a fusion protein provided herein in situ. These include but are not limited to microorganisms such as bacteria (e.g., E. coli and B. subtilis) transformed with recombinant bacteriophage DNA, plasmid DNA or cosmid DNA expression vectors containing coding sequences; yeast (e.g., Saccharomyces Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antibody molecule, can be used for the expression of a recombinant fusion protein. For example, mammalian cells such as Chinese hamster ovary cells (CHO), in conjunction with a vector such as the major intermediate early gene promoter element from human cytomegalovirus is an effective expression system for antibodies or variants thereof. In a specific embodiment, the expression of nucleotide sequences encoding the fusion proteins provided herein is regulated by a constitutive promoter, inducible promoter or tissue specific promoter.

In bacterial systems, a number of expression vectors may be advantageously selected depending upon the use intended for the fusion protein being expressed. For example, when a large quantity of such a fusion protein is to be produced, for the generation of pharmaceutical compositions of a fusion protein, vectors which direct the expression of high levels of fusion protein products that are readily purified may be desirable. Such vectors include, but are not limited to, the E. coli expression vector pUR278 (Ruther et al., EMBO 12:1791 (1983)), in which the coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region El or E3) will result in a recombinant virus that is viable and capable of expressing the fusion protein in infected hosts (e.g., see Logan & Shenk, Proc. Natl. Acad. Sci. USA 8 1:355-359 (1984)). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NSO (a murine myeloma cell line that does not endogenously produce any immunoglobulin chains), CRL7030 and HsS78Bst cells.

For long-term, high-yield production of recombinant proteins, stable expression can be utilized. For example, cell lines which stably express the fusion proteins may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the fusion protein. Such engineered cell lines may be particularly useful in screening and evaluation of compositions that interact directly or indirectly with the binding molecule.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8-17 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIB TECH 11(5):155-2 15 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., J. Mol. Biol. 150:1 (1981), which are incorporated by reference herein in their entireties.

The expression level of a fusion protein can be increased by vector amplification (for a review, see Bebbington and Hentschel. The use of vectors based on gene amplification for the expression of cloned genes in mammalian cells in DNA cloning, Vol. 3 (Academic Press, New York, 1987)). When a marker in the vector system expressing a fusion protein is amplifiable, increase in the level of inhibitor present in culture of host cell will increase the number of copies of the marker gene. Since the amplified region is associated with the fusion protein gene, production of the fusion protein will also increase (Crouse et al., Mol. Cell. Biol. 3:257 (1983)).

The host cell may be co-transfected with multiple expression vectors provided herein. The vectors may contain identical selectable markers which enable equal expression of respective encoding polypeptides. Alternatively, a single vector may be used which encodes, and is capable of expressing multiple polypeptides. The coding sequences may comprise cDNA or genomic DNA.

Once a fusion protein provided herein has been produced by recombinant expression, it may be purified by any method known in the art for purification of a polypeptide (e.g., an immunoglobulin molecule), for example, by chromatography (e.g., ion exchange, affinity, particularly by affinity for the specific antigen after Protein A, sizing column chromatography, and Kappa select affinity chromatography), centrifugation, differential solubility, or by any other standard technique for the purification of proteins. Further, the fusion protein molecules provided herein can be fused to heterologous polypeptide sequences described herein or otherwise known in the art to facilitate purification.

### Immunoconjugates

In some embodiments, the present disclosure also provides immunoconjugates comprising any of the antibodies described herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica Charantia inhibitor, curcin, crotin, sapaonaria Officinalis inhibitor, gelonin, mitogellin, restrictocin, phenomycin, enomycin, and the tricothecenes.

In some embodiments, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², p³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

The immunuoconjugates or ADCs herein contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).)

In other embodiments, antibodies provided herein are conjugated or recombinantly fused, e.g., to a diagnostic molecule. Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, 225Acγ-emitting, Auger-emitting, β-emitting, an alpha-emitting or positron-emitting radioactive isotope.

### 5.3. Polynucleotides

In certain embodiments, the disclosure provides polynucleotides that encode the present binding molecules (e.g., antibodies) described herein. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is in the form of cDNA. In some embodiments, the polynucleotide is a synthetic polynucleotide.

The present disclosure further relates to variants of the polynucleotides described herein, wherein the variant encodes, for example, fragments, analogs, and/or derivatives of the binding molecules of the disclosure. In certain embodiments, the present disclosure provides a polynucleotide comprising a polynucleotide having a nucleotide sequence at least about 75% identical, at least about 80% identical, at least about 85% identical, at least about 90% identical, at least about 95% identical, and in some embodiments, at least about 96%, 97%, 98% or 99% identical to a polynucleotide encoding the binding molecule of the disclosure. As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

Also provided are vectors comprising the nucleic acid molecules described herein. In an embodiment, the nucleic acid molecules can be incorporated into a recombinant expression vector. The present disclosure provides recombinant expression vectors comprising any of the nucleic acids of the disclosure. As used herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole; however, parts of the vectors can be naturally-occurring. The described recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. The non-naturally occurring or altered nucleotides or internucleotide linkages do not hinder the transcription or replication of the vector.

In an embodiment, the recombinant expression vector of the disclosure can be any suitable recombinant expression vector, and can be used to transform or transfect any suitable host. Suitable vectors include those designed for propagation and expansion or for expression or both, such as plasmids and viruses. The vector can be selected from the group consisting of the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as AGT10, AGT11, AEMBL4, and ANM1149, AZapII (Stratagene) can be used. Examples of plant expression vectors include pBI01, pBI01.2, pBI121, pBI101.3, and pBIN19 (Clontech). Examples of animal expression vectors include pEUK-C1, pMAM, and pMAMneo (Clontech). The recombinant expression vector may be a viral vector, e.g., a retroviral vector, e.g., a gamma retroviral vector.

In an embodiment, the recombinant expression vectors are prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from Co1E1 SV40, 2μ plasmid, 2, bovine papilloma virus, and the like.

The recombinant expression vector may comprise regulatory sequences, such as transcription and translation initiation and termination codons, which are specific to the type of host (e.g., bacterium, plant, fungus, or animal) into which the vector is to be introduced, as appropriate, and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the described expression vectors include, for instance, neomycin/G418 resistance genes, histidinol x resistance genes, histidinol resistance genes, tetracycline resistance genes, and ampicillin resistance genes.

The recombinant expression vector can comprise a native or normative promoter operably linked to the nucleotide sequence of the disclosure. The selection of promoters, e.g., strong, weak, tissue-specific, inducible and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an RSV promoter, an SV40 promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art and include, for example, the Herpes Simplex Virus (HSV) thymidine kinase (TK) gene, cytosine deaminase, purine nucleoside phosphorylase, and nitroreductase.

In certain embodiments, a polynucleotide is isolated. In certain embodiments, a polynucleotide is substantially pure.

Also provided are host cells comprising the nucleic acid molecules described herein. The host cell may be any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, Saos, PC12, SP2/0 (American Type Culture Collection (ATCC), Manassas, VA, CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATCC CRL-TIB-196). Other useful cell lines include those derived from Chinese Hamster Ovary (CHO) cells such as CHO-KISV (Lonza Biologics, Walkersville, MD), CHO-K1 (ATCC CRL-61) or DG44.

### 5.4. Preparation of Binding Molecules and Method of Making

Methods of preparing binding molecules (such as antibodies) have been described. See, e.g., Els Pardon et al, Nature Protocol, 9(3): 674 (2014). Antibodies (such as scFv fragments) may be obtained using methods known in the art such as by immunizing a Camelid species (such as camel or llama) and obtaining hybridomas therefrom, or by cloning a library of antibodies using molecular biology techniques known in the art and subsequent selection by ELISA with individual clones of unselected libraries or by using phage display.

Antibodies provided herein may be produced by culturing cells transformed or transfected with a vector containing an antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells or B cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Pluckthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (A1-Rubeai ed., 2011); and Therapeutic Monoclonal Antibodies: From Bench to Clinic (An ed., 2009).

It is, of course, contemplated that alternative methods, which are well known in the art, may be employed to prepare binding moleucles (such as antibodies). For instance, the appropriate amino acid sequence, or portions thereof, may be produced by direct peptide synthesis using solid-phase techniques (see, e.g., Stewart et al., Solid-Phase Peptide Synthesis (1969); and Merrifield, J. Am. Chem. Soc. 85:2149-54 (1963)). In vitro protein synthesis may be performed using manual techniques or by automation. Various portions of the antibody may be chemically synthesized separately and combined using chemical or enzymatic methods to produce the desired antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R1 are independently lower alkyl groups. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

For example, the animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to fourteen days later, the animals are bled and the serum is assayed for antibody titer. Animals are boosted until the titer plateaus. Conjugates also can be made in recombinant cell culture as protein fusions. Also, aggregating agents such as alum are suitable to enhance the immune response.

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al., Nature, 256:495 (1975), or may be made by recombinant DNA methods (U.S. Pat. No. 4,816,567).

In the hybridoma method, an appropriate host animal is immunized to elicit lymphocytes that produce or are capable of producing antibodies that will specifically bind the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986).

The immunizing agent will typically include the antigenic protein or a fusion variant thereof. Goding, Monoclonal Antibodies: Principles and Practice, Academic Press (1986), pp. 59-103. Immortalized cell lines are usually transformed mammalian cells. The hybridoma cells thus prepared are seeded and grown in a suitable culture medium that preferably contains one or more substances that inhibit the growth or survival of the unfused, parental myeloma cells. Preferred immortalized myeloma cells are those that fuse efficiently, support stable high-level production of antibody by the selected antibody-producing cells, and are sensitive to a medium such as HAT medium.

Culture medium in which hybridoma cells are growing is assayed for production of monoclonal antibodies directed against the antigen. The culture medium in which the hybridoma cells are cultured can be assayed for the presence of monoclonal antibodies directed against the desired antigen. Such techniques and assays are known in the in art. For example, binding affinity may be determined by the Scatchard analysis of Munson et al., Anal. Biochem., 107:220 (1980).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Monoclonal antibodies may also be made by recombinant DNA methods, such as those described in U.S. Pat. No. 4,816,567, and as described above. DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, in order to synthesize monoclonal antibodies in such recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991). Subsequent publications describe the production of high affinity (nM range) human antibodies by chain shuffling (Marks et al., Bio Technology, 10:779-783 (1992)), as well as combinatorial infection and in vivo recombination as a strategy for constructing very large phage libraries (Waterhouse et al., Nucl. Acids Res., 21:2265-2266 (1993)). Thus, these techniques are viable alternatives to traditional monoclonal antibody hybridoma techniques for isolation of monoclonal antibodies.

The DNA also may be modified, for example, by substituting the coding sequence (U.S. Pat. No. 4,816,567; Morrison, et al., Proc. Natl Acad. Sci. USA, 81:6851 (1984)), or by covalently joining to the coding sequence all or part of the coding sequence for a non-immunoglobulin polypeptide. Such non-immunoglobulin polypeptides can be substituted to create a chimeric bivalent antibody comprising one antigen-combining site having specificity for an antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Recombinant Production in Prokaryotic Cells

Polynucleic acid sequences encoding the binding molecules (such as antibodies) of the present disclosure can be obtained using standard recombinant techniques. Desired polynucleic acid sequences may be isolated and sequenced from antibody producing cells such as hybridoma cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in prokaryotic hosts. Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but are not limited to, an origin of replication, a selection marker gene, a promoter, a ribosome binding site (RBS), a signal sequence, the heterologous nucleic acid insert and a transcription termination sequence.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is typically transformed using pBR322, a plasmid derived from an E. coli species. Examples of pBR322 derivatives used for expression of particular antibodies are described in detail in Carter et al., U.S. Pat. No. 5,648,237.

In addition, phage vectors containing replicon and control sequences that are compatible with the host microorganism can be used as transforming vectors in connection with these hosts. For example, bacteriophage such as GEM™-11 may be utilized in making a recombinant vector which can be used to transform susceptible host cells such as E. coli LE392.

The expression vector of the present application may comprise two or more promoter-cistron pairs, encoding each of the polypeptide components. A promoter is an untranslated regulatory sequence located upstream (5′) to a cistron that modulates its expression. Prokaryotic promoters typically fall into two classes, inducible and constitutive. Inducible promoter is a promoter that initiates increased levels of transcription of the cistron under its control in response to changes in the culture condition, e.g. the presence or absence of a nutrient or a change in temperature.

A large number of promoters recognized by a variety of potential host cells are well known. The selected promoter can be operably linked to cistron DNA encoding the present antibody by removing the promoter from the source DNA via restriction enzyme digestion and inserting the isolated promoter sequence into the vector of the present application. Both the native promoter sequence and many heterologous promoters may be used to direct amplification and/or expression of the target genes. In some embodiments, heterologous promoters are utilized, as they generally permit greater transcription and higher yields of expressed target gene as compared to the native target polypeptide promoter.

Promoters suitable for use with prokaryotic hosts include the PhoA promoter, the-galactamase and lactose promoter systems, a tryptophan (trp) promoter system and hybrid promoters such as the tac or the trc promoter. However, other promoters that are functional in bacteria (such as other known bacterial or phage promoters) are suitable as well. Their nucleic acid sequences have been published, thereby enabling a skilled worker operably to ligate them to cistrons encoding the target peptide (Siebenlist et al. Cell 20: 269 (1980)) using linkers or adaptors to supply any required restriction sites.

In one aspect, each cistron within the recombinant vector comprises a secretion signal sequence component that directs translocation of the expressed polypeptides across a membrane. In general, the signal sequence may be a component of the vector, or it may be a part of the target polypeptide DNA that is inserted into the vector. The signal sequence selected for the purpose of this invention should be one that is recognized and processed (i.e. cleaved by a signal peptidase) by the host cell. For prokaryotic host cells that do not recognize and process the signal sequences native to the heterologous polypeptides, the signal sequence can be substituted by a prokaryotic signal sequence selected, for example, from the group consisting of the alkaline phosphatase, penicillinase, Ipp, or heat-stable enterotoxin II (STII) leaders, LamB, PhoE, PelB, OmpA and MBP.

In some embodiments, the production of the antibodies according to the present disclosure can occur in the cytoplasm of the host cell, and therefore does not require the presence of secretion signal sequences within each cistron. Certain host strains (e.g., the E. coli trxB” strains) provide cytoplasm conditions that are favorable for disulfide bond formation, thereby permitting proper folding and assembly of expressed protein subunits.

Prokaryotic host cells suitable for expressing the antibodies of the present disclosure include Archaebacteria and Eubacteria, such as Gram-negative or Gram-positive organisms. Examples of useful bacteria include Escherichia (e.g., E. coli), Bacilli (e.g., B. subtilis), Enterobacteria, Pseudomonas species (e.g., P. aeruginosa), Salmonella typhimurium, Serratia marcescans, Klebsiella, Proteus, Shigella, Rhizobia, Vitreoscilla, or Paracoccus. In some embodiments, gram-negative cells are used. In one embodiment, E. coli cells are used as hosts. Examples of E. coli strains include strain W3110 (Bachmann, Cellular and Molecular Biology, vol. 2 (Washington, D.C.: American Society for Microbiology, 1987), pp. 1190-1219; ATCC Deposit No. 27,325) and derivatives thereof, including strain 33D3 having genotype W3110 AfhuA (AtonA) ptr3 lac Iq lacL8 AompT A(nmpc-fepE) degP41 kanR (U.S. Pat. No. 5,639,635). Other strains and derivatives thereof, such as E. coli 294 (ATCC 31,446), E. coli B, E. coli 1776 (ATCC 31,537) and E. coli RV308 (ATCC 31,608) are also suitable. These examples are illustrative rather than limiting. Methods for constructing derivatives of any of the above-mentioned bacteria having defined genotypes are known in the art and described in, for example, Bass et al., Proteins, 8:309-314 (1990). It is generally necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYC177, or pKN410 are used to supply the replicon.

Typically the host cell should secrete minimal amounts of proteolytic enzymes, and additional protease inhibitors may desirably be incorporated in the cell culture.

Host cells are transformed with the above-described expression vectors and cultured in conventional nutrient media modified as appropriate for inducing promoters, selecting transformants, or amplifying the genes encoding the desired sequences. Transformation means introducing DNA into the prokaryotic host so that the DNA is replicable, either as an extrachromosomal element or by chromosomal integrant. Depending on the host cell used, transformation is done using standard techniques appropriate to such cells. The calcium treatment employing calcium chloride is generally used for bacterial cells that contain substantial cell-wall barriers. Another method for transformation employs polyethylene glycol/DMSO. Yet another technique used is electroporation.

Prokaryotic cells used to produce the antibodies of the present application are grown in media known in the art and suitable for culture of the selected host cells. Examples of suitable media include luria broth (LB) plus necessary nutrient supplements. In some embodiments, the media also contains a selection agent, chosen based on the construction of the expression vector, to selectively permit growth of prokaryotic cells containing the expression vector. For example, ampicillin is added to media for growth of cells expressing ampicillin resistant gene.

Any necessary supplements besides carbon, nitrogen, and inorganic phosphate sources may also be included at appropriate concentrations introduced alone or as a mixture with another supplement or medium such as a complex nitrogen source. Optionally the culture medium may contain one or more reducing agents selected from the group consisting of glutathione, cysteine, cystamine, thioglycollate, dithioerythritol and dithiothreitol. The prokaryotic host cells are cultured at suitable temperatures and pHs.

If an inducible promoter is used in the expression vector of the present application, protein expression is induced under conditions suitable for the activation of the promoter. In one aspect of the present application, PhoA promoters are used for controlling transcription of the polypeptides. Accordingly, the transformed host cells are cultured in a phosphate-limiting medium for induction. Preferably, the phosphate-limiting medium is the C.R.A.P medium (see, e.g., Simmons et al., J. Immunol. Methods 263: 133-147 (2002)). A variety of other inducers may be used, according to the vector construct employed, as is known in the art.

The expressed antibodies of the present disclosure are secreted into and recovered from the periplasm of the host cells. Protein recovery typically involves disrupting the microorganism, generally by such means as osmotic shock, sonication or lysis. Once cells are disrupted, cell debris or whole cells may be removed by centrifugation or filtration. The proteins may be further purified, for example, by affinity resin chromatography. Alternatively, proteins can be transported into the culture media and isolated therein. Cells may be removed from the culture and the culture supernatant being filtered and concentrated for further purification of the proteins produced. The expressed polypeptides can be further isolated and identified using commonly known methods such as polyacrylamide gel electrophoresis (PAGE) and Western blot assay.

Alternatively, protein production is conducted in large quantity by a fermentation process. Various large-scale fed-batch fermentation procedures are available for production of recombinant proteins. To improve the production yield and quality of the antibodies of the present disclosure, various fermentation conditions can be modified. For example, the chaperone proteins have been demonstrated to facilitate the proper folding and solubility of heterologous proteins produced in bacterial host cells. Chen et al. J Bio Chem 274:19601-19605 (1999); U.S. Pat. Nos. 6,083,715; 6,027,888; Bothmann and Pluckthun, J. Biol. Chem. 275:17100-17105 (2000); Ramm and Pluckthun, J. Biol. Chem. 275:17106-17113 (2000); Arie et al., Mol. Microbiol. 39:199-210 (2001).

To minimize proteolysis of expressed heterologous proteins (especially those that are proteolytically sensitive), certain host strains deficient for proteolytic enzymes can be used for the present invention, as described in, for example, U.S. Pat. Nos. 5,264,365; 5,508,192; Hara et al., Microbial Drug Resistance, 2:63-72 (1996). E. coli strains deficient for proteolytic enzymes and transformed with plasmids overexpressing one or more chaperone proteins may be used as host cells in the expression system encoding the antibodies of the present application.

The antibodies produced herein can be further purified to obtain preparations that are substantially homogeneous for further assays and uses. Standard protein purification methods known in the art can be employed. The following procedures are exemplary of suitable purification procedures: fractionation on immunoaffinity or ion-exchange columns, ethanol precipitation, reverse phase HPLC, chromatography on silica or on a cation-exchange resin such as DEAE, chromatofocusing, SDS-PAGE, ammonium sulfate precipitation, and gel filtration using, for example, Sephadex G-75. Protein A immobilized on a solid phase for example can be used in some embodiments for immunoaffinity purification of binding molecules of the present disclosure. The solid phase to which Protein A is immobilized is preferably a column comprising a glass or silica surface, more preferably a controlled pore glass column or a silicic acid column. In some embodiments, the column has been coated with a reagent, such as glycerol, in an attempt to prevent nonspecific adherence of contaminants. The solid phase is then washed to remove contaminants non-specifically bound to the solid phase. Finally the antibodies of interest is recovered from the solid phase by elution.

### Recombinant Production in Eukaryotic Cells

For eukaryotic expression, the vector components generally include, but are not limited to, one or more of the following, a signal sequence, an origin of replication, one or more marker genes, and enhancer element, a promoter, and a transcription termination sequence.

A vector for use in a eukaryotic host may also an insert that encodes a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available. The DNA for such precursor region can be ligated in reading frame to DNA encoding the antibodies of the present application.

Generally, the origin of replication component is not needed for mammalian expression vectors (the SV40 origin may typically be used only because it contains the early promoter).

Expression and cloning vectors may contain a selection gene, also termed a selectable marker. Selection genes may encode proteins that confer resistance to antibiotics or other toxins, e.g., ampicillin, neomycin, methotrexate, or tetracycline; complement auxotrophic deficiencies; or supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up nucleic acid encoding the antibodies of the present application. For example, cells transformed with the DHFR selection gene are first identified by culturing all of the transformants in a culture medium that contains methotrexate (Mtx), a competitive antagonist of DHFR. An exemplary appropriate host cell when wild-type DHFR is employed is the Chinese hamster ovary (CHO) cell line deficient in DHFR activity. Alternatively, host cells (particularly wild-type hosts that contain endogenous DHFR) transformed or co-transformed with the polypeptide encoding-DNA sequences, wild-type DHFR protein, and another selectable marker such as aminoglycoside 3′-phosphotransferase (APH) can be selected by cell growth in medium containing a selection agent for the selectable marker such as an aminoglycosidic antibiotic.

Expression and cloning vectors usually contain a promoter that is recognized by the host organism and is operably linked to the nucleic acid encoding the desired polypeptide sequences. Eukaryotic genes have an AT-rich region located approximately 25 to 30 based upstream from the site where transcription is initiated. Another sequence found 70 to 80 bases upstream from the start of the transcription of many genes may be included. The 3′ end of most eukaryotic may be the signal for addition of the poly A tail to the 3′ end of the coding sequence. All of these sequences may be inserted into eukaryotic expression vectors.

Polypeptide transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

Transcription of a DNA encoding the antibodies of the present disclosure by higher eukaryotes is often increased by inserting an enhancer sequence into the vector. Many enhancer sequences are now known from mammalian genes (globin, elastase, albumin, α-fetoprotein, and insulin). Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the polypeptide encoding sequence, but is preferably located at a site 5′ from the promoter.

Expression vectors used in eukaryotic host cells (yeast, fungi, insect, plant, animal, human, or nucleated cells from other multicellular organisms) also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the polypeptide-encoding mRNA. One useful transcription termination component is the bovine growth hormone polyadenylation region.

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CVI line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells can be transformed with the above-described expression or cloning vectors for antibodies production and cultured in conventional nutrient media modified as appropriate for inducing promoters, selecting transformants, or amplifying the genes encoding the desired sequences.

The host cells used to produce the antibodies of the present application may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

When using recombinant techniques, the antibodies can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein composition prepared from the cells can be purified using, for example, hydroxylapatite chromatography, gel electrophoresis, dialysis, and affinity chromatography, with affinity chromatography being the preferred purification technique. The matrix to which the affinity ligand is attached is most often agarose, but other matrices are available. Mechanically stable matrices such as controlled pore glass or poly (styrene-divinyl) benzene allow for faster flow rates and shorter processing times than can be achieved with agarose. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered. Following any preliminary purification step(s), the mixture comprising the antibody of interest and contaminants may be subjected to low pH hydrophobic interaction chromatography.

5.5. Pharmaceutical Compositions

In one aspect, the present disclosure further provides pharmaceutical compositions comprising at least one binding molecule (e.g., an antibody) of the present disclosure. In some embodiments, a pharmaceutical composition comprises therapeutically effective amount of a binding molecule provided herein and a pharmaceutically acceptable excipient.

Pharmaceutical compositions comprising a binding molecule are prepared for storage by mixing the binding molecules having the desired degree of purity with optional physiologically acceptable excipients (see, e.g., Remington, Remington's Pharmaceutical Sciences (18^{th} ed. 1980)) in the form of aqueous solutions or lyophilized or other dried forms.

The binding molecule of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

A binding molecule provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with a binding molecule as described herein, including, but not limited to, encapsulation in liposomes, microparticles, microcapsules, recombinant cells capable of expressing the antibody or antigen binding fragment thereof, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228: 190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6 Methods of Use

In one aspect, provided herein is a method of depleting HSCs, e.g., in a subject, comprising exposing the cells to an effective amount of a binding molecule (such as an antibody) provided herein. In some embodiments, the method provided herein is for depleting activated HSCs.

In one aspect, provided herein is a use of the binding molecule (such as an antibody) provided herein for depleting HSCs, e.g., in a subject. In some embodiments, the binding molecule is used for depleting activated HSCs.

In another aspect, provided herein is a method of treating a disease or disorder in a subject comprising administering to the subject an effective amount of a binding molecule (such as an antibody or antigen binding fragment thereof) provided herein. In one embodiment, the disease or disorder is an activated HSC related disease or disorder. In one embodiment, the disease or disorder is liver fibrosis.

In another aspect, provided herein is the use of a binding molecule (such as antibody or antigen binding fragment thereof) provided herein in the manufacture of a medicament for treating a disease or disorder in a subject. In one embodiment, the disease or disorder is an activated HSC related disease or disorder. In one embodiment, the disease or disorder is liver fibrosis.

In another aspect, provided herein is the use of a pharmaceutical composition provided herein in the manufacture of a medicament for treating a disease or disorder in a subject. In one embodiment, the disease or disorder is an activated HSC related disease or disorder. In one embodiment, the disease or disorder is liver fibrosis.

In one aspect, the binding molecule is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus macaque monkey, or a human). In one embodiment, the subject is a human. In another embodiment, the subject is a human with a disease or disorder.

Various delivery systems are known and can be used to administer a prophylactic or therapeutic agent (e.g., a binding molecule provided herein), including, but not limited to, encapsulation in liposomes, microparticles, microcapsules, recombinant cells capable of expressing the binding molecule, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., a binding molecule provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., a binding molecule provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering a binding molecule provided herein, care must be taken to use materials to which the binding molecule such as an antibody or antigen binding fragment thereof does not absorb.

In another embodiment, a prophylactic or therapeutic agent, or a composition provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system.

In a specific embodiment, where the composition provided herein is a nucleic acid encoding a prophylactic or therapeutic agent (e.g., a binding molecule provided herein), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition provided herein comprises one, two or more binding molecules provided herein. In another embodiment, a composition provided herein comprises one, two or more binding molecules provided herein and a prophylactic or therapeutic agent other than a binding molecule provided herein. In one embodiment, the agents are known to be useful for or have been or are currently used for the prevention, management, treatment and/or amelioration of a disease or disorder. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., a binding molecule provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the binding molecule provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

A binding molecule provided herein can be packaged in a hermetically sealed container such as an ampoule or sachette indicating the quantity of binding molecule. In one embodiment, the binding moleculeis supplied as a dry sterilized lyophilized powder or water free concentrate in a hermetically sealed container and can be reconstituted, e.g., with water or saline to the appropriate concentration for administration to a subject. The lyophilized binding moleculecan be stored at between 2 and 8° C. in its original container and the binding moleculecan be administered within 12 hours, such as within 6 hours, within 5 hours, within 3 hours, or within 1 hour after being reconstituted. In an alternative embodiment, a binding molecule provided herein is supplied in liquid form in a hermetically sealed container indicating the quantity and concentration of the antibody.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., a binding molecule provided herein), or a composition provided herein that will be effective in the prevention and/or treatment of a disease or disorder can be determined by standard clinical techniques. In addition, in vitro assays may optionally be employed to help identify optimal dosage ranges. The precise dose to be employed in the formulation will also depend on the route of administration, and the seriousness of a disease or disorder, and should be decided according to the judgment of the practitioner and each patient's circumstances.

Effective doses may be extrapolated from dose-response curves derived from in vitro or animal model test systems.

In certain embodiments, the route of administration for a dose of a binding molecule provided herein to a patient is intranasal, intramuscular, intravenous, subcutaneous, or a combination thereof, but other routes described herein are also acceptable. Each dose may or may not be administered by an identical route of administration. In some embodiments, a binding molecule provided herein may be administered via multiple routes of administration simultaneously or subsequently to other doses of the same or a different binding molecule provided herein.

In certain embodiments, the binding molecule provided herein are administered prophylactically or therapeutically to a subject. The binding molecule provided herein can be prophylactically or therapeutically administered to a subject so as to prevent, lessen or ameliorate a disease or symptom thereof.

### 5.7. Gene Therapy and Cellular Therapy

In a specific embodiment, nucleic acids comprising sequences encoding binding molecules or functional derivatives thereof, are administered to a subject for use in a method provided herein, for example, to prevent, manage, treat and/or ameliorate an activated HSC related disease or disorder, by way of gene therapy. Such therapy encompasses that performed by the administration to a subject of an expressed or expressible nucleic acid. In an embodiment, the nucleic acids produce their encoded antibody, and the antibody mediates a prophylactic or therapeutic effect.

Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5): 155-215. Methods commonly known in the art of recombinant DNA technology which can be used are described in Ausubel et al. (eds.), Current Protocols in Molecular Biology, John Wiley & Sons, N Y (1993); and Kriegler, Gene Transfer and Expression, A Laboratory Manual, Stockton Press, NY (1990).

In a specific embodiment, a composition comprises nucleic acids encoding a binding molecule (such as an antibody) provided herein, the nucleic acids being part of an expression vector that expresses the binding molecule (such as the antibody or chimeric proteins or heavy or light chains thereof) in a suitable host. In particular, such nucleic acids have promoters, such as heterologous promoters, operably linked to the coding region, the promoter being inducible or constitutive, and, optionally, tissue-specific. In another particular embodiment, nucleic acid molecules are used in which the coding sequences and any other desired sequences are flanked by regions that promote homologous recombination at a desired site in the genome, thus providing for intrachromosomal expression of the encoding nucleic acids (Koller and Smithies, 1989, Proc. Natl. Acad. Sci. USA 86:8932-8935; Zijlstra et al., 1989, Nature 342:435-438).

Delivery of the nucleic acids into a subject can be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding a peptide are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding a binding molecule to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDRI gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of binding molecules (such as antibodies). Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68: 143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an antibody as provided herein. In certain embodiments, the AAV comprises a nucleic acid encoding a VH domain. In other embodiments, the AAV comprises a nucleic acid encoding a V_{L} domain. In certain embodiments, the AAV comprises a nucleic acid encoding a VH domain and a V_{L} domain. In some embodiments of the methods provided herein, a subject is administered an AAV comprising a nucleic acid encoding a VH domain and an AAV comprising a nucleic acid encoding a V_{L} domain. In other embodiments, a subject is administered an AAV comprising a nucleic acid encoding a VH domain and a V_{L} domain. In certain embodiments, the VH and a V_{L} domains are over-expressed.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic acid to the cell, so that the nucleic acid is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for gene therapy is autologous to the subject.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

In another aspect, the antibody or antigen binding fragment thereof provided herein can be part of an engineered cell surface receptor such as a chimeric antigen receptor (CAR). Typically, a CAR comprises an extraceullar domain, a transmembrane domain, and an intracellular signaling domain.

In some embodiments, provided herein is a CAR comprising an extracelluar domain that comprises one or more antibody or fragment thereof provided herein.

The CARs of the present disclosure comprise a transmembrane domain that can be directly or indirectly fused to the extracellular antigen binding domain. The transmembrane domain may be derived either from a natural or from a synthetic source. As used herein, a “transmembrane domain” refers to any protein structure that is thermodynamically stable in a cell membrane, preferably an eukaryotic cell membrane. Transmembrane domains compatible for use in the CARs described herein may be obtained from a naturally occurring protein. Alternatively, it can be a synthetic, non-naturally occurring protein segment, e.g., a hydrophobic protein segment that is thermodynamically stable in a cell membrane. Transmembrane domains are classified based on the three dimensional structure of the transmembrane domain. For example, transmembrane domains may form an alpha helix, a complex of more than one alpha helix, a beta-barrel, or any other stable structure capable of spanning the phospholipid bilayer of a cell. Furthermore, transmembrane domains may also or alternatively be classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). Membrane proteins may be defined as Type I, Type II or Type III depending upon the topology of their termini and membrane-passing segment(s) relative to the inside and outside of the cell. Type I membrane proteins have a single membrane-spanning region and are oriented such that the N-terminus of the protein is present on the extracellular side of the lipid bilayer of the cell and the C-terminus of the protein is present on the cytoplasmic side. Type II membrane proteins also have a single membrane-spanning region but are oriented such that the C-terminus of the protein is present on the extracellular side of the lipid bilayer of the cell and the N-terminus of the protein is present on the cytoplasmic side. Type III membrane proteins have multiple membrane-spanning segments and may be further sub-classified based on the number of transmembrane segments and the location of N- and C-termini. In some embodiments, the transmembrane domain of the CAR described herein is derived from a Type I single-pass membrane protein. In some embodiments, transmembrane domains from multi-pass membrane proteins may also be compatible for use in the CARs described herein. Multi-pass membrane proteins may comprise a complex (at least 2, 3, 4, 5, 6, 7 or more) alpha helices or a beta sheet structure. In some embodiments, the N-terminus and the C-terminus of a multi-pass membrane protein are present on opposing sides of the lipid bilayer, e.g., the N-terminus of the protein is present on the cytoplasmic side of the lipid bilayer and the C-terminus of the protein is present on the extracellular side. In some embodiments, the transmembrane domain of the CAR comprises a transmembrane domain chosen from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 1a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CDI 1b, ITGAX, CDI Ic, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. In some embodiments, the transmembrane domain is derived from a molecule selected from the group consisting of CD8a, CD4, CD28, CD137, CD80, CD86, CD152 and PD1.

Transmembrane domains for use in the CARs described herein can also comprise at least a portion of a synthetic, non-naturally occurring protein segment. In some embodiments, the transmembrane domain is a synthetic, non-naturally occurring alpha helix or beta sheet. In some embodiments, the protein segment is at least approximately 20 amino acids, e.g., at least 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776, the relevant disclosures of which are incorporated by reference herein.

The transmembrane domain provided herein may comprise a transmembrane region and a cytoplasmic region located at the C-terminal side of the transmembrane domain. The cytoplasmic region of the transmembrane domain may comprise three or more amino acids and, in some embodiments, helps to orient the transmembrane domain in the lipid bilayer. In some embodiments, one or more cysteine residues are present in the transmembrane region of the transmembrane domain. In some embodiments, one or more cysteine residues are present in the cytoplasmic region of the transmembrane domain. In some embodiments, the cytoplasmic region of the transmembrane domain comprises positively charged amino acids. In some embodiments, the cytoplasmic region of the transmembrane domain comprises the amino acids arginine, serine, and lysine.

In some embodiments, the transmembrane region of the transmembrane domain comprises hydrophobic amino acid residues. In some embodiments, the transmembrane domain of the CAR provided herein comprises an artificial hydrophobic sequence. For example, a triplet of phenylalanine, tryptophan and valine may be present at the C terminus of the transmembrane domain. In some embodiments, the transmembrane region comprises mostly hydrophobic amino acid residues, such as alanine, leucine, isoleucine, methionine, phenylalanine, tryptophan, or valine. In some embodiments, the transmembrane region is hydrophobic. In some embodiments, the transmembrane region comprises a poly-leucine-alanine sequence. The hydropathy, or hydrophobic or hydrophilic characteristics of a protein or protein segment, can be assessed by any method known in the art, for example the Kyte and Doolittle hydropathy analysis.

The CARs of the present disclosure comprise an intracellular signaling domain. The intracellular signaling domain is responsible for activation of at least one of the normal effector functions of the immune effector cell expressing the CARs. The term “effector function” refers to a specialized function of a cell. Effector function of a T cell, for example, may be cytolytic activity or helper activity including the secretion of cytokines. Thus the term “cytoplasmic signaling domain” refers to the portion of a protein which transduces the effector function signal and directs the cell to perform a specialized function. While usually the entire cytoplasmic signaling domain can be employed, in many cases it is not necessary to use the entire chain. To the extent that a truncated portion of the cytoplasmic signaling domain is used, such truncated portion may be used in place of the intact chain as long as it transduces the effector function signal. The term cytoplasmic signaling domain is thus meant to include any truncated portion of the cytoplasmic signaling domain sufficient to transduce the effector function signal.

In some embodiments, the intracellular signaling domain comprises a primary intracellular signaling domain of an immune effector cell. In some embodiments, the CAR comprises an intracellular signaling domain consisting essentially of a primary intracellular signaling domain of an immune effector cell. “Primary intracellular signaling domain” refers to cytoplasmic signaling sequence that acts in a stimulatory manner to induce immune effector functions. In some embodiments, the primary intracellular signaling domain contains a signaling motif known as immunoreceptor tyrosine-based activation motif, or ITAM. An “ITAM,” as used herein, is a conserved protein motif that is generally present in the tail portion of signaling molecules expressed in many immune cells. The motif may comprises two repeats of the amino acid sequence YxxL/I separated by 6-8 amino acids, wherein each x is independently any amino acid, producing the conserved motif YxxL/Ix(6-8)YxxL/I. ITAMs within signaling molecules are important for signal transduction within the cell, which is mediated at least in part by phosphorylation of tyrosine residues in the ITAM following activation of the signaling molecule. ITAMs may also function as docking sites for other proteins involved in signaling pathways. Exemplary ITAM-containing primary cytoplasmic signaling sequences include those derived from CD3ζ, FcR gamma (FCERIG), FcR beta (Fc Epsilon Rib), CD3 gamma, CD3 delta, CD3 epsilon, CD5, CD22, CD79a, CD79b, and CD66d. In some embodiments, the primary intracellular signaling domain is derived from CD3ζ.

Many immune effector cells require co-stimulation, in addition to stimulation of an antigen-specific signal, to promote cell proliferation, differentiation and survival, as well as to activate effector functions of the cell. In some embodiments, the CAR comprises at least one co-stimulatory signaling domain. The term “co-stimulatory signaling domain,” as used herein, refers to at least a portion of a protein that mediates signal transduction within a cell to induce an immune response such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils. “Co-stimulatory signaling domain” can be the cytoplasmic portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival.

In some embodiments, the intracellular signaling domain comprises a single co-stimulatory signaling domain. In some embodiments, the intracellular signaling domain comprises two or more (such as about any of 2, 3, 4, or more) co-stimulatory signaling domains. In some embodiments, the intracellular signaling domain comprises two or more of the same co-stimulatory signaling domains. In some embodiments, the intracellular signaling domain comprises two or more co-stimulatory signaling domains from different co-stimulatory proteins, such as any two or more co-stimulatory proteins described herein. In some embodiments, the intracellular signaling domain comprises a primary intracellular signaling domain (such as cytoplasmic signaling domain of CD3° C.) and one or more co-stimulatory signaling domains. In some embodiments, the one or more co-stimulatory signaling domains and the primary intracellular signaling domain (such as cytoplasmic signaling domain of CD35) are fused to each other via optional peptide linkers. The primary intracellular signaling domain, and the one or more co-stimulatory signaling domains may be arranged in any suitable order. In some embodiments, the one or more co-stimulatory signaling domains are located between the transmembrane domain and the primary intracellular signaling domain (such as cytoplasmic signaling domain of CD3° C.). Multiple co-stimulatory signaling domains may provide additive or synergistic stimulatory effects.

Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory molecule may be compatible for use in the CARs described herein. The type(s) of co-stimulatory signaling domain is selected based on factors such as the type of the immune effector cells in which the effector molecules would be expressed (e.g., T cells, NK cells, macrophages, neutrophils, or eosinophils) and the desired immune effector function (e.g., ADCC effect). Examples of co-stimulatory signaling domains for use in the CARs can be the cytoplasmic signaling domain of co-stimulatory proteins, including, without limitation, members of the B7/CD28 family (e.g., B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (e.g., 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNF-beta, OX40/TNFRSF4, OX40 Ligand/TNFSF4, RELT/TNFRSF19L, TACI/TNFRSF13B, TL1A/TNFSF15, TNF-alpha, and TNF RII/TNFRSF1B); members of the SLAM family (e.g., 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); and any other co-stimulatory molecules, such as CD2, CD7, CD53, CD82/Kai-1, CD90/Thyl, CD96, CD160, Claudin18.20, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. In some embodiments, the one or more co-stimulatory signaling domains are selected from the group consisting of CD27, CD28, CD137, OX40, CD30, CD40, CD3, lymphocyte function-associated antigen-1(LFA-1), CD2, CD7, LIGHT, NKG2C, B7-H3 and ligands that specially bind to CD83 (such as CD83 and MD-2). In some embodiments, the intracellular signaling domain in the CAR of the present disclosure comprises a co-stimulatory signaling domain derived from CD137 (i.e., 4-1BB). Also within the scope of the present disclosure are variants of any of the co-stimulatory signaling domains described herein, such that the co-stimulatory signaling domain is capable of modulating the immune response of the immune cell. In some embodiments, the co-stimulatory signaling domains comprises up to 10 amino acid residue variations (e.g., 1, 2, 3, 4, 5, or 8) as compared to a wild-type counterpart. Such co-stimulatory signaling domains comprising one or more amino acid variations may be referred to as variants. Mutation of amino acid residues of the co-stimulatory signaling domain may result in an increase in signaling transduction and enhanced stimulation of immune responses relative to co-stimulatory signaling domains that do not comprise the mutation. Mutation of amino acid residues of the co-stimulatory signaling domain may result in a decrease in signaling transduction and reduced stimulation of immune responses relative to co-stimulatory signaling domains that do not comprise the mutation.

The CARs of the present disclosure may comprise a hinge domain that is located between the extracellular antigen binding domain and the transmembrane domain. A hinge domain is an amino acid segment that is generally found between two domains of a protein and may allow for flexibility of the protein and movement of one or both of the domains relative to one another. Any amino acid sequence that provides such flexibility and movement of the extracellular antigen binding domain relative to the transmembrane domain of the effector molecule can be used. The hinge domain may contain about 10⁻¹⁰⁰ amino acids, e.g., about any one of 15-75 amino acids, 20-50 amino acids, or 30-60 amino acids. In some embodiments, the hinge domain may be at least about any one of 10, 11, 12, 13, 14, 15, 16, 17, 18, 19, 20, 21, 22, 23, 24, 25, 26, 27, 28, 29, 30, 35, 40, 45, 50, 55, 60, 65, 70, or 75 amino acids in length. In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is derived from CD8a. Hinge domains of antibodies, such as an IgG, IgA, IgM, IgE, or IgD antibodies, are also compatible for use in the pH-dependent chimeric receptor systems described herein. In some embodiments, the hinge domain is the hinge domain that joins the constant domains CHI and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody. Non-naturally occurring peptides may also be used as hinge domains for the chimeric receptors described herein. In some embodiments, the hinge domain between the C-terminus of the extracellular ligand-binding domain of an Fc receptor and the N-terminus of the transmembrane domain is a peptide linker, such as a (GxS)n linker, wherein x and n, independently can be an integer between 3 and 12, including 3, 4, 5, 6, 7, 8, 9, 10, 11, 12, or more.

The CARs of the present disclosure may comprise a signal peptide (also known as a signal sequence) at the N-terminus of the polypeptide. In general, signal peptides are peptide sequences that target a polypeptide to the desired site in a cell. In some embodiments, the signal peptide targets the effector molecule to the secretory pathway of the cell and will allow for integration and anchoring of the effector molecule into the lipid bilayer. Signal peptides including signal sequences of naturally occurring proteins or synthetic, non-naturally occurring signal sequences, which are compatible for use in the CARs described herein will be evident to one of skill in the art. In some embodiments, the signal peptide is derived from a molecule selected from the group consisting of CD8a, GM-CSF receptor «, and IgG1 heavy chain. In some embodiments, the signal peptide is derived from CD8a.

In yet another aspect, provided herein are host cells (such as immune effector cells) comprising any one of the CARs described herein and uses thereof, e.g., for treating a disease or disorder.

5.8. Diagnostic Assays and Methods

Labeled binding molecules such as labeled antibodies and derivatives and analogs thereof, which immunospecifically bind to an antigen can be used for diagnostic purposes to detect, diagnose, or monitor a disease.

Antibodies provided herein can be used to assay an antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (1251, 1211), carbon (14C), sulfur (35S), tritium (3H), indium (121In), and technetium (99Tc); luminescent labels, such as luminol; and fluorescent labels, such as fluorescein and rhodamine, and biotin.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antibody will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

Presence of the labeled molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 5.9. Kits

Also provided herein are kits comprising a binding molecule (e.g., an antibody) provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Also provided herein are panels of binding molecules (such as antibodies) that immunospecifically bind to an antigen. In specific embodiments, provided herein are panels of antibodies having different association rate constants different dissociation rate constants, different affinities for the antigen, and/or different specificities for an antigen. In certain embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96 well or 384 well plates, such as for assays such as ELISAs.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

### 6. Embodiments

The present disclosure includes the following non-limiting embodiments:

Embodiment 1. A binding molecule comprising one or more binding domain(s) that bind(s) to one or more antigen(s) expressed on an activated hepatic stellate cells (HSC), and a functional domain that is capable of enhancing an antibody effector function toward activated HSCs, wherein optionally the antibody effector function is antibody-dependent cell-mediated cytotoxicity (ADCC).

Embodiment 2. The binding molecule of embodiment 1, wherein the functional domain is:
(i) an Fc region comprising one or more mutation(s) that enhances ADCC; or
(ii) a domain that activates an immune cell, and wherein optionally the immune cell is a NK cell

Embodiment 3. The binding molecule of embodiment 2, wherein the functional domain is the Fc region comprising one or more mutation(s) that enhances ADCC, and wherein optionally the one or more mutation(s) of the Fc region is at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering.

Embodiment 4. The binding molecule of embodiment 3, wherein the Fc region comprises S239D/1332E, S239D/A330L/1332E, L235V/F243L/R292P/Y300L/P396L, or F243L/R292P/Y300L mutations.

Embodiment 5. The binding molecule of embodiment 2, wherein the functional domain is the domain that activates the immune cell, and wherein the functional domain promotes immune cell activation signaling or suppresses immune cell inhibitory signaling.

Embodiment 6. The binding molecule of embodiment 5, wherein the functional domain binds to and/or modulates a receptor on an immune cell, and wherein optionally the receptor is selected from a group consisting of NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRGI, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, TIGIT, PVRIG, and A2a.

Embodiment 7. The binding molecule of embodiment 5 or embodiment 6, wherein the functional domain binds to NKG2D.

Embodiment 8. The binding molecule of embodiment 7, wherein the functional domain is derived from a NKG2D ligand.

Embodiment 9. The binding molecule of embodiment 8, wherein the functional domain is the extracellular domain of MICA, MICB, ULBP1, or ULBP2 or a fragment or a variant thereof, and wherein optionally the functional domain comprises an amino acid sequence of any one of SEQ ID NO: 90-93 or a fragment thereof.

Embodiment 10. The binding molecule of embodiment 5 or embodiment 6, wherein the functional domain binds to NKp46.

Embodiment 11. The binding molecule of embodiment 10, wherein the functional domain comprises an antibody that binds to NKp46.

Embodiment 12. The binding molecule of embodiment 11, wherein the antibody comprises an HCDR1, an HCDR2, and an HCDR3 as set forth in SEQ ID NO: 87, and a LCDR1, a LCDR2, and a LCDR3 as set forth in SEQ ID NO: 88.

Embodiment 13. The binding molecule of embodiment 12, wherein the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66.

Embodiment 14. The binding molecule of embodiment 12 or embodiment 13, wherein the antibody comprises a VH comprising the amino acid sequence of SEQ ID NO: 87 and a V_{L} comrpsiing the amino acid sequence of SEQ ID NO: 88.

Embodiment 15. The binding molecule of embodiment 5, wherein the functional domain binds to TGFb.

Embodiment 16. The binding molecule of embodiment 15, wherein the functional domain comprises TGFb receptor II extracellular domain (TRII) or a fragment or a variant thereof, and wherein optionally the functional domain comprises the amino acid sequence of SEQ ID NO: 94 or a fragment thereof.

Embodiment 17. The binding molecule of embodiment 5 or embodiment 6, wherein the functional domain binds to TIGIT.

Embodiment 18. The binding molecule of embodiment 17, wherein the antibody comprises an HCDR1, an HCDR2, and an HCDR3 as set forth in SEQ ID NO: 265, and a LCDR1, a LCDR2, and a LCDR3 as set forth in SEQ ID NO: 266.

Embodiment 19. The binding molecule of embodiment 18, wherein the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272.

Embodiment 20. The binding molecule of embodiment 18 or embodiment 19, wherein the antibody comprises a VH comprising the amino acid sequence of SEQ ID NO: 265 and a V_{L} comrpsiing the amino acid sequence of SEQ ID NO: 266.

Embodiment 21. The binding molecule of embodiment 5 or embodiment 6, wherein the functional domain binds to PVRIG.

Embodiment 22. The binding molecule of embodiment 21, wherein the antibody comprises an HCDR1, an HCDR2, and an HCDR3 as set forth in SEQ ID NO: 273, and a LCDR1, a LCDR2, and a LCDR3 as set forth in SEQ ID NO: 274.

Embodiment 23. The binding molecule of embodiment 22, wherein the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280.

Embodiment 24. The binding molecule of embodiment 21 or embodiment 22, wherein the antibody comprises a VH comprising the amino acid sequence of SEQ ID NO: 273 and a V_{L} comrpsiing the amino acid sequence of SEQ ID NO: 274.

Embodiment 25. The binding molecule of any one of embodiments 5 to 24, wherein the binding molecule further comprises an Fc region comprising one or more mutation(s) that enhances ADCC.

Embodiment 26. The binding molecule of embodiment 25, wherein the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering.

Embodiment 27. The binding molecule of embodiment 26, wherein the Fc region comprises S239D/1332E, S239D/A330L/1332E, L235V/F243L/R292P/Y300L/P396L, or F243L/R292P/Y300L mutations.

Embodiment 28. The binding molecule of any one of embodiments 1 to 27, wherein the antigen(s) expressed on the HSC is selected from a group consisting of 5HT1B, 5HTIF, 5HT2A, 5-HT2B, 5-HT7, A2a, A2b, a1b1 integrin, a2bi integrin, a5b1 integrin, a6b4 integrin, a8b1 integrin, avb1 integrin, avb3 integrin, ACVR2A, ACVR2B, AdipoR1, AdipoR2, ADRA1A, ADRA1B, ANTXR1, AT1, AT2, BAMB1, BMPR2, C5aR, CB1, CB2, CCR1, CCR2, CCR5, CCR7, CD105, CD112, CD14, CD146, CD155, CD248, CD36, CD38, CD40, CD44, CD49e, CD62e, CD73, CD95, c-MET, CNTFR, CXCR3, CXCR4, DDR1, DDR2, EGFR, ETA, ETB, FAP, FGFR2, FN, gp130, GPC3, GPR91, ICAM-1, IGF-1R, IGF-2R, IL-10R2, IL-1IRA, IL-17RA, IL-20R1, IL-20R2, IL-22R1, IL-6R, KCNE4, ITGA8, LRP, MICA, MICB, NCAM, NGFR, NPR-B, NPR3, OB-Ra, OB-Rb, OPRD1, P2X4, P2X7, P2Y6, p75NTR, PAFR, PARI PAR2 PAR4, PDGFRA, PDGFRB, PD-L1, PD-L2, Ptc, PTH-1R, RAGE, SIRPA, CD47, SYP, TGFBR1, TGFBR2, TGFBR3, TLR2, TLR3, TLR4, TLR7, TLR9, TNFR1, TRKB, TRKC, ULBP1, ULPB2, uPAR, VACM-1, VEGFR-1, and VEGFR-2.

Embodiment 29. The binding molecule of any one of embodiments 1 to 28, wherein the antigen is PDGFRb.

Embodiment 30. The binding molecule of embodiment 29, wherein the binding domain comprises an HCDR1, an HCDR2, and an HCDR3 as set forth in SEQ ID NO: 67, and a LCDR1, a LCDR2, and a LCDR3 as set forth in SEQ ID NO: 68.

Embodiment 31. The binding molecule of embodiment 30, wherein the HCDR1 comprises the amino acid sequence of SEQ ID NO: 1, the HCDR2 comprises the amino acid sequence of SEQ ID NO: 2, the HCDR3 comprises the amino acid sequence of SEQ ID NO: 3, the LCDR1 comprises the amino acid sequence of SEQ ID NO: 4, the LCDR2 comprises the amino acid sequence of SEQ ID NO: 5, and the LCDR3 comprises the amino acid sequence of SEQ ID NO: 6.

Embodiment 32. The binding molecule of embodiment 30 or embodiment 31, wherein the binding domain comprises a VH comprising the amino acid sequence of SEQ ID NO: 67 and a V_{L} comrpsiing the amino acid sequence of SEQ ID NO: 68.

Embodiment 33. The binding molecule of any one of embodiments 1 to 28, wherein the antigen is SIRPA.

Embodiment 34. The binding molecule of embodiment 33, wherein the binding domain comprises
(i) an HCDR1, an HCDR2, and an HCDR3 as set forth in SEQ ID NO: 69, and a LCDR1, a LCDR2, and a LCDR3 as set forth in SEQ ID NO: 70;
(ii) an HCDR1, an HCDR2, and an HCDR3 as set forth in SEQ ID NO: 71, and a LCDR1, a LCDR2, and a LCDR3 as set forth in SEQ ID NO: 72; or
(iii) an HCDR1, an HCDR2, and an HCDR3 as set forth in SEQ ID NO: 73, and a LCDR1, a LCDR2, and a LCDR3 as set forth in SEQ ID NO: 74.

Embodiment 35. The binding molecule of embodiment 34, wherein:

(i) the HCDR1 comprises the amino acid sequence of SEQ ID NO: 7, the HCDR2 comprises the amino acid sequence of SEQ ID NO: 8, the HCDR3 comprises the amino acid sequence of SEQ ID NO: 9, the LCDR1 comprises the amino acid sequence of SEQ ID NO: 10, the LCDR2 comprises the amino acid sequence of SEQ ID NO: 11, and the LCDR3 comprises the amino acid sequence of SEQ ID NO: 12;
(ii) the HCDR1 comprises the amino acid sequence of SEQ ID NO: 13, the HCDR2 comprises the amino acid sequence of SEQ ID NO: 14, the HCDR3 comprises the amino acid sequence of SEQ ID NO: 15, the LCDR1 comprises the amino acid sequence of SEQ ID NO: 16, the LCDR2 comprises the amino acid sequence of SEQ ID NO: 17, and the LCDR3 comprises the amino acid sequence of SEQ ID NO: 18; or
(iii) the HCDR1 comprises the amino acid sequence of SEQ ID NO: 19, the HCDR2 comprises the amino acid sequence of SEQ ID NO: 20, the HCDR3 comprises the amino acid sequence of SEQ ID NO: 21, the LCDR1 comprises the amino acid sequence of SEQ ID NO: 22, the LCDR2 comprises the amino acid sequence of SEQ ID NO: 23, and the LCDR3 comprises the amino acid sequence of SEQ ID NO: 24.

Embodiment 36. The binding molecule of embodiment 34 or embodiment 35, wherein the binding domain comprises:
(i) a VH comprising the amino acid sequence of SEQ ID NO: 69 and a V_{L} comrpsiing the amino acid sequence of SEQ ID NO: 70;
(ii) a VH comprising the amino acid sequence of SEQ ID NO: 71 and a V_{L} comrpsiing the amino acid sequence of SEQ ID NO: 72; or
(iii) a VH comprising the amino acid sequence of SEQ ID NO: 73 and a V_{L} comrpsiing the amino acid sequence of SEQ ID NO: 74.

Embodiment 37. The binding molecule of any one of embodiments 1 to 28, wherein the antigen is FAPα.

Embodiment 38. The binding molecule of embodiment 37, wherein the binding domain comprises
(i) an HCDR1, an HCDR2, and an HCDR3 as set forth in SEQ ID NO: 75, and a LCDR1, a LCDR2, and a LCDR3 as set forth in SEQ ID NO: 76; or
(ii) an HCDR1, an HCDR2, and an HCDR3 as set forth in SEQ ID NO: 77, and a LCDR1, a LCDR2, and a LCDR3 as set forth in SEQ ID NO: 78.

Embodiment 39. The binding molecule of embodiment 38, wherein:
(i) the HCDR1 comprises the amino acid sequence of SEQ ID NO: 25, the HCDR2 comprises the amino acid sequence of SEQ ID NO: 26, the HCDR3 comprises the amino acid sequence of SEQ ID NO: 27, the LCDR1 comprises the amino acid sequence of SEQ ID NO: 28, the LCDR2 comprises the amino acid sequence of SEQ ID NO: 29, and the LCDR3 comprises the amino acid sequence of SEQ ID NO: 30; or
(ii) the HCDR1 comprises the amino acid sequence of SEQ ID NO: 31, the HCDR2 comprises the amino acid sequence of SEQ ID NO: 32, the HCDR3 comprises the amino acid sequence of SEQ ID NO: 33, the LCDR1 comprises the amino acid sequence of SEQ ID NO: 34, the LCDR2 comprises the amino acid sequence of SEQ ID NO: 35, and the LCDR3 comprises the amino acid sequence of SEQ ID NO: 36.

Embodiment 40. The binding molecule of embodiment 38 or embodiment 39, wherein the binding domain comprises:
(i) a VH comprising the amino acid sequence of SEQ ID NO: 75 and a V_{L} comrpsiing the amino acid sequence of SEQ ID NO: 76; or
(ii) a VH comprising the amino acid sequence of SEQ ID NO: 77 and a V_{L} comrpsiing the amino acid sequence of SEQ ID NO: 78.

Embodiment 41. The binding molecule of any one of embodiments 1 to 28, wherein the antigen is PD-L1.

Embodiment 42. The binding molecule of embodiment 41, wherein the binding domain comprises an HCDR1, an HCDR2, and an HCDR3 as set forth in SEQ ID NO: 79, and a LCDR1, a LCDR2, and a LCDR3 as set forth in SEQ ID NO: 80.

Embodiment 43. The binding molecule of embodiment 42, wherein the HCDR1 comprises the amino acid sequence of SEQ ID NO: 37, the HCDR2 comprises the amino acid sequence of SEQ ID NO: 38, the HCDR3 comprises the amino acid sequence of SEQ ID NO: 39, the LCDR1 comprises the amino acid sequence of SEQ ID NO: 40, the LCDR2 comprises the amino acid sequence of SEQ ID NO: 41, and the LCDR3 comprises the amino acid sequence of SEQ ID NO: 42.

Embodiment 44. The binding molecule of embodiment 42 or embodiment 43, wherein the binding domain comprises a VH comprising the amino acid sequence of SEQ ID NO: 79 and a V_{L} comrpsiing the amino acid sequence of SEQ ID NO: 80.

Embodiment 45. The binding molecule of any one of embodiments 1 to 28, wherein the antigen is uPAR.

Embodiment 46. The binding molecule of embodiment 45, wherein the binding domain comprises an HCDR1, an HCDR2, and an HCDR3 as set forth in SEQ ID NO: 81, and a LCDR1, a LCDR2, and a LCDR3 as set forth in SEQ ID NO: 82.

Embodiment 47. The binding molecule of embodiment 46, wherein the HCDR1 comprises the amino acid sequence of SEQ ID NO: 43, the HCDR2 comprises the amino acid sequence of SEQ ID NO: 44, the HCDR3 comprises the amino acid sequence of SEQ ID NO: 45, the LCDR1 comprises the amino acid sequence of SEQ ID NO: 46, the LCDR2 comprises the amino acid sequence of SEQ ID NO: 47, and the LCDR3 comprises the amino acid sequence of SEQ ID NO: 48.

Embodiment 48. The binding molecule of embodiment 46 or embodiment 47, wherein the binding domain comprises a VH comprising the amino acid sequence of SEQ ID NO: 81 and a V_{L} comrpsiing the amino acid sequence of SEQ ID NO: 82.

Embodiment 49. The binding molecule of any one of embodiments 1 to 28, wherein the antigen is IGF-IR.

Embodiment 50. The binding molecule of embodiment 49, wherein the binding domain comprises:
(i) an HCDR1, an HCDR2, and an HCDR3 as set forth in SEQ ID NO: 83, and a LCDR1, a LCDR2, and a LCDR3 as set forth in SEQ ID NO: 84; or
(ii) an HCDR1, an HCDR2, and an HCDR3 as set forth in SEQ ID NO: 85, and a LCDR1, a LCDR2, and a LCDR3 as set forth in SEQ ID NO: 86.

Embodiment 51. The binding molecule of embodiment 50, wherein:
(i) the HCDR1 comprises the amino acid sequence of SEQ ID NO: 49, the HCDR2 comprises the amino acid sequence of SEQ ID NO: 50, the HCDR3 comprises the amino acid sequence of SEQ ID NO: 51, the LCDR1 comprises the amino acid sequence of SEQ ID NO: 52, the LCDR2 comprises the amino acid sequence of SEQ ID NO: 53, and the LCDR3 comprises the amino acid sequence of SEQ ID NO: 54; or
(ii) the HCDR1 comprises the amino acid sequence of SEQ ID NO: 55, the HCDR2 comprises the amino acid sequence of SEQ ID NO: 56, the HCDR3 comprises the amino acid sequence of SEQ ID NO: 57, the LCDR1 comprises the amino acid sequence of SEQ ID NO: 58, the LCDR2 comprises the amino acid sequence of SEQ ID NO: 59, and the LCDR3 comprises the amino acid sequence of SEQ ID NO: 60.

Embodiment 52. The binding molecule of embodiment 50 or embodiment 51, wherein the binding domain comprises:
(i) a VH comprising the amino acid sequence of SEQ ID NO: 83 and a V_{L} comrpsiing the amino acid sequence of SEQ ID NO: 84; or
(ii) a VH comprising the amino acid sequence of SEQ ID NO: 85 and a V_{L} comrpsiing the amino acid sequence of SEQ ID NO: 86.

Embodiment 53. The binding molecule of any one of embodiments 1 to 28, wherein the antigen is a NKG2D ligand, and wherein optionally the NKG2D ligand is MICA, MICB, ULBP1, or ULBP2.

Embodiment 54. The binding molecule of embodiment 53, wherein the binding domain comprises the NKG2D extracellular domain or a fragment or a variant thereof, and wherein optionally the binding domain comprises the amino acid sequence of SEQ ID NO: 89 or a fragment thereof.

Embodiment 55. The binding molecule of any one of embodiments 1 to 28, wherein the antigen is ANTXR1.

Embodiment 56. The binding molecule of embodiment 55, wherein the binding domain comprises:
(i) an HCDR1, an HCDR2, and an HCDR3 as set forth in SEQ ID NO: 225, and a LCDR1, a LCDR2, and a LCDR3 as set forth in SEQ ID NO: 226; or
(ii) an HCDR1, an HCDR2, and an HCDR3 as set forth in SEQ ID NO: 233, and a LCDR1, a LCDR2, and a LCDR3 as set forth in SEQ ID NO: 234.

Embodiment 57. The binding molecule of embodiment 56, wherein:

(i) the HCDR1 comprises the amino acid sequence of SEQ ID NO: 227, the HCDR2 comprises the amino acid sequence of SEQ ID NO: 228, the HCDR3 comprises the amino acid sequence of SEQ ID NO: 229, the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 232; or
(ii) the HCDR1 comprises the amino acid sequence of SEQ ID NO: 235, the HCDR2 comprises the amino acid sequence of SEQ ID NO: 236, the HCDR3 comprises the amino acid sequence of SEQ ID NO: 237, the LCDR1 comprises the amino acid sequence of SEQ ID NO: 238, the LCDR2 comprises the amino acid sequence of SEQ ID NO: 239, and the LCDR3 comprises the amino acid sequence of SEQ ID NO: 240.

Embodiment 58. The binding molecule of embodiment 56 or embodiment 57, wherein the binding domain comprises:
(i) a VH comprising the amino acid sequence of SEQ ID NO: 225 and a V_{L} comrpsiing the amino acid sequence of SEQ ID NO: 226; or
(ii) a VH comprising the amino acid sequence of SEQ ID NO: 233 and a V_{L} comrpsiing the amino acid sequence of SEQ ID NO: 232.

Embodiment 59. The binding molecule of any one of embodiments 1 to 28, wherein the antigen is CD248.

Embodiment 60. The binding molecule of embodiment 59, wherein the binding domain comprises:
(i) an HCDR1, an HCDR2, and an HCDR3 as set forth in SEQ ID NO: 241, and a LCDR1, a LCDR2, and a LCDR3 as set forth in SEQ ID NO: 242; or
(ii) an HCDR1, an HCDR2, and an HCDR3 as set forth in SEQ ID NO: 249, and a LCDR1, a LCDR2, and a LCDR3 as set forth in SEQ ID NO: 250.

Embodiment 61. The binding molecule of embodiment 60, wherein:

(i) the HCDR1 comprises the amino acid sequence of SEQ ID NO: 243, the HCDR2 comprises the amino acid sequence of SEQ ID NO: 244, the HCDR3 comprises the amino acid sequence of SEQ ID NO: 245, the LCDR1 comprises the amino acid sequence of SEQ ID NO: 246, the LCDR2 comprises the amino acid sequence of SEQ ID NO: 247, and the LCDR3 comprises the amino acid sequence of SEQ ID NO: 248; or
(ii) the HCDR1 comprises the amino acid sequence of SEQ ID NO: 251, the HCDR2 comprises the amino acid sequence of SEQ ID NO: 252, the HCDR3 comprises the amino acid sequence of SEQ ID NO: 253, the LCDR1 comprises the amino acid sequence of SEQ ID NO: 254, the LCDR2 comprises the amino acid sequence of SEQ ID NO: 255, and the LCDR3 comprises the amino acid sequence of SEQ ID NO: 256.

Embodiment 62. The binding molecule of embodiment 60 or embodiment 61, wherein the binding domain comprises:
(i) a VH comprising the amino acid sequence of SEQ ID NO: 241 and a V_{L} comrpsiing the amino acid sequence of SEQ ID NO: 242; or
(ii) a VH comprising the amino acid sequence of SEQ ID NO: 249 and a V_{L} comrpsiing the amino acid sequence of SEQ ID NO: 250.

Embodiment 63. The binding molecule of any one of embodiments 1 to 28, wherein the antigen is GPC3.

Embodiment 64. The binding molecule of embodiment 63, wherein the binding domain comprises an HCDR1, an HCDR2, and an HCDR3 as set forth in SEQ ID NO: 257, and a LCDR1, a LCDR2, and a LCDR3 as set forth in SEQ ID NO: 258.

Embodiment 65. The binding molecule of embodiment 64, wherein the HCDR1 comprises the amino acid sequence of SEQ ID NO: 259, the HCDR2 comprises the amino acid sequence of SEQ ID NO: 260, the HCDR3 comprises the amino acid sequence of SEQ ID NO: 261, the LCDR1 comprises the amino acid sequence of SEQ ID NO: 262, the LCDR2 comprises the amino acid sequence of SEQ ID NO: 263, and the LCDR3 comprises the amino acid sequence of SEQ ID NO: 264.

Embodiment 66. The binding molecule of embodiment 64 or embodiment 65, wherein the binding domain comprises a VH comprising the amino acid sequence of SEQ ID NO: 257 and a V_{L} comrpsiing the amino acid sequence of SEQ ID NO: 258.

Embodiment 67. The binding molecule of any one of embodiments 1 to 66, wherein the binding molecule is an IgG antibody or a fusion protein comprising the IgG antibody.

Embodiment 68. The binding molecule of embodiment 67, wherein the antibody is a humanized antibody.

Embodiment 69. A nucleic acid molecule encoding the binding molecule of any one of embodiments 1 to 68 or a fragment thereof.

Embodiment 70. A vector comprising the nucleic acid molecule of embodiment 69.

Embodiment 71. A host cell transformed with the vector of embodiment 70.

Embodiment 72. A composition comprising a therapeutically effective amount of the binding molecule of any one of embodiments 1 to 68, the nucleic acid molecule of embodiment 69, or the vector of embodiment 70, and a pharmaceutically acceptable excipient.

Embodiment 73. A method of treating a disease or disorder in a subject, comprising administering to the subject the composition of embodiment 72.

Embodiment 74. The method of embodiment 73, wherein the disease or disorder is associated with activiated HSCs.

Embodiment 75. The method of embodiment 74, wherein the disease or disorder is liver fibrosis.

Embodiment 76. A method of depleting activated HSCs in a subject, comprising administering to the subject the composition of embodiment 72.

Embodiment 77. The method of embodiment 76, wherein the subject has a disease or disorder associated with activiated HSCs.

Embodiment 78. The method of embodiment 77, wherein the disease or disorder is liver fibrosis.

A number of embodiments of the invention have been described. Nevertheless, it will be understood that various modifications may be made without departing from the spirit and scope of the invention. Accordingly, the following examples are intended to illustrate but not limit the scope of invention described in the claims or otherwise in the present disclosure.

### 7. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 7.1. Example 1: Constructs for Targeting Activated Hepatic Stellate Cells (HSC)

Activated hepatic stellate cells (HSC) are the key type of cells mediating fibrogenesis upon liver injury. Therapeutic approach targeting activated HSCs is highly needed to slow or prevent fibrosis formation and improve quality of life in the patients with liver fibrosis or cirrhosis. However, successful strategies via depletion of activated HSC by antibodies have not been developed. In the present studies, antibodies binding to antigens expressed on activated HSC cells were generated and demonstrated to attract the immune system for eliminating these cells in the liver. By reducing the number of activated HSC upon liver injury, the antibodies could efficiently slow the progression of liver fibrosis.

Surface antigens with sufficient extracellular domain that could be recognized by an antibody could be targeted according to the present disclosure. Exemplary non-limiting surface antigens provided herein include: 5HT1B, 5HTIF, 5HT2A, 5-HT2B, 5-HT7, A2a, A2b, a1b1 integrin, a2bi integrin, a5b1 integrin, a6b4 integrin, a8b1 integrin, avb1 integrin, avb3 integrin, ACVR2A, ACVR2B, AdipoR1, AdipoR2, ADRA1A, ADRA1B, ANTXR1, AT1, AT2, BAMB1, BMPR2, C5aR, CB1, CB2, CCR1, CCR2, CCR5, CCR7, CD105, CD112, CD14, CD146, CD155, CD248, CD36, CD38, CD40, CD44, CD49e, CD62e, CD73, CD95, c-MET, CNTFR, CXCR3, CXCR4, DDR1, DDR2, EGFR, ETA, ETB, FAP, FGFR2, FN, gp130, GPC3, GPR91, ICAM-1, IGF-1R, IGF-2R, IL-10R2, IL-11RA, IL-17RA, IL-20R1, IL-20R2, IL-22R1, IL-6R, KCNE4, ITGA8, LRP, MICA, MICB, NCAM, NGFR, NPR-B, NPR3, OB-Ra, OB-Rb, OPRD1, P2X4, P2X7, P2Y6, p75NTR, PAFR, PARI PAR2 PAR4, PDGFRA, PDGFRB, PD-L1, PD-L2, Ptc, PTH-1R, RAGE, SIRPA, CD47, SYP, TGFBR1, TGFBR2, TGFBR3, TLR2, TLR3, TLR4, TLR7, TLR9, TNFR1, TRKB, TRKC, ULBP1, ULPB2, uPAR, VACM-1, VEGFR-1, and VEGFR-2.

A number of exemplary surface antigens on HSC cells were studied in the present examples. Exemplary antibodies (including Fc containing binding molecules) targeting known antigens expressed on activated HSC cells (including PDGFRb, FAPα, NKG2D ligands, uPAR, IGF1R, CD248) were constructed and tested. Additionally, three novel HSC-expressing surface proteins (ANTXR1, SIRPA and PD-L1) were identified and studied.

Anti-FAPa antibody, anti-CD248 antibody, anti-ANTXR1 antibody and anti-GPC3 was engineered as mIgG2a. Other testing antibodies were recombinantly engineered as hIgG1. NKG2D-Fc recombinant proteins were engineered by fusing two copies of NKG2D extracelluar domain to hIgG1 Fc. Chimeric anti-SIRPA antibodies with variable regions from murine antibody and constant region from hIgG1 were engineered and expressed. Humanized anti-PD-L1 antibody was expressed as hIgG1 isotype. The sequences of these exemplary constructs are shown in the tables below.

TABLE 1
VH, VL and CDR sequences of exemplary antibodies

| | VH | VL | HCDR1 | HCDR2 | HCDR3 | LCDR1 | LCDR2 | LCDR3 |
|---|---|---|---|---|---|---|---|---|
| Exemplary anti- | QVQLQESGPGLVKP | DIQMTQSPSSLSASV | NYAIN | IIWTGG | TGTRGY | HASQNI | KASNLH | QQGQSF |
| PDGFRb | SETLSLTCTVSGFSL | GDRVTITCHASQNIN | (SEQ ID | GTSYNS | FFDY | NVWLS | T (SEQ | PFT (SEQ |
| antibody | TNYAINWVRQPPGK | VWLSWYQQKPGKA | NO: 1) | ALKS | (SEQ ID | (SEQ ID | ID NO: 5) | ID NO: 6) |
| (α-PDGFRb 1) | GLEWLGIIWTGGGT | PKLLIYKASNLHTG | | (SEQ ID | NO: 3) | NO: 4) | | |
| | SYNSALKSRLTISKD | VPSRFSGSGSGTGFT | | NO: 2) | | | | |
| | TSKNQVSLKLSSVT | LTISSLQPEDIATYY | | | | | | |
| | AADTAVYYCARTG | CQQGQSFPFTFGGG | | | | | | |
| | TRGYFFDYWGQGT | TKVEIK (SEQ ID NO: | | | | | | |
| | LVTVSS (SEQ ID NO: | 68) | | | | | | |
| | 67) | | | | | | | |
| Exemplary anti- | EVQLQQSGAELVKP | DIVLTQSPASLAVSL | DMYIH | IDPEND | GNYVH | RASESV | AASNVE | QQSRRV |
| SIRPA antibody | GASVKLSCTTSGEN | GQRATISCRASESVQ | (SEQ ID | DTKYDP | (SEQ ID | QYYGTS | S (SEQ | WT (SEQ |
| (α-SIRPA 1) | FKDMYIHWVKQRP | YYGTSLMQWYQQK | NO: 7) | KFQD | NO: 9) | LMQ | ID NO: | ID NO: |
| | QQGLEWIGRIDPEN | PGQPPKLLIYAASNV | | (SEQ ID | | (SEQ ID | 11) | 12) |
| | DDTKYDPKFQDKAT | ESGVPARFSGSGSGT | | NO: 8) | | NO: 10) | | |
| | ITADTSSNTAYLQLS | DFSLNIYPVEEDDIA | | | | | | |
| | NLPSEDTAVYYCAR | MYFCQQSRRVWTF | | | | | | |
| | GNYVHWGQGTLVT | GGGTKLEIK (SEQ ID | | | | | | |
| | VSA (SEQ ID NO: 69) | NO: 70) | | | | | | |
| Exemplary anti- | EVQLQQSGAELVKP | DIVLTQSPASLAVSL | DMYLH | IDPENDS | GNYVH | RASESV | AASNVG | QQTRRV |
| SIRPA antibody | GASVKVSCTGSGFN | GQRATISCRASESVQ | (SEQ ID | TKYDPK | (SEQ ID | QYYGTS | S (SEQ | WT (SEQ |
| (α-SIRPA 2) | FKDMYLHWVKQRP | YYGTSLMQWYQQK | NO: 13) | FQD | NO: 15) | LMQ | ID NO: | ID NO: |
| | EQGLEWIGRIDPEN | PGQPPKLLIYAASNV | | (SEQ ID | | (SEQ ID | 17) | 18) |
| | DSTKYDPKFQDKAT | GSGVPARFSGSGSG | | NO: 14) | | NO: 16) | | |
| | LTADTASNTAYLQIS | TDFSLNIHPVEEDDI | | | | | | |
| | SLTSEDTAVYYCAR | AMYFCQQTRRVWT | | | | | | |
| | GNYVHWGQGTLVT | FGGGTKLEIK (SEQ | | | | | | |
| | VSA (SEQ ID NO: 71) | ID NO: 72) | | | | | | |
| Exemlary anti- | EVQLQQSGAELVKP | QIVLTQSPAIMSASP | DSYMH | IDPEDGE | RGFY | SSSVSSS | STSNLA | YQWSTH |
| SIRPA antibody | GASVKLSCTASGFNI | GEKVTLTCSASSSVS | (SEQ ID | TKYAPK | (SEQ ID | YLY | S (SEQ | PYT |
| (α-SIRPA 3) | KDSYMHWVKQRTE | SSYLYWYQQKPGSS | NO: 19) | FQG | NO: 21) | (SEQ ID | ID NO: | (SEQ ID |
| | QGLEWIGRIDPEDG | PKLWIYSTSNLASG | | (SEQ ID | | NO: 22) | 23) | NO: 24) |
| | ETKYAPKFQGKATI | VPARFSGSGSGTSYS | | NO: 20) | | | | |
| | TADTSSNTAYLQLT | LTISSMEAEDAASYF | | | | | | |
| | SLTSEDTAVYYCAR | CYQWSTHPYTFGGG | | | | | | |
| | RGFYWGQGTTLTVS | TKLEIK (SEQ ID NO: | | | | | | |
| | S (SEQ ID NO: 73) | 74 | | | | | | |
| Exemplary anti- | QVQLVQSGAEVKKP | DIVMTQSPDSLAVS | EYTIH | GINPNN | RRIAYG | KSSQSL | WASTRE | QQYFSY |
| FAPa antibody | GASVKVSCKTSRYT | LGERATINCKSSQSL | (SEQ ID | GIPNYN | YDEGHA | LYSRNQ | S (SEQ | PLT (SEQ |
| (α-FAPα 1) | FTEYTIHWVRQAPG | LYSRNQKNYLAWY | NO: 25) | QKFKG | MDY | KNYLA | ID NO: | ID NO: |
| | QRLEWIGGINPNNGI | QQKPGQPPKLLIFW | | (SEQ ID | (SEQ ID | (SEQ ID | 29) | 30) |
| | PNYNQKFKGRVTIT | ASTRESGVPDRFSGS | | NO: 26) | NO: 27) | NO: 28) | | |
| | VDTSASTAYMELSS | GFGTDFTLTISSLQA | | | | | | |
| | LRSEDTAVYYCARR | EDVAVYYCQQYFS | | | | | | |
| | RIAYGYDEGHAMD | YPLTFGQGTKVEIK | | | | | | |
| | YWGQGTLVTVSS | (SEQ ID NO: 76) | | | | | | |
| | (SEQ ID NO: 75) | | | | | | | |
| Exemplary anti- | EVQLLESGGGLVQP | EIVLTQSPGTLSLSP | SYAMS | AIIGSGA | YCAKG | RASQSV | NVGSRR | CQQGIM |
| FAPa antibody | GGSLRLSCAASGFTF | GERATLSCRASQSV | (SEQ ID | STYYAD | WFG | TSSYL | A (SEQ | LPP (SEQ |
| (α-FAPα 2) | SSYAMSWVRQAPG | TSSYLAWYQQKPG | NO: 31) | SVK | (SEQ ID | (SEQ ID | ID NO: | ID NO: |
| | KGLEWVSAIIGSGAS | QAPRLLINVGSRRA | | (SEQ ID | NO: 33) | NO: 34) | 35) | 36) |
| | TYYADSVKGRFTIS | TGIPDRFSGSGSGTD | | NO: 32) | | | | |
| | RDNSKNTLYLQMNS | FTLTISRLEPEDFAV | | | | | | |
| | LRAEDTAVYYCAK | YYCQQGIMLPPTFG | | | | | | |
| | GWFGGFNYWGQGT | QGTKVEIK (SEQ ID | | | | | | |
| | LVTVSS (SEQ ID NO: | NO: 78) | | | | | | |
| | 77) | | | | | | | |
| Exemplary anti- | EVQLVQSGAEVKKP | AIQLTQSPSSLSASV | GYTFTS | RIDPNSG | DYRKGL | KASQDV | WASTRH | QQYNSY |
| PDL1 antibody | GATVKISCKVSGYT | GDRVTITCKASQDV | YWMY | STKYNE | YAMDY | GTAVA | T (SEQ | PLT (SEQ |
| (α-PDL1 1) | FTSYWMYWVRQAR | GTAVAWYLQKPGQ | (SEQ ID | KFKN | (SEQ ID | (SEQ ID | ID NO: | ID NO: |
| | GQRLEWIGRIDPNS | SPQLLIYWASTRHT | NO: 37) | (SEQ ID | NO: 39) | NO: 40) | 41) | 42) |
| | GSTKYNEKFKNRFTI | GVPSRFSGSGSGTDF | | NO: 38) | | | | |
| | SRDNSKNTLYLQMN | TFTISSLEAEDAATY | | | | | | |
| | SLRAEDTAVYYCAR | YCQQYNSYPLTFGQ | | | | | | |
| | DYRKGLYAMDYW | GTKVEIK (SEQ ID | | | | | | |
| | GQGTTVTVSS (SEQ | NO: 80) | | | | | | |
| | ID NO: 79) | | | | | | | |
| Exemplary anti- | QVQLVESGGGVVQP | DIVMTQSPDSLSVSL | SYGMH | VIWYDG | DQHIVV | KSSQSV | WASTRE | QQYYSI |
| uPAR antibody | GRSLRLSCAASGFTF | GERATINCKSSQSVL | (SEQ ID | SNKYYA | VTAILD | LYGSNN | S (SEQ | FPWT |
| (α-uPAR 1) | SSYGMHWVRQAPG | YGSNNRNSLAWYQ | NO: 43) | DSVK | Y (SEQ | RNSLA | ID NO: | (SEQ ID |
| | KGLEWVAVIWYDG | QKPGQPPKLLIYWA | | (SEQ ID | ID NO: | (SEQ ID | 47) | NO: 48) |
| | SNKYYADSVKGRFT | STRESGVPDRFSGSG | | NO: 44) | 45) | NO: 46) | | |
| | ISRDNSKNTLYLQM | SGTDFTLTISSLQAE | | | | | | |
| | NSLRAEDTAVYYCA | DVAVYYCQQYYSIF | | | | | | |
| | RDQHIVVVTAILDY | PWTFGQGTKVEIK | | | | | | |
| | WGQGTLVTVSS | (SEQ ID NO: 82) | | | | | | |
| | (SEQ ID NO: 81) | | | | | | | |
| Exemplary anti- | EVQLLESGGGLVQP | DIQMTQSPLSLSASV | IYRMQ | GISPSGG | WSGGSG | QASRDI | DASSLQ | QQFDSL |
| IGF1R antibody | GGSLRLSCAASGFTF | GDRVTITCQASRDIR | (SEQ ID | TTWYA | YAFDI | RNYLN | T (SEQ | PHT |
| (α-IGF1R 1) | SIYRMQWVRQAPG | NYLNWYQQKPGKA | NO: 49) | DSVKG | (SEQ ID | (SEQ ID | ID NO: | (SEQ ID |
| | KGLEWVSGISPSGG | PKLLIYDASSLQTGV | | (SEQ ID | NO: 51) | NO: 52) | 53) | NO: 54) |
| | TTWYADSVKGRFTI | PSRFGGSGSGTDFSF | | NO: 50) | | | | |
| | SRDNSKNTLYLQMN | TIGSLQPEDIATYYC | | | | | | |
| | SLRAEDTAVYYCAR | QQFDSLPHTFGQGT | | | | | | |
| | WSGGSGYAFDIWG | KLEIK (SEQ ID NO: | | | | | | |
| | QGTMVTVSS (SEQ | 84) | | | | | | |
| | ID NO: 83) | | | | | | | |
| Exemplary anti- | EVQLLESGGGLVQP | DIQMTQSPDSLAVS | NYPMY | RISSSGG | DRWSRS | KSSQSV | LASTRE | QQYYST |
| IGF1R antibody | GGSLRLSCAASGFTF | LGERATINCKSSQSV | (SEQ ID | RTVYAD | AAEYGL | LYSSNN | S (SEQ | WT (SEQ |
| (α-IGF1R 2) | SNYPMYWVRQAPG | LYSSNNKNYLAWY | NO: 55) | SVKG | GGY | KNYLA | ID NO: | ID NO: |
| | KGLEWVSRISSSGG | QQKPGQPPKLLIYL | | (SEQ ID | (SEQ ID | (SEQ ID | 59) | 60) |
| | RTVYADSVKGRFTI | ASTRESGVPDRFSGS | | NO: 56) | NO: 57) | NO: 58) | | |
| | SRDNSKNTLYLQMN | GSGTDFTLTISSLQA | | | | | | |
| | SLRAEDTAVYYCAR | EDVAVYYCQQYYS | | | | | | |
| | DRWSRSAAEYGLG | TWTFGQGTKVEIK | | | | | | |
| | GYWGQGTLVTVSS | (SEQ ID NO: 86) | | | | | | |
| | (SEQ ID NO: 85) | | | | | | | |
| Exemplary anti- | QVQLVQSGAEVKKP | SSELTQDPVVSVAL | SYAIS | GIIPIFGT | DTDYMF | QGDNLR | GKNRRP | SSRDNS |
| ANTXR1 | GTSVKVSCKVPGYT | GETVSITCQGDNLR | (SEQ ID | TNYAQK | DY (SEQ | DFYAS | S (SEQ | KHVV |
| antibody | FSSYAISWVRQAPG | DFYASWYQQKPGQ | NO: 227) | FQG | ID NO: | (SEQ ID | ID NO: | (SEQ ID |
| (α-ANTXR1 1) | QGLEWMGGIIPIFGT | APLLVMYGKNRRPS | | (SEQ ID | 229) | NO: 230) | 231) | NO: 232) |
| | TNYAQKFQGRVTIT | GIPDRFSGSTSGNTL | | NO: 228) | | | | |
| | GEESTSTVYMELSSL | SLTITGAQAEDEAD | | | | | | |
| | RSEDTAVYYCARDT | YYCSSRDNSKHVVF | | | | | | |
| | DYMFDYWGQGTLV | GGGTKVTVL (SEQ | | | | | | |
| | TVSS (SEQ ID NO: | ID NO: 226) | | | | | | |
| | 225) | | | | | | | |
| Exemplary anti- | QVQLKESPGALVKP | DIELTQPPSVSVAPG | TSGGGV | HIYSND | GGYFLD | SGDNIG | ADSKRP | QSYDITS |
| ANTXR1 | TQTLTLTCTFSGFSL | QTARISCSGDNIGGI | S (SEQ | DKSYST | Y (SEQ | GIYVH | S (SEQ | LV (SEQ |
| antibody | STSGGGVSWIRQPP | YVHWYQQKPGQAP | ID NO: | SLKT | ID NO: | (SEQ ID | ID NO: | ID NO: |
| (α-ANTXR1 2) | GKALEWLAHIYSND | VLVIYADSKRPSGIP | 235) | (SEQ ID | 237) | NO: 238) | 239) | 240) |
| | DKSYSTSLKTRLTIS | ERFSGSNSGNTATLT | | NO: 236) | | | | |
| | KDTSKNQVVLTMT | ISGTQAEDEADYYC | | | | | | |
| | NMDPVDTATYYCA | QSYDITSLVFGGGT | | | | | | |
| | RGGYFLDYWGQGT | KLTVL (SEQ ID NO: | | | | | | |
| | LVTVSS (SEQ ID NO: | 234) | | | | | | |
| | 233) | | | | | | | |
| Exemplary anti- | QVQLQESGPGLVAP | DIQMNQSPSSLSASL | GYGVN | MIWVD | GGYGA | HASQNI | KASNLH | QQGQSY |
| CD248 | SQSLSITCTVSGFSLT | GDTITITCHASQNIN | (SEQ ID | GSTDYN | MDY | NVWLT | T (SEQ | PWT |
| antibody | GYGVNWVRQPPEK | VWLTWYQQKPGNI | NO: 243) | SALKS | (SEQ ID | (SEQ ID | ID NO: | (SEQ ID |
| (α-CD248 1) | GLEWLGMIWVDGS | PKLLIYKASNLHTG | | (SEQ ID | NO: 245) | NO: 246) | 247) | NO: 248) |
| | TDYNSALKSRLNISK | VPSRFSGSGSGTGFT | | NO: 244) | | | | |
| | DKSKSQVFLKMNSL | LTISSLQPEDIATYY | | | | | | |
| | QTDDTARYYCARG | CQQGQSYPWTFGG | | | | | | |
| | GYGAMDYWGQGTS | GTKLEIK (SEQ ID | | | | | | |
| | VTVSS (SEQ ID NO: | NO: 242) | | | | | | |
| | 241) | | | | | | | |
| Exemplary anti- | QVQLQESGPGLVRP | DIQMTQSPSSLSASV | DYVIH | YINPYD | RGNSYD | RASQNV | SASNRY | QQYTNY |
| CD248 | SQTLSLTCTASGYTF | GDRVTITCRASQNV | (SEQ ID | DDTTYN | GYFDYS | GTAVA | T (SEQ | PMYT |
| antibody | TDYVIHWVKQPPGR | GTAVAWLQQTPGK | NO: 251) | QKFKG | MDY | (SEQ ID | ID NO: | (SEQ ID |
| (α-CD248 2) | GLEWIGYINPYDDD | APKLLIYSASNRYTG | | (SEQ ID | (SEQ ID | NO: 254) | 255) | NO: 256) |
| | TTYNQKFKGRVTM | VPSRFSGSGSGTDYT | | NO: 252) | NO: 253) | | | |
| | LVDTSSNTAYLRLSS | FTISSLQPEDIATYY | | | | | | |
| | VTAEDTAVYYCAR | CQQYTNYPMYTFG | | | | | | |
| | RGNSYDGYFDYSM | QGTKVQIK (SEQ ID | | | | | | |
| | DYWGSGTPVTVSS | NO: 250) | | | | | | |
| | (SEQ ID NO: 249) | | | | | | | |
| Exemplary anti- | QVQLQQSGAELVRP | DVVMTQTPLSLPVS | DYEMH | ALDPKT | FYSYTY | RSSQSL | KVSNRF | SQNTHV |
| GPC3 antibody | GASVKLSCKASGYT | LGDQASISCRSSQSL | (SEQ ID | GDTAYS | (SEQ ID | VHSNGN | S (SEQ | PPT (SEQ |
| (α-GPC3 1) | FTDYEMHWVKQTP | VHSNGNTYLHWYL | NO: 259) | QKFKG | NO: 261) | TYLH | ID NO: | ID NO: |
| | VHGLKWIGALDPKT | QKPGQSPKLLIYKVS | | (SEQ ID | | (SEQ ID | 263) | 264) |
| | GDTAYSQKFKGKAT | NRFSGVPDRFSGSGS | | NO: 260) | | NO: 262) | | |
| | LTADKSSSTAYMEL | GTDFTLKISRVEAED | | | | | | |
| | RSLTSEDSAVYYCT | LGVYFCSQNTHVPP | | | | | | |
| | RFYSYTYWGQGTL | TFGSGTKLEIK (SEQ | | | | | | |
| | VTVSA (SEQ ID NO: | ID NO: 258) | | | | | | |
| | 257) | | | | | | | |
| Exemplary anti- | EVQLQQSGPGLVKP | DIVMTQSPDSLAVS | SNSAAW | KTYYRF | ESTTYD | KSSQTV | WASTRE | QQYYST |
| TIGIT antibody | SQTLSLTCAISGDSV | LGERATINCKSSQTV | N (SEQ | KWYSD | LLAGPF | LYSSNN | S (SEQ | PFT (SEQ |
| (α-TIGIT 1) | SSNSAAWNWIRQSP | LYSSNNKKYLAWY | ID NO: | YAVSVK | DY (SEQ | KKYLA | ID NO: | ID NO: |
| | SRGLEWLGKTYYRF | QQKPGQPPNLLIYW | 267) | G (SEQ | ID NO: | (SEQ ID | 271) | 272) |
| | KWYSDYAVSVKGRI | ASTRESGVPDRFSGS | | ID NO: | 269) | NO: 270) | | |
| | TINPDTSKNQFSLQL | GSGTDFTLTISSLQA | | 268) | | | | |
| | NSVTPEDTAVFYCT | EDVAVYYCQQYYS | | | | | | |
| | RESTTYDLLAGPFD | TPFTFGPGTKVEIK | | | | | | |
| | YWGQGTLVTVSS | (SEQ ID NO: 266) | | | | | | |
| | (SEQ ID NO: 265) | | | | | | | |
| Exemplary anti- | QVQLVQSGAEVKKP | DIQMTQSPSSLSASV | DYNIN | YIYPYIG | EDKTAR | RVSENI | EATNLA | QHFWGT |
| PVRIG | GASVKVSCKASGYT | GDRVTITCRVSENIY | (SEQ ID | GSGYAQ | NAMDY | YSNLA | E (SEQ | PYT |
| antibody | FTDYNINWVRQAPG | SNLAWYQQKPGKA | NO: 275) | KFQG | (SEQ ID | (SEQ ID | ID NO: | (SEQ ID |
| (α-PVRIG 1) | QGLEWMGYIYPYIG | PKLLIYEATNLAEG | | (SEQ ID | NO: 277) | NO: 278) | 279) | NO: 280) |
| | GSGYAQKFQGRVT | VPSRFSGSGSGTDFT | | NO: 276) | | | | |
| | MTRDTSTSTVYMEL | LTISSLQPEDFATYY | | | | | | |
| | SSLRSEDTAVYYCA | CQHFWGTPYTFGQG | | | | | | |
| | REDKTARNAMDYW | TKLEIK (SEQ ID NO: | | | | | | |
| | GQGTLVTVSS (SEQ | 274) | | | | | | |
| | ID NO: 273) | | | | | | | |
| Exemplary anti- | QVQLVQSGAEVKKP | DIQMTQSPSSLSASV | DYVIN | EIYPGSG | RGRYGL | RASQDIS | YTSRLH | QQGNTR |
| NKp46 | GSSVKVSCKASGYT | GDRVTITCRASQDIS | (SEQ ID | TNYYNE | YAMDY | NYLN | S (SEQ | PWT |
| antibody | FTDYVINWGRQAPG | NYLNWYQQKPGKA | NO: 61) | KFKAKA | (SEQ ID | (SEQ ID | ID NO: | (SEQ ID |
| (α-NKp46 1) | QGLEWIGEIYPGSGT | PKLLIYYTSRLHSGV | | (SEQ ID | NO: 63) | NO: 64) | 65) | NO: 66) |
| | NYYNEKFKAKATIT | PSRFSGSGSGTDFTF | | NO: 62) | | | | |
| | ADKSTSTAYMELSS | TISSLQPEDIATYFC | | | | | | |
| | LRSEDTAVYFCARR | QQGNTRPWTFGGG | | | | | | |
| | GRYGLYAMDYWG | TKVEIK (SEQ ID NO: | | | | | | |
| | QGTTVTVSS (SEQ | 88) | | | | | | |
| | ID NO: 87) | | | | | | | |

TABLE 2
Sequences of exemplary extracellular domains

| | Sequence |
|---|---|
| NKG2D | FLNSLFNQEVQIPLTESYCGPCPKNWICYKNNCYQFFDESKNWYESQ |
| extracellular | ASCMSQNASLLKVYSKEDQDLLKLVKSYHWMGLVHIPTNGSWQWE |
| domain | DGSILSPNLLTIIEMQKGDCALYASSFKGYIENCSTPNTYICMQRTV |
| | (SEQ ID NO: 89) |
| ULBP1 | GWVDTHCLCYDFIITPKSRPEPQWCEVQGLVDERPFLHYDCVNHKA |
| extracellular | KAFASLGKKVNVTKTWEEQTETLRDVVDFLKGQLLDIQVENLIPIEP |
| domain | LTLQARMSCEHEAHGHGRGSWQFLFNGQKFLLFDSNNRKWTALHP |
| | GAKKMTEKWEKNRDVTMFFQKISLGDCKMWLEEFLMYWEQMLDP |
| | TKPPSLAP (SEQ ID NO: 90) |
| ULBP2 | GRADPHSLCYDITVIPKFRPGPRWCAVQGQVDEKTFLHYDCGNKTV |
| extracellular | TPVSPLGKKLNVTTAWKAQNPVLREVVDILTEQLRDIQLENYTPKEP |
| domain | LTLQARMSCEQKAEGHSSGSWQFSFDGQIFLLFDSEKRMWTTVHPG |
| | ARKMKEKWENDKVVAMSFHYFSMGDCIGWLEDFLMGMDSTLEPS |
| | AGAPLAM (SEQ ID NO: 91) |
| MICA | EPHSLRYNLTVLSWDGSVQSGFLTEVHLDGQPFLRCDRQKCRAKPQ |
| extracellular | GQWAEDVLGNKTWDRETRDLTGNGKDLRMTLAHIKDQKEGLHSLQ |
| domain | EIRVCEIHEDNSTRSSQHFYYDGELFLSQNLETKEWTMPQSSRAQTL |
| | AMNVRNFLKEDAMKTKTHYHAMHADCLQELRRYLKSGVVLRRTV |
| | PPMVNVTRSEASEGNITVTCRASGFYPWNITLSWRQDGVSLSHDTQQ |
| | WGDVLPDGNGTYQTWVATRICQGEEQRFTCYMEHSGNHSTHPVPS |
| | GKVLVLQSHW (SEQ ID NO: 92) |
| MICB | AEPHSLRYNLMVLSQDESVQSGFLAEGHLDGQPFLRYDRQKRRAKP |
| extracellular | QGQWAEDVLGAKTWDTETEDLTENGQDLRRTLTHIKDQKGGLHSL |
| domain | QEIRVCEIHEDSSTRGSRHFYYDGELFLSQNLETQESTVPQSSRAQTL |
| | AMNVTNFWKEDAMKTKTHYRAMQADCLQKLQRYLKSGVAIRRTV |
| | PPMVNVTCSEVSEGNITVTCRASSFYPRNITLTWRQDGVSLSHNTQQ |
| | WGDVLPDGNGTYQTWVATRIRQGEEQRFTCYMEHSGNHGTHPVPS |
| | GKVLVLQSQRTD (SEQ ID NO: 93) |
| TGFb receptor | IPPHVQKSVNNDMIVTDNNGAVKFPQLCKFCDVRFSTCDNQKSCMS |
| II extracellular | NCSITSICEKPQEVCVAVWRKNDENITLETVCHDPKLPYHDFILEDAA |
| domain | SPKCIMKEKKKPGETFFMCSCSSDECNDNIIFSEEYNTSNPD (SEQ ID |
| | NO: 94) |

TABLE 3
Sequences of exemplary linkers
Sequence

IEGR (SEQ ID NO: 95)
GGGGS (SEQ ID NO: 96)
GGSGGGSG (SEQ ID NO: 97)
GGGGSGGGGS (SEQ ID NO: 98)
GGGGSGGGGSGGGGS (SEQ ID NO: 99)
GGGGSGGGGSGGGGSGGGGS (SEQ ID NO: 100)
GGSGGGSGGGGSGGGGSGGGGSG (SEQ ID NO: 101)
AEAAAKEAAAKA (SEQ ID NO: 102)
AEAAAKEAAAKEAAAKA (SEQ ID NO: 103)
APAPAPAPA (SEQ ID NO: 104)
APAPAPAPAPAPAPAPAPAP (SEQ ID NO: 105)

TABLE 4
Full lengh sequences of exemplary constructs

| Construct | Chain 1 | Chain 2 |
|---|---|---|
| α-PDGFRb 1 | QVQLQESGPGLVKPSETLSLTCT | DIQMTQSPSSLSASVGDRVTI |
| S239D/1332E | VSGFSLTNYAINWVRQPPGKGL | TCHASQNINVWLSWYQQKP |
| | EWLGIIWTGGGTSYNSALKSRL | GKAPKLLIYKASNLHTGVPSR |
| | TISKDTSKNQVSLKLSSVTAADT | FSGSGSGTGFTLTISSLQPEDI |
| | AVYYCARTGTRGYFFDYWGQG | ATYYCQQGQSFPFTFGGGTK |
| | TLVTVSSASTKGPSVFPLAPSSK | VEIKRTVAAPSVFIFPPSDEQL |
| | STSGGTAALGCLVKDYFPEPVT | KSGTASVVCLLNNFYPREAK |
| | VSWNSGALTSGVHTFPAVLQSS | VQWKVDNALQSGNSQESVT |
| | GLYSLSSVVTVPSSSLGTQTYIC | EQDSKDSTYSLSSTLTLSKAD |
| | NVNHKPSNTKVDKKVEPKSCD | YEKHKVYACEVTHQGLSSPV |
| | KTHTCPPCPAPELLGGPDVFLFP | TKSFNRGEC (SEQ ID NO: 107) |
| | PKPKDTLMISRTPEVTCVVVDV | |
| | SHEDPEVKFNWYVDGVEVHNA | |
| | KTKPREEQYNSTYRVVSVLTVL | |
| | HQDWLNGKEYKCKVSNKALPA | |
| | PEEKTISKAKGQPREPQVYTLPP | |
| | SREEMTKNQVSLTCLVKGFYPS | |
| | DIAVEWESNGQPENNYKTTPPV | |
| | LDSDGSFFLYSKLTVDKSRWQQ | |
| | GNVFSCSVMHEALHNHYTQKS | |
| | LSLSPGK (SEQ ID NO: 106) | |
| α-SIRPA 1 | EVQLQQSGAELVKPGASVKLSC | DIVLTQSPASLAVSLGQRATIS |
| S239D/1332E | TTSGFNFKDMYIHWVKQRPQQ | CRASESVQYYGTSLMQWYQ |
| | GLEWIGRIDPENDDTKYDPKFQ | QKPGQPPKLLIYAASNVESGV |
| | DKATITADTSSNTAYLQLSNLPS | PARFSGSGSGTDFSLNIYPVEE |
| | EDTAVYYCARGNYVHWGQGTL | DDIAMYFCQQSRRVWTFGGG |
| | VTVSAASTKGPSVFPLAPSSKST | TKLEIKRTVAAPSVFIFPPSDE |
| | SGGTAALGCLVKDYFPEPVTVS | QLKSGTASVVCLLNNFYPRE |
| | WNSGALTSGVHTFPAVLQSSGL | AKVQWKVDNALQSGNSQES |
| | YSLSSVVTVPSSSLGTQTYICNV | VTEQDSKDSTYSLSSTLTLSK |
| | NHKPSNTKVDKKVEPKSCDKTH | ADYEKHKVYACEVTHQGLSS |
| | TCPPCPAPELLGGPDVFLFPPKP | PVTKSFNRGEC (SEQ ID NO: |
| | KDTLMISRTPEVTCVVVDVSHE | 109) |
| | DPEVKFNWYVDGVEVHNAKTK | |
| | PREEQYNSTYRVVSVLTVLHQD | |
| | WLNGKEYKCKVSNKALPAPEE | |
| | KTISKAKGQPREPQVYTLPPSRE | |
| | EMTKNQVSLTCLVKGFYPSDIA | |
| | VEWESNGQPENNYKTTPPVLDS | |
| | DGSFFLYSKLTVDKSRWQQGN | |
| | VFSCSVMHEALHNHYTQKSLSL | |
| | SPGK (SEQ ID NO: 108) | |
| α-SIRPA 2 | EVQLQQSGAELVKPGASVKVSC | DIVLTQSPASLAVSLGQRATIS |
| S239D/1332E | TGSGFNFKDMYLHWVKQRPEQ | CRASESVQYYGTSLMQWYQ |
| | GLEWIGRIDPENDSTKYDPKFQ | QKPGQPPKLLIYAASNVGSG |
| | DKATLTADTASNTAYLQISSLTS | VPARFSGSGSGTDFSLNIHPV |
| | EDTAVYYCARGNYVHWGQGTL | EEDDIAMYFCQQTRRVWTFG |
| | VTVSAASTKGPSVFPLAPSSKST | GGTKLEIKRTVAAPSVFIFPPS |
| | SGGTAALGCLVKDYFPEPVTVS | DEQLKSGTASVVCLLNNFYP |
| | WNSGALTSGVHTFPAVLQSSGL | REAKVQWKVDNALQSGNSQ |
| | YSLSSVVTVPSSSLGTQTYICNV | ESVTEQDSKDSTYSLSSTLTL |
| | NHKPSNTKVDKKVEPKSCDKTH | SKADYEKHKVYACEVTHQG |
| | TCPPCPAPELLGGPDVFLFPPKP | LSSPVTKSFNRGEC (SEQ ID |
| | KDTLMISRTPEVTCVVVDVSHE | NO: 111) |
| | DPEVKFNWYVDGVEVHNAKTK | |
| | PREEQYNSTYRVVSVLTVLHQD | |
| | WLNGKEYKCKVSNKALPAPEE | |
| | KTISKAKGQPREPQVYTLPPSRE | |
| | EMTKNQVSLTCLVKGFYPSDIA | |
| | VEWESNGQPENNYKTTPPVLDS | |
| | DGSFFLYSKLTVDKSRWQQGN | |
| | VFSCSVMHEALHNHYTQKSLSL | |
| | SPGK (SEQ ID NO: 110) | |
| α-SIRPA 3 | EVQLQQSGAELVKPGASVKLSC | QIVLTQSPAIMSASPGEKVTL |
| S239D/1332E | TASGFNIKDSYMHWVKQRTEQ | TCSASSSVSSSYLYWYQQKP |
| | GLEWIGRIDPEDGETKYAPKFQ | GSSPKLWIYSTSNLASGVPAR |
| | GKATITADTSSNTAYLQLTSLTS | FSGSGSGTSYSLTISSMEAED |
| | EDTAVYYCARRGFYWGQGTTL | AASYFCYQWSTHPYTFGGGT |
| | TVSSASTKGPSVFPLAPSSKSTS | KLEIKRTVAAPSVFIFPPSDEQ |
| | GGTAALGCLVKDYFPEPVTVSW | LKSGTASVVCLLNNFYPREA |
| | NSGALTSGVHTFPAVLQSSGLY | KVQWKVDNALQSGNSQESV |
| | SLSSVVTVPSSSLGTQTYICNVN | TEQDSKDSTYSLSSTLTLSKA |
| | HKPSNTKVDKKVEPKSCDKTHT | DYEKHKVYACEVTHQGLSSP |
| | CPPCPAPELLGGPDVFLFPPKPK | VTKSFNRGEC (SEQ ID NO: |
| | DTLMISRTPEVTCVVVDVSHED | 113) |
| | PEVKFNWYVDGVEVHNAKTKP | |
| | REEQYNSTYRVVSVLTVLHQD | |
| | WLNGKEYKCKVSNKALPAPEE | |
| | KTISKAKGQPREPQVYTLPPSRE | |
| | EMTKNQVSLTCLVKGFYPSDIA | |
| | VEWESNGQPENNYKTTPPVLDS | |
| | DGSFFLYSKLTVDKSRWQQGN | |
| | VFSCSVMHEALHNHYTQKSLSL | |
| | SPGK (SEQ ID NO: 112) | |
| α-FAPα 1 | QVQLVQSGAEVKKPGASVKVS | DIVMTQSPDSLAVSLGERATI |
| S239D/1332E | CKTSRYTFTEYTIHWVRQAPGQ | NCKSSQSLLYSRNQKNYLAW |
| | RLEWIGGINPNNGIPNYNQKFK | YQQKPGQPPKLLIFWASTRES |
| | GRVTITVDTSASTAYMELSSLRS | GVPDRFSGSGFGTDFTLTISSL |
| | EDTAVYYCARRRIAYGYDEGH | QAEDVAVYYCQQYFSYPLTF |
| | AMDYWGQGTLVTVSSAKTTAP | GQGTKVEIKRADAAPTVSIFP |
| | SVYPLAPVCGDTTGSSVTLGCL | PSSEQLTSGGASVVCFLNNFY |
| | VKGYFPEPVTLTWNSGSLSSGV | PKDINVKWKIDGSERQNGVL |
| | HTFPAVLQSDLYTLSSSVTVTSS | NSWTDQDSKDSTYSMSSTLT |
| | TWPSQSITCNVAHPASSTKVDK | LTKDEYERHNSYTCEATHKT |
| | KIEPRGPTIKPCPPCKCPAPNLLG | STSPIVKSFNRNEC (SEQ ID |
| | GPDVFIFPPKIKDVLMISLSPIVT | NO: 115) |
| | CVVVDVSEDDPDVQISWFVNN | |
| | VEVHTAQTQTHREDYNSTLRVV | |
| | SALPIQHQDWMSGKEFKCKVN | |
| | NKDLPAPEERTISKPKGSVRAPQ | |
| | VYVLPPPEEEMTKKQVTLTCMV | |
| | TDFMPEDIYVEWTNNGKTELNY | |
| | KNTEPVLDSDGSYFMYSKLRVE | |
| | KKNWVERNSYSCSVVHEGLHN | |
| | HHTTKSFSRTPGK (SEQ ID NO: | |
| | 114) | |
| α-FAPα 2 | EVQLLESGGGLVQPGGSLRLSC | EIVLTQSPGTLSLSPGERATLS |
| | AASGFTFSSYAMSWVRQAPGK | CRASQSVTSSYLAWYQQKPG |
| | GLEWVSAIIGSGASTYYADSVK | QAPRLLINVGSRRATGIPDRF |
| | GRFTISRDNSKNTLYLQMNSLR | SGSGSGTDFTLTISRLEPEDFA |
| | AEDTAVYYCAKGWFGGFNYW | VYYCQQGIMLPPTFGQGTKV |
| | GQGTLVTVSSASTKGPSVFPLAP | EIKRTVAAPSVFIFPPSDEQLK |
| | SSKSTSGGTAALGCLVKDYFPEP | SGTASVVCLLNNFYPREAKV |
| | VTVSWNSGALTSGVHTFPAVLQ | QWKVDNALQSGNSQESVTE |
| | SSGLYSLSSVVTVPSSSLGTQTYI | QDSKDSTYSLSSTLTLSKADY |
| | CNVNHKPSNTKVDKKVEPKSC | EKHKVYACEVTHQGLSSPVT |
| | DKTHTCPPCPAPELLGGPSVFLF | KSFNRGEC (SEQ ID NO: 117) |
| | PPKPKDTLMISRTPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APIEKTISKAKGQPREPQVYTLP | |
| | PSRDELTKNQVSLTCLVKGFYPS | |
| | DIAVEWESNGQPENNYKTTPPV | |
| | LDSDGSFFLYSKLTVDKSRWQQ | |
| | GNVFSCSVMHEALHNHYTQKS | |
| | LSLSPGK (SEQ ID NO: 116) | |
| α-PDL1 1 | EVQLVQSGAEVKKPGATVKISC | AIQLTQSPSSLSASVGDRVTIT |
| S239D/1332E | KVSGYTFTSYWMYWVRQARG | CKASQDVGTAVAWYLQKPG |
| | QRLEWIGRIDPNSGSTKYNEKFK | QSPQLLIYWASTRHTGVPSRF |
| | NRFTISRDNSKNTLYLQMNSLR | SGSGSGTDFTFTISSLEAEDAA |
| | AEDTAVYYCARDYRKGLYAMD | TYYCQQYNSYPLTFGQGTKV |
| | YWGQGTTVTVSSASTKGPSVFP | EIKRTVAAPSVFIFPPSDEQLK |
| | LAPSSKSTSGGTAALGCLVKDY | SGTASVVCLLNNFYPREAKV |
| | FPEPVTVSWNSGALTSGVHTFP | QWKVDNALQSGNSQESVTE |
| | AVLQSSGLYSLSSVVTVPSSSLG | QDSKDSTYSLSSTLTLSKADY |
| | TQTYICNVNHKPSNTKVDKKVE | EKHKVYACEVTHQGLSSPVT |
| | PKSCDKTHTCPPCPAPELLGGPD | KSFNRGEC (SEQ ID NO: 119) |
| | VFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCKVSNK | |
| | ALPAPEEKTISKAKGQPREPQVY | |
| | TLPPSREEMTKNQVSLTCLVKG | |
| | FYPSDIAVEWESNGQPENNYKT | |
| | TPPVLDSDGSFFLYSKLTVDKSR | |
| | WQQGNVFSCSVMHEALHNHYT | |
| | QKSLSLSPGK (SEQ ID NO: 118) | |
| α-uPAR 1 | QVQLVESGGGVVQPGRSLRLSC | DIVMTQSPDSLSVSLGERATI |
| S239D/1332E | AASGFTFSSYGMHWVRQAPGK | NCKSSQSVLYGSNNRNSLAW |
| | GLEWVAVIWYDGSNKYYADSV | YQQKPGQPPKLLIYWASTRES |
| | KGRFTISRDNSKNTLYLQMNSL | GVPDRFSGSGSGTDFTLTISSL |
| | RAEDTAVYYCARDQHIVVVTAI | QAEDVAVYYCQQYYSIFPWT |
| | LDYWGQGTLVTVSSASTKGPSV | FGQGTKVEIKRTVAAPSVFIF |
| | FPLAPSSKSTSGGTAALGCLVKD | PPSDEQLKSGTASVVCLLNNF |
| | YFPEPVTVSWNSGALTSGVHTF | YPREAKVQWKVDNALQSGN |
| | PAVLQSSGLYSLSSVVTVPSSSL | SQESVTEQDSKDSTYSLSSTL |
| | GTQTYICNVNHKPSNTKVDKKV | TLSKADYEKHKVYACEVTHQ |
| | EPKSCDKTHTCPPCPAPELLGGP | GLSSPVTKSFNRGEC (SEQ ID |
| | DVFLFPPKPKDTLMISRTPEVTC | NO: 121) |
| | VVVDVSHEDPEVKFNWYVDGV | |
| | EVHNAKTKPREEQYNSTYRVVS | |
| | VLTVLHQDWLNGKEYKCKVSN | |
| | KALPAPEEKTISKAKGQPREPQV | |
| | YTLPPSREEMTKNQVSLTCLVK | |
| | GFYPSDIAVEWESNGQPENNYK | |
| | TTPPVLDSDGSFFLYSKLTVDKS | |
| | RWQQGNVFSCSVMHEALHNHY | |
| | TQKSLSLSPGK (SEQ ID NO: 120) | |
| α-IGF1R 1 | EVQLLESGGGLVQPGGSLRLSC | DIQMTQSPLSLSASVGDRVTI |
| S239D/1332E | AASGFTFSIYRMQWVRQAPGKG | TCQASRDIRNYLNWYQQKPG |
| | LEWVSGISPSGGTTWYADSVKG | KAPKLLIYDASSLQTGVPSRF |
| | RFTISRDNSKNTLYLQMNSLRA | GGSGSGTDFSFTIGSLQPEDIA |
| | EDTAVYYCARWSGGSGYAFDI | TYYCQQFDSLPHTFGQGTKL |
| | WGQGTMVTVSSASTKGPSVFPL | EIKRTVAAPSVFIFPPSDEQLK |
| | APSSKSTSGGTAALGCLVKDYF | SGTASVVCLLNNFYPREAKV |
| | PEPVTVSWNSGALTSGVHTFPA | QWKVDNALQSGNSQESVTE |
| | VLQSSGLYSLSSVVTVPSSSLGT | QDSKDSTYSLSSTLTLSKADY |
| | QTYICNVNHKPSNTKVDKKVEP | EKHKVYACEVTHQGLSSPVT |
| | KSCDKTHTCPPCPAPELLGGPD | KSFNRGEC (SEQ ID NO: 123) |
| | VFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCKVSNK | |
| | ALPAPEEKTISKAKGQPREPQVY | |
| | TLPPSREEMTKNQVSLTCLVKG | |
| | FYPSDIAVEWESNGQPENNYKT | |
| | TPPVLDSDGSFFLYSKLTVDKSR | |
| | WQQGNVFSCSVMHEALHNHYT | |
| | QKSLSLSPGK (SEQ ID NO: 122) | |
| α-IGF1R 2 | EVQLLESGGGLVQPGGSLRLSC | DIQMTQSPDSLAVSLGERATI |
| S239D/1332E | AASGFTFSNYPMYWVRQAPGK | NCKSSQSVLYSSNNKNYLAW |
| | GLEWVSRISSSGGRTVYADSVK | YQQKPGQPPKLLIYLASTRES |
| | GRFTISRDNSKNTLYLQMNSLR | GVPDRFSGSGSGTDFTLTISSL |
| | AEDTAVYYCARDRWSRSAAEY | QAEDVAVYYCQQYYSTWTF |
| | GLGGYWGQGTLVTVSSASTKG | GQGTKVEIKRTVAAPSVFIFP |
| | PSVFPLAPSSKSTSGGTAALGCL | PSDEQLKSGTASVVCLLNNF |
| | VKDYFPEPVTVSWNSGALTSGV | YPREAKVQWKVDNALQSGN |
| | HTFPAVLQSSGLYSLSSVVTVPS | SQESVTEQDSKDSTYSLSSTL |
| | SSLGTQTYICNVNHKPSNTKVD | TLSKADYEKHKVYACEVTHQ |
| | KKVEPKSCDKTHTCPPCPAPELL | GLSSPVTKSFNRGEC (SEQ ID |
| | GGPDVFLFPPKPKDTLMISRTPE | NO: 125) |
| | VTCVVVDVSHEDPEVKFNWYV | |
| | DGVEVHNAKTKPREEQYNSTY | |
| | RVVSVLTVLHQDWLNGKEYKC | |
| | KVSNKALPAPEEKTISKAKGQP | |
| | REPQVYTLPPSREEMTKNQVSL | |
| | TCLVKGFYPSDIAVEWESNGQP | |
| | ENNYKTTPPVLDSDGSFFLYSKL | |
| | TVDKSRWQQGNVFSCSVMHEA | |
| | LHNHYTQKSLSLSPGK (SEQ ID | |
| | NO: 124) | |
| α-ANTXR1 1 | QVQLVQSGAEVKKPGTSVKVSC | SSELTQDPVVSVALGETVSIT |
| mIgG2a | KVPGYTFSSYAISWVRQAPGQG | CQGDNLRDFYASWYQQKPG |
| S239D/1332E | LEWMGGIIPIFGTTNYAQKFQGR | QAPLLVMYGKNRRPSGIPDR |
| | VTITGEESTSTVYMELSSLRSED | FSGSTSGNTLSLTITGAQAED |
| | TAVYYCARDTDYMFDYWGQG | EADYYCSSRDNSKHVVFGGG |
| | TLVTVSSAKTTAPSVYPLAPVC | TKVTVLGQPKSSPSVTLFPPSS |
| | GDTTGSSVTLGCLVKGYFPEPV | EELETNKATLVCTITDFYPGV |
| | TLTWNSGSLSSGVHTFPAVLQS | VTVDWKVDGTPVTQGMETT |
| | DLYTLSSSVTVTSSTWPSQSITC | QPSKQSNNKYMASSYLTLTA |
| | NVAHPASSTKVDKKIEPRGPTIK | RAWERHSSYSCQVTHEGHTV |
| | PCPPCKCPAPNLLGGPDVFIFPP | EKSLSRADCS (SEQ ID NO: |
| | KIKDVLMISLSPIVTCVVVDVSE | 282) |
| | DDPDVQISWFVNNVEVHTAQT | |
| | QTHREDYNSTLRVVSALPIQHQ | |
| | DWMSGKEFKCKVNNKDLPAPE | |
| | ERTISKPKGSVRAPQVYVLPPPE | |
| | EEMTKKQVTLTCMVTDFMPEDI | |
| | YVEWTNNGKTELNYKNTEPVL | |
| | DSDGSYFMYSKLRVEKKNWVE | |
| | RNSYSCSVVHEGLHNHHTTKSF | |
| | SRTPGK (SEQ ID NO: 281) | |
| α-ANTXR1 2 | QVQLKESPGALVKPTQTLTLTC | DIELTQPPSVSVAPGQTARISC |
| mIgG2a | TFSGFSLSTSGGGVSWIRQPPGK | SGDNIGGIYVHWYQQKPGQA |
| S239D/1332E | ALEWLAHIYSNDDKSYSTSLKT | PVLVIYADSKRPSGIPERFSGS |
| | RLTISKDTSKNQVVLTMTNMDP | NSGNTATLTISGTQAEDEADY |
| | VDTATYYCARGGYFLDYWGQG | YCQSYDITSLVFGGGTKLTVL |
| | TLVTVSSAKTTAPSVYPLAPVC | GQPKSSPSVTLFPPSSEELETN |
| | GDTTGSSVTLGCLVKGYFPEPV | KATLVCTITDFYPGVVTVDW |
| | TLTWNSGSLSSGVHTFPAVLQS | KVDGTPVTQGMETTQPSKQS |
| | DLYTLSSSVTVTSSTWPSQSITC | NNKYMASSYLTLTARAWER |
| | NVAHPASSTKVDKKIEPRGPTIK | HSSYSCQVTHEGHTVEKSLSR |
| | PCPPCKCPAPNLLGGPDVFIFPP | ADCS (SEQ ID NO: 284) |
| | KIKDVLMISLSPIVTCVVVDVSE | |
| | DDPDVQISWFVNNVEVHTAQT | |
| | QTHREDYNSTLRVVSALPIQHQ | |
| | DWMSGKEFKCKVNNKDLPAPE | |
| | ERTISKPKGSVRAPQVYVLPPPE | |
| | EEMTKKQVTLTCMVTDFMPEDI | |
| | YVEWTNNGKTELNYKNTEPVL | |
| | DSDGSYFMYSKLRVEKKNWVE | |
| | RNSYSCSVVHEGLHNHHTTKSF | |
| | SRTPGK (SEQ ID NO: 283) | |
| α-CD248 1 | QVQLQESGPGLVAPSQSLSITCT | DIQMNQSPSSLSASLGDTITIT |
| mIgG2a | VSGFSLTGYGVNWVRQPPEKGL | CHASQNINVWLTWYQQKPG |
| S239D/1332E | EWLGMIWVDGSTDYNSALKSR | NIPKLLIYKASNLHTGVPSRFS |
| | LNISKDKSKSQVFLKMNSLQTD | GSGSGTGFTLTISSLQPEDIAT |
| | DTARYYCARGGYGAMDYWGQ | YYCQQGQSYPWTFGGGTKLE |
| | GTSVTVSSAKTTAPSVYPLAPVC | IKRADAAPTVSIFPPSSEQLTS |
| | GDTTGSSVTLGCLVKGYFPEPV | GGASVVCFLNNFYPKDINVK |
| | TLTWNSGSLSSGVHTFPAVLQS | WKIDGSERQNGVLNSWTDQ |
| | DLYTLSSSVTVTSSTWPSQSITC | DSKDSTYSMSSTLTLTKDEYE |
| | NVAHPASSTKVDKKIEPRGPTIK | RHNSYTCEATHKTSTSPIVKS |
| | PCPPCKCPAPNLLGGPDVFIFPP | FNRNEC (SEQ ID NO: 286) |
| | KIKDVLMISLSPIVTCVVVDVSE | |
| | DDPDVQISWFVNNVEVHTAQT | |
| | QTHREDYNSTLRVVSALPIQHQ | |
| | DWMSGKEFKCKVNNKDLPAPE | |
| | ERTISKPKGSVRAPQVYVLPPPE | |
| | EEMTKKQVTLTCMVTDFMPEDI | |
| | YVEWTNNGKTELNYKNTEPVL | |
| | DSDGSYFMYSKLRVEKKNWVE | |
| | RNSYSCSVVHEGLHNHHTTKSF | |
| | SRTPGK (SEQ ID NO: 285) | |
| α-CD248 2 | QVQLQESGPGLVRPSQTLSLTCT | DIQMTQSPSSLSASVGDRVTI |
| mIgG2a | ASGYTFTDYVIHWVKQPPGRGL | TCRASQNVGTAVAWLQQTP |
| S239D/1332E | EWIGYINPYDDDTTYNQKFKGR | GKAPKLLIYSASNRYTGVPSR |
| | VTMLVDTSSNTAYLRLSSVTAE | FSGSGSGTDYTFTISSLQPEDI |
| | DTAVYYCARRGNSYDGYFDYS | ATYYCQQYTNYPMYTFGQG |
| | MDYWGSGTPVTVSSAKTTAPSV | TKVQIKRADAAPTVSIFPPSSE |
| | YPLAPVCGDTTGSSVTLGCLVK | QLTSGGASVVCFLNNFYPKDI |
| | GYFPEPVTLTWNSGSLSSGVHTF | NVKWKIDGSERQNGVLNSW |
| | PAVLQSDLYTLSSSVTVTSSTWP | TDQDSKDSTYSMSSTLTLTK |
| | SQSITCNVAHPASSTKVDKKIEP | DEYERHNSYTCEATHKTSTSP |
| | RGPTIKPCPPCKCPAPNLLGGPD | IVKSFNRNEC (SEQ ID NO: |
| | VFIFPPKIKDVLMISLSPIVTCVV | 288) |
| | VDVSEDDPDVQISWFVNNVEVH | |
| | TAQTQTHREDYNSTLRVVSALPI | |
| | QHQDWMSGKEFKCKVNNKDLP | |
| | APEERTISKPKGSVRAPQVYVLP | |
| | PPEEEMTKKQVTLTCMVTDFMP | |
| | EDIYVEWTNNGKTELNYKNTEP | |
| | VLDSDGSYFMYSKLRVEKKNW | |
| | VERNSYSCSVVHEGLHNHHTTK | |
| | SFSRTPGK (SEQ ID NO: 287) | |
| α-GPC3 1 mIgG2a | QVQLQQSGAELVRPGASVKLSC | DVVMTQTPLSLPVSLGDQASI |
| S239D/1332E | KASGYTFTDYEMHWVKQTPVH | SCRSSQSLVHSNGNTYLHWY |
| | GLKWIGALDPKTGDTAYSQKFK | LQKPGQSPKLLIYKVSNRFSG |
| | GKATLTADKSSSTAYMELRSLT | VPDRFSGSGSGTDFTLKISRV |
| | SEDSAVYYCTRFYSYTYWGQG | EAEDLGVYFCSQNTHVPPTF |
| | TLVTVSAAKTTAPSVYPLAPVC | GSGTKLEIKRADAAPTVSIFPP |
| | GDTTGSSVTLGCLVKGYFPEPV | SSEQLTSGGASVVCFLNNFYP |
| | TLTWNSGSLSSGVHTFPAVLQS | KDINVKWKIDGSERQNGVLN |
| | DLYTLSSSVTVTSSTWPSQSITC | SWTDQDSKDSTYSMSSTLTL |
| | NVAHPASSTKVDKKIEPRGPTIK | TKDEYERHNSYTCEATHKTS |
| | PCPPCKCPAPNLLGGPDVFIFPP | TSPIVKSFNRNEC (SEQ ID |
| | KIKDVLMISLSPIVTCVVVDVSE | NO: 290) |
| | DDPDVQISWFVNNVEVHTAQT | |
| | QTHREDYNSTLRVVSALPIQHQ | |
| | DWMSGKEFKCKVNNKDLPAPE | |
| | ERTISKPKGSVRAPQVYVLPPPE | |
| | EEMTKKQVTLTCMVTDFMPEDI | |
| | YVEWTNNGKTELNYKNTEPVL | |
| | DSDGSYFMYSKLRVEKKNWVE | |
| | RNSYSCSVVHEGLHNHHTTKSF | |
| | SRTPGK (SEQ ID NO: 289) | |
| α-SIRPA 2 | EVQLQQSGAELVKPGASVKVSC | DIVLTQSPASLAVSLGQRATIS |
| mIgG2a | TGSGFNFKDMYLHWVKQRPEQ | CRASESVQYYGTSLMQWYQ |
| S239D/1332E | GLEWIGRIDPENDSTKYDPKFQ | QKPGQPPKLLIYAASNVGSG |
| | DKATLTADTASNTAYLQISSLTS | VPARFSGSGSGTDFSLNIHPV |
| | EDTAVYYCARGNYVHWGQGTL | EEDDIAMYFCQQTRRVWTFG |
| | VTVSAAKTTAPSVYPLAPVCGD | GGTKLEIKRADAAPTVSIFPPS |
| | TTGSSVTLGCLVKGYFPEPVTLT | SEQLTSGGASVVCFLNNFYPK |
| | WNSGSLSSGVHTFPAVLQSDLY | DINVKWKIDGSERQNGVLNS |
| | TLSSSVTVTSSTWPSQSITCNVA | WTDQDSKDSTYSMSSTLTLT |
| | HPASSTKVDKKIEPRGPTIKPCPP | KDEYERHNSYTCEATHKTST |
| | CKCPAPNLLGGPDVFIFPPKIKD | SPIVKSFNRNEC (SEQ ID NO: |
| | VLMISLSPIVTCVVVDVSEDDPD | 292) |
| | VQISWFVNNVEVHTAQTQTHRE | |
| | DYNSTLRVVSALPIQHQDWMSG | |
| | KEFKCKVNNKDLPAPEERTISKP | |
| | KGSVRAPQVYVLPPPEEEMTKK | |
| | QVTLTCMVTDFMPEDIYVEWT | |
| | NNGKTELNYKNTEPVLDSDGSY | |
| | FMYSKLRVEKKNWVERNSYSC | |
| | SVVHEGLHNHHTTKSFSRTPGK | |
| | (SEQ ID NO: 291) | |
| α-PDGFRb 1 | QVQLQESGPGLVKPSETLSLTCT | DIQMTQSPSSLSASVGDRVTI |
| L234A/L235A | VSGFSLTNYAINWVRQPPGKGL | TCHASQNINVWLSWYQQKP |
| | EWLGIIWTGGGTSYNSALKSRL | GKAPKLLIYKASNLHTGVPSR |
| | TISKDTSKNQVSLKLSSVTAADT | FSGSGSGTGFTLTISSLQPEDI |
| | AVYYCARTGTRGYFFDYWGQG | ATYYCQQGQSFPFTFGGGTK |
| | TLVTVSSASTKGPSVFPLAPSSK | VEIKRTVAAPSVFIFPPSDEQL |
| | STSGGTAALGCLVKDYFPEPVT | KSGTASVVCLLNNFYPREAK |
| | VSWNSGALTSGVHTFPAVLQSS | VQWKVDNALQSGNSQESVT |
| | GLYSLSSVVTVPSSSLGTQTYIC | EQDSKDSTYSLSSTLTLSKAD |
| | NVNHKPSNTKVDKKVEPKSCD | YEKHKVYACEVTHQGLSSPV |
| | KTHTCPPCPAPEAAGGPSVFLFP | TKSFNRGEC (SEQ ID NO: 127) |
| | PKPKDTLMISRTPEVTCVVVDV | |
| | SHEDPEVKFNWYVDGVEVHNA | |
| | KTKPREEQYNSTYRVVSVLTVL | |
| | HQDWLNGKEYKCKVSNKALPA | |
| | PIEKTISKAKGQPREPQVYTLPPS | |
| | REEMTKNQVSLTCLVKGFYPSD | |
| | IAVEWESNGQPENNYKTTPPVL | |
| | DSDGSFFLYSKLTVDKSRWQQG | |
| | NVFSCSVMHEALHNHYTQKSLS | |
| | LSPGK (SEQ ID NO: 126) | |
| α-SIRPA 2 | EVQLQQSGAELVKPGASVKVSC | DIVLTQSPASLAVSLGQRATIS |
| | TGSGFNFKDMYLHWVKQRPEQ | CRASESVQYYGTSLMQWYQ |
| | GLEWIGRIDPENDSTKYDPKFQ | QKPGQPPKLLIYAASNVGSG |
| | DKATLTADTASNTAYLQISSLTS | VPARFSGSGSGTDFSLNIHPV |
| | EDTAVYYCARGNYVHWGQGTL | EEDDIAMYFCQQTRRVWTFG |
| | VTVSAASTKGPSVFPLAPSSKST | GGTKLEIKRTVAAPSVFIFPPS |
| | SGGTAALGCLVKDYFPEPVTVS | DEQLKSGTASVVCLLNNFYP |
| | WNSGALTSGVHTFPAVLQSSGL | REAKVQWKVDNALQSGNSQ |
| | YSLSSVVTVPSSSLGTQTYICNV | ESVTEQDSKDSTYSLSSTLTL |
| | NHKPSNTKVDKKVEPKSCDKTH | SKADYEKHKVYACEVTHQG |
| | TCPPCPAPELLGGPSVFLFPPKPK | LSSPVTKSFNRGEC (SEQ ID |
| | DTLMISRTPEVTCVVVDVSHED | NO: 129) |
| | PEVKFNWYVDGVEVHNAKTKP | |
| | REEQYNSTYRVVSVLTVLHQD | |
| | WLNGKEYKCKVSNKALPAPIEK | |
| | TISKAKGQPREPQVYTLPPSREE | |
| | MTKNQVSLTCLVKGFYPSDIAV | |
| | EWESNGQPENNYKTTPPVLDSD | |
| | GSFFLYSKLTVDKSRWQQGNVF | |
| | SCSVMHEALHNHYTQKSLSLSP | |
| | GK (SEQ ID NO: 128) | |
| α-SIRPA 2 | EVQLQQSGAELVKPGASVKVSC | DIVLTQSPASLAVSLGQRATIS |
| L234A/L235A | TGSGFNFKDMYLHWVKQRPEQ | CRASESVQYYGTSLMQWYQ |
| | GLEWIGRIDPENDSTKYDPKFQ | QKPGQPPKLLIYAASNVGSG |
| | DKATLTADTASNTAYLQISSLTS | VPARFSGSGSGTDFSLNIHPV |
| | EDTAVYYCARGNYVHWGQGTL | EEDDIAMYFCQQTRRVWTFG |
| | VTVSAASTKGPSVFPLAPSSKST | GGTKLEIKRTVAAPSVFIFPPS |
| | SGGTAALGCLVKDYFPEPVTVS | DEQLKSGTASVVCLLNNFYP |
| | WNSGALTSGVHTFPAVLQSSGL | REAKVQWKVDNALQSGNSQ |
| | YSLSSVVTVPSSSLGTQTYICNV | ESVTEQDSKDSTYSLSSTLTL |
| | NHKPSNTKVDKKVEPKSCDKTH | SKADYEKHKVYACEVTHQG |
| | TCPPCPAPEAAGGPSVFLFPPKP | LSSPVTKSFNRGEC (SEQ ID |
| | KDTLMISRTPEVTCVVVDVSHE | NO: 131) |
| | DPEVKFNWYVDGVEVHNAKTK | |
| | PREEQYNSTYRVVSVLTVLHQD | |
| | WLNGKEYKCKVSNKALPAPIEK | |
| | TISKAKGQPREPQVYTLPPSREE | |
| | MTKNQVSLTCLVKGFYPSDIAV | |
| | EWESNGQPENNYKTTPPVLDSD | |
| | GSFFLYSKLTVDKSRWQQGNVF | |
| | SCSVMHEALHNHYTQKSLSLSP | |
| | GK (SEQ ID NO: 130) | |
| α-SIRPA 2 | EVQLQQSGAELVKPGASVKVSC | DIVLTQSPASLAVSLGQRATIS |
| S239D/A330L/ | TGSGFNFKDMYLHWVKQRPEQ | CRASESVQYYGTSLMQWYQ |
| I332E | GLEWIGRIDPENDSTKYDPKFQ | QKPGQPPKLLIYAASNVGSG |
| | DKATLTADTASNTAYLQISSLTS | VPARFSGSGSGTDFSLNIHPV |
| | EDTAVYYCARGNYVHWGQGTL | EEDDIAMYFCQQTRRVWTFG |
| | VTVSAASTKGPSVFPLAPSSKST | GGTKLEIKRTVAAPSVFIFPPS |
| | SGGTAALGCLVKDYFPEPVTVS | DEQLKSGTASVVCLLNNFYP |
| | WNSGALTSGVHTFPAVLQSSGL | REAKVQWKVDNALQSGNSQ |
| | YSLSSVVTVPSSSLGTQTYICNV | ESVTEQDSKDSTYSLSSTLTL |
| | NHKPSNTKVDKKVEPKSCDKTH | SKADYEKHKVYACEVTHQG |
| | TCPPCPAPELLGGPDVFLFPPKP | LSSPVTKSFNRGEC (SEQ ID |
| | KDTLMISRTPEVTCVVVDVSHE | NO: 133) |
| | DPEVKFNWYVDGVEVHNAKTK | |
| | PREEQYNSTYRVVSVLTVLHQD | |
| | WLNGKEYKCKVSNKALPLPEE | |
| | KTISKAKGQPREPQVYTLPPSRE | |
| | EMTKNQVSLTCLVKGFYPSDIA | |
| | VEWESNGQPENNYKTTPPVLDS | |
| | DGSFFLYSKLTVDKSRWQQGN | |
| | VFSCSVMHEALHNHYTQKSLSL | |
| | SPGK (SEQ ID NO: 132) | |
| α-SIRPA 2 | EVQLQQSGAELVKPGASVKVSC | DIVLTQSPASLAVSLGQRATIS |
| F243L/R292P/ | TGSGFNFKDMYLHWVKQRPEQ | CRASESVQYYGTSLMQWYQ |
| Y300L | GLEWIGRIDPENDSTKYDPKFQ | QKPGQPPKLLIYAASNVGSG |
| | DKATLTADTASNTAYLQISSLTS | VPARFSGSGSGTDFSLNIHPV |
| | EDTAVYYCARGNYVHWGQGTL | EEDDIAMYFCQQTRRVWTFG |
| | VTVSAASTKGPSVFPLAPSSKST | GGTKLEIKRTVAAPSVFIFPPS |
| | SGGTAALGCLVKDYFPEPVTVS | DEQLKSGTASVVCLLNNFYP |
| | WNSGALTSGVHTFPAVLQSSGL | REAKVQWKVDNALQSGNSQ |
| | YSLSSVVTVPSSSLGTQTYICNV | ESVTEQDSKDSTYSLSSTLTL |
| | NHKPSNTKVDKKVEPKSCDKTH | SKADYEKHKVYACEVTHQG |
| | TCPPCPAPELLGGPSVFLLPPKP | LSSPVTKSFNRGEC (SEQ ID |
| | KDTLMISRTPEVTCVVVDVSHE | NO: 135) |
| | DPEVKFNWYVDGVEVHNAKTK | |
| | PPEEQYNSTLRVVSVLTVLHQD | |
| | WLNGKEYKCKVSNKALPAPIEK | |
| | TISKAKGQPREPQVYTLPPSREE | |
| | MTKNQVSLTCLVKGFYPSDIAV | |
| | EWESNGQPENNYKTTPPVLDSD | |
| | GSFFLYSKLTVDKSRWQQGNVF | |
| | SCSVMHEALHNHYTQKSLSLSP | |
| | GK (SEQ ID NO: 134) | |
| α-SIRPA 2 | EVQLQQSGAELVKPGASVKVSC | DIVLTQSPASLAVSLGQRATIS |
| L235V/F243L/ | TGSGFNFKDMYLHWVKQRPEQ | CRASESVQYYGTSLMQWYQ |
| R292P/Y300L/ | GLEWIGRIDPENDSTKYDPKFQ | QKPGQPPKLLIYAASNVGSG |
| P396L | DKATLTADTASNTAYLQISSLTS | VPARFSGSGSGTDFSLNIHPV |
| | EDTAVYYCARGNYVHWGQGTL | EEDDIAMYFCQQTRRVWTFG |
| | VTVSAASTKGPSVFPLAPSSKST | GGTKLEIKRTVAAPSVFIFPPS |
| | SGGTAALGCLVKDYFPEPVTVS | DEQLKSGTASVVCLLNNFYP |
| | WNSGALTSGVHTFPAVLQSSGL | REAKVQWKVDNALQSGNSQ |
| | YSLSSVVTVPSSSLGTQTYICNV | ESVTEQDSKDSTYSLSSTLTL |
| | NHKPSNTKVDKKVEPKSCDKTH | SKADYEKHKVYACEVTHQG |
| | TCPPCPAPELVGGPSVFLLPPKP | LSSPVTKSFNRGEC (SEQ ID |
| | KDTLMISRTPEVTCVVVDVSHE | NO: 137) |
| | DPEVKFNWYVDGVEVHNAKTK | |
| | PPEEQYNSTLRVVSVLTVLHQD | |
| | WLNGKEYKCKVSNKALPAPIEK | |
| | TISKAKGQPREPQVYTLPPSREE | |
| | MTKNQVSLTCLVKGFYPSDIAV | |
| | EWESNGQPENNYKTTPLVLDSD | |
| | GSFFLYSKLTVDKSRWQQGNVF | |
| | SCSVMHEALHNHYTQKSLSLSP | |
| | GK (SEQ ID NO: 136) | |
| hIgG1 Fc-NKG2D | PKSSDKTHTCPPCPAPELLGGPS | N/A |
| | VFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCKVSNK | |
| | ALPAPIEKTISKAKGQPREPQVY | |
| | TLPPSRDELTKNQVSLTCLVKGF | |
| | YPSDIAVEWESNGQPENNYKTT | |
| | PPVLDSDGSFFLYSKLTVDKSR | |
| | WQQGNVFSCSVMHEALHNHYT | |
| | QKSLSLSPGKIEGRFLNSLFNQE | |
| | VQIPLTESYCGPCPKNWICYKN | |
| | NCYQFFDESKNWYESQASCMS | |
| | QNASLLKVYSKEDQDLLKLVKS | |
| | YHWMGLVHIPTNGSWQWEDGS | |
| | ILSPNLLTIIEMQKGDCALYASSF | |
| | KGYIENCSTPNTYICMQRTV | |
| | (SEQ ID NO: 138) | |
| hIgG1 Fc-NKG2D | PKSSDKTHTCPPCPAPELLGGPD | N/A |
| S239D/I332E | VFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCKVSNK | |
| | ALPAPEEKTISKAKGQPREPQVY | |
| | TLPPSRDELTKNQVSLTCLVKGF | |
| | YPSDIAVEWESNGQPENNYKTT | |
| | PPVLDSDGSFFLYSKLTVDKSR | |
| | WQQGNVFSCSVMHEALHNHYT | |
| | QKSLSLSPGKIEGRFLNSLFNQE | |
| | VQIPLTESYCGPCPKNWICYKN | |
| | NCYQFFDESKNWYESQASCMS | |
| | QNASLLKVYSKEDQDLLKLVKS | |
| | YHWMGLVHIPTNGSWQWEDGS | |
| | ILSPNLLTIIEMQKGDCALYASSF | |
| | KGYIENCSTPNTYICMQRTV | |
| | (SEQ ID NO: 139) | |
| hIgG1 Fc-NKG2D | PKSSDKTHTCPPCPAPEAAGGPS | N/A |
| L234A/L235A | VFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCKVSNK | |
| | ALPAPIEKTISKAKGQPREPQVY | |
| | TLPPSRDELTKNQVSLTCLVKGF | |
| | YPSDIAVEWESNGQPENNYKTT | |
| | PPVLDSDGSFFLYSKLTVDKSR | |
| | WQQGNVFSCSVMHEALHNHYT | |
| | QKSLSLSPGKIEGRFLNSLFNQE | |
| | VQIPLTESYCGPCPKNWICYKN | |
| | NCYQFFDESKNWYESQASCMS | |
| | QNASLLKVYSKEDQDLLKLVKS | |
| | YHWMGLVHIPTNGSWQWEDGS | N/A |
| | ILSPNLLTIIEMQKGDCALYASSF | |
| | KGYIENCSTPNTYICMQRTV | |
| | (SEQ ID NO: 140) | |
| NKG2D-hIgG1 Fc | FLNSLFNQEVQIPLTESYCGPCP | |
| S239D/I332E | KNWICYKNNCYQFFDESKNWY | |
| | ESQASCMSQNASLLKVYSKEDQ | |
| | DLLKLVKSYHWMGLVHIPTNGS | |
| | WQWEDGSILSPNLLTIIEMQKG | |
| | DCALYASSFKGYIENCSTPNTYI | |
| | CMQRTVGGSGGGSGFLNSLFNQ | |
| | EVQIPLTESYCGPCPKNWICYKN | |
| | NCYQFFDESKNWYESQASCMS | |
| | QNASLLKVYSKEDQDLLKLVKS | |
| | YHWMGLVHIPTNGSWQWEDGS | |
| | ILSPNLLTIIEMQKGDCALYASSF | |
| | KGYIENCSTPNTYICMQRTVGG | |
| | GGSGGGGSGGGGSGGGGSPKSS | |
| | DKTHTCPPCPAPELLGGPDVFLF | |
| | PPKPKDTLMISRTPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APEEKTISKAKGQPREPQVYTLP | |
| | PSRDELTKNQVSLTCLVKGFYPS | |
| | DIAVEWESNGQPENNYKTTPPV | |
| | LDSDGSFFLYSKLTVDKSRWQQ | |
| | GNVFSCSVMHEALHNHYTQKS | |
| | LSLSPGK (SEQ ID NO: 141) | |
| NKG2D-hIgG1 Fc | FLNSLFNQEVQIPLTESYCGPCP | N/A |
| L234A/L235A | KNWICYKNNCYQFFDESKNWY | |
| | ESQASCMSQNASLLKVYSKEDQ | |
| | DLLKLVKSYHWMGLVHIPTNGS | |
| | WQWEDGSILSPNLLTIIEMQKG | |
| | DCALYASSFKGYIENCSTPNTYI | |
| | CMQRTVGGSGGGSGFLNSLFNQ | |
| | EVQIPLTESYCGPCPKNWICYKN | |
| | NCYQFFDESKNWYESQASCMS | |
| | QNASLLKVYSKEDQDLLKLVKS | |
| | YHWMGLVHIPTNGSWQWEDGS | |
| | ILSPNLLTIIEMQKGDCALYASSF | |
| | KGYIENCSTPNTYICMQRTVGG | |
| | GGSGGGGSGGGGSGGGGSPKSS | |
| | DKTHTCPPCPAPEAAGGPSVFLF | |
| | PPKPKDTLMISRTPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APIEKTISKAKGQPREPQVYTLP | |
| | PSRDELTKNQVSLTCLVKGFYPS | |
| | DIAVEWESNGQPENNYKTTPPV | |
| | LDSDGSFFLYSKLTVDKSRWQQ | |
| | GNVFSCSVMHEALHNHYTQKS | |
| | LSLSPGK (SEQ ID NO: 142) | |
| α-TIGIT 1 IgG1 | EVQLQQSGPGLVKPSQTLSLTC | DIVMTQSPDSLAVSLGERATI |
| | AISGDSVSSNSAAWNWIRQSPSR | NCKSSQTVLYSSNNKKYLAW |
| | GLEWLGKTYYRFKWYSDYAVS | YQQKPGQPPNLLIYWASTRES |
| | VKGRITINPDTSKNQFSLQLNSV | GVPDRFSGSGSGTDFTLTISSL |
| | TPEDTAVFYCTRESTTYDLLAGP | QAEDVAVYYCQQYYSTPFTF |
| | FDYWGQGTLVTVSSASTKGPSV | GPGTKVEIKRTVAAPSVFIFPP |
| | FPLAPSSKSTSGGTAALGCLVKD | SDEQLKSGTASVVCLLNNFY |
| | YFPEPVTVSWNSGALTSGVHTF | PREAKVQWKVDNALQSGNS |
| | PAVLQSSGLYSLSSVVTVPSSSL | QESVTEQDSKDSTYSLSSTLT |
| | GTQTYICNVNHKPSNTKVDKKV | LSKADYEKHKVYACEVTHQ |
| | EPKSCDKTHTCPPCPAPELLGGP | GLSSPVTKSFNRGEC (SEQ ID |
| | SVFLFPPKPKDTLMISRTPEVTC | NO: 294) |
| | VVVDVSHEDPEVKFNWYVDGV | |
| | EVHNAKTKPREEQYNSTYRVVS | |
| | VLTVLHQDWLNGKEYKCKVSN | |
| | KALPAPIEKTISKAKGQPREPQV | |
| | YTLPPSREEMTKNQVSLTCLVK | |
| | GFYPSDIAVEWESNGQPENNYK | |
| | TTPPVLDSDGSFFLYSKLTVDKS | |
| | RWQQGNVFSCSVMHEALHNHY | |
| | TQKSLSLSPGK (SEQ ID NO: 293) | |
| α-PVRIG 1 IgG4 | QVQLVQSGAEVKKPGASVKVS | DIQMTQSPSSLSASVGDRVTI |
| | CKASGYTFTDYNINWVRQAPG | TCRVSENIYSNLAWYQQKPG |
| | QGLEWMGYIYPYIGGSGYAQKF | KAPKLLIYEATNLAEGVPSRF |
| | QGRVTMTRDTSTSTVYMELSSL | SGSGSGTDFTLTISSLQPEDFA |
| | RSEDTAVYYCAREDKTARNAM | TYYCQHFWGTPYTFGQGTKL |
| | DYWGQGTLVTVSSASTKGPSVF | EIKRTVAAPSVFIFPPSDEQLK |
| | PLAPCSRSTSESTAALGCLVKDY | SGTASVVCLLNNFYPREAKV |
| | FPEPVTVSWNSGALTSGVHTFP | QWKVDNALQSGNSQESVTE |
| | AVLQSSGLYSLSSVVTVPSSSLG | QDSKDSTYSLSSTLTLSKADY |
| | TKTYTCNVDHKPSNTKVDKRV | EKHKVYACEVTHQGLSSPVT |
| | ESKYGPPCPPCPAPEFLGGPSVF | KSFNRGEC (SEQ ID NO: 296) |
| | LFPPKPKDTLMISRTPEVTCVVV | |
| | DVSQEDPEVQFNWYVDGVEVH | |
| | NAKTKPREEQFNSTYRVVSVLT | |
| | VLHQDWLNGKEYKCKVSNKGL | |
| | PSSIEKTISKAKGQPREPQVYTLP | |
| | PSQEEMTKNQVSLTCLVKGFYP | |
| | SDIAVEWESNGQPENNYKTTPP | |
| | VLDSDGSFFLYSRLTVDKSRWQ | |
| | EGNVFSCSVMHEALHNHYTQK | |
| | SLSLSLG (SEQ ID NO: 295) | |

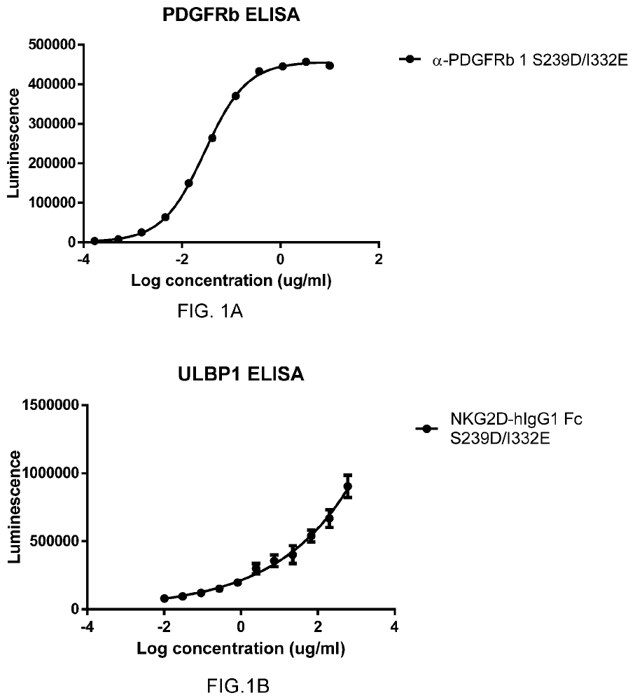

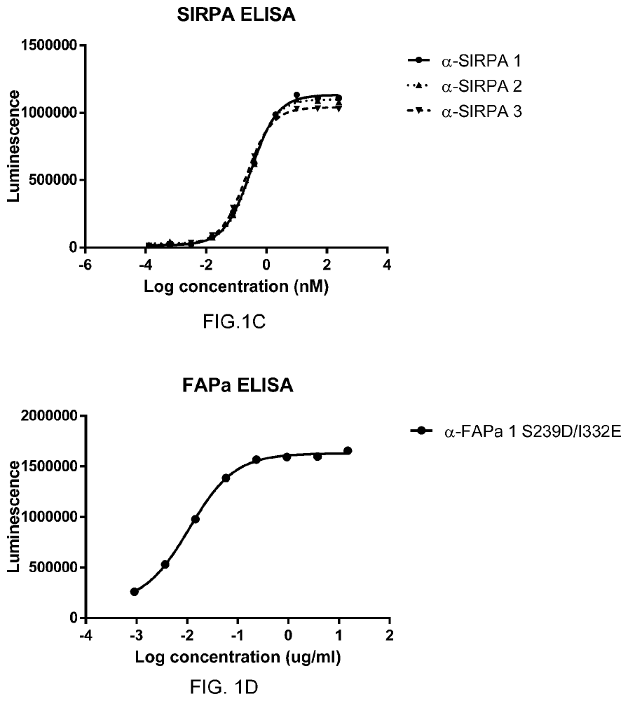

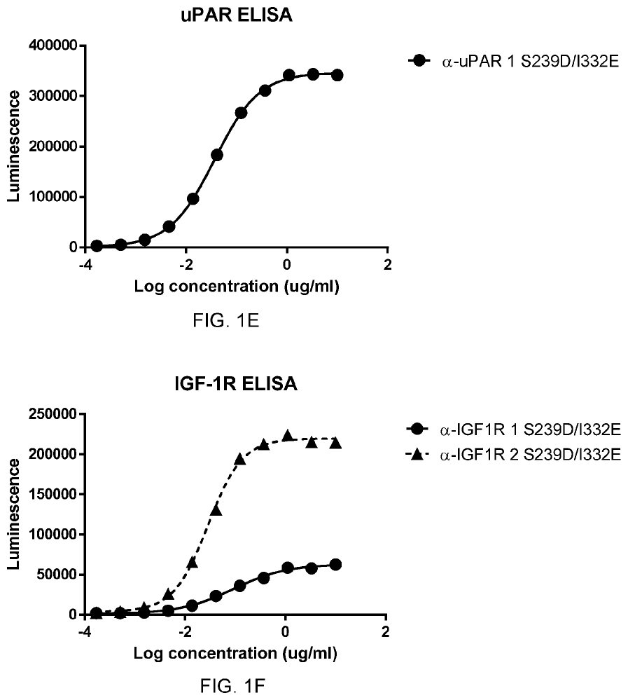

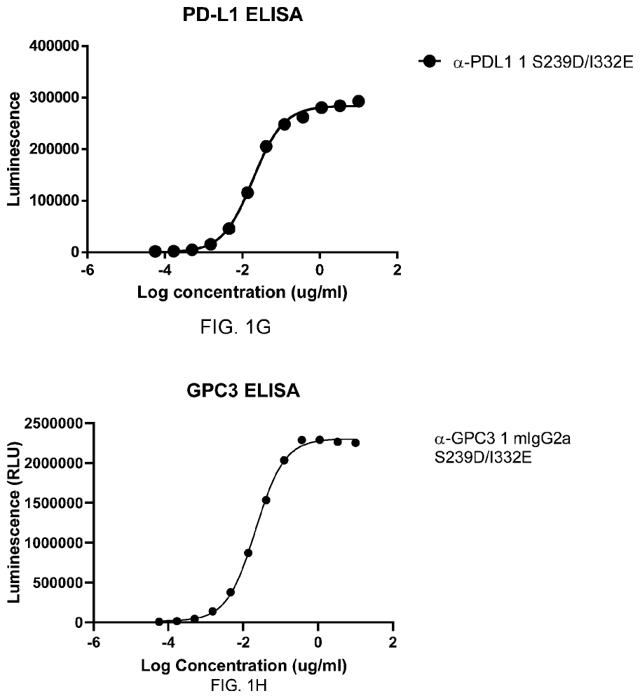

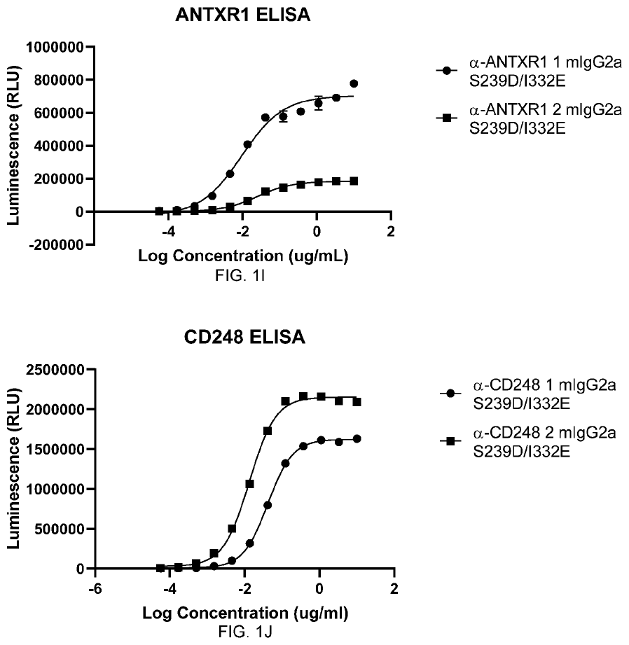

The binding activity of these exemplary constructs to their respective antigens were demonstrated in FIGS. 1A-1J. Briefly, recombinant extracellular domain of PDGFRb, FAPα, ULBP1, uPAR, IGF1R, SIRPA, PD-L1, ANTXR1, CD248, or GPC3 (30 ng/well) was coated on white optiplate™-384 well HB plate. After overnight coating, standard direct ELISA assay was performed. Luminescence signal was detected after using secondary antibody with HRP conjugated. Binding activity of each antibody was determined based on dose dependent binding curve.

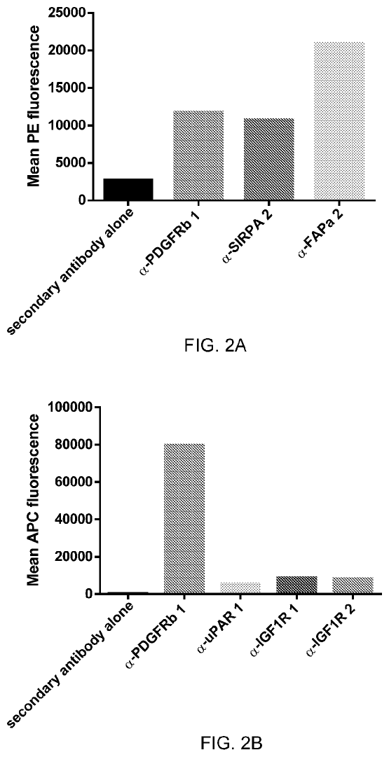

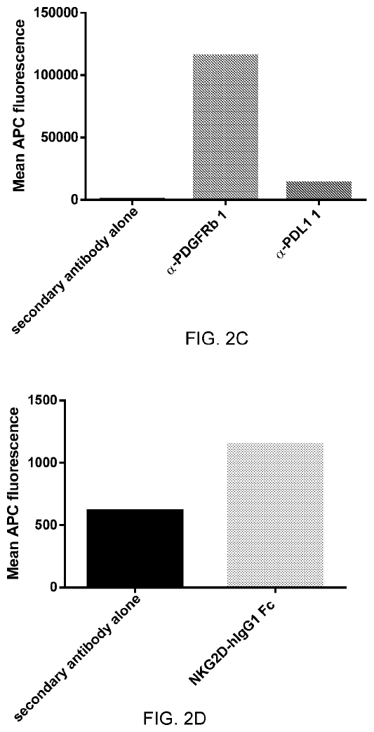

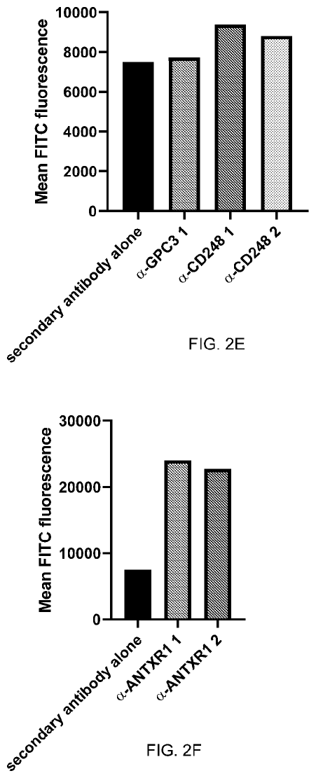

Relative expression levels of the antigens on activated HSC were assayed using FACS. Human primary hepatic stellate cells (ScienCell, 5301) were cultured in culture medium (ScienCell, 5300). The cells were activated using 2 ng/ml human TGFb1 (Sinobiological, 10804-HNAC) for 24 hours. These cells were then collected for FACS assay. Briefly, the cells were incubated with 10 μg/ml primary antibody in blocking buffer (10% FBS in PBS) at 4ºC for 1 hour. After two rounds of PBS washes, the cells were stained with secondary antibody conjugated with a fluorophore. The mean fluorescence intensity was measured using FACS. The FACS results were summarized in FIGS. 2A-2F. Among the antigens tested, fibroblast activation protein alpha (FAPα) was expressed at relatively high levels, while uPAR, IGF-1R, GPC3, CD248 and NKG2D ligands were expressed at relatively low levels on TGFb1-activated HSC cells.

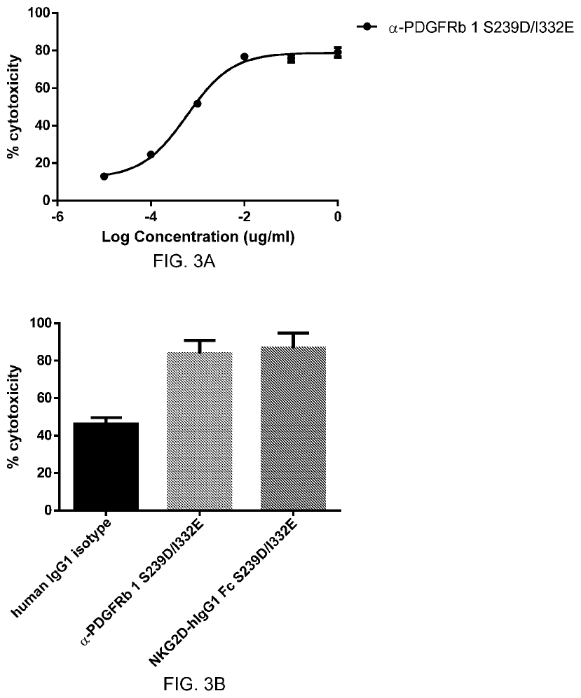

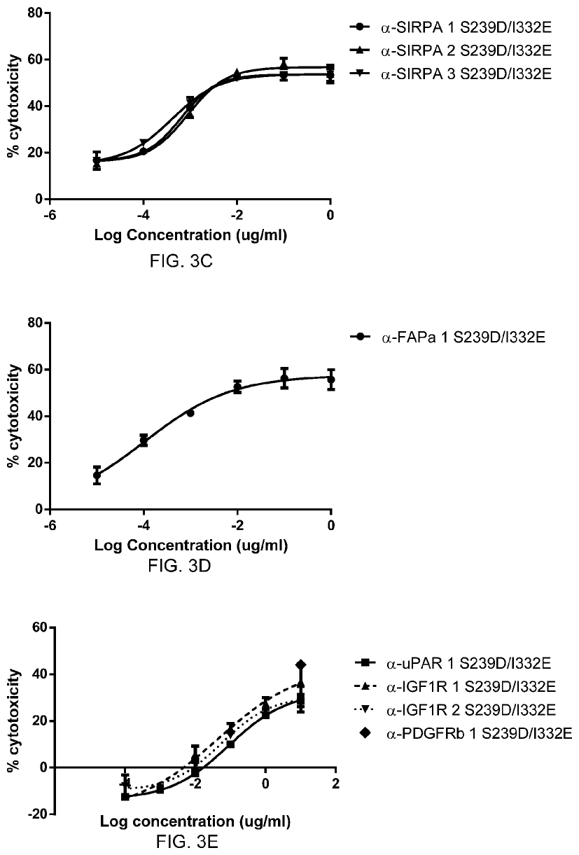

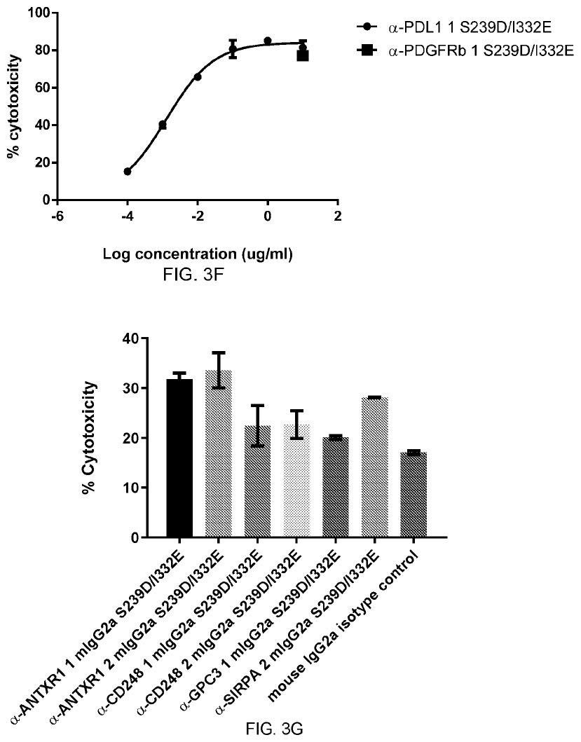

In vitro antibody dependent cell-mediated cytotoxicity assay (ADCC) assay was performed by co-culture of human primary NK or PBMC cells with TGFb1-activated HSC cells. Briefly, human primary NK or PBMC cells were activated by 100 U/ml IL-2 overnight in RPMI 1640 medium containing 10% FBS. Human primary HSC cells were activated with 2 ng/ml hTGFb1 overnight. On the day of ADCC assay, activated HSC cells were collected and seeded onto U-shape 96 well plate. These cells were pretreated with testing antibodies (all contained S239D/1332E mutation in the Fc region) at different concentrations for 30 minutes at 37° C. After pre-treatment, the activated NK or PBMC cells were collected and seeded onto the plate at E:T ratio=4:1 (for NK cells) or ET ratio=25:1 or 50:1 (for PBMC cells). The co-culture was further cultured for additional 4 hours at 37ºC. After co-culture, the culture medium was collected and LDH released from the dying cells were measured by CytoTox 96® Non-Radioactive Cytotoxicity Assay Kit (Promega, G1780). As shown in FIGS. 3A-3G, all antibodies targeting to the surface antigens exerted ADCC cytotoxicity to HSC after co-culturing human NK/PBMC with HSC cells. The magnitude of cytotoxicity induced by testing antibodies or proteins varied due to the expression levels of the target proteins. As long as the target antigen expresses on activated HSC cells, there is an ADCC effect either using antibody or recombinant protein.

### 7.2 Example 2: Fc effector function is required for ADCC cytotoxicity to activiated HSCs

The interaction between Fc region of an antibody with Fc gamma receptors on the immune cells is critical for the effector functions including ADCC, antibody dependent cellular phagocytosis (ADCP), etc. Both non-fucosylation and certain mutations on the Fc region of human IgG1 enhance the effector function of ADCC. Such exemplary non-limiting Fc mutations include Fc variants contain one or more of alterations of the amino acids at the positions 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, 447 according to EU numbering (see, e.g., Sondermann et al., Nature, 406: 267-273 (2000); U.S. Pat. No. 8,951,517B2; U.S. Pat. No. 9,714,282B2; and US20090208500A1, each of which is incorporated herein by reference), which can be introduced in the present Fc containing binding moleucles to enhance ADCC.

Exemplary mutations were introduced to Fc region to either enhance or abolish the effector function especially ADCC activity. S239D/1332E, S239D/A330L/1332E, L235V/F243L/R292P/Y300L/P396L, or F243L/R292P/Y300L mutations were engineered to enhance the effector function. L234A/L235A or N297S was engineered to eliminate the effector function. Exemplary constant regions and variants thereof are shown in Table 5 below.

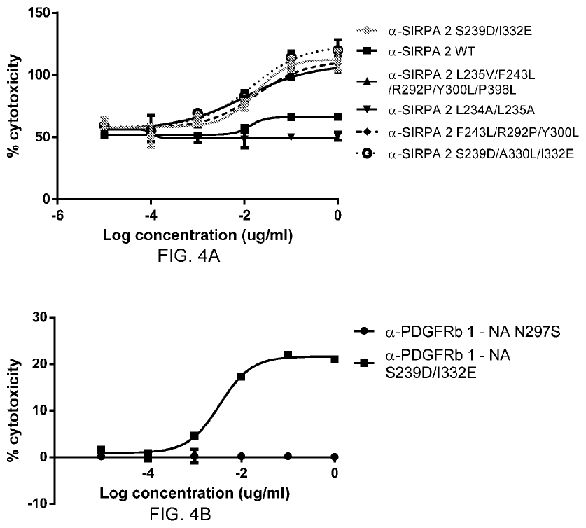

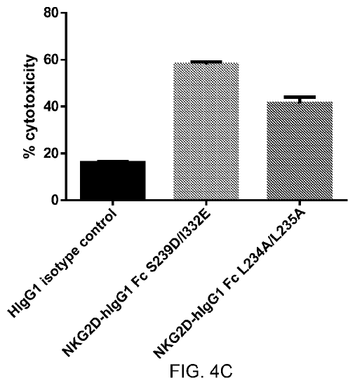

In vitro ADCC assay was performed by co-culturing IL-2-activated NK or PBMC with hTGFb1-activated human primary HSC cells. As shown in FIG. 4A, anti-SIRPA antibody α-SIRPA 2 with wild type hIgG1 Fc induced dose dependent ADCC cytotoxicity to HSC cells. This effect was enhanced by the mutations (S239D/1332E, S239D/A330L/1332E, L235V/F243L/R292P/Y300L/P396L, or F243L/R292P/Y300L). ADCC effect was completely diminished by the silencing mutation L234A/L235A. Fc dependent ADCC effect was also observed for anti-PDGFRb antibody and NKG2D-Fc fusion protein. As shown in FIGS. 4B and 4C, S239D/1332E mutation with enhanced effector function increased ADCC cytotoxicity while silencing mutations (N297S for anti-PDGFRb antibody, L234A/L235A for NKG2D-Fc protein) abolished ADCC effect.

TABLE 5
Exemplary constant region sequences

| | Sequence |
|---|---|
| CL kappa | RTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGL |
| | SSPVTKSFNRGEC (SEQ ID NO: 143) |
| CH wild type | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSP |
| | GK (SEQ ID NO: 144) |
| CH | ASTKGPSVFPLAPSSKSTSGGTAALGCLVKDYFPEPVTVSWNSGALT |
| S239D/1332E | SGVHTFPAVLQSSGLYSLSSVVTVPSSSLGTQTYICNVNHKPSNTKVD |
| | KKVEPKSCDKTHTCPPCPAPELLGGPDVFLFPPKPKDTLMISRTPEVT |
| | CVVVDVSHEDPEVKFNWYVDGVEVHNAKTKPREEQYNSTYRVVSV |
| | LTVLHQDWLNGKEYKCKVSNKALPAPEEKTISKAKGQPREPQVYTL |
| | PPSREEMTKNQVSLTCLVKGFYPSDIAVEWESNGQPENNYKTTPPVL |
| | DSDGSFFLYSKLTVDKSRWQQGNVFSCSVMHEALHNHYTQKSLSLS |
| | PGK (SEQ ID NO: 145) |
| CH | ASTKGPSVFPLAPSSKSTSGGTAALGCLVKDYFPEPVTVSWNSGALT |
| L234A/L235A | SGVHTFPAVLQSSGLYSLSSVVTVPSSSLGTQTYICNVNHKPSNTKVD |
| | KKVEPKSCDKTHTCPPCPAPEAAGGPSVFLFPPKPKDTLMISRTPEVT |
| | CVVVDVSHEDPEVKFNWYVDGVEVHNAKTKPREEQYNSTYRVVSV |
| | LTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSP |
| | GK (SEQ ID NO: 146) |
| CH | ASTKGPSVFPLAPSSKSTSGGTAALGCLVKDYFPEPVTVSWNSGALT |
| S239D/A330L/ | SGVHTFPAVLQSSGLYSLSSVVTVPSSSLGTQTYICNVNHKPSNTKVD |
| I332E | KKVEPKSCDKTHTCPPCPAPELLGGPDVFLFPPKPKDTLMISRTPEVT |
| | CVVVDVSHEDPEVKFNWYVDGVEVHNAKTKPREEQYNSTYRVVSV |
| | LTVLHQDWLNGKEYKCKVSNKALPLPEEKTISKAKGQPREPQVYTL |
| | PPSREEMTKNQVSLTCLVKGFYPSDIAVEWESNGQPENNYKTTPPVL |
| | DSDGSFFLYSKLTVDKSRWQQGNVFSCSVMHEALHNHYTQKSLSLS |
| | PGK (SEQ ID NO: 147) |
| CH | ASTKGPSVFPLAPSSKSTSGGTAALGCLVKDYFPEPVTVSWNSGALT |
| L235V/F243L/ | SGVHTFPAVLQSSGLYSLSSVVTVPSSSLGTQTYICNVNHKPSNTKVD |
| R292P/Y300L/ | KKVEPKSCDKTHTCPPCPAPELVGGPSVFLLPPKPKDTLMISRTPEVT |
| P396L | CVVVDVSHEDPEVKFNWYVDGVEVHNAKTKPPEEQYNSTLRVVSV |
| | LTVLHQDWLNGKEYKCKVSNKALPAPIEKTISKAKGQPREPQVYTLP |
| | PSREEMTKNQVSLTCLVKGFYPSDIAVEWESNGQPENNYKTTPLVLD |
| | SDGSFFLYSKLTVDKSRWQQGNVFSCSVMHEALHNHYTQKSLSLSP |
| | GK (SEQ ID NO: 148) |
| CH | ASTKGPSVFPLAPSSKSTSGGTAALGCLVKDYFPEPVTVSWNSGALT |
| F243L/R292P/ | SGVHTFPAVLQSSGLYSLSSVVTVPSSSLGTQTYICNVNHKPSNTKVD |
| Y300L | KKVEPKSCDKTHTCPPCPAPELLGGPSVFLLPPKPKDTLMISRTPEVT |
| | CVVVDVSHEDPEVKFNWYVDGVEVHNAKTKPPEEQYNSTLRVVSV |
| | LTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSP |
| | GK (SEQ ID NO: 149) |
| CH N297S | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKVEPKSCDKTHTCPPCPAPELLGGPSVFLFPPKPKDTLMISRTPEVT |
| | CVVVDVSHEDPEVKFNWYVDGVEVHNAKTKPREEQYSSTYRVVSV |
| | LTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSP |
| | GK (SEQ ID NO: 150) |
| CH2-CH3 | EPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| | (SEQ ID NO: 151) |
| CH1 | ASTKGPSVFPLAPSSKSTSGGTAALGCLVKDYFPEPVTVSWNSGALT |
| | SGVHTFPAVLQSSGLYSLSSVVTVPSSSLGTQTYICNVNHKPSNTKVD |
| | KKV (SEQ ID NO: 152) |
| CH2-CH3 | EPKSCDKTHTCPPCPAPELLGGPDVFLFPPKPKDTLMISRTPEVTCVV |
| S239D/I332E | VDVSHEDPEVKFNWYVDGVEVHNAKTKPREEQYNSTYRVVSVLTV |
| | LHQDWLNGKEYKCKVSNKALPAPEEKTISKAKGQPREPQVYTLPPS |
| | REEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPG |
| | K (SEQ ID NO: 153) |
| CH2-CH3 | EPKSCDKTHTCPPCPAPELLGGPSVFLFPPKPKDTLMISRTPEVTCVV |
| N297S | VDVSHEDPEVKFNWYVDGVEVHNAKTKPREEQYSSTYRVVSVLTV |
| | LHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| | (SEQ ID NO: 154) |

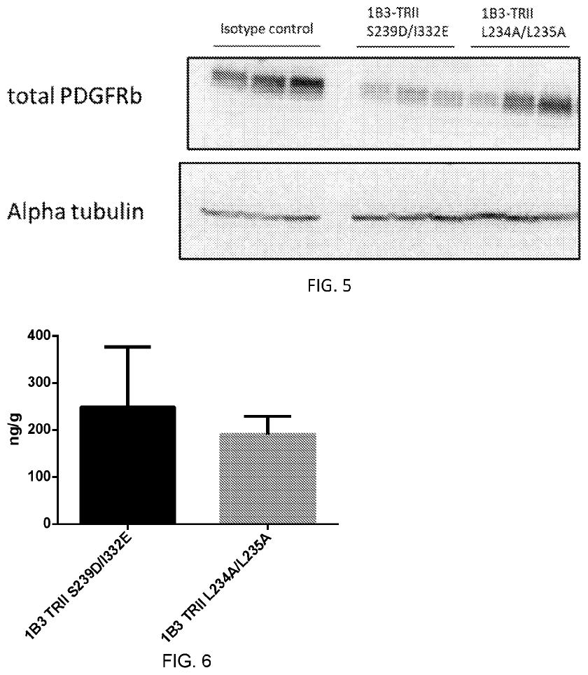

It was demonsterated here that Fc effector function was also required for the depletion of hepatic activated HSC in vivo. Antibody with enhanced ADCC effector function could efficiently eliminate activated HSC in mouse liver, while comparable antibody with abolished ADCC effector function had no effect in mouse model for liver fibrosis. Briefly, male wild type C57 mice were treated with CCL4 (0.7 ml/kg, i.p. injection, three times a week) for three weeks to induce HSC cells activation and liver fibrosis. These animals were given a single dose (45 mg/kg, i.v. injection) of bispecific antibodies of anti-mouse PDGFRb antibody (1B3, see U.S. Pat. No. 7,740,850B2) with TGFb1 receptor II extracellular domain (TRII) fused to heavy chain C terminus (1B3-TRII S239D/1332E and 1B3-TRII L234A/L235A). The antibodies were all engineered as mIgG2α and had Fc mutations of either S239D/1332E as effector function enhancing mutation or L234A/L235A as effector function silencing mutation. Liver tissues were collected at 48 hours after antibody dosing. Liver samples were homogenized in RIPA lysis buffer supplemented with protease and phosphatase inhibitor. After centrifugation, the supernatant was collected as liver lysate. Total PDGFRb protein levels in the liver lysates were measured by western blot as an indication of activated HSC cells depletion in the liver. As shown in FIG. 5, 1B3-TRII S239D/1332E (anti-mPDGFRb antibody with heavy chain C-term linked TRII, mIgG2a, S239D/1332E mutation in Fc) treatment significantly reduced total PDGFRb protein levels in the liver while 1B3-TRII L234A/L235A (anti-mPDGFRb antibody with heavy chain C-term linked TRII, mIgG2a, L234A/L235A mutation in Fc) had no effect on total PDGFRb protein levels. Additional liver samples were homogenized in lysis buffer (PBS: MeOH=3:1). After centrifugation, the supernatant was collected for the determination of antibody concentration in the liver. The testing antibody levels in the liver were measured using direct ELISA with mouse PDGFRb ECD as coating antigen and anti-TRII antibody as detection antibody. Comparable antibody concentration in the liver was observed between 1B3-TRII S239D/1332E and 1B3-TRII L234A/L235A treatment groups (FIG. 6), suggesting the differential effect on total PDGFRb levels by two treatment groups was not due to different antibody exposure in the liver.

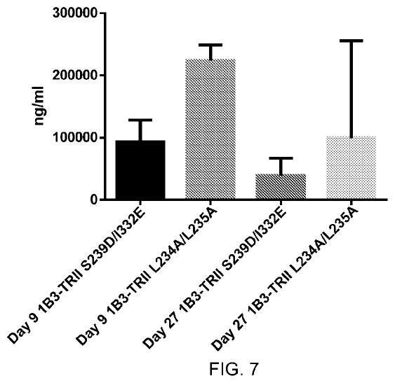

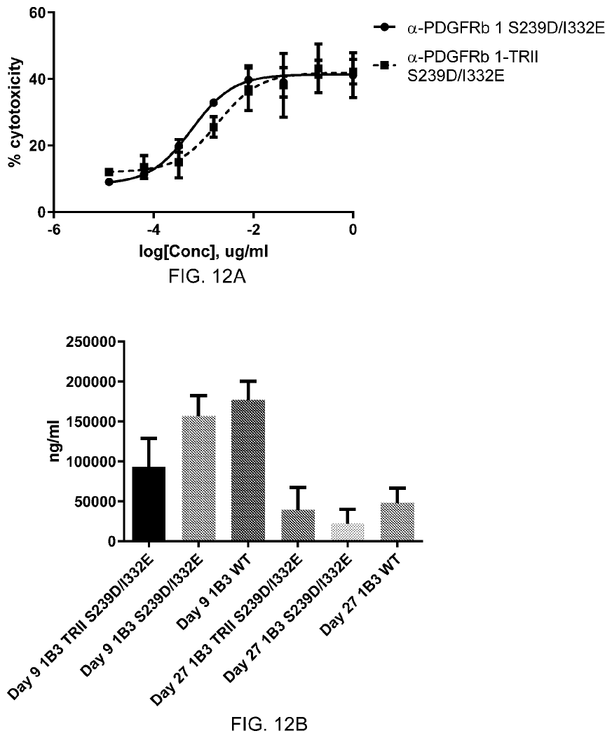

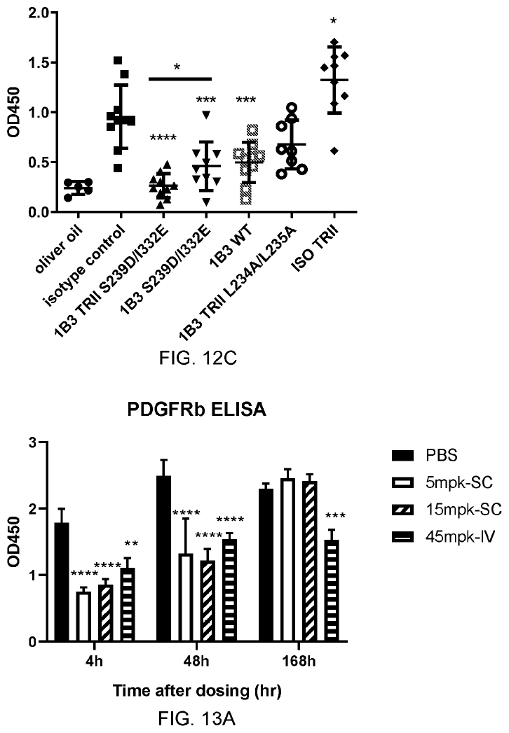

Next, multiple dose study of anti-mouse PDGFRb antibodies was conducted in CCL4-treated mice. Male wild type C57BL/6J mice were treated with CCL4 (0.7 ml/kg, i.p. injection, three times a week) for three weeks to induce HSC cells activation. These mice were then given 45 mg/kg of anti-mouse PDGFRb bispecific antibodies (1B3-TRII S239D/1332E, 1B3-TRII L234A/L235A) (i.v., twice a week for 4 weeks, 8 doses in total) along with CCL4 injections for additional 4 weeks. Liver samples were collected and homogenized in RIPA lysis buffer supplemented with protease and phosphatase inhibitor. After centrifugation, the supernatant was collected as liver lysate. Total PDGFRb levels in the liver lysate were measured by sandwich ELISA. Anti-PDGFRb antibody (abcam Ab32570) was used as capture antibody. 1B3 mIgG2a was used as detection antibody. As shown in FIG. 12C, consistent with single dose study, multiple doses of 1B3-TRII S239D/1332E treatment significantly reduced total PDGFRb protein levels in the liver, while multiple doses of 1B3-TRII L234A/L235A had no significant effect on total PDGFRb protein levels, confirming the critical role of antibody Fc effector function on the depletion of activated HSC in vivo. Antibody exposure was not likely to contribute to this differential effect on HSC depletion. Serum antibody levels were determined using direct ELISA with mouse PDGFRb as coating antigen and anti-TRII antibody as detection antibody. 1B3-TRII S239D/1332E concentration in the serum was lower than 1B3-TRII L234A/L235A at day 9 and day 27 after the start of treatment (FIG. 7).

These results demonstrate that activated HSCs-targeting antibodies with enhanced ADCC effector function could efficiently deplete activated HSCs both in vitro and in vivo, and thus may be employed as effective therapeutics for activated HSC-associated fibrotic diseases or disorders.

### 7.3. Example 3: Increased Cytotoxicity to Activated HSCs by Targeting to Multiple Surface Proteins

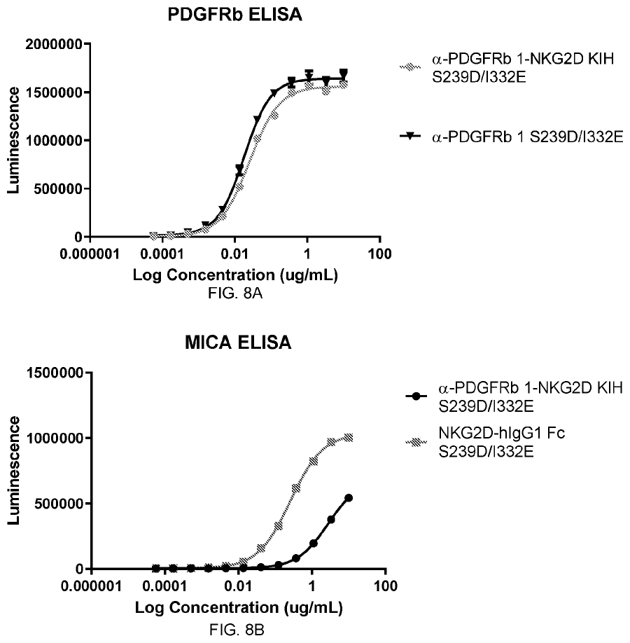

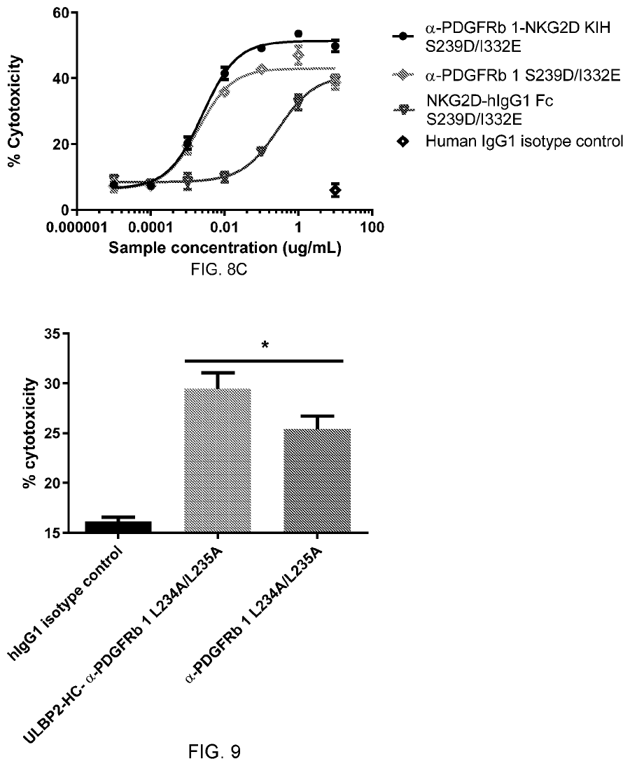

Bispecific antibody was designed to target multiple targets on aHSC cells. Extracellular domain of NKG2D was fused to α-PDGFRb 1 S239D/1332E to make a knob-into-hole bispecific antibody (α-PDGFRb 1-NKG2D KIH S239D/1332E). This bispecific antibody could recognize human PDGFRb and NKG2D ligands (e.g. MICA, MICB) expressed on aHSC cells. Exemplary construct thereof is shown in Table 6 below. The bispecific antibody α-PDGFRb 1-NKG2D KIH S239D/1332E was purified. As shown in FIGS. 8A and 8B, the bispecific antibody α-PDGFRb 1-NKG2D KIH S239D/1332E bind to both human PDGFRb ECD and MICA ECD in a dose dependent manner in ELISA assays. In in vitro ADCC assay, α-PDGFRb 1-NKG2D KIH S239D/1332E, α—PDGFRb 1 S239D/1332E and NKG2D hIgG1 Fc S239D/1332E all dose dependently increased cytotoxicity against aHSC cells. α-PDGFRb 1-NKG2D KIH S239D/1332E further increased maximum aHSC killing comparing to α—PDGFRb 1 S239D/1332E or NKG2D hIgG1 Fc S239D/1332E treatment alone (FIG. 8C).

TABLE 6
Full lengh sequences of exemplary α-PDGFRb 1-NKG2D KIH
S239D/I332E construct

| Construct | Chain 1 | Chain 2 | Chain 3 |
|---|---|---|---|
| α-PDGFRb 1- | QVQLQESGPGLVKPS | FLNSLFNQEVQIP | DIQMTQSPSSLSAS |
| NKG2D KIH | ETLSLTCTVSGFSLTN | LTESYCGPCPKN | VGDRVTITCHASQN |
| S239D/1332E | YAINWVRQPPGKGL | WICYKNNCYQFF | INVWLSWYQQKPG |
| | EWLGIIWTGGGTSYN | DESKNWYESQAS | KAPKLLIYKASNLH |
| | SALKSRLTISKDTSK | CMSQNASLLKVY | TGVPSRFSGSGSGT |
| | NQVSLKLSSVTAADT | SKEDQDLLKLVK | GFTLTISSLQPEDIA |
| | AVYYCARTGTRGYF | SYHWMGLVHIPT | TYYCQQGQSFPFTF |
| | FDYWGQGTLVTVSS | NGSWQWEDGSIL | GGGTKVEIKRTVA |
| | ASTKGPSVFPLAPSS | SPNLLTIIEMQKG | APSVFIFPPSDEQLK |
| | KSTSGGTAALGCLV | DCALYASSFKGYI | SGTASVVCLLNNFY |
| | KDYFPEPVTVSWNS | ENCSTPNTYICMQ | PREAKVQWKVDN |
| | GALTSGVHTFPAVLQ | RTVGGSGGGSGF | ALQSGNSQESVTEQ |
| | SSGLYSLSSVVTVPSS | LNSLFNQEVQIPL | DSKDSTYSLSSTLT |
| | SLGTQTYICNVNHKP | TESYCGPCPKNWI | LSKADYEKHKVYA |
| | SNTKVDKKVEPKSC | CYKNNCYQFFDE | CEVTHQGLSSPVTK |
| | DKTHTCPPCPAPELL | SKNWYESQASCM | SFNRGEC (SEQ ID |
| | GGPDVFLFPPKPKDT | SQNASLLKVYSK | NO: 299) |
| | LMISRTPEVTCVVVD | EDQDLLKLVKSY | |
| | VSHEDPEVKFNWYV | HWMGLVHIPTNG | |
| | DGVEVHNAKTKPRE | SWQWEDGSILSP | |
| | EQYNSTYRVVSVLT | NLLTIIEMQKGDC | |
| | VLHQDWLNGKEYKC | ALYASSFKGYIEN | |
| | KVSNKALPAPEEKTI | CSTPNTYICMQRT | |
| | SKAKGQPREPQVCTL | VGGGGSGGGGSG | |
| | PPSREEMTKNQVSLS | GGGSGGGGSPKS | |
| | CAVKGFYPSDIAVE | SDKTHTCPPCPAP | |
| | WESNGQPENNYKTT | ELLGGPDVFLFPP | |
| | PPVLDSDGSFFLVSK | KPKDTLMISRTPE | |
| | LTVDKSRWQQGNVF | VTCVVVDVSHED | |
| | SCSVMHEALHNHYT | PEVKFNWYVDGV | |
| | QKSLSLSPGK (SEQ | EVHNAKTKPREE | |
| | ID NO: 297) | QYNSTYRVVSVL | |
| | | TVLHQDWLNGKE | |
| | | YKCKVSNKALPA | |
| | | PEEKTISKAKGQP | |
| | | REPQVYTLPPCRD | |
| | | ELTKNQVSLWCL | |
| | | VKGFYPSDIAVE | |
| | | WESNGQPENNYK | |
| | | TTPPVLDSDGSFF | |
| | | LYSKLTVDKSRW | |
| | | QQGNVFSCSVMH | |
| | | EALHNHYTQKSL | |
| | | SLSPGK (SEQ ID | |
| | | NO: 298) | |

### 7.4. Example 4: Increased Cytotoxicity to Activated HSCs by Activating Immune Cells

In addition to Fc dependent effector function, activation of immune cells (such as NK cells) by manipulating the activity of immune receptors on the cells could be another approach to further enhance cytotoxicity to activated HSCs. In a non-limiting aspect, these immune checkpoint receptors include: NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRG1, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, , and A2a.

To further investigate the effect of modulating the activity of immune cell receptors on cell-mediated cytotoxicity for depleting HSCs, various designs and constructs were developed. Bispecific antibodies were designed with one motif to bind to activated HSC and the other motif to activate immune cells. NKG2D is an activating receptor expressed on immune cells with cytotoxic activity. Engagement of NKG2D could activate the immune cells to exert cytolytic function to the target cells. Bispecific antibodies by fusion ECD of NKG2D ligands (e.g., MICA, MICB, ULBP1, ULBP2) to either N-term or C-term of heavy chain of anti-PDGFRb antibody were designed and purified. Exemplary constructs thereof are shown in Table 7 below.

The effects of these bi-specific constructs were evaluated in in vitro ADCC cytotoxicity assay. To avoid the interference of antibody Fc effector ADCC function, these antibodies were engineered to contain an effector function silencing mutation (L234A/L235A) to abolish the interaction between Fc and Fc gamma receptors. As shown in FIG. 9, the bispecific antibody with ECD of ULBP2 fused to N-term of anti-PDGFRb antibody heavy chain could efficiently induce more cytotoxicity to activated HSC cells comparing to anti-PDGFRb antibody alone, suggesting activation of NKG2D signaling pathway could further enhance the antibody-dependent cytotoxicity to activated HSCs.

TABLE 7
Full lengh sequences of exemplary α-PDGFRb 1-protein fusion constructs

| Construct | Chain 1 | Chain 2 |
|---|---|---|
| α-PDGFRb 1-TRII | QVQLQESGPGLVKPSETLSLTCT | DIQMTQSPSSLSASVGDRVTI |
| S239D/I332E | VSGFSLTNYAINWVRQPPGKGL | TCHASQNINVWLSWYQQKP |
| | EWLGIIWTGGGTSYNSALKSRL | GKAPKLLIYKASNLHTGVPSR |
| | TISKDTSKNQVSLKLSSVTAADT | FSGSGSGTGFTLTISSLQPEDI |
| | AVYYCARTGTRGYFFDYWGQG | ATYYCQQGQSFPFTFGGGTK |
| | TLVTVSSASTKGPSVFPLAPSSK | VEIKRTVAAPSVFIFPPSDEQL |
| | STSGGTAALGCLVKDYFPEPVT | KSGTASVVCLLNNFYPREAK |
| | VSWNSGALTSGVHTFPAVLQSS | VQWKVDNALQSGNSQESVT |
| | GLYSLSSVVTVPSSSLGTQTYIC | EQDSKDSTYSLSSTLTLSKAD |
| | NVNHKPSNTKVDKKVEPKSCD | YEKHKVYACEVTHQGLSSPV |
| | KTHTCPPCPAPELLGGPDVFLFP | TKSFNRGEC (SEQ ID NO: 156) |
| | PKPKDTLMISRTPEVTCVVVDV | |
| | SHEDPEVKFNWYVDGVEVHNA | |
| | KTKPREEQYNSTYRVVSVLTVL | |
| | HQDWLNGKEYKCKVSNKALPA | |
| | PEEKTISKAKGQPREPQVYTLPP | |
| | SREEMTKNQVSLTCLVKGFYPS | |
| | DIAVEWESNGQPENNYKTTPPV | |
| | LDSDGSFFLYSKLTVDKSRWQQ | |
| | GNVFSCSVMHEALHNHYTQKS | |
| | LSLSPGKGGGGSGGGGSGGGGS | |
| | GGGGSGIPPHVQKSVNNDMIVT | |
| | DNNGAVKFPQLCKFCDVRFSTC | |
| | DNQKSCMSNCSITSICEKPQEVC | |
| | VAVWRKNDENITLETVCHDPKL | |
| | PYHDFILEDAASPKCIMKEKKKP | |
| | GETFFMCSCSSDECNDNIIFSEEY | |
| | NTSNPD (SEQ ID NO: 155) | |
| MICA-GS-HC-α- | EPHSLRYNLTVLSWDGSVQSGF | DIQMTQSPSSLSASVGDRVTI |
| PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 158) |
| | TYQTWVATRICQGEEQRFTCYM | |
| | EHSGNHSTHPVPSGKVLVLQSH | |
| | WGGGGSQVQLQESGPGLVKPSE | |
| | TLSLTCTVSGFSLTNYAINWVR | |
| | QPPGKGLEWLGIIWTGGGTSYN | |
| | SALKSRLTISKDTSKNQVSLKLS | |
| | SVTAADTAVYYCARTGTRGYFF | |
| | DYWGQGTLVTVSSASTKGPSVF | |
| | PLAPSSKSTSGGTAALGCLVKD | |
| | YFPEPVTVSWNSGALTSGVHTF | |
| | PAVLQSSGLYSLSSVVTVPSSSL | |
| | GTQTYICNVNHKPSNTKVDKKV | |
| | EPKSCDKTHTCPPCPAPELLGGP | |
| | DVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCKVSN | |
| | KALPAPEEKTISKAKGQPREPQV | |
| | YTLPPSREEMTKNQVSLTCLVK | |
| | GFYPSDIAVEWESNGQPENNYK | |
| | TTPPVLDSDGSFFLYSKLTVDKS | |
| | RWQQGNVFSCSVMHEALHNHY | |
| | TQKSLSLSPGK (SEQ ID NO: 157) | |
| MICA-HC-α- | EPHSLRYNLTVLSWDGSVQSGF | DIQMTQSPSSLSASVGDRVTI |
| PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 160) |
| | TYQTWVATRICQGEEQRFTCYM | |
| | EHSGNHSTHPVPSGKVLVLQSH | |
| | WQVQLQESGPGLVKPSETLSLT | |
| | CTVSGFSLTNYAINWVRQPPGK | |
| | GLEWLGIIWTGGGTSYNSALKS | |
| | RLTISKDTSKNQVSLKLSSVTAA | |
| | DTAVYYCARTGTRGYFFDYWG | |
| | QGTLVTVSSASTKGPSVFPLAPS | |
| | SKSTSGGTAALGCLVKDYFPEP | |
| | VTVSWNSGALTSGVHTFPAVLQ | |
| | SSGLYSLSSVVTVPSSSLGTQTYI | |
| | CNVNHKPSNTKVDKKVEPKSC | |
| | DKTHTCPPCPAPELLGGPDVFLF | |
| | PPKPKDTLMISRTPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APEEKTISKAKGQPREPQVYTLP | |
| | PSREEMTKNQVSLTCLVKGFYP | |
| | SDIAVEWESNGQPENNYKTTPP | |
| | VLDSDGSFFLYSKLTVDKSRWQ | |
| | QGNVFSCSVMHEALHNHYTQK | |
| | SLSLSPGK (SEQ ID NO: 159) | |
| MICA-GS2-HC- | EPHSLRYNLTVLSWDGSVQSGF | DIQMTQSPSSLSASVGDRVTI |
| α-PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 162) |
| | TYQTWVATRICQGEEQRFTCYM | |
| | EHSGNHSTHPVPSGKVLVLQSH | |
| | WGGGGSGGGGSQVQLQESGPG | |
| | LVKPSETLSLTCTVSGFSLTNYA | |
| | INWVRQPPGKGLEWLGIIWTGG | |
| | GTSYNSALKSRLTISKDTSKNQV | |
| | SLKLSSVTAADTAVYYCARTGT | |
| | RGYFFDYWGQGTLVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKV | |
| | DKKVEPKSCDKTHTCPPCPAPE | |
| | LLGGPDVFLFPPKPKDTLMISRT | |
| | PEVTCVVVDVSHEDPEVKFNW | |
| | YVDGVEVHNAKTKPREEQYNS | |
| | TYRVVSVLTVLHQDWLNGKEY | |
| | KCKVSNKALPAPEEKTISKAKG | |
| | QPREPQVYTLPPSREEMTKNQV | |
| | SLTCLVKGFYPSDIAVEWESNG | |
| | QPENNYKTTPPVLDSDGSFFLYS | |
| | KLTVDKSRWQQGNVFSCSVMH | |
| | EALHNHYTQKSLSLSPGK (SEQ | |
| | ID NO: 161) | |
| MICA-GS3-HC- | EPHSLRYNLTVLSWDGSVQSGF | DIQMTQSPSSLSASVGDRVTI |
| α-PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 164) |
| | TYQTWVATRICQGEEQRFTCYM | |
| | EHSGNHSTHPVPSGKVLVLQSH | |
| | WGGGGSGGGGSGGGGSQVQLQ | |
| | ESGPGLVKPSETLSLTCTVSGFS | |
| | LTNYAINWVRQPPGKGLEWLGI | |
| | IWTGGGTSYNSALKSRLTISKDT | |
| | SKNQVSLKLSSVTAADTAVYYC | |
| | ARTGTRGYFFDYWGQGTLVTV | |
| | SSASTKGPSVFPLAPSSKSTSGG | |
| | TAALGCLVKDYFPEPVTVSWNS | |
| | GALTSGVHTFPAVLQSSGLYSLS | |
| | SVVTVPSSSLGTQTYICNVNHKP | |
| | SNTKVDKKVEPKSCDKTHTCPP | |
| | CPAPELLGGPDVFLFPPKPKDTL | |
| | MISRTPEVTCVVVDVSHEDPEV | |
| | KFNWYVDGVEVHNAKTKPREE | |
| | QYNSTYRVVSVLTVLHQDWLN | |
| | GKEYKCKVSNKALPAPEEKTIS | |
| | KAKGQPREPQVYTLPPSREEMT | |
| | KNQVSLTCLVKGFYPSDIAVEW | |
| | ESNGQPENNYKTTPPVLDSDGS | |
| | FFLYSKLTVDKSRWQQGNVFSC | |
| | SVMHEALHNHYTQKSLSLSPGK | |
| | (SEQ ID NO: 163) | |
| MICA-GS4-HC- | EPHSLRYNLTVLSWDGSVQSGF | DIQMTQSPSSLSASVGDRVTI |
| α-PDGFRb 1 | LTEVHLDGQPFLRCDRQKCRAK | TCHASQNINVWLSWYQQKP |
| S239D/1332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 166) |
| | TYQTWVATRICQGEEQRFTCYM | |
| | EHSGNHSTHPVPSGKVLVLQSH | |
| | WGGGGSGGGGSGGGGSGGGGS | |
| | QVQLQESGPGLVKPSETLSLTCT | |
| | VSGFSLTNYAINWVRQPPGKGL | |
| | EWLGIIWTGGGTSYNSALKSRL | |
| | TISKDTSKNQVSLKLSSVTAADT | |
| | AVYYCARTGTRGYFFDYWGQG | |
| | TLVTVSSASTKGPSVFPLAPSSK | |
| | STSGGTAALGCLVKDYFPEPVT | |
| | VSWNSGALTSGVHTFPAVLQSS | |
| | GLYSLSSVVTVPSSSLGTQTYIC | |
| | NVNHKPSNTKVDKKVEPKSCD | |
| | KTHTCPPCPAPELLGGPDVFLFP | |
| | PKPKDTLMISRTPEVTCVVVDV | |
| | SHEDPEVKFNWYVDGVEVHNA | |
| | KTKPREEQYNSTYRVVSVLTVL | |
| | HQDWLNGKEYKCKVSNKALPA | |
| | PEEKTISKAKGQPREPQVYTLPP | |
| | SREEMTKNQVSLTCLVKGFYPS | |
| | DIAVEWESNGQPENNYKTTPPV | |
| | LDSDGSFFLYSKLTVDKSRWQQ | |
| | GNVFSCSVMHEALHNHYTQKS | |
| | LSLSPGK (SEQ ID NO: 165) | |
| MICA-EAK2-HC- | EPHSLRYNLTVLSWDGSVQSGF | DIQMTQSPSSLSASVGDRVTI |
| α-PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 168) |
| | TYQTWVATRICQGEEQRFTCYM | |
| | EHSGNHSTHPVPSGKVLVLQSH | |
| | WAEAAAKEAAAKAQVQLQESG | |
| | PGLVKPSETLSLTCTVSGFSLTN | |
| | YAINWVRQPPGKGLEWLGIIWT | |
| | GGGTSYNSALKSRLTISKDTSKN | |
| | QVSLKLSSVTAADTAVYYCART | |
| | GTRGYFFDYWGQGTLVTVSSAS | |
| | TKGPSVFPLAPSSKSTSGGTAAL | |
| | GCLVKDYFPEPVTVSWNSGALT | |
| | SGVHTFPAVLQSSGLYSLSSVVT | |
| | VPSSSLGTQTYICNVNHKPSNTK | |
| | VDKKVEPKSCDKTHTCPPCPAP | |
| | ELLGGPDVFLFPPKPKDTLMISR | |
| | TPEVTCVVVDVSHEDPEVKFNW | |
| | YVDGVEVHNAKTKPREEQYNS | |
| | TYRVVSVLTVLHQDWLNGKEY | |
| | KCKVSNKALPAPEEKTISKAKG | |
| | QPREPQVYTLPPSREEMTKNQV | |
| | SLTCLVKGFYPSDIAVEWESNG | |
| | QPENNYKTTPPVLDSDGSFFLYS | |
| | KLTVDKSRWQQGNVFSCSVMH | |
| | EALHNHYTQKSLSLSPGK (SEQ | |
| | ID NO: 167) | |
| MICA-EAK3-HC- | EPHSLRYNLTVLSWDGSVQSGF | DIQMTQSPSSLSASVGDRVTI |
| α-PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 170) |
| | TYQTWVATRICQGEEQRFTCYM | |
| | EHSGNHSTHPVPSGKVLVLQSH | |
| | WAEAAAKEAAAKEAAAKAQV | |
| | QLQESGPGLVKPSETLSLTCTVS | |
| | GFSLTNYAINWVRQPPGKGLEW | |
| | LGIIWTGGGTSYNSALKSRLTIS | |
| | KDTSKNQVSLKLSSVTAADTAV | |
| | YYCARTGTRGYFFDYWGQGTL | |
| | VTVSSASTKGPSVFPLAPSSKST | |
| | SGGTAALGCLVKDYFPEPVTVS | |
| | WNSGALTSGVHTFPAVLQSSGL | |
| | YSLSSVVTVPSSSLGTQTYICNV | |
| | NHKPSNTKVDKKVEPKSCDKTH | |
| | TCPPCPAPELLGGPDVFLFPPKP | |
| | KDTLMISRTPEVTCVVVDVSHE | |
| | DPEVKFNWYVDGVEVHNAKTK | |
| | PREEQYNSTYRVVSVLTVLHQD | |
| | WLNGKEYKCKVSNKALPAPEE | |
| | KTISKAKGQPREPQVYTLPPSRE | |
| | EMTKNQVSLTCLVKGFYPSDIA | |
| | VEWESNGQPENNYKTTPPVLDS | |
| | DGSFFLYSKLTVDKSRWQQGN | |
| | VFSCSVMHEALHNHYTQKSLSL | |
| | SPGK (SEQ ID NO: 169) | |
| MICA-AP-HC-α- | EPHSLRYNLTVLSWDGSVQSGF | DIQMTQSPSSLSASVGDRVTI |
| PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 172) |
| | TYQTWVATRICQGEEQRFTCYM | |
| | EHSGNHSTHPVPSGKVLVLQSH | |
| | WAPAPAPAPAQVQLQESGPGLV | |
| | KPSETLSLTCTVSGFSLTNYAIN | |
| | WVRQPPGKGLEWLGIIWTGGGT | |
| | SYNSALKSRLTISKDTSKNQVSL | |
| | KLSSVTAADTAVYYCARTGTRG | |
| | YFFDYWGQGTLVTVSSASTKGP | |
| | SVFPLAPSSKSTSGGTAALGCLV | |
| | KDYFPEPVTVSWNSGALTSGVH | |
| | TFPAVLQSSGLYSLSSVVTVPSS | |
| | SLGTQTYICNVNHKPSNTKVDK | |
| | KVEPKSCDKTHTCPPCPAPELLG | |
| | GPDVFLFPPKPKDTLMISRTPEV | |
| | TCVVVDVSHEDPEVKFNWYVD | |
| | GVEVHNAKTKPREEQYNSTYR | |
| | VVSVLTVLHQDWLNGKEYKCK | |
| | VSNKALPAPEEKTISKAKGQPRE | |
| | PQVYTLPPSREEMTKNQVSLTC | |
| | LVKGFYPSDIAVEWESNGQPEN | |
| | NYKTTPPVLDSDGSFFLYSKLTV | |
| | DKSRWQQGNVFSCSVMHEALH | |
| | NHYTQKSLSLSPGK (SEQ ID | |
| | NO: 171) | |
| NKG2D-GS1- | FLNSLFNQEVQIPLTESYCGPCP | DIQMTQSPSSLSASVGDRVTI |
| NKG2D-GS4-HC | KNWICYKNNCYQFFDESKNWY | TCHASQNINVWLSWYQQKP |
| α-PDGFRb 1 | ESQASCMSQNASLLKVYSKEDQ | GKAPKLLIYKASNLHTGVPSR |
| S239D/I332E | DLLKLVKSYHWMGLVHIPTNGS | FSGSGSGTGFTLTISSLQPEDI |
| | WQWEDGSILSPNLLTIIEMQKG | ATYYCQQGQSFPFTFGGGTK |
| | DCALYASSFKGYIENCSTPNTYI | VEIKRTVAAPSVFIFPPSDEQL |
| | CMQRTVGGSGGGSGFLNSLFNQ | KSGTASVVCLLNNFYPREAK |
| | EVQIPLTESYCGPCPKNWICYKN | VQWKVDNALQSGNSQESVT |
| | NCYQFFDESKNWYESQASCMS | EQDSKDSTYSLSSTLTLSKAD |
| | QNASLLKVYSKEDQDLLKLVKS | YEKHKVYACEVTHQGLSSPV |
| | YHWMGLVHIPTNGSWQWEDGS | TKSFNRGEC (SEQ ID NO: 174) |
| | ILSPNLLTIIEMQKGDCALYASSF | |
| | KGYIENCSTPNTYICMQRTVGG | |
| | GGSGGGGSGGGGSGGGGQVQL | |
| | QESGPGLVKPSETLSLTCTVSGF | |
| | SLTNYAINWVRQPPGKGLEWLG | |
| | IIWTGGGTSYNSALKSRLTISKD | |
| | TSKNQVSLKLSSVTAADTAVYY | |
| | CARTGTRGYFFDYWGQGTLVT | |
| | VSSASTKGPSVFPLAPSSKSTSG | |
| | GTAALGCLVKDYFPEPVTVSWN | |
| | SGALTSGVHTFPAVLQSSGLYSL | |
| | SSVVTVPSSSLGTQTYICNVNHK | |
| | PSNTKVDKKVEPKSCDKTHTCP | |
| | PCPAPELLGGPDVFLFPPKPKDT | |
| | LMISRTPEVTCVVVDVSHEDPE | |
| | VKFNWYVDGVEVHNAKTKPRE | |
| | EQYNSTYRVVSVLTVLHQDWL | |
| | NGKEYKCKVSNKALPAPEEKTI | |
| | SKAKGQPREPQVYTLPPSREEM | |
| | TKNQVSLTCLVKGFYPSDIAVE | |
| | WESNGQPENNYKTTPPVLDSDG | |
| | SFFLYSKLTVDKSRWQQGNVFS | |
| | CSVMHEALHNHYTQKSLSLSPG | |
| | K (SEQ ID NO: 173) | |
| NKG2D-GS4- | FLNSLFNQEVQIPLTESYCGPCP | DIQMTQSPSSLSASVGDRVTI |
| NKG2D-GS4-HC | KNWICYKNNCYQFFDESKNWY | TCHASQNINVWLSWYQQKP |
| α-PDGFRb 1 | ESQASCMSQNASLLKVYSKEDQ | GKAPKLLIYKASNLHTGVPSR |
| S239D/I332E | DLLKLVKSYHWMGLVHIPTNGS | FSGSGSGTGFTLTISSLQPEDI |
| | WQWEDGSILSPNLLTIIEMQKG | ATYYCQQGQSFPFTFGGGTK |
| | DCALYASSFKGYIENCSTPNTYI | VEIKRTVAAPSVFIFPPSDEQL |
| | CMQRTVGGSGGGSGGGGSGGG | KSGTASVVCLLNNFYPREAK |
| | GSGGGGSGFLNSLFNQEVQIPLT | VQWKVDNALQSGNSQESVT |
| | ESYCGPCPKNWICYKNNCYQFF | EQDSKDSTYSLSSTLTLSKAD |
| | DESKNWYESQASCMSQNASLL | YEKHKVYACEVTHQGLSSPV |
| | KVYSKEDQDLLKLVKSYHWMG | TKSFNRGEC (SEQ ID NO: 176) |
| | LVHIPTNGSWQWEDGSILSPNLL | |
| | TIIEMQKGDCALYASSFKGYIEN | |
| | CSTPNTYICMQRTVGGGGSGGG | |
| | GSGGGGSGGGGQVQLQESGPG | |
| | LVKPSETLSLTCTVSGFSLTNYA | |
| | INWVRQPPGKGLEWLGIIWTGG | |
| | GTSYNSALKSRLTISKDTSKNQV | |
| | SLKLSSVTAADTAVYYCARTGT | |
| | RGYFFDYWGQGTLVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKV | |
| | DKKVEPKSCDKTHTCPPCPAPE | |
| | LLGGPDVFLFPPKPKDTLMISRT | |
| | PEVTCVVVDVSHEDPEVKFNW | |
| | YVDGVEVHNAKTKPREEQYNS | |
| | TYRVVSVLTVLHQDWLNGKEY | |
| | KCKVSNKALPAPEEKTISKAKG | |
| | QPREPQVYTLPPSREEMTKNQV | |
| | SLTCLVKGFYPSDIAVEWESNG | |
| | QPENNYKTTPPVLDSDGSFFLYS | |
| | KLTVDKSRWQQGNVFSCSVMH | |
| | EALHNHYTQKSLSLSPGK (SEQ | |
| | ID NO: 175) | |
| NKG2D-GS1- | FLNSLFNQEVQIPLTESYCGPCP | DIQMTQSPSSLSASVGDRVTI |
| NKG2D-EAK4- | KNWICYKNNCYQFFDESKNWY | TCHASQNINVWLSWYQQKP |
| HC α-PDGFRb 1 | ESQASCMSQNASLLKVYSKEDQ | GKAPKLLIYKASNLHTGVPSR |
| S239D/1332E | DLLKLVKSYHWMGLVHIPTNGS | FSGSGSGTGFTLTISSLQPEDI |
| | WQWEDGSILSPNLLTIIEMQKG | ATYYCQQGQSFPFTFGGGTK |
| | DCALYASSFKGYIENCSTPNTYI | VEIKRTVAAPSVFIFPPSDEQL |
| | CMQRTVGGSGGGSGFLNSLFNQ | KSGTASVVCLLNNFYPREAK |
| | EVQIPLTESYCGPCPKNWICYKN | VQWKVDNALQSGNSQESVT |
| | NCYQFFDESKNWYESQASCMS | EQDSKDSTYSLSSTLTLSKAD |
| | QNASLLKVYSKEDQDLLKLVKS | YEKHKVYACEVTHQGLSSPV |
| | YHWMGLVHIPTNGSWQWEDGS | TKSFNRGEC (SEQ ID NO: 178) |
| | ILSPNLLTIIEMQKGDCALYASSF | |
| | KGYIENCSTPNTYICMQRTVAE | |
| | AAAKEAAAKEAAAKEAAAKAQ | |
| | VQLQESGPGLVKPSETLSLTCTV | |
| | SGFSLTNYAINWVRQPPGKGLE | |
| | WLGIIWTGGGTSYNSALKSRLTI | |
| | SKDTSKNQVSLKLSSVTAADTA | |
| | VYYCARTGTRGYFFDYWGQGT | |
| | LVTVSSASTKGPSVFPLAPSSKS | |
| | TSGGTAALGCLVKDYFPEPVTV | |
| | SWNSGALTSGVHTFPAVLQSSG | |
| | LYSLSSVVTVPSSSLGTQTYICN | |
| | VNHKPSNTKVDKKVEPKSCDKT | |
| | HTCPPCPAPELLGGPDVFLFPPK | |
| | PKDTLMISRTPEVTCVVVDVSH | |
| | EDPEVKFNWYVDGVEVHNAKT | |
| | KPREEQYNSTYRVVSVLTVLHQ | |
| | DWLNGKEYKCKVSNKALPAPE | |
| | EKTISKAKGQPREPQVYTLPPSR | |
| | EEMTKNQVSLTCLVKGFYPSDI | |
| | AVEWESNGQPENNYKTTPPVLD | |
| | SDGSFFLYSKLTVDKSRWQQGN | |
| | VFSCSVMHEALHNHYTQKSLSL | |
| | SPGK (SEQ ID NO: 177) | |
| NKG2D-GS4- | FLNSLFNQEVQIPLTESYCGPCP | DIQMTQSPSSLSASVGDRVTI |
| NKG2D-EAK4- | KNWICYKNNCYQFFDESKNWY | TCHASQNINVWLSWYQQKP |
| HC α-PDGFRb 1 | ESQASCMSQNASLLKVYSKEDQ | GKAPKLLIYKASNLHTGVPSR |
| S239D/I332E | DLLKLVKSYHWMGLVHIPTNGS | FSGSGSGTGFTLTISSLQPEDI |
| | WQWEDGSILSPNLLTIIEMQKG | ATYYCQQGQSFPFTFGGGTK |
| | DCALYASSFKGYIENCSTPNTYI | VEIKRTVAAPSVFIFPPSDEQL |
| | CMQRTVGGSGGGSGGGGSGGG | KSGTASVVCLLNNFYPREAK |
| | GSGFLNSLFNQEVQIPLTESYCG | VQWKVDNALQSGNSQESVT |
| | PCPKNWICYKNNCYQFFDESKN | EQDSKDSTYSLSSTLTLSKAD |
| | WYESQASCMSQNASLLKVYSK | YEKHKVYACEVTHQGLSSPV |
| | EDQDLLKLVKSYHWMGLVHIP | TKSFNRGEC (SEQ ID NO: 180) |
| | TNGSWQWEDGSILSPNLLTIIEM | |
| | QKGDCALYASSFKGYIENCSTP | |
| | NTYICMQRTVAEAAAKEAAAK | |
| | EAAAKEAAAKAQVQLQESGPG | |
| | LVKPSETLSLTCTVSGFSLTNYA | |
| | INWVRQPPGKGLEWLGIIWTGG | |
| | GTSYNSALKSRLTISKDTSKNQV | |
| | SLKLSSVTAADTAVYYCARTGT | |
| | RGYFFDYWGQGTLVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKV | |
| | DKKVEPKSCDKTHTCPPCPAPE | |
| | LLGGPDVFLFPPKPKDTLMISRT | |
| | PEVTCVVVDVSHEDPEVKFNW | |
| | YVDGVEVHNAKTKPREEQYNS | |
| | TYRVVSVLTVLHQDWLNGKEY | |
| | KCKVSNKALPAPEEKTISKAKG | |
| | QPREPQVYTLPPSREEMTKNQV | |
| | SLTCLVKGFYPSDIAVEWESNG | |
| | QPENNYKTTPPVLDSDGSFFLYS | |
| | KLTVDKSRWQQGNVFSCSVMH | |
| | EALHNHYTQKSLSLSPGK (SEQ | |
| | ID NO: 179) | |
| NKG2D-GS1- | FLNSLFNQEVQIPLTESYCGPCP | DIQMTQSPSSLSASVGDRVTI |
| NKG2D-AP-HC | KNWICYKNNCYQFFDESKNWY | TCHASQNINVWLSWYQQKP |
| α-PDGFRb 1 | ESQASCMSQNASLLKVYSKEDQ | GKAPKLLIYKASNLHTGVPSR |
| S239D/I332E | DLLKLVKSYHWMGLVHIPTNGS | FSGSGSGTGFTLTISSLQPEDI |
| | WQWEDGSILSPNLLTIIEMQKG | ATYYCQQGQSFPFTFGGGTK |
| | DCALYASSFKGYIENCSTPNTYI | VEIKRTVAAPSVFIFPPSDEQL |
| | CMQRTVGGSGGGSGFLNSLENQ | KSGTASVVCLLNNFYPREAK |
| | EVQIPLTESYCGPCPKNWICYKN | VQWKVDNALQSGNSQESVT |
| | NCYQFFDESKNWYESQASCMS | EQDSKDSTYSLSSTLTLSKAD |
| | QNASLLKVYSKEDQDLLKLVKS | YEKHKVYACEVTHQGLSSPV |
| | YHWMGLVHIPTNGSWQWEDGS | TKSFNRGEC (SEQ ID NO: 182) |
| | ILSPNLLTIIEMQKGDCALYASSF | |
| | KGYIENCSTPNTYICMQRTVAP | |
| | APAPAPAPAPAPAPAPAPQVQL | |
| | QESGPGLVKPSETLSLTCTVSGF | |
| | SLTNYAINWVRQPPGKGLEWLG | |
| | IIWTGGGTSYNSALKSRLTISKD | |
| | TSKNQVSLKLSSVTAADTAVYY | |
| | CARTGTRGYFFDYWGQGTLVT | |
| | VSSASTKGPSVFPLAPSSKSTSG | |
| | GTAALGCLVKDYFPEPVTVSWN | |
| | SGALTSGVHTFPAVLQSSGLYSL | |
| | SSVVTVPSSSLGTQTYICNVNHK | |
| | PSNTKVDKKVEPKSCDKTHTCP | |
| | PCPAPELLGGPDVFLFPPKPKDT | |
| | LMISRTPEVTCVVVDVSHEDPE | |
| | VKFNWYVDGVEVHNAKTKPRE | |
| | EQYNSTYRVVSVLTVLHQDWL | |
| | NGKEYKCKVSNKALPAPEEKTI | |
| | SKAKGQPREPQVYTLPPSREEM | |
| | TKNQVSLTCLVKGFYPSDIAVE | |
| | WESNGQPENNYKTTPPVLDSDG | |
| | SFFLYSKLTVDKSRWQQGNVFS | |
| | CSVMHEALHNHYTQKSLSLSPG | |
| | K (SEQ ID NO: 181) | |
| NKG2D-GS4- | FLNSLFNQEVQIPLTESYCGPCP | DIQMTQSPSSLSASVGDRVTI |
| NKG2D-AP-HC | KNWICYKNNCYQFFDESKNWY | TCHASQNINVWLSWYQQKP |
| α-PDGFRb 1 | ESQASCMSQNASLLKVYSKEDQ | GKAPKLLIYKASNLHTGVPSR |
| S239D/I332E | DLLKLVKSYHWMGLVHIPTNGS | FSGSGSGTGFTLTISSLQPEDI |
| | WQWEDGSILSPNLLTIIEMQKG | ATYYCQQGQSFPFTFGGGTK |
| | DCALYASSFKGYIENCSTPNTYI | VEIKRTVAAPSVFIFPPSDEQL |
| | CMQRTVGGSGGGSGGGGSGGG | KSGTASVVCLLNNFYPREAK |
| | GSGFLNSLFNQEVQIPLTESYCG | VQWKVDNALQSGNSQESVT |
| | PCPKNWICYKNNCYQFFDESKN | EQDSKDSTYSLSSTLTLSKAD |
| | WYESQASCMSQNASLLKVYSK | YEKHKVYACEVTHQGLSSPV |
| | EDQDLLKLVKSYHWMGLVHIP | TKSFNRGEC (SEQ ID NO: 184) |
| | TNGSWQWEDGSILSPNLLTIIEM | |
| | QKGDCALYASSFKGYIENCSTP | |
| | NTYICMQRTVAPAPAPAPAPAP | |
| | APAPAPAPQVQLQESGPGLVKP | |
| | SETLSLTCTVSGFSLTNYAINWV | |
| | RQPPGKGLEWLGIIWTGGGTSY | |
| | NSALKSRLTISKDTSKNQVSLKL | |
| | SSVTAADTAVYYCARTGTRGYF | |
| | FDYWGQGTLVTVSSASTKGPSV | |
| | FPLAPSSKSTSGGTAALGCLVKD | |
| | YFPEPVTVSWNSGALTSGVHTF | |
| | PAVLQSSGLYSLSSVVTVPSSSL | |
| | GTQTYICNVNHKPSNTKVDKKV | |
| | EPKSCDKTHTCPPCPAPELLGGP | |
| | DVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCKVSN | |
| | KALPAPEEKTISKAKGQPREPQV | |
| | YTLPPSREEMTKNQVSLTCLVK | |
| | GFYPSDIAVEWESNGQPENNYK | |
| | TTPPVLDSDGSFFLYSKLTVDKS | |
| | RWQQGNVFSCSVMHEALHNHY | |
| | TQKSLSLSPGK (SEQ ID NO: 183) | |
| MICB-EAK2-HC- | AEPHSLRYNLMVLSQDESVQSG | DIQMTQSPSSLSASVGDRVTI |
| α-PDGFRb 1 | FLAEGHLDGQPFLRYDRQKRRA | TCHASQNINVWLSWYQQKP |
| S239D/I332E | KPQGQWAEDVLGAKTWDTETE | GKAPKLLIYKASNLHTGVPSR |
| | DLTENGQDLRRTLTHIKDQKGG | FSGSGSGTGFTLTISSLQPEDI |
| | LHSLQEIRVCEIHEDSSTRGSRHF | ATYYCQQGQSFPFTFGGGTK |
| | YYDGELFLSQNLETQESTVPQSS | VEIKRTVAAPSVFIFPPSDEQL |
| | RAQTLAMNVTNFWKEDAMKT | KSGTASVVCLLNNFYPREAK |
| | KTHYRAMQADCLQKLQRYLKS | VQWKVDNALQSGNSQESVT |
| | GVAIRRTVPPMVNVTCSEVSEG | EQDSKDSTYSLSSTLTLSKAD |
| | NITVTCRASSFYPRNITLTWRQD | YEKHKVYACEVTHQGLSSPV |
| | GVSLSHNTQQWGDVLPDGNGT | TKSFNRGEC (SEQ ID NO: 186) |
| | YQTWVATRIRQGEEQRFTCYME | |
| | HSGNHGTHPVPSGKVLVLQSQR | |
| | TDAEAAAKEAAAKAQVQLQES | |
| | GPGLVKPSETLSLTCTVSGFSLT | |
| | NYAINWVRQPPGKGLEWLGIIW | |
| | TGGGTSYNSALKSRLTISKDTSK | |
| | NQVSLKLSSVTAADTAVYYCAR | |
| | TGTRGYFFDYWGQGTLVTVSSA | |
| | STKGPSVFPLAPSSKSTSGGTAA | |
| | LGCLVKDYFPEPVTVSWNSGAL | |
| | TSGVHTFPAVLQSSGLYSLSSVV | |
| | TVPSSSLGTQTYICNVNHKPSNT | |
| | KVDKKVEPKSCDKTHTCPPCPA | |
| | PELLGGPDVFLFPPKPKDTLMIS | |
| | RTPEVTCVVVDVSHEDPEVKFN | |
| | WYVDGVEVHNAKTKPREEQYN | |
| | STYRVVSVLTVLHQDWLNGKE | |
| | YKCKVSNKALPAPEEKTISKAK | |
| | GQPREPQVYTLPPSREEMTKNQ | |
| | VSLTCLVKGFYPSDIAVEWESN | |
| | GQPENNYKTTPPVLDSDGSFFL | |
| | YSKLTVDKSRWQQGNVFSCSV | |
| | MHEALHNHYTQKSLSLSPGK | |
| | (SEQ ID NO: 185) | |
| ULBP1-EAK2- | GWVDTHCLCYDFIITPKSRPEPQ | DIQMTQSPSSLSASVGDRVTI |
| HC-α-PDGFRb 1 | WCEVQGLVDERPFLHYDCVNH | TCHASQNINVWLSWYQQKP |
| S239D/I332E | KAKAFASLGKKVNVTKTWEEQ | GKAPKLLIYKASNLHTGVPSR |
| | TETLRDVVDFLKGQLLDIQVEN | FSGSGSGTGFTLTISSLQPEDI |
| | LIPIEPLTLQARMSCEHEAHGHG | ATYYCQQGQSFPFTFGGGTK |
| | RGSWQFLFNGQKFLLFDSNNRK | VEIKRTVAAPSVFIFPPSDEQL |
| | WTALHPGAKKMTEKWEKNRD | KSGTASVVCLLNNFYPREAK |
| | VTMFFQKISLGDCKMWLEEFLM | VQWKVDNALQSGNSQESVT |
| | YWEQMLDPTKPPSLAPAEAAA | EQDSKDSTYSLSSTLTLSKAD |
| | KEAAAKAQVQLQESGPGLVKPS | YEKHKVYACEVTHQGLSSPV |
| | ETLSLTCTVSGFSLTNYAINWVR | TKSFNRGEC (SEQ ID NO: 188) |
| | QPPGKGLEWLGIIWTGGGTSYN | |
| | SALKSRLTISKDTSKNQVSLKLS | |
| | SVTAADTAVYYCARTGTRGYFF | |
| | DYWGQGTLVTVSSASTKGPSVF | |
| | PLAPSSKSTSGGTAALGCLVKD | |
| | YFPEPVTVSWNSGALTSGVHTF | |
| | PAVLQSSGLYSLSSVVTVPSSSL | |
| | GTQTYICNVNHKPSNTKVDKKV | |
| | EPKSCDKTHTCPPCPAPELLGGP | |
| | DVFLFPPKPKDTLMISRTPEVTC | |
| | VVVDVSHEDPEVKFNWYVDGV | |
| | EVHNAKTKPREEQYNSTYRVVS | |
| | VLTVLHQDWLNGKEYKCKVSN | |
| | KALPAPEEKTISKAKGQPREPQV | |
| | YTLPPSREEMTKNQVSLTCLVK | |
| | GFYPSDIAVEWESNGQPENNYK | |
| | TTPPVLDSDGSFFLYSKLTVDKS | |
| | RWQQGNVFSCSVMHEALHNHY | |
| | TQKSLSLSPGK (SEQ ID NO: 187) | |
| ULBP2-EAK2- | GRADPHSLCYDITVIPKFRPGPR | DIQMTQSPSSLSASVGDRVTI |
| HC-α-PDGFRb 1 | WCAVQGQVDEKTFLHYDCGNK | TCHASQNINVWLSWYQQKP |
| S239D/I332E | TVTPVSPLGKKLNVTTAWKAQ | GKAPKLLIYKASNLHTGVPSR |
| | NPVLREVVDILTEQLRDIQLENY | FSGSGSGTGFTLTISSLQPEDI |
| | TPKEPLTLQARMSCEQKAEGHS | ATYYCQQGQSFPFTFGGGTK |
| | SGSWQFSFDGQIFLLFDSEKRM | VEIKRTVAAPSVFIFPPSDEQL |
| | WTTVHPGARKMKEKWENDKV | KSGTASVVCLLNNFYPREAK |
| | VAMSFHYFSMGDCIGWLEDFL | VQWKVDNALQSGNSQESVT |
| | MGMDSTLEPSAGAPLAMAEAA | EQDSKDSTYSLSSTLTLSKAD |
| | AKEAAAKAQVQLQESGPGLVK | YEKHKVYACEVTHQGLSSPV |
| | PSETLSLTCTVSGFSLTNYAINW | TKSFNRGEC (SEQ ID NO: 190) |
| | VRQPPGKGLEWLGIIWTGGGTS | |
| | YNSALKSRLTISKDTSKNQVSLK | |
| | LSSVTAADTAVYYCARTGTRGY | |
| | FFDYWGQGTLVTVSSASTKGPS | |
| | VFPLAPSSKSTSGGTAALGCLVK | |
| | DYFPEPVTVSWNSGALTSGVHT | |
| | FPAVLQSSGLYSLSSVVTVPSSS | |
| | LGTQTYICNVNHKPSNTKVDKK | |
| | VEPKSCDKTHTCPPCPAPELLGG | |
| | PDVFLFPPKPKDTLMISRTPEVT | |
| | CVVVDVSHEDPEVKFNWYVDG | |
| | VEVHNAKTKPREEQYNSTYRV | |
| | VSVLTVLHQDWLNGKEYKCKV | |
| | SNKALPAPEEKTISKAKGQPREP | |
| | QVYTLPPSREEMTKNQVSLTCL | |
| | VKGFYPSDIAVEWESNGQPENN | |
| | YKTTPPVLDSDGSFFLYSKLTVD | |
| | KSRWQQGNVFSCSVMHEALHN | |
| | HYTQKSLSLSPGK (SEQ ID NO: | |
| | 189) | |
| MICB-AP-HC-α- | AEPHSLRYNLMVLSQDESVQSG | DIQMTQSPSSLSASVGDRVTI |
| PDGFRb 1 | FLAEGHLDGQPFLRYDRQKRRA | TCHASQNINVWLSWYQQKP |
| S239D/I332E | KPQGQWAEDVLGAKTWDTETE | GKAPKLLIYKASNLHTGVPSR |
| | DLTENGQDLRRTLTHIKDQKGG | FSGSGSGTGFTLTISSLQPEDI |
| | LHSLQEIRVCEIHEDSSTRGSRHF | ATYYCQQGQSFPFTFGGGTK |
| | YYDGELFLSQNLETQESTVPQSS | VEIKRTVAAPSVFIFPPSDEQL |
| | RAQTLAMNVTNFWKEDAMKT | KSGTASVVCLLNNFYPREAK |
| | KTHYRAMQADCLQKLQRYLKS | VQWKVDNALQSGNSQESVT |
| | GVAIRRTVPPMVNVTCSEVSEG | EQDSKDSTYSLSSTLTLSKAD |
| | NITVTCRASSFYPRNITLTWRQD | YEKHKVYACEVTHQGLSSPV |
| | GVSLSHNTQQWGDVLPDGNGT | TKSFNRGEC (SEQ ID NO: 192) |
| | YQTWVATRIRQGEEQRFTCYME | |
| | HSGNHGTHPVPSGKVLVLQSQR | |
| | TDAPAPAPAPAQVQLQESGPGL | |
| | VKPSETLSLTCTVSGFSLTNYAI | |
| | NWVRQPPGKGLEWLGIIWTGG | |
| | GTSYNSALKSRLTISKDTSKNQV | |
| | SLKLSSVTAADTAVYYCARTGT | |
| | RGYFFDYWGQGTLVTVSSASTK | |
| | GPSVFPLAPSSKSTSGGTAALGC | |
| | LVKDYFPEPVTVSWNSGALTSG | |
| | VHTFPAVLQSSGLYSLSSVVTVP | |
| | SSSLGTQTYICNVNHKPSNTKV | |
| | DKKVEPKSCDKTHTCPPCPAPE | |
| | LLGGPDVFLFPPKPKDTLMISRT | |
| | PEVTCVVVDVSHEDPEVKFNW | |
| | YVDGVEVHNAKTKPREEQYNS | |
| | TYRVVSVLTVLHQDWLNGKEY | |
| | KCKVSNKALPAPEEKTISKAKG | |
| | QPREPQVYTLPPSREEMTKNQV | |
| | SLTCLVKGFYPSDIAVEWESNG | |
| | QPENNYKTTPPVLDSDGSFFLYS | |
| | KLTVDKSRWQQGNVFSCSVMH | |
| | EALHNHYTQKSLSLSPGK (SEQ | |
| | ID NO: 191) | |
| ULBP1-AP-HC- | GWVDTHCLCYDFIITPKSRPEPQ | DIQMTQSPSSLSASVGDRVTI |
| α-PDGFRb 1 | WCEVQGLVDERPFLHYDCVNH | TCHASQNINVWLSWYQQKP |
| S239D/I332E | KAKAFASLGKKVNVTKTWEEQ | GKAPKLLIYKASNLHTGVPSR |
| | TETLRDVVDFLKGQLLDIQVEN | FSGSGSGTGFTLTISSLQPEDI |
| | LIPIEPLTLQARMSCEHEAHGHG | ATYYCQQGQSFPFTFGGGTK |
| | RGSWQFLFNGQKFLLFDSNNRK | VEIKRTVAAPSVFIFPPSDEQL |
| | WTALHPGAKKMTEKWEKNRD | KSGTASVVCLLNNFYPREAK |
| | VTMFFQKISLGDCKMWLEEFLM | VQWKVDNALQSGNSQESVT |
| | YWEQMLDPTKPPSLAPAPAPAP | EQDSKDSTYSLSSTLTLSKAD |
| | APAQVQLQESGPGLVKPSETLSL | YEKHKVYACEVTHQGLSSPV |
| | TCTVSGFSLTNYAINWVRQPPG | TKSFNRGEC (SEQ ID NO: 194) |
| | KGLEWLGIIWTGGGTSYNSALK | |
| | SRLTISKDTSKNQVSLKLSSVTA | |
| | ADTAVYYCARTGTRGYFFDYW | |
| | GQGTLVTVSSASTKGPSVFPLAP | |
| | SSKSTSGGTAALGCLVKDYFPEP | |
| | VTVSWNSGALTSGVHTFPAVLQ | |
| | SSGLYSLSSVVTVPSSSLGTQTYI | |
| | CNVNHKPSNTKVDKKVEPKSC | |
| | DKTHTCPPCPAPELLGGPDVFLF | |
| | PPKPKDTLMISRTPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APEEKTISKAKGQPREPQVYTLP | |
| | PSREEMTKNQVSLTCLVKGFYP | |
| | SDIAVEWESNGQPENNYKTTPP | |
| | VLDSDGSFFLYSKLTVDKSRWQ | |
| | QGNVFSCSVMHEALHNHYTQK | |
| | SLSLSPGK (SEQ ID NO: 193) | |
| ULBP2-AP-HC- | GRADPHSLCYDITVIPKFRPGPR | DIQMTQSPSSLSASVGDRVTI |
| α-PDGFRb 1 | WCAVQGQVDEKTFLHYDCGNK | TCHASQNINVWLSWYQQKP |
| S239D/I332E | TVTPVSPLGKKLNVTTAWKAQ | GKAPKLLIYKASNLHTGVPSR |
| | NPVLREVVDILTEQLRDIQLENY | FSGSGSGTGFTLTISSLQPEDI |
| | TPKEPLTLQARMSCEQKAEGHS | ATYYCQQGQSFPFTFGGGTK |
| | SGSWQFSFDGQIFLLFDSEKRM | VEIKRTVAAPSVFIFPPSDEQL |
| | WTTVHPGARKMKEKWENDKV | KSGTASVVCLLNNFYPREAK |
| | VAMSFHYFSMGDCIGWLEDFL | VQWKVDNALQSGNSQESVT |
| | MGMDSTLEPSAGAPLAMAPAP | EQDSKDSTYSLSSTLTLSKAD |
| | APAPAQVQLQESGPGLVKPSET | YEKHKVYACEVTHQGLSSPV |
| | LSLTCTVSGFSLTNYAINWVRQP | TKSFNRGEC (SEQ ID NO: 196) |
| | PGKGLEWLGIIWTGGGTSYNSA | |
| | LKSRLTISKDTSKNQVSLKLSSV | |
| | TAADTAVYYCARTGTRGYFFD | |
| | YWGQGTLVTVSSASTKGPSVFP | |
| | LAPSSKSTSGGTAALGCLVKDY | |
| | FPEPVTVSWNSGALTSGVHTFP | |
| | AVLQSSGLYSLSSVVTVPSSSLG | |
| | TQTYICNVNHKPSNTKVDKKVE | |
| | PKSCDKTHTCPPCPAPELLGGPD | |
| | VFLFPPKPKDTLMISRTPEVTCV | |
| | VVDVSHEDPEVKFNWYVDGVE | |
| | VHNAKTKPREEQYNSTYRVVSV | |
| | LTVLHQDWLNGKEYKCKVSNK | |
| | ALPAPEEKTISKAKGQPREPQVY | |
| | TLPPSREEMTKNQVSLTCLVKG | |
| | FYPSDIAVEWESNGQPENNYKT | |
| | TPPVLDSDGSFFLYSKLTVDKSR | |
| | WQQGNVFSCSVMHEALHNHYT | |
| | QKSLSLSPGK (SEQ ID NO: 195) | |
| α-PDGFRb 1-HC- | QVQLQESGPGLVKPSETLSLTCT | DIQMTQSPSSLSASVGDRVTI |
| AP-MICA | VSGFSLTNYAINWVRQPPGKGL | TCHASQNINVWLSWYQQKP |
| S239D/I332E | EWLGIIWTGGGTSYNSALKSRL | GKAPKLLIYKASNLHTGVPSR |
| | TISKDTSKNQVSLKLSSVTAADT | FSGSGSGTGFTLTISSLQPEDI |
| | AVYYCARTGTRGYFFDYWGQG | ATYYCQQGQSFPFTFGGGTK |
| | TLVTVSSASTKGPSVFPLAPSSK | VEIKRTVAAPSVFIFPPSDEQL |
| | STSGGTAALGCLVKDYFPEPVT | KSGTASVVCLLNNFYPREAK |
| | VSWNSGALTSGVHTFPAVLQSS | VQWKVDNALQSGNSQESVT |
| | GLYSLSSVVTVPSSSLGTQTYIC | EQDSKDSTYSLSSTLTLSKAD |
| | NVNHKPSNTKVDKKVEPKSCD | YEKHKVYACEVTHQGLSSPV |
| | KTHTCPPCPAPELLGGPDVFLFP | TKSFNRGEC (SEQ ID NO: 198) |
| | PKPKDTLMISRTPEVTCVVVDV | |
| | SHEDPEVKFNWYVDGVEVHNA | |
| | KTKPREEQYNSTYRVVSVLTVL | |
| | HQDWLNGKEYKCKVSNKALPA | |
| | PEEKTISKAKGQPREPQVYTLPP | |
| | SREEMTKNQVSLTCLVKGFYPS | |
| | DIAVEWESNGQPENNYKTTPPV | |
| | LDSDGSFFLYSKLTVDKSRWQQ | |
| | GNVFSCSVMHEALHNHYTQKS | |
| | LSLSPGKAPAPAPAPAEPHSLRY | |
| | NLTVLSWDGSVQSGFLTEVHLD | |
| | GQPFLRCDRQKCRAKPQGQWA | |
| | EDVLGNKTWDRETRDLTGNGK | |
| | DLRMTLAHIKDQKEGLHSLQEI | |
| | RVCEIHEDNSTRSSQHFYYDGEL | |
| | FLSQNLETKEWTMPQSSRAQTL | |
| | AMNVRNFLKEDAMKTKTHYHA | |
| | MHADCLQELRRYLKSGVVLRR | |
| | TVPPMVNVTRSEASEGNITVTC | |
| | RASGFYPWNITLSWRQDGVSLS | |
| | HDTQQWGDVLPDGNGTYQTW | |
| | VATRICQGEEQRFTCYMEHSGN | |
| | HSTHPVPSGKVLVLQSHW (SEQ | |
| | ID NO: 197) | |
| MICA-CL-HC-α- | EPHSLRYNLTVLSWDGSVQSGF | DIQMTQSPSSLSASVGDRVTI |
| PDGFRb 1 | LTEVHLDGQPFLRCDRQKCRAK | TCHASQNINVWLSWYQQKP |
| S239D/I332E | PQGQWAEDVLGNKTWDRETRD | GKAPKLLIYKASNLHTGVPSR |
| | LTGNGKDLRMTLAHIKDQKEGL | FSGSGSGTGFTLTISSLQPEDI |
| | HSLQEIRVCEIHEDNSTRSSQHF | ATYYCQQGQSFPFTFGGGTK |
| | YYDGELFLSQNLETKEWTMPQS | VEIKRTVAAPSVFIFPPSDEQL |
| | SRAQTLAMNVRNFLKEDAMKT | KSGTASVVCLLNNFYPREAK |
| | KTHYHAMHADCLQELRRYLKS | VQWKVDNALQSGNSQESVT |
| | GVVLRRTVPPMVNVTRSEASEG | EQDSKDSTYSLSSTLTLSKAD |
| | NITVTCRASGFYPWNITLSWRQ | YEKHKVYACEVTHQGLSSPV |
| | DGVSLSHDTQQWGDVLPDGNG | TKSFNRGEC (SEQ ID NO: 200) |
| | TYQTWVATRICQGEEQRFTCYM | |
| | EHSGNHSTHPVPSGKVLVLQSH | |
| | WRTVAAPSVFIFPPSDEQLKSGT | |
| | ASVVCLLNNFYPREAKVQWKV | |
| | DNALQSGNSQESVTEQDSKDST | |
| | YSLSSTLTLSKADYEKHKVYAC | |
| | EVTHQGLSSPVTKSFNRGECQV | |
| | QLQESGPGLVKPSETLSLTCTVS | |
| | GFSLTNYAINWVRQPPGKGLEW | |
| | LGIIWTGGGTSYNSALKSRLTIS | |
| | KDTSKNQVSLKLSSVTAADTAV | |
| | YYCARTGTRGYFFDYWGQGTL | |
| | VTVSSASTKGPSVFPLAPSSKST | |
| | SGGTAALGCLVKDYFPEPVTVS | |
| | WNSGALTSGVHTFPAVLQSSGL | |
| | YSLSSVVTVPSSSLGTQTYICNV | |
| | NHKPSNTKVDKKVEPKSCDKTH | |
| | TCPPCPAPELLGGPDVFLFPPKP | |
| | KDTLMISRTPEVTCVVVDVSHE | |
| | DPEVKFNWYVDGVEVHNAKTK | |
| | PREEQYNSTYRVVSVLTVLHQD | |
| | WLNGKEYKCKVSNKALPAPEE | |
| | KTISKAKGQPREPQVYTLPPSRE | |
| | EMTKNQVSLTCLVKGFYPSDIA | |
| | VEWESNGQPENNYKTTPPVLDS | |
| | DGSFFLYSKLTVDKSRWQQGN | |
| | VFSCSVMHEALHNHYTQKSLSL | |
| | SPGK (SEQ ID NO: 199) | |
| MICB-CL-HC-α- | AEPHSLRYNLMVLSQDESVQSG | DIQMTQSPSSLSASVGDRVTI |
| PDGFRb 1 | FLAEGHLDGQPFLRYDRQKRRA | TCHASQNINVWLSWYQQKP |
| S239D/I332E | KPQGQWAEDVLGAKTWDTETE | GKAPKLLIYKASNLHTGVPSR |
| | DLTENGQDLRRTLTHIKDQKGG | FSGSGSGTGFTLTISSLQPEDI |
| | LHSLQEIRVCEIHEDSSTRGSRHF | ATYYCQQGQSFPFTFGGGTK |
| | YYDGELFLSQNLETQESTVPQSS | VEIKRTVAAPSVFIFPPSDEQL |
| | RAQTLAMNVTNFWKEDAMKT | KSGTASVVCLLNNFYPREAK |
| | KTHYRAMQADCLQKLQRYLKS | VQWKVDNALQSGNSQESVT |
| | GVAIRRTVPPMVNVTCSEVSEG | EQDSKDSTYSLSSTLTLSKAD |
| | NITVTCRASSFYPRNITLTWRQD | YEKHKVYACEVTHQGLSSPV |
| | GVSLSHNTQQWGDVLPDGNGT | TKSFNRGEC (SEQ ID NO: 202) |
| | YQTWVATRIRQGEEQRFTCYME | |
| | HSGNHGTHPVPSGKVLVLQSQR | |
| | TDRTVAAPSVFIFPPSDEQLKSG | |
| | TASVVCLLNNFYPREAKVQWK | |
| | VDNALQSGNSQESVTEQDSKDS | |
| | TYSLSSTLTLSKADYEKHKVYA | |
| | CEVTHQGLSSPVTKSFNRGECQ | |
| | VQLQESGPGLVKPSETLSLTCTV | |
| | SGFSLTNYAINWVRQPPGKGLE | |
| | WLGIIWTGGGTSYNSALKSRLTI | |
| | SKDTSKNQVSLKLSSVTAADTA | |
| | VYYCARTGTRGYFFDYWGQGT | |
| | LVTVSSASTKGPSVFPLAPSSKS | |
| | TSGGTAALGCLVKDYFPEPVTV | |
| | SWNSGALTSGVHTFPAVLQSSG | |
| | LYSLSSVVTVPSSSLGTQTYICN | |
| | VNHKPSNTKVDKKVEPKSCDKT | |
| | HTCPPCPAPELLGGPDVFLFPPK | |
| | PKDTLMISRTPEVTCVVVDVSH | |
| | EDPEVKFNWYVDGVEVHNAKT | |
| | KPREEQYNSTYRVVSVLTVLHQ | |
| | DWLNGKEYKCKVSNKALPAPE | |
| | EKTISKAKGQPREPQVYTLPPSR | |
| | EEMTKNQVSLTCLVKGFYPSDI | |
| | AVEWESNGQPENNYKTTPPVLD | |
| | SDGSFFLYSKLTVDKSRWQQGN | |
| | VFSCSVMHEALHNHYTQKSLSL | |
| | SPGK (SEQ ID NO: 201) | |
| ULBP1-CL-HC- | GWVDTHCLCYDFIITPKSRPEPQ | DIQMTQSPSSLSASVGDRVTI |
| α-PDGFRb 1 | WCEVQGLVDERPFLHYDCVNH | TCHASQNINVWLSWYQQKP |
| S239D/I332E | KAKAFASLGKKVNVTKTWEEQ | GKAPKLLIYKASNLHTGVPSR |
| | TETLRDVVDFLKGQLLDIQVEN | FSGSGSGTGFTLTISSLQPEDI |
| | LIPIEPLTLQARMSCEHEAHGHG | ATYYCQQGQSFPFTFGGGTK |
| | RGSWQFLFNGQKFLLFDSNNRK | VEIKRTVAAPSVFIFPPSDEQL |
| | WTALHPGAKKMTEKWEKNRD | KSGTASVVCLLNNFYPREAK |
| | VTMFFQKISLGDCKMWLEEFLM | VQWKVDNALQSGNSQESVT |
| | YWEQMLDPTKPPSLAPRTVAAP | EQDSKDSTYSLSSTLTLSKAD |
| | SVFIFPPSDEQLKSGTASVVCLL | YEKHKVYACEVTHQGLSSPV |
| | NNFYPREAKVQWKVDNALQSG | TKSFNRGEC (SEQ ID NO: 204) |
| | NSQESVTEQDSKDSTYSLSSTLT | |
| | LSKADYEKHKVYACEVTHQGL | |
| | SSPVTKSFNRGECQVQLQESGP | |
| | GLVKPSETLSLTCTVSGFSLTNY | |
| | AINWVRQPPGKGLEWLGIIWTG | |
| | GGTSYNSALKSRLTISKDTSKNQ | |
| | VSLKLSSVTAADTAVYYCARTG | |
| | TRGYFFDYWGQGTLVTVSSAST | |
| | KGPSVFPLAPSSKSTSGGTAALG | |
| | CLVKDYFPEPVTVSWNSGALTS | |
| | GVHTFPAVLQSSGLYSLSSVVT | |
| | VPSSSLGTQTYICNVNHKPSNTK | |
| | VDKKVEPKSCDKTHTCPPCPAP | |
| | ELLGGPDVFLFPPKPKDTLMISR | |
| | TPEVTCVVVDVSHEDPEVKFNW | |
| | YVDGVEVHNAKTKPREEQYNS | |
| | TYRVVSVLTVLHQDWLNGKEY | |
| | KCKVSNKALPAPEEKTISKAKG | |
| | QPREPQVYTLPPSREEMTKNQV | |
| | SLTCLVKGFYPSDIAVEWESNG | |
| | QPENNYKTTPPVLDSDGSFFLYS | |
| | KLTVDKSRWQQGNVFSCSVMH | |
| | EALHNHYTQKSLSLSPGK (SEQ | |
| | ID NO: 203) | |
| ULBP2-CL-HC- | GRADPHSLCYDITVIPKFRPGPR | DIQMTQSPSSLSASVGDRVTI |
| α-PDGFRb 1 | WCAVQGQVDEKTFLHYDCGNK | TCHASQNINVWLSWYQQKP |
| S239D/I332E | TVTPVSPLGKKLNVTTAWKAQ | GKAPKLLIYKASNLHTGVPSR |
| | NPVLREVVDILTEQLRDIQLENY | FSGSGSGTGFTLTISSLQPEDI |
| | TPKEPLTLQARMSCEQKAEGHS | ATYYCQQGQSFPFTFGGGTK |
| | SGSWQFSFDGQIFLLFDSEKRM | VEIKRTVAAPSVFIFPPSDEQL |
| | WTTVHPGARKMKEKWENDKV | KSGTASVVCLLNNFYPREAK |
| | VAMSFHYFSMGDCIGWLEDFL | VQWKVDNALQSGNSQESVT |
| | MGMDSTLEPSAGAPLAMRTVA | EQDSKDSTYSLSSTLTLSKAD |
| | APSVFIFPPSDEQLKSGTASVVC | YEKHKVYACEVTHQGLSSPV |
| | LLNNFYPREAKVQWKVDNALQ | TKSFNRGEC (SEQ ID NO: 206) |
| | SGNSQESVTEQDSKDSTYSLSST | |
| | LTLSKADYEKHKVYACEVTHQ | |
| | GLSSPVTKSFNRGECQVQLQES | |
| | GPGLVKPSETLSLTCTVSGFSLT | |
| | NYAINWVRQPPGKGLEWLGIIW | |
| | TGGGTSYNSALKSRLTISKDTSK | |
| | NQVSLKLSSVTAADTAVYYCAR | |
| | TGTRGYFFDYWGQGTLVTVSSA | |
| | STKGPSVFPLAPSSKSTSGGTAA | |
| | LGCLVKDYFPEPVTVSWNSGAL | |
| | TSGVHTFPAVLQSSGLYSLSSVV | |
| | TVPSSSLGTQTYICNVNHKPSNT | |
| | KVDKKVEPKSCDKTHTCPPCPA | |
| | PELLGGPDVFLFPPKPKDTLMIS | |
| | RTPEVTCVVVDVSHEDPEVKFN | |
| | WYVDGVEVHNAKTKPREEQYN | |
| | STYRVVSVLTVLHQDWLNGKE | |
| | YKCKVSNKALPAPEEKTISKAK | |
| | GQPREPQVYTLPPSREEMTKNQ | |
| | VSLTCLVKGFYPSDIAVEWESN | |
| | GQPENNYKTTPPVLDSDGSFFL | |
| | YSKLTVDKSRWQQGNVFSCSV | |
| | MHEALHNHYTQKSLSLSPGK | |
| | (SEQ ID NO: 205) | |
| MICB-HC-α- | AEPHSLRYNLMVLSQDESVQSG | DIQMTQSPSSLSASVGDRVTI |
| PDGFRb 1 | FLAEGHLDGQPFLRYDRQKRRA | TCHASQNINVWLSWYQQKP |
| S239D/I332E | KPQGQWAEDVLGAKTWDTETE | GKAPKLLIYKASNLHTGVPSR |
| | DLTENGQDLRRTLTHIKDQKGG | FSGSGSGTGFTLTISSLQPEDI |
| | LHSLQEIRVCEIHEDSSTRGSRHF | ATYYCQQGQSFPFTFGGGTK |
| | YYDGELFLSQNLETQESTVPQSS | VEIKRTVAAPSVFIFPPSDEQL |
| | RAQTLAMNVTNFWKEDAMKT | KSGTASVVCLLNNFYPREAK |
| | KTHYRAMQADCLQKLQRYLKS | VQWKVDNALQSGNSQESVT |
| | GVAIRRTVPPMVNVTCSEVSEG | EQDSKDSTYSLSSTLTLSKAD |
| | NITVTCRASSFYPRNITLTWRQD | YEKHKVYACEVTHQGLSSPV |
| | GVSLSHNTQQWGDVLPDGNGT | TKSFNRGEC (SEQ ID NO: 208) |
| | YQTWVATRIRQGEEQRFTCYME | |
| | HSGNHGTHPVPSGKVLVLQSQR | |
| | TDQVQLQESGPGLVKPSETLSLT | |
| | CTVSGFSLTNYAINWVRQPPGK | |
| | GLEWLGIIWTGGGTSYNSALKS | |
| | RLTISKDTSKNQVSLKLSSVTAA | |
| | DTAVYYCARTGTRGYFFDYWG | |
| | QGTLVTVSSASTKGPSVFPLAPS | |
| | SKSTSGGTAALGCLVKDYFPEP | |
| | VTVSWNSGALTSGVHTFPAVLQ | |
| | SSGLYSLSSVVTVPSSSLGTQTYI | |
| | CNVNHKPSNTKVDKKVEPKSC | |
| | DKTHTCPPCPAPELLGGPDVFLF | |
| | PPKPKDTLMISRTPEVTCVVVD | |
| | VSHEDPEVKFNWYVDGVEVHN | |
| | AKTKPREEQYNSTYRVVSVLTV | |
| | LHQDWLNGKEYKCKVSNKALP | |
| | APEEKTISKAKGQPREPQVYTLP | |
| | PSREEMTKNQVSLTCLVKGFYP | |
| | SDIAVEWESNGQPENNYKTTPP | |
| | VLDSDGSFFLYSKLTVDKSRWQ | |
| | QGNVFSCSVMHEALHNHYTQK | |
| | SLSLSPGK (SEQ ID NO: 207) | |
| ULBP1-HC-α- | GWVDTHCLCYDFIITPKSRPEPQ | DIQMTQSPSSLSASVGDRVTI |
| PDGFRb 1 | WCEVQGLVDERPFLHYDCVNH | TCHASQNINVWLSWYQQKP |
| S239D/I332E | KAKAFASLGKKVNVTKTWEEQ | GKAPKLLIYKASNLHTGVPSR |
| | TETLRDVVDFLKGQLLDIQVEN | FSGSGSGTGFTLTISSLQPEDI |
| | LIPIEPLTLQARMSCEHEAHGHG | ATYYCQQGQSFPFTFGGGTK |
| | RGSWQFLFNGQKFLLFDSNNRK | VEIKRTVAAPSVFIFPPSDEQL |
| | WTALHPGAKKMTEKWEKNRD | KSGTASVVCLLNNFYPREAK |
| | VTMFFQKISLGDCKMWLEEFLM | VQWKVDNALQSGNSQESVT |
| | YWEQMLDPTKPPSLAPQVQLQE | EQDSKDSTYSLSSTLTLSKAD |
| | SGPGLVKPSETLSLTCTVSGFSL | YEKHKVYACEVTHQGLSSPV |
| | TNYAINWVRQPPGKGLEWLGII | TKSFNRGEC (SEQ ID NO: 210) |
| | WTGGGTSYNSALKSRLTISKDT | |
| | SKNQVSLKLSSVTAADTAVYYC | |
| | ARTGTRGYFFDYWGQGTLVTV | |
| | SSASTKGPSVFPLAPSSKSTSGG | |
| | TAALGCLVKDYFPEPVTVSWNS | |
| | GALTSGVHTFPAVLQSSGLYSLS | |
| | SVVTVPSSSLGTQTYICNVNHKP | |
| | SNTKVDKKVEPKSCDKTHTCPP | |
| | CPAPELLGGPDVFLFPPKPKDTL | |
| | MISRTPEVTCVVVDVSHEDPEV | |
| | KFNWYVDGVEVHNAKTKPREE | |
| | QYNSTYRVVSVLTVLHQDWLN | |
| | GKEYKCKVSNKALPAPEEKTIS | |
| | KAKGQPREPQVYTLPPSREEMT | |
| | KNQVSLTCLVKGFYPSDIAVEW | |
| | ESNGQPENNYKTTPPVLDSDGS | |
| | FFLYSKLTVDKSRWQQGNVFSC | |
| | SVMHEALHNHYTQKSLSLSPGK | |
| | (SEQ ID NO: 209) | |
| ULBP2-HC-α- | GRADPHSLCYDITVIPKFRPGPR | DIQMTQSPSSLSASVGDRVTI |
| PDGFRb 1 | WCAVQGQVDEKTFLHYDCGNK | TCHASQNINVWLSWYQQKP |
| S239D/I332E | TVTPVSPLGKKLNVTTAWKAQ | GKAPKLLIYKASNLHTGVPSR |
| | NPVLREVVDILTEQLRDIQLENY | FSGSGSGTGFTLTISSLQPEDI |
| | TPKEPLTLQARMSCEQKAEGHS | ATYYCQQGQSFPFTFGGGTK |
| | SGSWQFSFDGQIFLLFDSEKRM | VEIKRTVAAPSVFIFPPSDEQL |
| | WTTVHPGARKMKEKWENDKV | KSGTASVVCLLNNFYPREAK |
| | VAMSFHYFSMGDCIGWLEDFL | VQWKVDNALQSGNSQESVT |
| | MGMDSTLEPSAGAPLAMQVQL | EQDSKDSTYSLSSTLTLSKAD |
| | QESGPGLVKPSETLSLTCTVSGF | YEKHKVYACEVTHQGLSSPV |
| | SLTNYAINWVRQPPGKGLEWLG | TKSFNRGEC (SEQ ID NO: 212) |
| | IIWTGGGTSYNSALKSRLTISKD | |
| | TSKNQVSLKLSSVTAADTAVYY | |
| | CARTGTRGYFFDYWGQGTLVT | |
| | VSSASTKGPSVFPLAPSSKSTSG | |
| | GTAALGCLVKDYFPEPVTVSWN | |
| | SGALTSGVHTFPAVLQSSGLYSL | |
| | SSVVTVPSSSLGTQTYICNVNHK | |
| | PSNTKVDKKVEPKSCDKTHTCP | |
| | PCPAPELLGGPDVFLFPPKPKDT | |
| | LMISRTPEVTCVVVDVSHEDPE | |
| | VKFNWYVDGVEVHNAKTKPRE | |
| | EQYNSTYRVVSVLTVLHQDWL | |
| | NGKEYKCKVSNKALPAPEEKTI | |
| | SKAKGQPREPQVYTLPPSREEM | |
| | TKNQVSLTCLVKGFYPSDIAVE | |
| | WESNGQPENNYKTTPPVLDSDG | |
| | SFFLYSKLTVDKSRWQQGNVFS | |
| | CSVMHEALHNHYTQKSLSLSPG | |
| | K (SEQ ID NO: 211) | |
| ULBP2-HC-α- | GRADPHSLCYDITVIPKFRPGPR | DIQMTQSPSSLSASVGDRVTI |
| PDGFRb 1 | WCAVQGQVDEKTFLHYDCGNK | TCHASQNINVWLSWYQQKP |
| L234A/L235A | TVTPVSPLGKKLNVTTAWKAQ | GKAPKLLIYKASNLHTGVPSR |
| | NPVLREVVDILTEQLRDIQLENY | FSGSGSGTGFTLTISSLQPEDI |
| | TPKEPLTLQARMSCEQKAEGHS | ATYYCQQGQSFPFTFGGGTK |
| | SGSWQFSFDGQIFLLFDSEKRM | VEIKRTVAAPSVFIFPPSDEQL |
| | WTTVHPGARKMKEKWENDKV | KSGTASVVCLLNNFYPREAK |
| | VAMSFHYFSMGDCIGWLEDFL | VQWKVDNALQSGNSQESVT |
| | MGMDSTLEPSAGAPLAMQVQL | EQDSKDSTYSLSSTLTLSKAD |
| | QESGPGLVKPSETLSLTCTVSGF | YEKHKVYACEVTHQGLSSPV |
| | SLTNYAINWVRQPPGKGLEWLG | TKSFNRGEC (SEQ ID NO: 214) |
| | IIWTGGGTSYNSALKSRLTISKD | |
| | TSKNQVSLKLSSVTAADTAVYY | |
| | CARTGTRGYFFDYWGQGTLVT | |
| | VSSASTKGPSVFPLAPSSKSTSG | |
| | GTAALGCLVKDYFPEPVTVSWN | |
| | SGALTSGVHTFPAVLQSSGLYSL | |
| | SSVVTVPSSSLGTQTYICNVNHK | |
| | PSNTKVDKKVEPKSCDKTHTCP | |
| | PCPAPEAAGGPSVFLFPPKPKDT | |
| | LMISRTPEVTCVVVDVSHEDPE | |
| | VKFNWYVDGVEVHNAKTKPRE | |
| | EQYNSTYRVVSVLTVLHQDWL | |
| | NGKEYKCKVSNKALPAPIEKTIS | |
| | KAKGQPREPQVYTLPPSREEMT | |
| | KNQVSLTCLVKGFYPSDIAVEW | |
| | ESNGQPENNYKTTPPVLDSDGS | |
| | FFLYSKLTVDKSRWQQGNVFSC | |
| | SVMHEALHNHYTQKSLSLSPGK | |
| | (SEQ ID NO: 213) | |

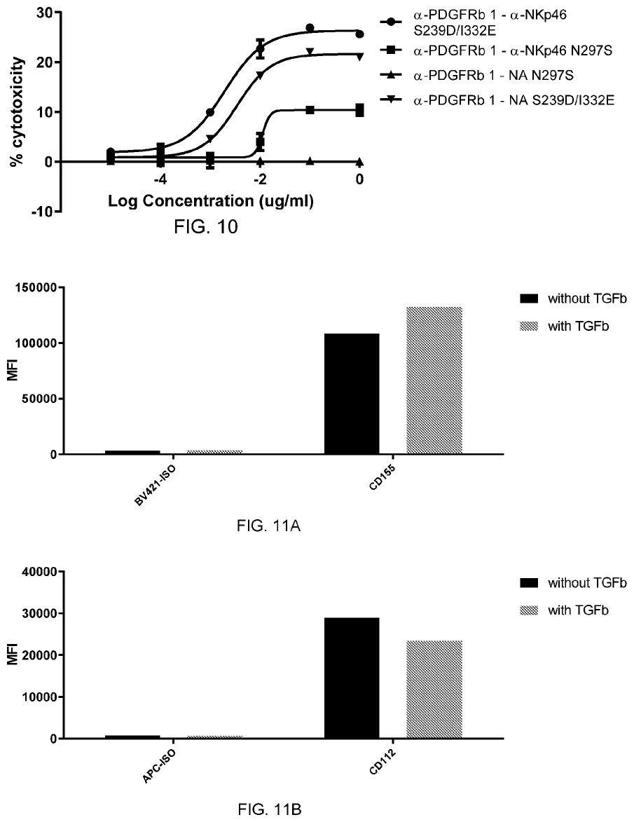

Activation of NKp46 signaling could also enhance ADCC cytotoxicity to activated HSCs. NKp46 is another activating receptor expressed on NK cells. Bispecific antibody by fusion anti-NKp46 agonist antibody to C-term of heavy chain of anti-PDGFRb antibody were designed. Exemplary constructs thereof are shown in Table 8 below. Fc mutations (S239D/1332E to enhance the effector function or N297S to abolish the effector function) were introduced into the bispecific antibodies. These antibodies were expressed, purified and evaluated in in vitro ADCC cytotoxicity assay. As shown in FIG. 10, fusion anti-NKp46 agonist antibody to anti-PDGFRb antibody significantly enhanced ADCC cytotoxicity to TGFb1-activated human HSC cells. This effect was more obvious when Fc effector function was silenced by N297S mutation.

TABLE 8
Full lengh sequences of exemplary
α-PDGFRb 1- α-NKp46 1 fusion constructs

| Construct | Chain 1 | Chain 2 | Chain 3 |
|---|---|---|---|
| α-PDGFRb 1-α- | QVQLQESGPG | DIQMTQSPSS | DIQMTQSPSS |
| NKp46 N297S | LVKPSETLSL | LSASVGDRVT | LSASVGDRVT |
| | TCTVSGFSLT | ITCHASQNIN | ITCRASQDIS |
| | NYAINWVRQP | VWLSWYQQKP | NYLNWYQQKP |
| | PGKGLEWLGI | GKAPKLLIYK | GKAPKLLIYY |
| | IWTGGGTSYN | ASNLHTGVPS | TSRLHSGVPS |
| | SALKSRLTIS | RFSGSGSGTG | RFSGSGSGTD |
| | KDTSKNQVSL | FTLTISSLQP | FTFTISSLQP |
| | KLSSVTAADT | EDIATYYCQQ | EDIATYFCQQ |
| | AVYYCARTGT | GQSFPFTFGG | GNTRPWTFGG |
| | RGYFFDYWGQ | GTKVEIKRTV | GTKVEIKAST |
| | GTLVTVSSAS | AAPSVFIFPP | KGPSVFPLAP |
| | TKGPSVFPLA | SDEQLKSGTA | SSKSTSGGTA |
| | PSSKSTSGGT | SVVCLLNNFY | ALGCLVKDYF |
| | AALGCLVKDY | PREAKVQWKV | PEPVTVSWNS |
| | FPEPVTVSWN | DNALQSGNSQ | GALTSGVHTF |
| | SGALTSGVHT | ESVTEQDSKD | PAVLQSSGLY |
| | FPAVLQSSGL | STYSLSSTLT | SLSSVVTVPS |
| | YSLSSVVTVP | LSKADYEKHK | SSLGTQTYIC |
| | SSSLGTQTYI | VYACEVTHQG | NVNHKPSNTK |
| | CNVNHKPSNT | LSSPVTKSFN | VDKKV |
| | KVDKKVEPKS | RGECEPKSCD | (SEQ ID |
| | CDKTHTCPPC | KTHTCPPCPA | NO: 217) |
| | PAPELLGGPS | PELLGGPSVF | |
| | VFLFPPKPKD | LFPPKPKDTL | |
| | TLMISRTPEV | MISRTPEVTC | |
| | TCVVVDVSHE | VVVDVSHEDP | |
| | DPEVKFNWYV | EVKFNWYVDG | |
| | DGVEVHNAKT | VEVHNAKTKP | |
| | KPREEQYSST | REEQYSSTYR | |
| | YRVVSVLTVL | VVSVLTVLHQ | |
| | HQDWLNGKEY | DWLNGKEYKC | |
| | KCKVSNKALP | KVSNKALPAP | |
| | APIEKTISKA | IEKTISKAKG | |
| | KGQPREPQVY | QPREPQVYTL | |
| | TLPPSREEMT | PPSREEMTKN | |
| | KNQVSLTCLV | QVSLTCLVKG | |
| | KGFYPSDIAV | FYPSDIAVEW | |
| | EWESNGQPEN | ESNGQPENNY | |
| | NYKTTPPVLD | KTTPPVLDSD | |
| | SDGSFFLYSK | GSFFLYSKLT | |
| | LTVDKSRWQQ | VDKSRWQQGN | |
| | GNVFSCSVMH | VFSCSVMHEA | |
| | EALHNHYTQK | LHNHYTQKSL | |
| | SLSLSPGSTG | SLSPGK | |
| | SQVQLVQSGA | (SEQ ID | |
| | EVKKPGSSVK | NO: 216) | |
| | VSCKASGYTF | | |
| | TDYVINWGRQ | | |
| | APGQGLEWIG | | |
| | EIYPGSGTNY | | |
| | YNEKFKAKAT | | |
| | ITADKSTSTA | | |
| | YMELSSLRSE | | |
| | DTAVYFCARR | | |
| | GRYGLYAMDY | | |
| | WGQGTTVTVS | | |
| | SRTVAAPSVF | | |
| | IFPPSDEQLK | | |
| | SGTASVVCLL | | |
| | NNFYPREAKV | | |
| | QWKVDNALQS | | |
| | GNSQESVTEQ | | |
| | DSKDSTYSLS | | |
| | STLTLSKADY | | |
| | EKHKVYACEV | | |
| | THQGLSSPVT | | |
| | KSFNRGEC | | |
| | (SEQ ID | | |
| | NO: 215) | | |
| α-PDGFRb 1-α- | QVQLQESGPG | DIQMTQSPSS | DIQMTQSPSS |
| NKp46 | LVKPSETLSL | LSASVGDRVT | LSASVGDRVT |
| S239D/1332E | TCTVSGFSLT | ITCHASQNIN | ITCRASQDIS |
| | NYAINWVRQP | VWLSWYQQKP | NYLNWYQQKP |
| | PGKGLEWLGI | GKAPKLLIYK | GKAPKLLIYY |
| | IWTGGGTSYN | ASNLHTGVPS | TSRLHSGVPS |
| | SALKSRLTIS | RFSGSGSGTG | RFSGSGSGTD |
| | KDTSKNQVSL | FTLTISSLQP | FTFTISSLQP |
| | KLSSVTAADT | EDIATYYCQQ | EDIATYFCQQ |
| | AVYYCARTGT | GQSFPFTFGG | GNTRPWTFGG |
| | RGYFFDYWGQ | GTKVEIKRTV | GTKVEIKAST |
| | GTLVTVSSAS | AAPSVFIFPP | KGPSVFPLAP |
| | TKGPSVFPLA | SDEQLKSGTA | SSKSTSGGTA |
| | PSSKSTSGGT | SVVCLLNNFY | ALGCLVKDYF |
| | AALGCLVKDY | PREAKVQWKV | PEPVTVSWNS |
| | FPEPVTVSWN | DNALQSGNSQ | GALTSGVHTF |
| | SGALTSGVHT | ESVTEQDSKD | PAVLQSSGLY |
| | FPAVLQSSGL | STYSLSSTLT | SLSSVVTVPS |
| | YSLSSVVTVP | LSKADYEKHK | SSLGTQTYIC |
| | SSSLGTQTYI | VYACEVTHQG | NVNHKPSNTK |
| | CNVNHKPSNT | LSSPVTKSFN | VDKKV |
| | KVDKKVEPKS | RGECEPKSCD | (SEQ ID |
| | CDKTHTCPPC | KTHTCPPCPA | NO: 220) |
| | PAPELLGGPD | PELLGGPDVF | |
| | VFLFPPKPKD | LFPPKPKDTL | |
| | TLMISRTPEV | MISRTPEVTC | |
| | TCVVVDVSHE | VVVDVSHEDP | |
| | DPEVKFNWYV | EVKFNWYVDG | |
| | DGVEVHNAKT | VEVHNAKTKP | |
| | KPREEQYNST | REEQYNSTYR | |
| | YRVVSVLTVL | VVSVLTVLHQ | |
| | HQDWLNGKEY | DWLNGKEYKC | |
| | KCKVSNKALP | KVSNKALPAP | |
| | APEEKTISKA | EEKTISKAKG | |
| | KGQPREPQVY | QPREPQVYTL | |
| | TLPPSREEMT | PPSREEMTKN | |
| | KNQVSLTCLV | QVSLTCLVKG | |
| | KGFYPSDIAV | FYPSDIAVEW | |
| | EWESNGQPEN | ESNGQPENNY | |
| | NYKTTPPVLD | KTTPPVLDSD | |
| | SDGSFFLYSK | GSFFLYSKLT | |
| | LTVDKSRWQQ | VDKSRWQQGN | |
| | GNVFSCSVMH | VFSCSVMHEA | |
| | EALHNHYTQK | LHNHYTQKSL | |
| | SLSLSPGSTG | SLSPGK | |
| | SQVQLVQSGA | (SEQ ID | |
| | EVKKPGSSVK | NO: 219) | |
| | VSCKASGYTF | | |
| | TDYVINWGRQ | | |
| | APGQGLEWIG | | |
| | EIYPGSGTNY | | |
| | YNEKFKAKAT | | |
| | ITADKSTSTA | | |
| | YMELSSLRSE | | |
| | DTAVYFCARR | | |
| | GRYGLYAMDY | | |
| | WGQGTTVTVS | | |
| | SRTVAAPSVF | | |
| | IFPPSDEQLK | | |
| | SGTASVVCLL | | |
| | NNFYPREAKV | | |
| | QWKVDNALQS | | |
| | GNSQESVTEQ | | |
| | DSKDSTYSLS | | |
| | STLTLSKADY | | |
| | EKHKVYACEV | | |
| | THQGLSSPVT | | |
| | KSFNRGEC | | |
| | (SEQ ID | | |
| | NO: 218) | | |
| a-PDGFRb 1- | QVQLQESGPG | DIQMTQSPSS | |
| NA N297S | LVKPSETLSL | LSASVGDRVT | |
| | TCTVSGFSLT | ITCHASQNIN | |
| | NYAINWVRQP | VWLSWYQQKP | |
| | PGKGLEWLGI | GKAPKLLIYK | |
| | IWTGGGTSYN | ASNLHTGVPS | |
| | SALKSRLTIS | RFSGSGSGTG | |
| | KDTSKNQVSL | FTLTISSLQP | |
| | KLSSVTAADT | EDIATYYCQQ | |
| | AVYYCARTGT | GQSFPFTFGG | |
| | RGYFFDYWGQ | GTKVEIKRTV | |
| | GTLVTVSSAS | AAPSVFIFPP | |
| | TKGPSVFPLA | SDEQLKSGTA | |
| | PSSKSTSGGT | SVVCLLNNFY | |
| | AALGCLVKDY | PREAKVQWKV | |
| | FPEPVTVSWN | DNALQSGNSQ | |
| | SGALTSGVHT | ESVTEQDSKD | |
| | FPAVLQSSGL | STYSLSSTLT | |
| | YSLSSVVTVP | LSKADYEKHK | |
| | SSSLGTQTYI | VYACEVTHQG | |
| | CNVNHKPSNT | LSSPVTKSFN | |
| | KVDKKVEPKS | RGECEPKSCD | |
| | CDKTHTCPPC | KTHTCPPCPA | |
| | PAPELLGGPS | PELLGGPSVF | |
| | VFLFPPKPKD | LFPPKPKDTL | |
| | TLMISRTPEV | MISRTPEVTC | |
| | TCVVVDVSHE | VVVDVSHEDP | |
| | DPEVKFNWYV | EVKFNWYVDG | |
| | DGVEVHNAKT | VEVHNAKTKP | |
| | KPREEQYSST | REEQYSSTYR | |
| | YRVVSVLTVL | VVSVLTVLHQ | |
| | HQDWLNGKEY | DWLNGKEYKC | |
| | KCKVSNKALP | KVSNKALPAP | |
| | APIEKTISKA | IEKTISKAKG | |
| | KGQPREPQVY | QPREPQVYTL | |
| | TLPPSREEMT | PPSREEMTKN | |
| | KNQVSLTCLV | QVSLTCLVKG | |
| | KGFYPSDIAV | FYPSDIAVEW | |
| | EWESNGQPEN | ESNGQPENNY | |
| | NYKTTPPVLD | KTTPPVLDSD | |
| | SDGSFFLYSK | GSFFLYSKLT | |
| | LTVDKSRWQQ | VDKSRWQQGN | |
| | GNVFSCSVMH | VFSCSVMHEA | |
| | EALHNHYTQK | LHNHYTQKSL | |
| | SLSLSPGK | SLSPGK | |
| | (SEQ ID | (SEQ ID | |
| | NO: 221) | NO: 222) | |
| α-PDGFRb 1- | QVQLQESGPG | DIQMTQSPSS | |
| NA S239D/ | LVKPSETLSL | LSASVGDRVT | |
| 1332E | TCTVSGFSLT | ITCHASQNIN | |
| | NYAINWVRQP | VWLSWYQQKP | |
| | PGKGLEWLGI | GKAPKLLIYK | |
| | IWTGGGTSYN | ASNLHTGVPS | |
| | SALKSRLTIS | RFSGSGSGTG | |
| | KDTSKNQVSL | FTLTISSLQP | |
| | KLSSVTAADT | EDIATYYCQQ | |
| | AVYYCARTGT | GQSFPFTFGG | |
| | RGYFFDYWGQ | GTKVEIKRTV | |
| | GTLVTVSSAS | AAPSVFIFPP | |
| | TKGPSVFPLA | SDEQLKSGTA | |
| | PSSKSTSGGT | SVVCLLNNFY | |
| | AALGCLVKDY | PREAKVQWKV | |
| | FPEPVTVSWN | DNALQSGNSQ | |
| | SGALTSGVHT | ESVTEQDSKD | |
| | FPAVLQSSGL | STYSLSSTLT | |
| | YSLSSVVTVP | LSKADYEKHK | |
| | SSSLGTQTYI | VYACEVTHQG | |
| | CNVNHKPSNT | LSSPVTKSFN | |
| | KVDKKVEPKS | RGECEPKSCD | |
| | CDKTHTCPPC | KTHTCPPCPA | |
| | PAPELLGGPD | PELLGGPDVF | |
| | VFLFPPKPKD | LFPPKPKDTL | |
| | TLMISRTPEV | MISRTPEVTC | |
| | TCVVVDVSHE | VVVDVSHEDP | |
| | DPEVKFNWYV | EVKFNWYVDG | |
| | DGVEVHNAKT | VEVHNAKTKP | |
| | KPREEQYNST | REEQYNSTYR | |
| | YRVVSVLTVL | VVSVLTVLHQ | |
| | HQDWLNGKEY | DWLNGKEYKC | |
| | KCKVSNKALP | KVSNKALPAP | |
| | APEEKTISKA | EEKTISKAKG | |
| | KGQPREPQVY | QPREPQVYTL | |
| | TLPPSREEMT | PPSREEMTKN | |
| | KNQVSLTCLV | QVSLTCLVKG | |
| | KGFYPSDIAV | FYPSDIAVEW | |
| | EWESNGQPEN | ESNGQPENNY | |
| | NYKTTPPVLD | KTTPPVLDSD | |
| | SDGSFFLYSK | GSFFLYSKLT | |
| | LTVDKSRWQQ | VDKSRWQQGN | |
| | GNVFSCSVMH | VFSCSVMHEA | |
| | EALHNHYTQK | LHNHYTQKSL | |
| | SLSLSPGK | SLSPGK | |
| | (SEQ ID | (SEQ ID | |
| | NO: 223) | NO: 224) | |

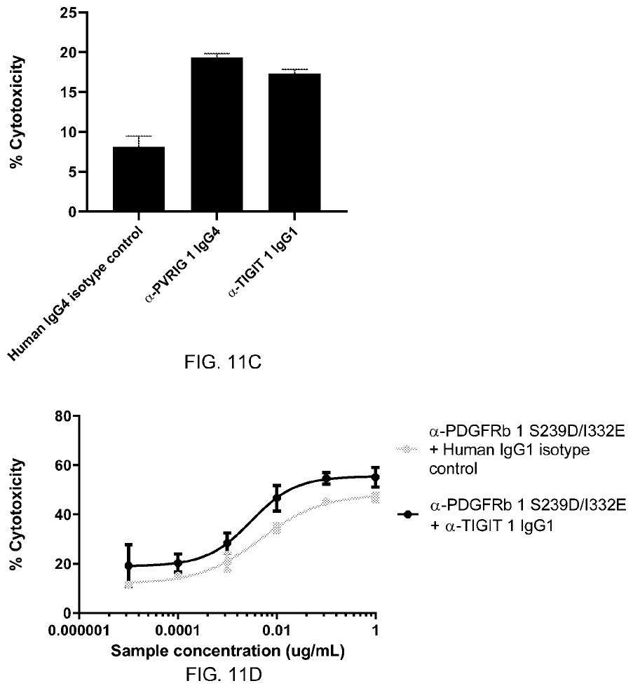

aHSCs, by expressing multiple ligands for inhibitory immune checkpoint receptors, inhibit the activity of NK cells in the liver. Blocking the interaction between these ligands and their inhibitory immune checkpoint receptors is another way to modulate NK cell activity. As shown in FIGS. 11A and 11B, CD155 (ligand for TIGIT) and CD112 (ligand for PVRIG) expressed highly on human HSC cells both with or without 2 ng/ml TGFb1 treatment. TGFb1-activated HSC cells were labeled with Calcein-AM fluorescent dye. ADCC cytotoxicity was measured by the level of Calcei-AM released to the culture medium. Blocking the interaction between CD155 and TIGIT by α-TIGIT 1 IgG1 antibody or the interaction between CD112 and PVRIG by α-PVRIG 1 IgG4 significantly increased the baseline ADCC cytotoxic activity of NK cells to activated HSC cells (FIG. 11C). Importantly, 3 μg/ml α-TIGIT 1 IgG1 combined with a range concentration of α-PDGFRb 1 S239D/1332E induced more ADCC cytotoxicity to aHSCs than α-PDGFRb 1 S239D/1332E treatment alone (FIG. 11D).

TGFb is an immune suppressive cytokine. It is known to inhibit cytotoxic activity of immune cells. TGFb has high binding affinity to its receptor. Soluble TGFb receptor II ECD (TRII) has been used as a decoy receptor to block TGFb signaling. To block the immune suppressive function of TGFb, bispecific antibody was designed by fusion TRII to the heavy chain C-terminus of anti-human PDGFRb antibody. Exemplary construct is shown in Table 7. This bispecific antibody was tested in in vitro ADCC cytotoxicity assay together with anti-PDGFRb antibody alone. Human primary HSC was activated by hTGFb1 for 24 hours. On the day of ADCC assay, activated HSC cells were collected and co-cultured with primary NK cells in RPMI 1640 medium. No TGFb1 was included in the culture medium during the course of ADCC assay. No significant difference of ADCC cytotoxicity was observed between anti-PDGFRb antibody with TRII treated groups and anti-PDGFRb antibody alone treated group (FIG. 12A).

Although no significant difference was observed in in vitro ADCC assay, differential in vivo effects were observed between anti-mouse PDGFRb antibody linked-TRII treatment group and anti-mouse PDGFRb antibody group. In vivo activity of anti-mouse PDGFRb antibody TRII bispecific was evaluated in CCL4-treated mouse model. Male wild type C57BL/6J mice were treated with CCL4 for three weeks to induce HSC cells activation. These mice were then given 45 mg/kg of anti-mPDGFRb antibody TRII bispecific (1B3-TRII S239D/1332E) (i.v., twice a week for 4 weeks, 8 doses in total), anti-mPDGFRb antibody (1B3 S239D/1332E) (i.v., once a week for 4 weeks, 4 doses in total) along with CCL4 injections for additional 4 weeks. Fc mutation S239D/1332E was engineered into the antibody to enhance the effector function. An mIgG2a isotype control antibody linked TRII bispecific antibody group (ISO-TRII) (i.v., once a week for 4 weeks, 4 doses in total) was also included in the study. Serum samples were collected on day 9 and day 27 after the start of antibody treatment. Liver samples were collected at the end of the study and homogenized in RIPA lysis buffer supplemented with protease and phosphatase inhibitor. After centrifugation, the supernatant was collected as the liver lysate. Total PDGFRb levels in the liver lysate were measured by sandwich ELISA. Anti-PDGFRb antibody (abcam Ab32570) was used as capture antibody. 1B3 mIgG2a was used as detection antibody. Antibody levels in the serum was determined using direct ELISA with mouse PDGFRb as coating antigen and anti-TRII antibody as detection antibody. As shown in FIG. 12B, serum antibody levels were comparable between of 1B3-TRII S239D/1332 treated group and 1B3 S239D/1332E treated group. As the indication of activated HSC depletion, liver total PDGFRb levels were significantly reduced by both treatment groups (FIG. 12C). 1B3-TRII S239D/1332E treatment caused more total PDGFRb reduction than 1B3 S239D/1332E treatment. These results demonstrated that blocking TGFb signaling could further enhance antibody-mediated depletion of activated HSC cells in the liver.

These results further confirmed that modulating immune cells activity either by promoting immune cell activation signaling or suppressing immune cell inhibitory signaling could further enhance antibody-mediated cytotoxicity to effectively deplete activated HSCs in vitro and in vivo.

### 7.5. Example 5: Depletion of Activated HSC in the Liver was Associated with the Changes of HSC Markers and NK Activation Markers In Vivo

Male wild type C57BL/6J mice were treated with CCL4 (0.7 ml/kg, i.p. injection, three times a week) for three weeks to induce HSC cells activation. These mice were then given CCL4 together with 45 mg/kg of anti-mPDGFRb antibody 1B3 S239D/1332E (I.V.), 15 mg/kg of anti-mPDGFRb antibody 1B3 S239D/1332E (S.C.), or 5 mg/kg anti-mPDGFRb antibody 1B3 S239D/1332E (S.C.). Liver samples were collected at 4, 48 and 168 hours after the start of antibody dosing. Half of the liver was used for protein or mRNA extraction and the other half of the liver was used for pathology study.

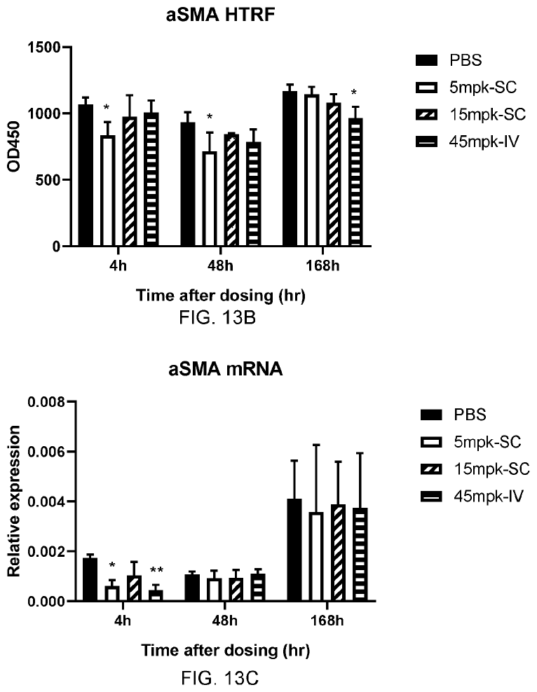

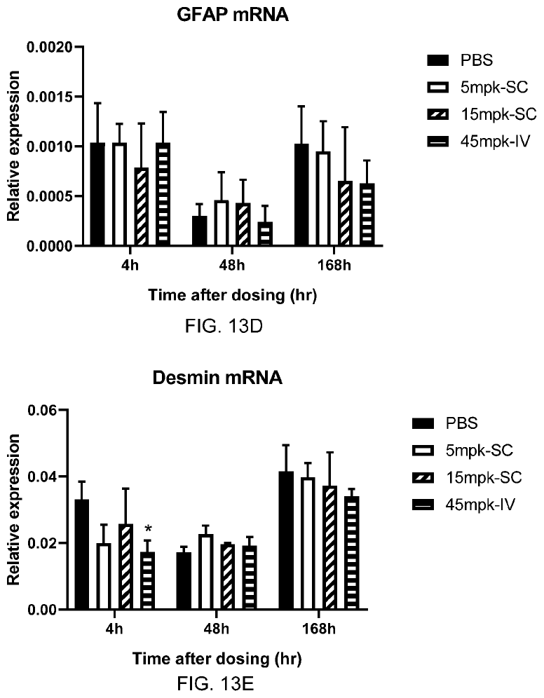

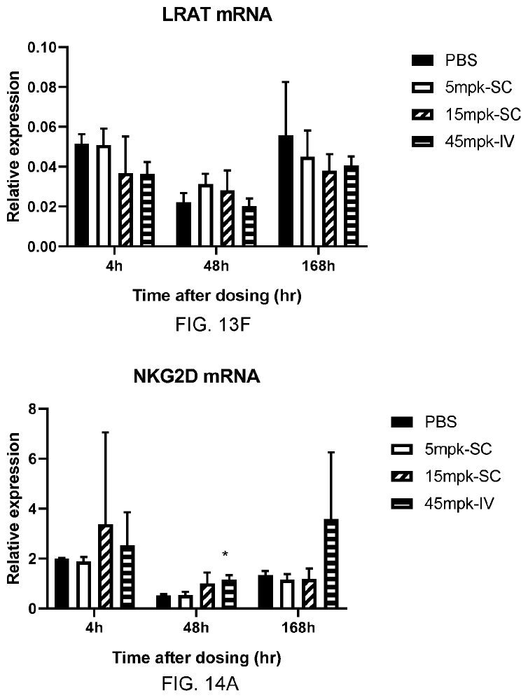

To explore the marker changes associated with in vivo HSC depletion, multiple HSC markers were studied. As shown in FIG. 13A, 1B3 S239D/1332E treatment time dependently reduced liver total PDGFRb levels as measured by ELISA. All three dosing groups induced significant reduction of PDGFRb at 4 hours and 48 hours after antibody dosing. Only 45 mg/kg group reduced liver PDGFRb levels at 168 hours after antibody dosing, which is consistent with antibody serum PK. The protein level of activated HSC marker aSMA were reduced most significantly by 5 mg/kg 1B3 S239D/1332E group at 4 hours and 48 hours after antibody dosing (FIG. 13B). Using quantitative RT-PCR, aSMA mRNA levels were also reduced significantly by 5 mg/kg and 45 mg/kg 1B3 S239D/1332E group at 4 hours after dosing (FIG. 13C). Pan HSC markers (expressed on both quiescent and activated HSC) such as GFAP, Desmin and LRAT were not changed significantly by antibody treatment at mRNA levels (FIGS. 13D to 13F).

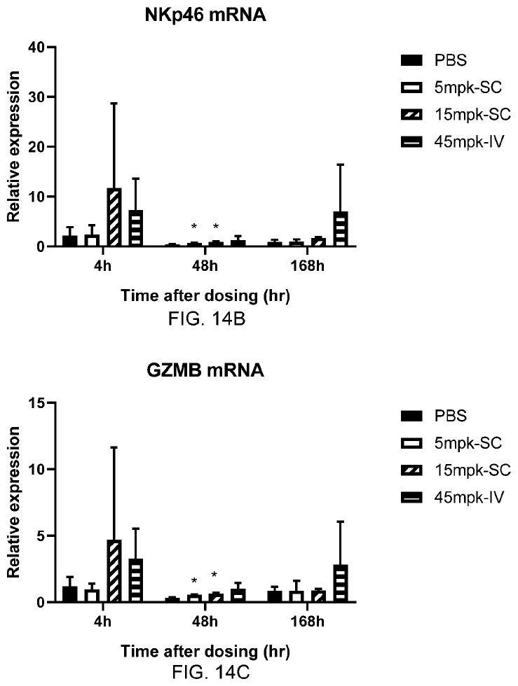

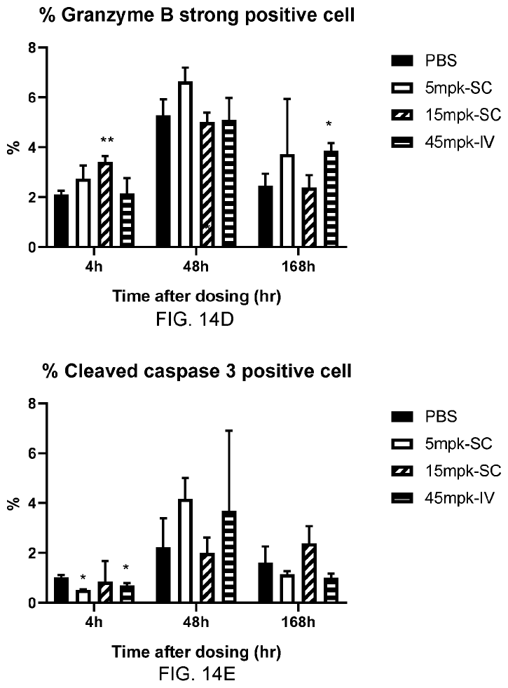

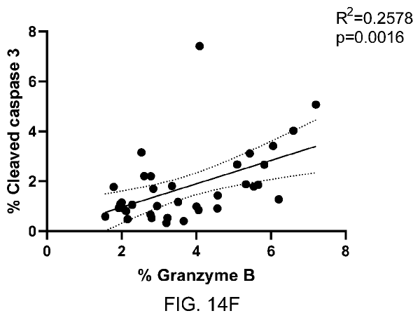

Immune cell and NK cell activation markers were also studied. As shown in FIGS. 14A and 14B, 1B3 S239D/1332E treatment increased mRNA levels of NK markers NKG2D and NKp46 at 48 hours after antibody dosing. Immune cell activation marker Granzyme B mRNA levels were also significantly increased at 48 hours after antibody dosing (FIG. 14C). Using IHC, 1B3 S239D/1332E treatment significantly increased % Granzyme B strong positive cells in the liver at 4 and 168 hours after the start of antibody dosing. A trend of increased % Granzyme B strong positive cells by 5 mg/kg 1B3 S239D/1332E was also observed at 48 hours. % apoptotic cells in the liver quantified by cleaved caspase 3 IHC was decreased by 5 mg/kg and 45 mg/kg 1B3 S239D/1332E at 4 hours after the start of dosing and increased by 5 mg/kg 1B3 S239D/1332E at 48 hours. There were a good correlation between % Granzyme B strong positive cells and % cleaved caspase 3 positive cells, suggesting 1B3 S239D/1332E activated immune cells in the liver to exert HSC killing effect.

### 7.6. Example 6: Depletion of activated HSCs reduces liver fibrosis

Whether depletion of activated HSC could reduce liver fibrosis was evaluated in multiple dose study of anti-mPDGFRb antibodies in CCL4-treated mice. Male wild type C57BL/6J mice were treated with CCL4 (0.7 ml/kg, i.p. injection, three times a week) for three weeks to induce HSC cells activation. These mice were then given 45 mg/kg of anti-mPDGFRb antibody TRII bispecific (1B3-TRII S239D/1332E, 1B3-TRII L234A/L235A) (i.v., twice a week for 4 weeks, 8 doses in total), anti-mPDGFRb antibody (1B3 S239D/1332E, 1B3 WT) (i.v., once a week for 4 weeks, 4 doses in total) together with CCL4 injections for additional 4 weeks. Fc mutation S239D/1332E was engineered into the antibody to enhance the effector function. Fc mutation L234A/L235A was engineered into the antibody to abolish effector function. An mIgG2a isotype control antibody linked TRII bispecific group (ISO-TRII) (i.v., once a week for 4 weeks, 4 doses in total) was also included in the study. Liver samples were collected at the end of the study and half of the samples were homogenized in RIPA lysis buffer supplemented with protease and phosphatase inhibitor. Total PDGFRb levels in liver lysate were measured by ELISA as an indication of in vivo depletion of activated HSC cells. FFPE tissue blocks were generated using the rest of liver samples. Liver sections were stained with Sirius red dye (SR) to determine the extent of liver fibrosis. The SR staining intensity and staining area was quantified by image software HALO.

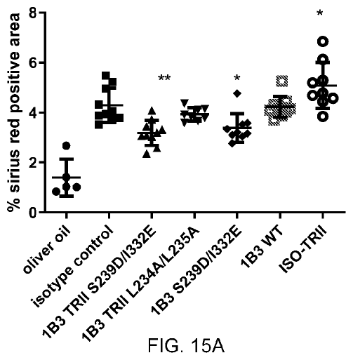

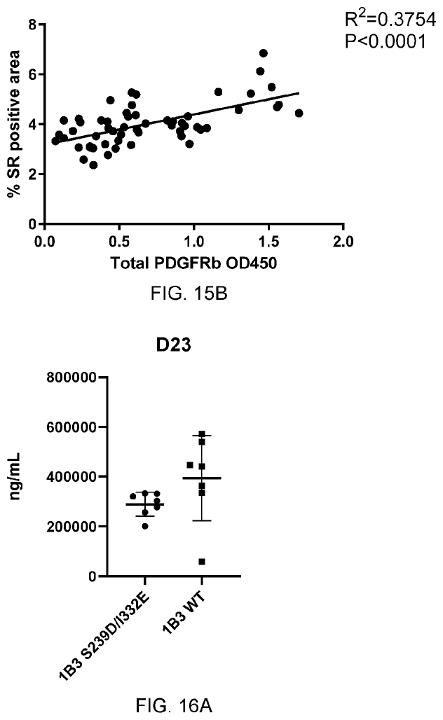

As shown in FIG. 12C, depletion of activated HSCs indicated as total PDGFRb protein levels were observed in the treatment groups of antibodies with normal or enhanced effector function (1B3 TRII S239D/1332E, 1B3 S239D/1332E and 1B3 WT). Intriguingly, liver fibrosis (as quantified as % SR area) was only significantly reduced by 1B3 TRII S239D/1332E and 1B3 S239D/1332E treatment (FIG. 15A). Although 1B3 WT treatment reduced total PDGFRb levels, it could not inhibit liver fibrosis progression. 1B3 TRII L234A/L235A treatment had no effect on total PDGFRb protein levels and liver fibrosis. A significant correlation between total PDGFRb protein levels and liver fibrosis as measured by SR staining was observed (FIG. 15B). These results suggested that depletion of activated HSC could efficiently reduce liver fibrosis in vivo. Enhanced effector function, e.g., by Fc mutations, was required for an HSC-targeting antibody to exert therapeutic effect against diseases with liver fibrosis.

The effect anti-mPDGFRb antibodies was further evaluated in choline-deficient L-amino acid high fat diet (CDAA diet)-treated mice. Male wild type C57BL/6J mice were given CDAA diet (choline deficient, 0.1% Methionine, 1% cholesterol, 45% high fat diet) together with 45 mg/kg 1B3 S239D/1332E or 45 mg/kg 1B3 WT (I.V., once a week) for 6 weeks. PBS was used as the control. Serum samples were collected at day 23 and day 42 after the starting of antibody dosing. Liver samples were collected at the end of the study and half of the liver were homogenized in RIPA lysis buffer supplemented with protease and phosphatase inhibitor. Total PDGFRb levels in liver lysate were measured by ELISA as an indication of in vivo depletion of activated HSC cells. FFPE tissue blocks were generated using the other half of the livers. Liver sections were stained with Sirius red dye (SR) to determine the extent of liver fibrosis. The SR staining intensity and staining area was quantified by image software HALO.

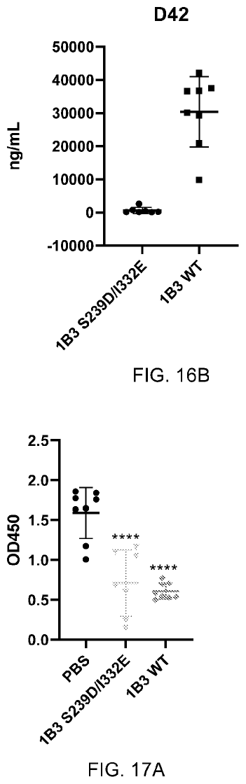

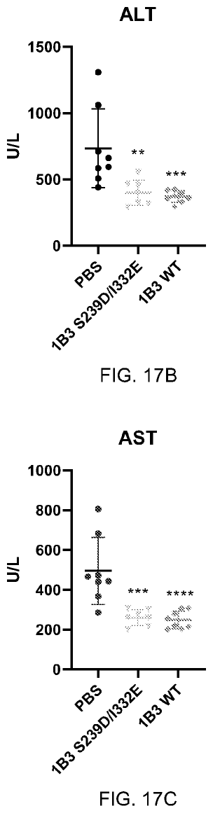

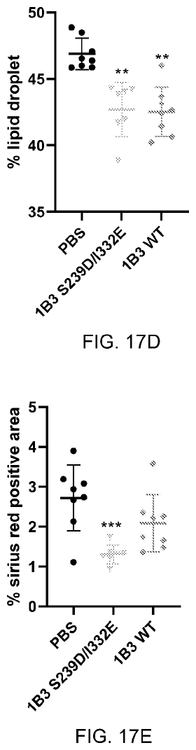

As shown in FIGS. 16A and 16B, 1B3 WT group had higher serum antibody concentration comparing to 1B3 S239D/1332E group at both day 23 and day 42 time points. After 6 weeks antibody administration, both 1B3 S239D/1332E and 1B3 WT treatment significantly reduced serum ALT/AST levels measured by ADVIA 2400 chemistry system (FIGS. 17B and 17C). Liver sample analysis showed that both 1B3 S239D/1332E and 1B3 WT treatment significantly decreased liver total PDGFRb protein levels (FIG. 17A) and % lipid droplet in the liver (FIG. 17D). Interestingly, only 1B3 S239D/1332E treatment significantly reduced liver fibrosis as measured by % SR positive area in the liver (FIG. 17E). This is consistent with the findings from CCL4-treated animal study (FIG. 15A). Fc with enhanced effector function was required for anti-fibrosis effect of aHSC-targeting antibody.

What is claimed:

1. A binding molecule comprising one or more binding domain(s) that bind(s) to one or more antigen(s) expressed on an activated hepatic stellate cells (HSC), and a functional domain that is capable of enhancing an antibody effector function toward activated HSCs, wherein optionally the antibody effector function is antibody-dependent cell-mediated cytotoxicity (ADCC).

2. The binding molecule of claim 1, wherein the functional domain is:
(i) an Fc region comprising one or more mutation(s) that enhances ADCC; or
(ii) a domain that activates an immune cell, and wherein optionally the immune cell is a NK cell.

3. The binding molecule of claim 2, wherein the functional domain is the Fc region comprising one or more mutation(s) that enhances ADCC, and wherein optionally the one or more mutation(s) of the Fc region is at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering.

4. The binding molecule of claim 3, wherein the Fc region comprises S239D/1332E, S239D/A330L/1332E, L235V/F243L/R292P/Y300L/P396L, or F243L/R292P/Y300L mutations.

5. The binding molecule of claim 2, wherein the functional domain is the domain that activates the immune cell, and wherein the functional domain promotes immune cell activation signaling or suppresses immune cell inhibitory signaling.

6. The binding molecule of claim 5, wherein the functional domain binds to and/or modulates a receptor on an immune cell, and wherein optionally the receptor is selected from a group consisting of NKp46, NKp30, NKp44, NKG2C, CD94, KIR2DS1, KIR2DS4, KIR2DS2, KIR2DL4, KIR3DS1, CD160, NKG2D, NKp80, DNAM1, 2B4, NTB-A, CRACC, 4-1BB, OX40, CRTAM, CD16, LFA1, NKG2A, KIR2DL1, KIR2DL2, KIR2DL3, KIR3DL1, KIR3DL2, LILRB1, KIR2DL5, KLRG1, CD161, SIGLEC7, SIGLEC9, LAIR1, TIGIT, CD96, PD-1, PVRIG, CD122, CD132, CD218a, CD218b, IL12Rb1, IL12Rb2, IL21R, TGFBR1, TGFBR2, TGFBR3, ACVR2A, ACVR2B, ALK4, , and A2a.

7. The binding molecule of claim 5 or claim 6, wherein the functional domain binds to NKG2D.

8. The binding molecule of claim 7, wherein the functional domain is derived from a NKG2D ligand.

9. The binding molecule of claim 8, wherein the functional domain is the extracellular domain of MICA, MICB, ULBP1, or ULBP2 or a fragment or a variant thereof, and wherein optionally the functional domain comprises an amino acid sequence of any one of SEQ ID NO: 90-93 or a fragment thereof.

10. The binding molecule of claim 5 or claim 6, wherein the functional domain binds to NKp46.

11. The binding molecule of claim 10, wherein the functional domain comprises an antibody that binds to NKp46.

12. The binding molecule of claim 11, wherein the antibody comprises an HCDR1, an HCDR2, and an HCDR3 as set forth in SEQ ID NO: 87, and a LCDR1, a LCDR2, and a LCDR3 as set forth in SEQ ID NO: 88.

13. The binding molecule of claim 12, wherein the HCDR1 comprises the amino acid sequence of SEQ ID NO: 61, the HCDR2 comprises the amino acid sequence of SEQ ID NO: 62, the HCDR3 comprises the amino acid sequence of SEQ ID NO: 63, the LCDR1 comprises the amino acid sequence of SEQ ID NO: 64, the LCDR2 comprises the amino acid sequence of SEQ ID NO: 65, and the LCDR3 comprises the amino acid sequence of SEQ ID NO: 66.

14. The binding molecule of claim 12 or claim 13, wherein the antibody comprises a VH comprising the amino acid sequence of SEQ ID NO: 87 and a V_{L} comrpsiing the amino acid sequence of SEQ ID NO: 88.

15. The binding molecule of claim 5, wherein the functional domain binds to TGFb.

16. The binding molecule of claim 15, wherein the functional domain comprises TGFb receptor II extracellular domain (TRII) or a fragment or a variant thereof, and wherein optionally the functional domain comprises the amino acid sequence of SEQ ID NO: 94 or a fragment thereof.

17. The binding molecule of claim 5 or claim 6, wherein the functional domain binds to TIGIT.

18. The binding molecule of claim 17, wherein the antibody comprises an HCDR1, an HCDR2, and an HCDR3 as set forth in SEQ ID NO: 265, and a LCDR1, a LCDR2, and a LCDR3 as set forth in SEQ ID NO: 266.

19. The binding molecule of claim 18, wherein the HCDR1 comprises the amino acid sequence of SEQ ID NO: 267, the HCDR2 comprises the amino acid sequence of SEQ ID NO: 268, the HCDR3 comprises the amino acid sequence of SEQ ID NO: 269, the LCDR1 comprises the amino acid sequence of SEQ ID NO: 270, the LCDR2 comprises the amino acid sequence of SEQ ID NO: 271, and the LCDR3 comprises the amino acid sequence of SEQ ID NO: 272.

20. The binding molecule of claim 18 or claim 19, wherein the antibody comprises a VH comprising the amino acid sequence of SEQ ID NO: 265 and a VL comrpsiing the amino acid sequence of SEQ ID NO: 266.

21. The binding molecule of claim 5 or claim 6, wherein the functional domain binds to PVRIG.

22. The binding molecule of claim 21, wherein the antibody comprises an HCDR1, an HCDR2, and an HCDR3 as set forth in SEQ ID NO: 273, and a LCDR1, a LCDR2, and a LCDR3 as set forth in SEQ ID NO: 274.

23. The binding molecule of claim 22, wherein the HCDR1 comprises the amino acid sequence of SEQ ID NO: 275, the HCDR2 comprises the amino acid sequence of SEQ ID NO: 276, the HCDR3 comprises the amino acid sequence of SEQ ID NO: 277, the LCDR1 comprises the amino acid sequence of SEQ ID NO: 278, the LCDR2 comprises the amino acid sequence of SEQ ID NO: 279, and the LCDR3 comprises the amino acid sequence of SEQ ID NO: 280.

24. The binding molecule of claim 21 or claim 22, wherein the antibody comprises a VH comprising the amino acid sequence of SEQ ID NO: 273 and a V_{L} comrpsiing the amino acid sequence of SEQ ID NO: 274.

25. The binding molecule of any one of claims 5 to 24, wherein the binding molecule further comprises an Fc region comprising one or more mutation(s) that enhances ADCC.

26. The binding molecule of claim 25, wherein the Fc region comprising one or more mutation(s) at the position of 233, 234, 235, 236, 237, 238, 239, 240, 241, 243, 244, 245, 246, 247, 248, 250, 253, 254, 255, 256, 258, 260, 262, 263, 264, 265, 266, 267, 268, 268, 269, 270, 272, 274, 276, 280, 282, 283, 285, 286, 288, 290, 292, 293, 294, 295, 296, 297, 298, 299, 300, 301, 303, 307, 311, 312, 313, 315, 317, 318, 320, 322, 325, 326, 327, 328, 329, 330, 331, 332, 333, 334, 335, 337, 338, 339, 340, 342, 344, 345, 347, 355, 356, 358, 359, 360, 361, 362, 373, 375, 376, 378, 380, 382, 383, 384, 386, 388, 389, 390, 391, 392, 396, 398, 400, 401, 413, 414, 415, 416, 418, 419, 421, 422, 424, 428, 430, 433, 434, 435, 436, 437, 438, 439, 440, 442, 444, and/or 447 according to EU numbering.

27. The binding molecule of claim 26, wherein the Fc region comprises S239D/1332E, S239D/A330L/1332E, L235V/F243L/R292P/Y300L/P396L, or F243L/R292P/Y300L mutations.

28. The binding molecule of any one of claims 1 to 27, wherein the antigen(s) expressed on the HSC is selected from a group consisting of 5HT1B, 5HTIF, 5HT2A, 5-HT2B, 5-HT7, A2a, A2b, a1b1 integrin, a2bi integrin, a5b1 integrin, a6b4 integrin, a8b1 integrin, avb1 integrin, avb3 integrin, ACVR2A, ACVR2B, AdipoR1, AdipoR2, ADRA1A, ADRA1B, ANTXR1, AT1, AT2, BAMB1, BMPR2, C5aR, CB1, CB2, CCR1, CCR2, CCR5, CCR7, CD105, CD112, CD14, CD146, CD155, CD248, CD36, CD38, CD40, CD44, CD49e, CD62e, CD73, CD95, c-MET, CNTFR, CXCR3, CXCR4, DDR1, DDR2, EGFR, ETA, ETB, FAP, FGFR2, FN, gp130, GPC3, GPR91, ICAM-1, IGF-1R, IGF-2R, IL-10R2, IL-11RA, IL-17RA, IL-20R1, IL-20R2, IL-22R1, IL-6R, KCNE4, ITGA8, LRP, MICA, MICB, NCAM, NGFR, NPR-B, NPR3, OB-Ra, OB-Rb, OPRD1, P2X4, P2X7, P2Y6, p75NTR, PAFR, PAR1 PAR2 PAR4, PDGFRA, PDGFRB, PD-L1, PD-L2, Ptc, PTH-1R, RAGE, SIRPA, CD47, SYP, TGFBR1, TGFBR2, TGFBR3, TLR2, TLR3, TLR4, TLR7, TLR9, TNFR1, TRKB, TRKC, ULBP1, ULPB2, uPAR, VACM-1, VEGFR-1, and VEGFR-2.

29. The binding molecule of any one of claims 1 to 28, wherein the antigen is PDGFRb.

30. The binding molecule of claim 29, wherein the binding domain comprises an HCDR1, an HCDR2, and an HCDR3 as set forth in SEQ ID NO: 67, and a LCDR1, a LCDR2, and a LCDR3 as set forth in SEQ ID NO: 68.

31. The binding molecule of claim 30, wherein the HCDR1 comprises the amino acid sequence of SEQ ID NO: 1, the HCDR2 comprises the amino acid sequence of SEQ ID NO: 2, the HCDR3 comprises the amino acid sequence of SEQ ID NO: 3, the LCDR1 comprises the amino acid sequence of SEQ ID NO: 4, the LCDR2 comprises the amino acid sequence of SEQ ID NO: 5, and the LCDR3 comprises the amino acid sequence of SEQ ID NO: 6.

32. The binding molecule of claim 30 or claim 31, wherein the binding domain comprises a VH comprising the amino acid sequence of SEQ ID NO: 67 and a V_{L} comrpsiing the amino acid sequence of SEQ ID NO: 68.

33. The binding molecule of any one of claims 1 to 28, wherein the antigen is SIRPA.

34. The binding molecule of claim 33, wherein the binding domain comprises (i) an HCDR1, an HCDR2, and an HCDR3 as set forth in SEQ ID NO: 69, and a LCDR1, a LCDR2, and a LCDR3 as set forth in SEQ ID NO: 70;
(ii) an HCDR1, an HCDR2, and an HCDR3 as set forth in SEQ ID NO: 71, and a LCDR1, a LCDR2, and a LCDR3 as set forth in SEQ ID NO: 72; or
(iii) an HCDR1, an HCDR2, and an HCDR3 as set forth in SEQ ID NO: 73, and a LCDR1, a LCDR2, and a LCDR3 as set forth in SEQ ID NO: 74.

35. The binding molecule of claim 34, wherein:
(i) the HCDR1 comprises the amino acid sequence of SEQ ID NO: 7, the HCDR2 comprises the amino acid sequence of SEQ ID NO: 8, the HCDR3 comprises the amino acid sequence of SEQ ID NO: 9, the LCDR1 comprises the amino acid sequence of SEQ ID NO:
10, the LCDR2 comprises the amino acid sequence of SEQ ID NO: 11, and the LCDR3 comprises the amino acid sequence of SEQ ID NO: 12;
(ii) the HCDR1 comprises the amino acid sequence of SEQ ID NO: 13, the HCDR2 comprises the amino acid sequence of SEQ ID NO: 14, the HCDR3 comprises the amino acid sequence of SEQ ID NO: 15, the LCDR1 comprises the amino acid sequence of SEQ ID NO:
16, the LCDR2 comprises the amino acid sequence of SEQ ID NO: 17, and the LCDR3 comprises the amino acid sequence of SEQ ID NO: 18; or
(iii) the HCDR1 comprises the amino acid sequence of SEQ ID NO: 19, the HCDR2 comprises the amino acid sequence of SEQ ID NO: 20, the HCDR3 comprises the amino acid sequence of SEQ ID NO: 21, the LCDR1 comprises the amino acid sequence of SEQ ID NO:
22, the LCDR2 comprises the amino acid sequence of SEQ ID NO: 23, and the LCDR3 comprises the amino acid sequence of SEQ ID NO: 24.

36. The binding molecule of claim 34 or claim 35, wherein the binding domain comprises:
(i) a VH comprising the amino acid sequence of SEQ ID NO: 69 and a V_{L} comrpsiing the amino acid sequence of SEQ ID NO: 70;
(ii) a VH comprising the amino acid sequence of SEQ ID NO: 71 and a V_{L} comrpsiing the amino acid sequence of SEQ ID NO: 72; or
(iii) a VH comprising the amino acid sequence of SEQ ID NO: 73 and a V_{L} comrpsiing the amino acid sequence of SEQ ID NO: 74.

37. The binding molecule of any one of claims 1 to 28, wherein the antigen is FAPα.

38. The binding molecule of claim 37, wherein the binding domain comprises
(i) an HCDR1, an HCDR2, and an HCDR3 as set forth in SEQ ID NO: 75, and a LCDR1, a LCDR2, and a LCDR3 as set forth in SEQ ID NO: 76; or
(ii) an HCDR1, an HCDR2, and an HCDR3 as set forth in SEQ ID NO: 77, and a LCDR1, a LCDR2, and a LCDR3 as set forth in SEQ ID NO: 78.

39. The binding molecule of claim 38, wherein:
(i) the HCDR1 comprises the amino acid sequence of SEQ ID NO: 25, the HCDR2 comprises the amino acid sequence of SEQ ID NO: 26, the HCDR3 comprises the amino acid sequence of SEQ ID NO: 27, the LCDR1 comprises the amino acid sequence of SEQ ID NO:
28, the LCDR2 comprises the amino acid sequence of SEQ ID NO: 29, and the LCDR3 comprises the amino acid sequence of SEQ ID NO: 30; or
(ii) the HCDR1 comprises the amino acid sequence of SEQ ID NO: 31, the HCDR2 comprises the amino acid sequence of SEQ ID NO: 32, the HCDR3 comprises the amino acid sequence of SEQ ID NO: 33, the LCDR1 comprises the amino acid sequence of SEQ ID NO:
34, the LCDR2 comprises the amino acid sequence of SEQ ID NO: 35, and the LCDR3 comprises the amino acid sequence of SEQ ID NO: 36.

40. The binding molecule of claim 38 or claim 39, wherein the binding domain comprises:
(i) a VH comprising the amino acid sequence of SEQ ID NO: 75 and a V_{L} comrpsiing the amino acid sequence of SEQ ID NO: 76; or
(ii) a VH comprising the amino acid sequence of SEQ ID NO: 77 and a V_{L} comrpsiing the amino acid sequence of SEQ ID NO: 78.

41. The binding molecule of any one of claims 1 to 28, wherein the antigen is PD-L1.

42. The binding molecule of claim 41, wherein the binding domain comprises an HCDR1, an HCDR2, and an HCDR3 as set forth in SEQ ID NO: 79, and a LCDR1, a LCDR2, and a LCDR3 as set forth in SEQ ID NO: 80.

43. The binding molecule of claim 42, wherein the HCDR1 comprises the amino acid sequence of SEQ ID NO: 37, the HCDR2 comprises the amino acid sequence of SEQ ID NO: 38, the HCDR3 comprises the amino acid sequence of SEQ ID NO: 39, the LCDR1 comprises the amino acid sequence of SEQ ID NO: 40, the LCDR2 comprises the amino acid sequence of SEQ ID NO: 41, and the LCDR3 comprises the amino acid sequence of SEQ ID NO: 42.

44. The binding molecule of claim 42 or claim 43, wherein the binding domain comprises a VH comprising the amino acid sequence of SEQ ID NO: 79 and a V_{L} comrpsiing the amino acid sequence of SEQ ID NO: 80.

45. The binding molecule of any one of claims 1 to 28, wherein the antigen is uPAR.

46. The binding molecule of claim 45, wherein the binding domain comprises an HCDR1, an HCDR2, and an HCDR3 as set forth in SEQ ID NO: 81, and a LCDR1, a LCDR2, and a LCDR3 as set forth in SEQ ID NO: 82.

47. The binding molecule of claim 46, wherein the HCDR1 comprises the amino acid sequence of SEQ ID NO: 43, the HCDR2 comprises the amino acid sequence of SEQ ID NO: 44, the HCDR3 comprises the amino acid sequence of SEQ ID NO: 45, the LCDR1 comprises the amino acid sequence of SEQ ID NO: 46, the LCDR2 comprises the amino acid sequence of SEQ ID NO: 47, and the LCDR3 comprises the amino acid sequence of SEQ ID NO: 48.

48. The binding molecule of claim 46 or claim 47, wherein the binding domain comprises a VH comprising the amino acid sequence of SEQ ID NO: 81 and a V_{L} comrpsiing the amino acid sequence of SEQ ID NO: 82.

49. The binding molecule of any one of claims 1 to 28, wherein the antigen is IGF-1R.

50. The binding molecule of claim 49, wherein the binding domain comprises:
(i) an HCDR1, an HCDR2, and an HCDR3 as set forth in SEQ ID NO: 83, and a LCDR1, a LCDR2, and a LCDR3 as set forth in SEQ ID NO: 84; or
(ii) an HCDR1, an HCDR2, and an HCDR3 as set forth in SEQ ID NO: 85, and a LCDR1, a LCDR2, and a LCDR3 as set forth in SEQ ID NO: 86.

51. The binding molecule of claim 50, wherein:
(i) the HCDR1 comprises the amino acid sequence of SEQ ID NO: 49, the HCDR2 comprises the amino acid sequence of SEQ ID NO: 50, the HCDR3 comprises the amino acid sequence of SEQ ID NO: 51, the LCDR1 comprises the amino acid sequence of SEQ ID NO:
52, the LCDR2 comprises the amino acid sequence of SEQ ID NO: 53, and the LCDR3 comprises the amino acid sequence of SEQ ID NO: 54; or
(ii) the HCDR1 comprises the amino acid sequence of SEQ ID NO: 55, the HCDR2 comprises the amino acid sequence of SEQ ID NO: 56, the HCDR3 comprises the amino acid sequence of SEQ ID NO: 57, the LCDR1 comprises the amino acid sequence of SEQ ID NO:
58, the LCDR2 comprises the amino acid sequence of SEQ ID NO: 59, and the LCDR3 comprises the amino acid sequence of SEQ ID NO: 60.

52. The binding molecule of claim 50 or claim 51, wherein the binding domain comprises:
(i) a VH comprising the amino acid sequence of SEQ ID NO: 83 and a V_{L} comrpsiing the amino acid sequence of SEQ ID NO: 84; or
(ii) a VH comprising the amino acid sequence of SEQ ID NO: 85 and a V_{L} comrpsiing the amino acid sequence of SEQ ID NO: 86.

53. The binding molecule of any one of claims 1 to 28, wherein the antigen is a NKG2D ligand, and wherein optionally the NKG2D ligand is MICA, MICB, ULBP1, or ULBP2.

54. The binding molecule of claim 53, wherein the binding domain comprises the NKG2D extracellular domain or a fragment or a variant thereof, and wherein optionally the binding domain comprises the amino acid sequence of SEQ ID NO: 89 or a fragment thereof.

55. The binding molecule of any one of claims 1 to 28, wherein the antigen is ANTXR1.

56. The binding molecule of claim 55, wherein the binding domain comprises:
(i) an HCDR1, an HCDR2, and an HCDR3 as set forth in SEQ ID NO: 225, and a LCDR1, a LCDR2, and a LCDR3 as set forth in SEQ ID NO: 226; or
(ii) an HCDR1, an HCDR2, and an HCDR3 as set forth in SEQ ID NO: 233, and a LCDR1, a LCDR2, and a LCDR3 as set forth in SEQ ID NO: 234.

57. The binding molecule of claim 56, wherein:
(i) the HCDR1 comprises the amino acid sequence of SEQ ID NO: 227, the HCDR2 comprises the amino acid sequence of SEQ ID NO: 228, the HCDR3 comprises the amino acid sequence of SEQ ID NO: 229, the LCDR1 comprises the amino acid sequence of SEQ ID NO: 230, the LCDR2 comprises the amino acid sequence of SEQ ID NO: 231, and the LCDR3 comprises the amino acid sequence of SEQ ID NO: 232; or
(ii) the HCDR1 comprises the amino acid sequence of SEQ ID NO: 235, the HCDR2 comprises the amino acid sequence of SEQ ID NO: 236, the HCDR3 comprises the amino acid sequence of SEQ ID NO: 237, the LCDR1 comprises the amino acid sequence of SEQ ID NO: 238, the LCDR2 comprises the amino acid sequence of SEQ ID NO: 239, and the LCDR3 comprises the amino acid sequence of SEQ ID NO: 240.

58. The binding molecule of claim 56 or claim 57, wherein the binding domain comprises:
(i) a VH comprising the amino acid sequence of SEQ ID NO: 225 and a V_{L} comrpsiing the amino acid sequence of SEQ ID NO: 226; or
(ii) a VH comprising the amino acid sequence of SEQ ID NO: 233 and a V_{L} comrpsiing the amino acid sequence of SEQ ID NO: 232.

59. The binding molecule of any one of claims 1 to 28, wherein the antigen is CD248.

60. The binding molecule of claim 59, wherein the binding domain comprises:
(i) an HCDR1, an HCDR2, and an HCDR3 as set forth in SEQ ID NO: 241, and a LCDR1, a LCDR2, and a LCDR3 as set forth in SEQ ID NO: 242; or
(ii) an HCDR1, an HCDR2, and an HCDR3 as set forth in SEQ ID NO: 249, and a LCDR1, a LCDR2, and a LCDR3 as set forth in SEQ ID NO: 250.

61. The binding molecule of claim 60, wherein:
(i) the HCDR1 comprises the amino acid sequence of SEQ ID NO: 243, the HCDR2 comprises the amino acid sequence of SEQ ID NO: 244, the HCDR3 comprises the amino acid sequence of SEQ ID NO: 245, the LCDR1 comprises the amino acid sequence of SEQ ID NO: 246, the LCDR2 comprises the amino acid sequence of SEQ ID NO: 247, and the LCDR3 comprises the amino acid sequence of SEQ ID NO: 248; or
(ii) the HCDR1 comprises the amino acid sequence of SEQ ID NO: 251, the HCDR2 comprises the amino acid sequence of SEQ ID NO: 252, the HCDR3 comprises the amino acid sequence of SEQ ID NO: 253, the LCDR1 comprises the amino acid sequence of SEQ ID NO: 254, the LCDR2 comprises the amino acid sequence of SEQ ID NO: 255, and the LCDR3 comprises the amino acid sequence of SEQ ID NO: 256.

62. The binding molecule of claim 60 or claim 61, wherein the binding domain comprises:
(i) a VH comprising the amino acid sequence of SEQ ID NO: 241 and a V_{L} comrpsiing the amino acid sequence of SEQ ID NO: 242; or
(ii) a VH comprising the amino acid sequence of SEQ ID NO: 249 and a V_{L} comrpsiing the amino acid sequence of SEQ ID NO: 250.

63. The binding molecule of any one of claims 1 to 28, wherein the antigen is GPC3.

64. The binding molecule of claim 63, wherein the binding domain comprises an HCDR1, an HCDR2, and an HCDR3 as set forth in SEQ ID NO: 257, and a LCDR1, a LCDR2, and a LCDR3 as set forth in SEQ ID NO: 258.

65. The binding molecule of claim 64, wherein the HCDR1 comprises the amino acid sequence of SEQ ID NO: 259, the HCDR2 comprises the amino acid sequence of SEQ ID NO: 260, the HCDR3 comprises the amino acid sequence of SEQ ID NO: 261, the LCDR1 comprises the amino acid sequence of SEQ ID NO: 262, the LCDR2 comprises the amino acid sequence of SEQ ID NO: 263, and the LCDR3 comprises the amino acid sequence of SEQ ID NO: 264.

66. The binding molecule of claim 64 or claim 65, wherein the binding domain comprises a VH comprising the amino acid sequence of SEQ ID NO: 257 and a V_{L} comrpsiing the amino acid sequence of SEQ ID NO: 258.

67. The binding molecule of any one of claims 1 to 66, wherein the binding molecule is an IgG antibody or a fusion protein comprising the IgG antibody.

68. The binding molecule of claim 67, wherein the antibody is a humanized antibody.

69. A nucleic acid molecule encoding the binding molecule of any one of claims 1 to 68 or a fragment thereof.

70. A vector comprising the nucleic acid molecule of claim 69.

71. A host cell transformed with the vector of claim 70.

72. A composition comprising a therapeutically effective amount of the binding molecule of any one of claims 1 to 68, the nucleic acid molecule of claim 69, or the vector of claim 70, and a pharmaceutically acceptable excipient.

73. A method of treating a disease or disorder in a subject, comprising administering to the subject the composition of claim 72.

74. The method of claim 73, wherein the disease or disorder is associated with activiated HSCs.

75. The method of claim 74, wherein the disease or disorder is liver fibrosis.

76. A method of depleting activated HSCs in a subject, comprising administering to the subject the composition of claim 72.

77. The method of claim 76, wherein the subject has a disease or disorder associated with activiated HSCs.

78. The method of claim 77, wherein the disease or disorder is liver fibrosis.


SEQUENCE LISTING (299 sequences)

SEQ ID NO: 1 - protein, 5 aa, Artificial Sequence
  Exemplary anti-PDGFRb antibody (alpha-PDGFRb 1) HCDR1
NYAIN

SEQ ID NO: 2 - protein, 16 aa, Artificial Sequence
  Exemplary anti-PDGFRb antibody (alpha-PDGFRb 1) HCDR2
IIWTGGGTSYNSALKS

SEQ ID NO: 3 - protein, 10 aa, Artificial Sequence
  Exemplary anti-PDGFRb antibody (alpha-PDGFRb 1) HCDR3
TGTRGYFFDY

SEQ ID NO: 4 - protein, 11 aa, Artificial Sequence
  Exemplary anti-PDGFRb antibody (alpha-PDGFRb 1) LCDR1
HASQNINVWLS

SEQ ID NO: 5 - protein, 7 aa, Artificial Sequence
  Exemplary anti-PDGFRb antibody (alpha-PDGFRb 1) LCDR2
KASNLHT

SEQ ID NO: 6 - protein, 9 aa, Artificial Sequence
  Exemplary anti-PDGFRb antibody (alpha-PDGFRb 1) LCDR3
QQGQSFPFT

SEQ ID NO: 7 - protein, 5 aa, Artificial Sequence
  Exemplary anti-SIRPA antibody (alpha-SIRPA 1) HCDR1
DMYIH

SEQ ID NO: 8 - protein, 16 aa, Artificial Sequence
  Exemplary anti-SIRPA antibody (alpha-SIRPA 1) HCDR2
IDPENDDTKYDPKFQD

SEQ ID NO: 9 - protein, 5 aa, Artificial Sequence
  Exemplary anti-SIRPA antibody (alpha-SIRPA 1) HCDR3
GNYVH

SEQ ID NO: 10 - protein, 15 aa, Artificial Sequence
  Exemplary anti-SIRPA antibody (alpha-SIRPA 1) LCDR1
RASESVQYYGTSLMQ

SEQ ID NO: 11 - protein, 7 aa, Artificial Sequence
  Exemplary anti-SIRPA antibody (alpha-SIRPA 1) LCDR2
AASNVES

SEQ ID NO: 12 - protein, 8 aa, Artificial Sequence
  Exemplary anti-SIRPA antibody (alpha-SIRPA 1) LCDR3
QQSRRVWT

SEQ ID NO: 13 - protein, 5 aa, Artificial Sequence
  Exemplary anti-SIRPA antibody (alpha-SIRPA 2) HCDR1
DMYLH

SEQ ID NO: 14 - protein, 16 aa, Artificial Sequence
  Exemplary anti-SIRPA antibody (alpha-SIRPA 2) HCDR2
IDPENDSTKYDPKFQD

SEQ ID NO: 15 - protein, 5 aa, Artificial Sequence
  Exemplary anti-SIRPA antibody (alpha-SIRPA 2) HCDR3
GNYVH

SEQ ID NO: 16 - protein, 15 aa, Artificial Sequence
  Exemplary anti-SIRPA antibody (alpha-SIRPA 2) LCDR1
RASESVQYYGTSLMQ

SEQ ID NO: 17 - protein, 7 aa, Artificial Sequence
  Exemplary anti-SIRPA antibody (alpha-SIRPA 2) LCDR2
AASNVGS

SEQ ID NO: 18 - protein, 8 aa, Artificial Sequence
  Exemplary anti-SIRPA antibody (alpha-SIRPA 2) LCDR3
QQTRRVWT

SEQ ID NO: 19 - protein, 5 aa, Artificial Sequence
  Exemplary anti-SIRPA antibody (alpha-SIRPA 3) HCDR1
DSYMH

SEQ ID NO: 20 - protein, 16 aa, Artificial Sequence
  Exemplary anti-SIRPA antibody (alpha-SIRPA 3) HCDR2
IDPEDGETKYAPKFQG

SEQ ID NO: 21 - protein, 4 aa, Artificial Sequence
  Exemplary anti-SIRPA antibody (alpha-SIRPA 3) HCDR3
RGFY

SEQ ID NO: 22 - protein, 10 aa, Artificial Sequence
  Exemplary anti-SIRPA antibody (alpha-SIRPA 3) LCDR1
SSSVSSSYLY

SEQ ID NO: 23 - protein, 7 aa, Artificial Sequence
  Exemplary anti-SIRPA antibody (alpha-SIRPA 3) LCDR2
STSNLAS

SEQ ID NO: 24 - protein, 9 aa, Artificial Sequence
  Exemplary anti-SIRPA antibody (alpha-SIRPA 3) LCDR3
YQWSTHPYT

SEQ ID NO: 25 - protein, 5 aa, Artificial Sequence
  Exemplary anti-FAPalpha antibody (alpha-FAPalpha 1) HCDR1
EYTIH

SEQ ID NO: 26 - protein, 17 aa, Artificial Sequence
  Exemplary anti-FAPalpha antibody (alpha-FAPalpha 1) HCDR2
GINPNNGIPNYNQKFKG

SEQ ID NO: 27 - protein, 15 aa, Artificial Sequence
  Exemplary anti-FAPalpha antibody (alpha-FAPalpha 1) HCDR3
RRIAYGYDEGHAMDY

SEQ ID NO: 28 - protein, 17 aa, Artificial Sequence
  Exemplary anti-FAPalpha antibody (alpha-FAPalpha 1) LCDR1
KSSQSLLYSRNQKNYLA

SEQ ID NO: 29 - protein, 7 aa, Artificial Sequence
  Exemplary anti-FAPalpha antibody (alpha-FAPalpha 1) LCDR2
WASTRES

SEQ ID NO: 30 - protein, 9 aa, Artificial Sequence
  Exemplary anti-FAPalpha antibody (alpha-FAPalpha 1) LCDR3
QQYFSYPLT

SEQ ID NO: 31 - protein, 5 aa, Artificial Sequence
  Exemplary anti-FAPalpha antibody (alpha-FAPalpha 2) HCDR1
SYAMS

SEQ ID NO: 32 - protein, 16 aa, Artificial Sequence
  Exemplary anti-FAPalpha antibody (alpha-FAPalpha 2) HCDR2
AIIGSGASTYYADSVK

SEQ ID NO: 33 - protein, 8 aa, Artificial Sequence
  Exemplary anti-FAPalpha antibody (alpha-FAPalpha 2) HCDR3
YCAKGWFG

SEQ ID NO: 34 - protein, 11 aa, Artificial Sequence
  Exemplary anti-FAPalpha antibody (alpha-FAPalpha 2) LCDR1
RASQSVTSSYL

SEQ ID NO: 35 - protein, 7 aa, Artificial Sequence
  Exemplary anti-FAPalpha antibody (alpha-FAPalpha 2) LCDR2
NVGSRRA

SEQ ID NO: 36 - protein, 9 aa, Artificial Sequence
  Exemplary anti-FAPalpha antibody (alpha-FAPalpha 2) LCDR3
CQQGIMLPP

SEQ ID NO: 37 - protein, 10 aa, Artificial Sequence
  Exemplary anti-PDL1 antibody (alpha-PDL1 1) HCDR1
GYTFTSYWMY

SEQ ID NO: 38 - protein, 17 aa, Artificial Sequence
  Exemplary anti-PDL1 antibody (alpha-PDL1 1) HCDR2
RIDPNSGSTKYNEKFKN

SEQ ID NO: 39 - protein, 11 aa, Artificial Sequence
  Exemplary anti-PDL1 antibody (alpha-PDL1 1) HCDR3
DYRKGLYAMDY

SEQ ID NO: 40 - protein, 11 aa, Artificial Sequence
  Exemplary anti-PDL1 antibody (alpha-PDL1 1) LCDR1
KASQDVGTAVA

SEQ ID NO: 41 - protein, 7 aa, Artificial Sequence
  Exemplary anti-PDL1 antibody (alpha-PDL1 1) LCDR2
WASTRHT

SEQ ID NO: 42 - protein, 9 aa, Artificial Sequence
  Exemplary anti-PDL1 antibody (alpha-PDL1 1) LCDR3
QQYNSYPLT

SEQ ID NO: 43 - protein, 5 aa, Artificial Sequence
  Exemplary anti-uPAR antibody (alpha-uPAR 1) HCDR1
SYGMH

SEQ ID NO: 44 - protein, 16 aa, Artificial Sequence
  Exemplary anti-uPAR antibody (alpha-uPAR 1) HCDR2
VIWYDGSNKYYADSVK

SEQ ID NO: 45 - protein, 13 aa, Artificial Sequence
  Exemplary anti-uPAR antibody (alpha-uPAR 1) HCDR3
DQHIVVVTAILDY

SEQ ID NO: 46 - protein, 17 aa, Artificial Sequence
  Exemplary anti-uPAR antibody (alpha-uPAR 1) LCDR1
KSSQSVLYGSNNRNSLA

SEQ ID NO: 47 - protein, 7 aa, Artificial Sequence
  Exemplary anti-uPAR antibody (alpha-uPAR 1) LCDR2
WASTRES

SEQ ID NO: 48 - protein, 10 aa, Artificial Sequence
  Exemplary anti-uPAR antibody (alpha-uPAR 1) LCDR3
QQYYSIFPWT

SEQ ID NO: 49 - protein, 5 aa, Artificial Sequence
  Exemplary anti-IGF1R antibody (alpha-IGF1R 1) HCDR1
IYRMQ

SEQ ID NO: 50 - protein, 17 aa, Artificial Sequence
  Exemplary anti-IGF1R antibody (alpha-IGF1R 1) HCDR2
GISPSGGTTWYADSVKG

SEQ ID NO: 51 - protein, 11 aa, Artificial Sequence
  Exemplary anti-IGF1R antibody (alpha-IGF1R 1) HCDR3
WSGGSGYAFDI

SEQ ID NO: 52 - protein, 11 aa, Artificial Sequence
  Exemplary anti-IGF1R antibody (alpha-IGF1R 1) LCDR1
QASRDIRNYLN

SEQ ID NO: 53 - protein, 7 aa, Artificial Sequence
  Exemplary anti-IGF1R antibody (alpha-IGF1R 1) LCDR2
DASSLQT

SEQ ID NO: 54 - protein, 9 aa, Artificial Sequence
  Exemplary anti-IGF1R antibody (alpha-IGF1R 1) LCDR3
QQFDSLPHT

SEQ ID NO: 55 - protein, 5 aa, Artificial Sequence
  Exemplary anti-IGF1R antibody (alpha-IGF1R 2) HCDR1
NYPMY

SEQ ID NO: 56 - protein, 17 aa, Artificial Sequence
  Exemplary anti-IGF1R antibody (alpha-IGF1R 2) HCDR2
RISSSGGRTVYADSVKG

SEQ ID NO: 57 - protein, 15 aa, Artificial Sequence
  Exemplary anti-IGF1R antibody (alpha-IGF1R 2) HCDR3
DRWSRSAAEYGLGGY

SEQ ID NO: 58 - protein, 17 aa, Artificial Sequence
  Exemplary anti-IGF1R antibody (alpha-IGF1R 2) LCDR1
KSSQSVLYSSNNKNYLA

SEQ ID NO: 59 - protein, 7 aa, Artificial Sequence
  Exemplary anti-IGF1R antibody (alpha-IGF1R 2) LCDR2
LASTRES

SEQ ID NO: 60 - protein, 8 aa, Artificial Sequence
  Exemplary anti-IGF1R antibody (alpha-IGF1R 2) LCDR3
QQYYSTWT

SEQ ID NO: 61 - protein, 5 aa, Artificial Sequence
  Exemplary anti-NKp46 antibody (alpha-NKp46 1) HCDR1
DYVIN

SEQ ID NO: 62 - protein, 19 aa, Artificial Sequence
  Exemplary anti-NKp46 antibody (alpha-NKp46 1) HCDR2
EIYPGSGTNYYNEKFKAKA

SEQ ID NO: 63 - protein, 11 aa, Artificial Sequence
  Exemplary anti-NKp46 antibody (alpha-NKp46 1) HCDR3
RGRYGLYAMDY

SEQ ID NO: 64 - protein, 11 aa, Artificial Sequence
  Exemplary anti-NKp46 antibody (alpha-NKp46 1) LCDR1
RASQDISNYLN

SEQ ID NO: 65 - protein, 7 aa, Artificial Sequence
  Exemplary anti-NKp46 antibody (alpha-NKp46 1) LCDR2
YTSRLHS

SEQ ID NO: 66 - protein, 9 aa, Artificial Sequence
  Exemplary anti-NKp46 antibody (alpha-NKp46 1) LCDR3
QQGNTRPWT

SEQ ID NO: 67 - protein, 118 aa, Artificial Sequence
  Exemplary anti-PDGFRb antibody (alpha-PDGFRb 1) VH
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSS

SEQ ID NO: 68 - protein, 107 aa, Artificial Sequence
  Exemplary anti-PDGFRb antibody (alpha-PDGFRb 1) VL
DIQMTQSPSSLSASVGDRVTITCHASQNINVWLSWYQQKPGKAPKLLIYKASNLHTGVPSRFSGSGSGTGFTLTISSLQPEDIATYYCQQGQSFPFTFGGGTKVEIK

SEQ ID NO: 69 - protein, 114 aa, Artificial Sequence
  Exemplary anti-SIRPA antibody (alpha-SIRPA 1) VH
EVQLQQSGAELVKPGASVKLSCTTSGFNFKDMYIHWVKQRPQQGLEWIGRIDPENDDTKYDPKFQDKATITADTSSNTAYLQLSNLPSEDTAVYYCARGNYVHWGQGTLVTVSA

SEQ ID NO: 70 - protein, 110 aa, Artificial Sequence
  Exemplary anti-SIRPA antibody (alpha-SIRPA 1) VL
DIVLTQSPASLAVSLGQRATISCRASESVQYYGTSLMQWYQQKPGQPPKLLIYAASNVESGVPARFSGSGSGTDFSLNIYPVEEDDIAMYFCQQSRRVWTFGGGTKLEIK

SEQ ID NO: 71 - protein, 114 aa, Artificial Sequence
  Exemplary anti-SIRPA antibody (alpha-SIRPA 2) VH
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSA

SEQ ID NO: 72 - protein, 110 aa, Artificial Sequence
  Exemplary anti-SIRPA antibody (alpha-SIRPA 2) VL
DIVLTQSPASLAVSLGQRATISCRASESVQYYGTSLMQWYQQKPGQPPKLLIYAASNVGSGVPARFSGSGSGTDFSLNIHPVEEDDIAMYFCQQTRRVWTFGGGTKLEIK

SEQ ID NO: 73 - protein, 113 aa, Artificial Sequence
  Exemplary anti-SIRPA antibody (alpha-SIRPA 3) VH
EVQLQQSGAELVKPGASVKLSCTASGFNIKDSYMHWVKQRTEQGLEWIGRIDPEDGETKYAPKFQGKATITADTSSNTAYLQLTSLTSEDTAVYYCARRGFYWGQGTTLTVSS

SEQ ID NO: 74 - protein, 108 aa, Artificial Sequence
  Exemplary anti-SIRPA antibody (alpha-SIRPA 3) VL
QIVLTQSPAIMSASPGEKVTLTCSASSSVSSSYLYWYQQKPGSSPKLWIYSTSNLASGVPARFSGSGSGTSYSLTISSMEAEDAASYFCYQWSTHPYTFGGGTKLEIK

SEQ ID NO: 75 - protein, 124 aa, Artificial Sequence
  Exemplary anti-FAPalpha antibody (alpha-FAPalpha 1) VH
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 76 - protein, 113 aa, Artificial Sequence
  Exemplary anti-FAPalpha antibody (alpha-FAPalpha 1) VL
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 77 - protein, 117 aa, Artificial Sequence
  Exemplary anti-FAPalpha antibody (alpha-FAPalpha 2) VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 78 - protein, 108 aa, Artificial Sequence
  Exemplary anti-FAPalpha antibody (alpha-FAPalpha 2) VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 79 - protein, 120 aa, Artificial Sequence
  Exemplary anti-PDL1 antibody (alpha-PDL1 1) VH
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSS

SEQ ID NO: 80 - protein, 107 aa, Artificial Sequence
  Exemplary anti-PDL1 antibody (alpha-PDL1 1) VL
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIK

SEQ ID NO: 81 - protein, 122 aa, Artificial Sequence
  Exemplary anti-uPAR antibody (alpha-uPAR 1) VH
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDQHIVVVTAILDYWGQGTLVTVSS

SEQ ID NO: 82 - protein, 114 aa, Artificial Sequence
  Exemplary anti-uPAR antibody (alpha-uPAR 1) VL
DIVMTQSPDSLSVSLGERATINCKSSQSVLYGSNNRNSLAWYQQKPGQPPKLLIYWASTRESGVPDRFSGSGSGTDFTLTISSLQAEDVAVYYCQQYYSIFPWTFGQGTKVEIK

SEQ ID NO: 83 - protein, 120 aa, Artificial Sequence
  Exemplary anti-IGF1R antibody (alpha-IGF1R 1) VH
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSS

SEQ ID NO: 84 - protein, 107 aa, Artificial Sequence
  Exemplary anti-IGF1R antibody (alpha-IGF1R 1) VL
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIK

SEQ ID NO: 85 - protein, 124 aa, Artificial Sequence
  Exemplary anti-IGF1R antibody (alpha-IGF1R 2) VH
EVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSS

SEQ ID NO: 86 - protein, 112 aa, Artificial Sequence
  Exemplary anti-IGF1R antibody (alpha-IGF1R 2) VL
DIQMTQSPDSLAVSLGERATINCKSSQSVLYSSNNKNYLAWYQQKPGQPPKLLIYLASTRESGVPDRFSGSGSGTDFTLTISSLQAEDVAVYYCQQYYSTWTFGQGTKVEIK

SEQ ID NO: 87 - protein, 120 aa, Artificial Sequence
  Exemplary anti-NKp46 antibody (alpha-NKp46 1) VH
QVQLVQSGAEVKKPGSSVKVSCKASGYTFTDYVINWGRQAPGQGLEWIGEIYPGSGTNYYNEKFKAKATITADKSTSTAYMELSSLRSEDTAVYFCARRGRYGLYAMDYWGQGTTVTVSS

SEQ ID NO: 88 - protein, 107 aa, Artificial Sequence
  Exemplary anti-NKp46 antibody (alpha-NKp46 1) VL
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 89 - protein, 139 aa, Artificial Sequence
  NKG2D extracellular domain
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 90 - protein, 190 aa, Artificial Sequence
  ULBP1 extracellular domain
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAP

SEQ ID NO: 91 - protein, 190 aa, Artificial Sequence
  ULBP2 extracellular domain
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAM

SEQ ID NO: 92 - protein, 284 aa, Artificial Sequence
  MICA extracellular domain
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 93 - protein, 287 aa, Artificial Sequence
  MICB extracellular domain
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTD

SEQ ID NO: 94 - protein, 136 aa, Artificial Sequence
  TGFb receptor II extracellular domain
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 95 - protein, 4 aa, Artificial Sequence
  Exemplary linker
IEGR

SEQ ID NO: 96 - protein, 5 aa, Artificial Sequence
  Exemplary linker
GGGGS

SEQ ID NO: 97 - protein, 8 aa, Artificial Sequence
  Exemplary linker
GGSGGGSG

SEQ ID NO: 98 - protein, 10 aa, Artificial Sequence
  Exemplary linker
GGGGSGGGGS

SEQ ID NO: 99 - protein, 15 aa, Artificial Sequence
  Exemplary linker
GGGGSGGGGSGGGGS

SEQ ID NO: 100 - protein, 20 aa, Artificial Sequence
  Exemplary linker
GGGGSGGGGSGGGGSGGGGS

SEQ ID NO: 101 - protein, 23 aa, Artificial Sequence
  Exemplary linker
GGSGGGSGGGGSGGGGSGGGGSG

SEQ ID NO: 102 - protein, 12 aa, Artificial Sequence
  Exemplary linker
AEAAAKEAAAKA

SEQ ID NO: 103 - protein, 17 aa, Artificial Sequence
  Exemplary linker
AEAAAKEAAAKEAAAKA

SEQ ID NO: 104 - protein, 9 aa, Artificial Sequence
  Exemplary linker
APAPAPAPA

SEQ ID NO: 105 - protein, 20 aa, Artificial Sequence
  Exemplary linker
APAPAPAPAPAPAPAPAPAP

SEQ ID NO: 106 - protein, 448 aa, Artificial Sequence
  alpha-PDGFRb 1 S239D/I332E Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 214 aa, Artificial Sequence
  alpha-PDGFRb 1 S239D/I332E Chain 2
DIQMTQSPSSLSASVGDRVTITCHASQNINVWLSWYQQKPGKAPKLLIYKASNLHTGVPSRFSGSGSGTGFTLTISSLQPEDIATYYCQQGQSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 444 aa, Artificial Sequence
  alpha-SIRPA 1 S239D/I332E Chain 1
EVQLQQSGAELVKPGASVKLSCTTSGFNFKDMYIHWVKQRPQQGLEWIGRIDPENDDTKYDPKFQDKATITADTSSNTAYLQLSNLPSEDTAVYYCARGNYVHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 217 aa, Artificial Sequence
  alpha-SIRPA 1 S239D/I332E Chain 2
DIVLTQSPASLAVSLGQRATISCRASESVQYYGTSLMQWYQQKPGQPPKLLIYAASNVESGVPARFSGSGSGTDFSLNIYPVEEDDIAMYFCQQSRRVWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 444 aa, Artificial Sequence
  alpha-SIRPA 2 S239D/I332E Chain 1
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 217 aa, Artificial Sequence
  alpha-SIRPA 2 S239D/I332E Chain 2
DIVLTQSPASLAVSLGQRATISCRASESVQYYGTSLMQWYQQKPGQPPKLLIYAASNVGSGVPARFSGSGSGTDFSLNIHPVEEDDIAMYFCQQTRRVWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 443 aa, Artificial Sequence
  alpha-SIRPA 3 S239D/I332E Chain 1
EVQLQQSGAELVKPGASVKLSCTASGFNIKDSYMHWVKQRTEQGLEWIGRIDPEDGETKYAPKFQGKATITADTSSNTAYLQLTSLTSEDTAVYYCARRGFYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 215 aa, Artificial Sequence
  alpha-SIRPA 3 S239D/I332E Chain 2
QIVLTQSPAIMSASPGEKVTLTCSASSSVSSSYLYWYQQKPGSSPKLWIYSTSNLASGVPARFSGSGSGTSYSLTISSMEAEDAASYFCYQWSTHPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 454 aa, Artificial Sequence
  alpha-FAPa 1 S239D/I332E Chain 1
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 115 - protein, 220 aa, Artificial Sequence
  alpha-FAPa 1 S239D/I332E Chain 2
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 116 - protein, 447 aa, Artificial Sequence
  alpha-FAPa 2 Chain 1
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 215 aa, Artificial Sequence
  alpha-FAPa 2 Chain 2
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 450 aa, Artificial Sequence
  alpha-PDL1 1 S239D/I332E Chain 1
EVQLVQSGAEVKKPGATVKISCKVSGYTFTSYWMYWVRQARGQRLEWIGRIDPNSGSTKYNEKFKNRFTISRDNSKNTLYLQMNSLRAEDTAVYYCARDYRKGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 214 aa, Artificial Sequence
  alpha-PDL1 1 S239D/I332E Chain 2
AIQLTQSPSSLSASVGDRVTITCKASQDVGTAVAWYLQKPGQSPQLLIYWASTRHTGVPSRFSGSGSGTDFTFTISSLEAEDAATYYCQQYNSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 452 aa, Artificial Sequence
  alpha-uPAR 1 S239D/I332E Chain 1
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDQHIVVVTAILDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 221 aa, Artificial Sequence
  alpha-uPAR 1 S239D/I332E Chain 2
DIVMTQSPDSLSVSLGERATINCKSSQSVLYGSNNRNSLAWYQQKPGQPPKLLIYWASTRESGVPDRFSGSGSGTDFTLTISSLQAEDVAVYYCQQYYSIFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 450 aa, Artificial Sequence
  alpha-IGF1R 1 S239D/I332E Chain 1
EVQLLESGGGLVQPGGSLRLSCAASGFTFSIYRMQWVRQAPGKGLEWVSGISPSGGTTWYADSVKGRFTISRDNSKNTLYLQMNSLRAEDTAVYYCARWSGGSGYAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 214 aa, Artificial Sequence
  alpha-IGF1R 1 S239D/I332E Chain 2
DIQMTQSPLSLSASVGDRVTITCQASRDIRNYLNWYQQKPGKAPKLLIYDASSLQTGVPSRFGGSGSGTDFSFTIGSLQPEDIATYYCQQFDSLPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 454 aa, Artificial Sequence
  alpha-IGF1R 2 S239D/I332E Chain 1
EVQLLESGGGLVQPGGSLRLSCAASGFTFSNYPMYWVRQAPGKGLEWVSRISSSGGRTVYADSVKGRFTISRDNSKNTLYLQMNSLRAEDTAVYYCARDRWSRSAAEYGLGGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 219 aa, Artificial Sequence
  alpha-IGF1R 2 S239D/I332E Chain 2
DIQMTQSPDSLAVSLGERATINCKSSQSVLYSSNNKNYLAWYQQKPGQPPKLLIYLASTRESGVPDRFSGSGSGTDFTLTISSLQAEDVAVYYCQQYYSTWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 448 aa, Artificial Sequence
  alpha-PDGFRb 1 L234A/L235A Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 214 aa, Artificial Sequence; identical to SEQ ID NO: 107
  alpha-PDGFRb 1 L234A/L235A Chain 2

SEQ ID NO: 128 - protein, 444 aa, Artificial Sequence
  alpha-SIRPA 2 Chain 1
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 217 aa, Artificial Sequence; identical to SEQ ID NO: 111
  alpha-SIRPA 2 Chain 2

SEQ ID NO: 130 - protein, 444 aa, Artificial Sequence
  alpha-SIRPA 2 L234A/L235A Chain 1
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 217 aa, Artificial Sequence; identical to SEQ ID NO: 111
  alpha-SIRPA 2 L234A/L235A Chain 2

SEQ ID NO: 132 - protein, 444 aa, Artificial Sequence
  alpha-SIRPA 2 S239D/A330L/I332E Chain 1
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 217 aa, Artificial Sequence; identical to SEQ ID NO: 111
  alpha-SIRPA 2 S239D/A330L/I332E Chain 2

SEQ ID NO: 134 - protein, 444 aa, Artificial Sequence
  alpha-SIRPA 2 F243L/R292P/Y300L Chain 1
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 217 aa, Artificial Sequence; identical to SEQ ID NO: 111
  alpha-SIRPA 2 F243L/R292P/Y300L Chain 2

SEQ ID NO: 136 - protein, 444 aa, Artificial Sequence
  alpha-SIRPA 2 L235V/F243L/R292P/Y300L/P396L Chain 1
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 137 - protein, 217 aa, Artificial Sequence; identical to SEQ ID NO: 111
  alpha-SIRPA 2 L235V/F243L/R292P/Y300L/P396L Chain 2

SEQ ID NO: 138 - protein, 374 aa, Artificial Sequence
  hIgG1 Fc-NKG2D Chain 1
PKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 139 - protein, 374 aa, Artificial Sequence
  hIgG1 Fc-NKG2D S239D/I332E Chain 1
PKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 140 - protein, 374 aa, Artificial Sequence
  hIgG1 Fc-NKG2D L234A/L235A Chain 1
PKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 141 - protein, 537 aa, Artificial Sequence
  NKG2D-hIgG1 Fc S239D/I332E Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGSPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 537 aa, Artificial Sequence
  NKG2D-hIgG1 Fc L234A/L235A Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 107 aa, Artificial Sequence [sequence omitted]

SEQ ID NO: 144 - protein, 330 aa, Artificial Sequence
  CH wild type
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 330 aa, Artificial Sequence
  CH S239D/I332E
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 330 aa, Artificial Sequence
  CH L234A/L235A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 330 aa, Artificial Sequence
  CH S239D/A330L/I332E
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 330 aa, Artificial Sequence
  CH L235V/F243L/R292P/Y300L/P396L
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 149 - protein, 330 aa, Artificial Sequence
  CH F243L/R292P/Y300L
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 330 aa, Artificial Sequence
  CH N297S
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 232 aa, Artificial Sequence
  CH2-CH3
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 98 aa, Artificial Sequence
  CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 153 - protein, 232 aa, Artificial Sequence
  CH2-CH3 S239D/I332E
EPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 232 aa, Artificial Sequence
  CH2-CH3 N297S
EPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 605 aa, Artificial Sequence
  alpha-PDGFRb 1-TRII S239D/I332E Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 156 - protein, 214 aa, Artificial Sequence; identical to SEQ ID NO: 107
  alpha-PDGFRb 1-TRII S239D/I332E Chain 2

SEQ ID NO: 157 - protein, 737 aa, Artificial Sequence
  MICA-GS-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWGGGGSQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 214 aa, Artificial Sequence; identical to SEQ ID NO: 107
  MICA-GS-HC- alpha-PDGFRb 1 S239D/I332E Chain 2

SEQ ID NO: 159 - protein, 732 aa, Artificial Sequence
  MICA-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 214 aa, Artificial Sequence; identical to SEQ ID NO: 107
  MICA-HC- alpha-PDGFRb 1 S239D/I332E Chain 2

SEQ ID NO: 161 - protein, 742 aa, Artificial Sequence
  MICA-GS2-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWGGGGSGGGGSQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 214 aa, Artificial Sequence; identical to SEQ ID NO: 107
  MICA-GS2-HC- alpha-PDGFRb 1 S239D/I332E Chain 2

SEQ ID NO: 163 - protein, 747 aa, Artificial Sequence
  MICA-GS3-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWGGGGSGGGGSGGGGSQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 214 aa, Artificial Sequence; identical to SEQ ID NO: 107
  MICA-GS3-HC- alpha-PDGFRb 1 S239D/I332E Chain 2

SEQ ID NO: 165 - protein, 752 aa, Artificial Sequence
  MICA-GS4-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWGGGGSGGGGSGGGGSGGGGSQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 214 aa, Artificial Sequence; identical to SEQ ID NO: 107
  MICA-GS4-HC- alpha-PDGFRb 1 S239D/I332E Chain 2

SEQ ID NO: 167 - protein, 744 aa, Artificial Sequence
  MICA-EAK2-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWAEAAAKEAAAKAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 214 aa, Artificial Sequence; identical to SEQ ID NO: 107
  MICA-EAK2-HC- alpha-PDGFRb 1 S239D/I332E Chain 2

SEQ ID NO: 169 - protein, 749 aa, Artificial Sequence
  MICA-EAK3-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWAEAAAKEAAAKEAAAKAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 214 aa, Artificial Sequence; identical to SEQ ID NO: 107
  MICA-EAK3-HC- alpha-PDGFRb 1 S239D/I332E Chain 2

SEQ ID NO: 171 - protein, 741 aa, Artificial Sequence
  MICA-AP-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWAPAPAPAPAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 214 aa, Artificial Sequence; identical to SEQ ID NO: 107
  MICA-AP-HC- alpha-PDGFRb 1 S239D/I332E Chain 2

SEQ ID NO: 173 - protein, 753 aa, Artificial Sequence
  NKG2D-GS1-NKG2D-GS4-HC alpha-PDGFRb 1 S239D/I332E Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 214 aa, Artificial Sequence; identical to SEQ ID NO: 107
  NKG2D-GS1-NKG2D-GS4-HC alpha-PDGFRb 1 S239D/I332E Chain 2

SEQ ID NO: 175 - protein, 768 aa, Artificial Sequence
  NKG2D-GS4-NKG2D-GS4-HC alpha-PDGFRb 1 S239D/I332E Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGGGGSGGGGSGGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - protein, 214 aa, Artificial Sequence; identical to SEQ ID NO: 107
  NKG2D-GS4-NKG2D-GS4-HC alpha-PDGFRb 1 S239D/I332E Chain 2

SEQ ID NO: 177 - protein, 756 aa, Artificial Sequence
  NKG2D-GS1-NKG2D-EAK4-HC alpha-PDGFRb 1 S239D/I332E Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAEAAAKEAAAKEAAAKEAAAKAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - protein, 214 aa, Artificial Sequence; identical to SEQ ID NO: 107
  NKG2D-GS1-NKG2D-EAK4-HC alpha-PDGFRb 1 S239D/I332E Chain 2

SEQ ID NO: 179 - protein, 766 aa, Artificial Sequence
  NKG2D-GS4-NKG2D-EAK4-HC alpha-PDGFRb 1 S239D/I332E Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGGGGSGGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAEAAAKEAAAKEAAAKEAAAKAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - protein, 214 aa, Artificial Sequence; identical to SEQ ID NO: 107
  NKG2D-GS4-NKG2D-EAK4-HC alpha-PDGFRb 1 S239D/I332E Chain 2

SEQ ID NO: 181 - protein, 754 aa, Artificial Sequence
  NKG2D-GS1-NKG2D-AP-HC alpha-PDGFRb 1 S239D/I332E Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAPAPAPAPAPAPAPAPAPAPQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - protein, 214 aa, Artificial Sequence; identical to SEQ ID NO: 107
  NKG2D-GS1-NKG2D-AP-HC alpha-PDGFRb 1 S239D/I332E Chain 2

SEQ ID NO: 183 - protein, 764 aa, Artificial Sequence
  NKG2D-GS4-NKG2D-AP-HC alpha-PDGFRb 1 S239D/I332E Chain 1
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGGGGSGGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVAPAPAPAPAPAPAPAPAPAPQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - protein, 214 aa, Artificial Sequence; identical to SEQ ID NO: 107
  NKG2D-GS4-NKG2D-AP-HC alpha-PDGFRb 1 S239D/I332E Chain 2

SEQ ID NO: 185 - protein, 747 aa, Artificial Sequence
  MICB-EAK2-HC- alpha-PDGFRb 1 S239D/I332E Chain 1
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTDAEAAAKEAAAKAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 214 aa, Artificial Sequence; identical to SEQ ID NO: 107
  MICB-EAK2-HC- alpha-PDGFRb 1 S239D/I332E Chain 2

SEQ ID NO: 187 - protein, 650 aa, Artificial Sequence
  ULBP1-EAK2-HC- alpha-PDGFRb 1 S239D/I332E Chain 1
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPAEAAAKEAAAKAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 214 aa, Artificial Sequence; identical to SEQ ID NO: 107
  ULBP1-EAK2-HC- alpha-PDGFRb 1 S239D/I332E Chain 2

SEQ ID NO: 189 - protein, 650 aa, Artificial Sequence
  ULBP2-EAK2-HC- alpha-PDGFRb 1 S239D/I332E Chain 1
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMAEAAAKEAAAKAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 214 aa, Artificial Sequence; identical to SEQ ID NO: 107
  ULBP2-EAK2-HC- alpha-PDGFRb 1 S239D/I332E Chain 2

SEQ ID NO: 191 - protein, 744 aa, Artificial Sequence
  MICB-AP-HC- alpha-PDGFRb 1 S239D/I332E Chain 1
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTDAPAPAPAPAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 214 aa, Artificial Sequence; identical to SEQ ID NO: 107
  MICB-AP-HC- alpha-PDGFRb 1 S239D/I332E Chain 2

SEQ ID NO: 193 - protein, 647 aa, Artificial Sequence
  ULBP1-AP-HC- alpha-PDGFRb 1 S239D/I332E Chain 1
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPAPAPAPAPAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 214 aa, Artificial Sequence; identical to SEQ ID NO: 107
  ULBP1-AP-HC- alpha-PDGFRb 1 S239D/I332E Chain 2

SEQ ID NO: 195 - protein, 647 aa, Artificial Sequence
  ULBP2-AP-HC- alpha-PDGFRb 1 S239D/I332E Chain 1
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMAPAPAPAPAQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 214 aa, Artificial Sequence; identical to SEQ ID NO: 107
  ULBP2-AP-HC- alpha-PDGFRb 1 S239D/I332E Chain 2

SEQ ID NO: 197 - protein, 741 aa, Artificial Sequence
  alpha-PDGFRb 1-HC-AP-MICA S239D/I332E Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAPAPAPAPAEPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHW

SEQ ID NO: 198 - protein, 214 aa, Artificial Sequence; identical to SEQ ID NO: 107
  alpha-PDGFRb 1-HC-AP-MICA S239D/I332E Chain 2

SEQ ID NO: 199 - protein, 839 aa, Artificial Sequence
  MICA-CL-HC- alpha-PDGFRb 1 S239D/I332E Chain 1
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 214 aa, Artificial Sequence; identical to SEQ ID NO: 107
  MICA-CL-HC- alpha-PDGFRb 1 S239D/I332E Chain 2

SEQ ID NO: 201 - protein, 842 aa, Artificial Sequence
  MICB-CL-HC- alpha-PDGFRb 1 S239D/I332E Chain 1
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTDRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 214 aa, Artificial Sequence; identical to SEQ ID NO: 107
  MICB-CL-HC- alpha-PDGFRb 1 S239D/I332E Chain 2

SEQ ID NO: 203 - protein, 745 aa, Artificial Sequence
  ULBP1-CL-HC- alpha-PDGFRb 1 S239D/I332E Chain 1
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 214 aa, Artificial Sequence; identical to SEQ ID NO: 107
  ULBP1-CL-HC- alpha-PDGFRb 1 S239D/I332E Chain 2

SEQ ID NO: 205 - protein, 745 aa, Artificial Sequence
  ULBP2-CL-HC- alpha-PDGFRb 1 S239D/I332E Chain 1
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 214 aa, Artificial Sequence; identical to SEQ ID NO: 107
  ULBP2-CL-HC- alpha-PDGFRb 1 S239D/I332E Chain 2

SEQ ID NO: 207 - protein, 735 aa, Artificial Sequence
  MICB-HC- alpha-PDGFRb 1 S239D/I332E Chain 1
AEPHSLRYNLMVLSQDESVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKVLVLQSQRTDQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 214 aa, Artificial Sequence; identical to SEQ ID NO: 107
  MICB-HC- alpha-PDGFRb 1 S239D/I332E Chain 2

SEQ ID NO: 209 - protein, 638 aa, Artificial Sequence
  ULBP1-HC- alpha-PDGFRb 1 S239D/I332E Chain 1
GWVDTHCLCYDFIITPKSRPEPQWCEVQGLVDERPFLHYDCVNHKAKAFASLGKKVNVTKTWEEQTETLRDVVDFLKGQLLDIQVENLIPIEPLTLQARMSCEHEAHGHGRGSWQFLFNGQKFLLFDSNNRKWTALHPGAKKMTEKWEKNRDVTMFFQKISLGDCKMWLEEFLMYWEQMLDPTKPPSLAPQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 214 aa, Artificial Sequence; identical to SEQ ID NO: 107
  ULBP1-HC- alpha-PDGFRb 1 S239D/I332E Chain 2

SEQ ID NO: 211 - protein, 638 aa, Artificial Sequence
  ULBP2-HC- alpha-PDGFRb 1 S239D/I332E Chain 1
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 214 aa, Artificial Sequence; identical to SEQ ID NO: 107
  ULBP2-HC- alpha-PDGFRb 1 S239D/I332E Chain 2

SEQ ID NO: 213 - protein, 638 aa, Artificial Sequence
  ULBP2-HC- alpha-PDGFRb 1 L234A/L235A Chain 1
GRADPHSLCYDITVIPKFRPGPRWCAVQGQVDEKTFLHYDCGNKTVTPVSPLGKKLNVTTAWKAQNPVLREVVDILTEQLRDIQLENYTPKEPLTLQARMSCEQKAEGHSSGSWQFSFDGQIFLLFDSEKRMWTTVHPGARKMKEKWENDKVVAMSFHYFSMGDCIGWLEDFLMGMDSTLEPSAGAPLAMQVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 214 aa, Artificial Sequence; identical to SEQ ID NO: 107
  ULBP2-HC- alpha-PDGFRb 1 L234A/L235A Chain 2

SEQ ID NO: 215 - protein, 678 aa, Artificial Sequence
  alpha-PDGFRb 1 - alpha-NKp46 N297S Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFTDYVINWGRQAPGQGLEWIGEIYPGSGTNYYNEKFKAKATITADKSTSTAYMELSSLRSEDTAVYFCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 446 aa, Artificial Sequence
  alpha-PDGFRb 1 - alpha-NKp46 N297S Chain 2
DIQMTQSPSSLSASVGDRVTITCHASQNINVWLSWYQQKPGKAPKLLIYKASNLHTGVPSRFSGSGSGTGFTLTISSLQPEDIATYYCQQGQSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 205 aa, Artificial Sequence
  alpha-PDGFRb 1 - alpha-NKp46 N297S Chain 3
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 218 - protein, 678 aa, Artificial Sequence
  alpha-PDGFRb 1 - alpha-NKp46 S239D/I332E Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFTDYVINWGRQAPGQGLEWIGEIYPGSGTNYYNEKFKAKATITADKSTSTAYMELSSLRSEDTAVYFCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - protein, 446 aa, Artificial Sequence
  alpha-PDGFRb 1 - alpha-NKp46 S239D/I332E Chain 2
DIQMTQSPSSLSASVGDRVTITCHASQNINVWLSWYQQKPGKAPKLLIYKASNLHTGVPSRFSGSGSGTGFTLTISSLQPEDIATYYCQQGQSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 205 aa, Artificial Sequence; identical to SEQ ID NO: 217
  alpha-PDGFRb 1 - alpha-NKp46 S239D/I332E Chain 3

SEQ ID NO: 221 - protein, 448 aa, Artificial Sequence
  alpha-PDGFRb 1 - NA N297S Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 446 aa, Artificial Sequence; identical to SEQ ID NO: 216
  alpha-PDGFRb 1 - NA N297S Chain 2

SEQ ID NO: 223 - protein, 448 aa, Artificial Sequence; identical to SEQ ID NO: 106
  alpha-PDGFRb 1 - NA S239D/I332E Chain 1

SEQ ID NO: 224 - protein, 446 aa, Artificial Sequence; identical to SEQ ID NO: 219
  alpha-PDGFRb 1 - NA S239D/I332E Chain 2

SEQ ID NO: 225 - protein, 117 aa, Artificial Sequence
  Exemplary anti-ANTXR1 antibody (alpha-ANTXR1 1) VH
QVQLVQSGAEVKKPGTSVKVSCKVPGYTFSSYAISWVRQAPGQGLEWMGGIIPIFGTTNYAQKFQGRVTITGEESTSTVYMELSSLRSEDTAVYYCARDTDYMFDYWGQGTLVTVSS

SEQ ID NO: 226 - protein, 107 aa, Artificial Sequence [sequence omitted]

SEQ ID NO: 227 - protein, 5 aa, Artificial Sequence
  Exemplary anti-ANTXR1 antibody (alpha-ANTXR1 1) HCDR1
SYAIS

SEQ ID NO: 228 - protein, 17 aa, Artificial Sequence
  Exemplary anti-ANTXR1 antibody (alpha-ANTXR1 1) HCDR2
GIIPIFGTTNYAQKFQG

SEQ ID NO: 229 - protein, 8 aa, Artificial Sequence
  Exemplary anti-ANTXR1 antibody (alpha-ANTXR1 1) HCDR3
DTDYMFDY

SEQ ID NO: 230 - protein, 11 aa, Artificial Sequence
  Exemplary anti-ANTXR1 antibody (alpha-ANTXR1 1) LCDR1
QGDNLRDFYAS

SEQ ID NO: 231 - protein, 7 aa, Artificial Sequence
  Exemplary anti-ANTXR1 antibody (alpha-ANTXR1 1) LCDR2
GKNRRPS

SEQ ID NO: 232 - protein, 10 aa, Artificial Sequence
  Exemplary anti-ANTXR1 antibody (alpha-ANTXR1 1) LCDR3
SSRDNSKHVV

SEQ ID NO: 233 - protein, 117 aa, Artificial Sequence
  Exemplary anti-ANTXR1 antibody (alpha-ANTXR1 2) VH
QVQLKESPGALVKPTQTLTLTCTFSGFSLSTSGGGVSWIRQPPGKALEWLAHIYSNDDKSYSTSLKTRLTISKDTSKNQVVLTMTNMDPVDTATYYCARGGYFLDYWGQGTLVTVSS

SEQ ID NO: 234 - protein, 106 aa, Artificial Sequence
  Exemplary anti-ANTXR1 antibody (alpha-ANTXR1 2) VL
DIELTQPPSVSVAPGQTARISCSGDNIGGIYVHWYQQKPGQAPVLVIYADSKRPSGIPERFSGSNSGNTATLTISGTQAEDEADYYCQSYDITSLVFGGGTKLTVL

SEQ ID NO: 235 - protein, 7 aa, Artificial Sequence
  Exemplary anti-ANTXR1 antibody (alpha-ANTXR1 2) HCDR1
TSGGGVS

SEQ ID NO: 236 - protein, 16 aa, Artificial Sequence
  Exemplary anti-ANTXR1 antibody (alpha-ANTXR1 2) HCDR2
HIYSNDDKSYSTSLKT

SEQ ID NO: 237 - protein, 7 aa, Artificial Sequence
  Exemplary anti-ANTXR1 antibody (alpha-ANTXR1 2) HCDR3
GGYFLDY

SEQ ID NO: 238 - protein, 11 aa, Artificial Sequence
  Exemplary anti-ANTXR1 antibody (alpha-ANTXR1 2) LCDR1
SGDNIGGIYVH

SEQ ID NO: 239 - protein, 7 aa, Artificial Sequence
  Exemplary anti-ANTXR1 antibody (alpha-ANTXR1 2) LCDR2
ADSKRPS

SEQ ID NO: 240 - protein, 9 aa, Artificial Sequence
  Exemplary anti-ANTXR1 antibody (alpha-ANTXR1 2) LCDR3
QSYDITSLV

SEQ ID NO: 241 - protein, 116 aa, Artificial Sequence
  Exemplary anti-CD248 antibody (alpha-CD248 1) VH
QVQLQESGPGLVAPSQSLSITCTVSGFSLTGYGVNWVRQPPEKGLEWLGMIWVDGSTDYNSALKSRLNISKDKSKSQVFLKMNSLQTDDTARYYCARGGYGAMDYWGQGTSVTVSS

SEQ ID NO: 242 - protein, 107 aa, Artificial Sequence
  Exemplary anti-CD248 antibody (alpha-CD248 1) VL
DIQMNQSPSSLSASLGDTITITCHASQNINVWLTWYQQKPGNIPKLLIYKASNLHTGVPSRFSGSGSGTGFTLTISSLQPEDIATYYCQQGQSYPWTFGGGTKLEIK

SEQ ID NO: 243 - protein, 5 aa, Artificial Sequence
  Exemplary anti-CD248 antibody (alpha-CD248 1) HCDR1
GYGVN

SEQ ID NO: 244 - protein, 16 aa, Artificial Sequence
  Exemplary anti-CD248 antibody (alpha-CD248 1) HCDR2
MIWVDGSTDYNSALKS

SEQ ID NO: 245 - protein, 8 aa, Artificial Sequence
  Exemplary anti-CD248 antibody (alpha-CD248 1) HCDR3
GGYGAMDY

SEQ ID NO: 246 - protein, 11 aa, Artificial Sequence
  Exemplary anti-CD248 antibody (alpha-CD248 1) LCDR1
HASQNINVWLT

SEQ ID NO: 247 - protein, 7 aa, Artificial Sequence
  Exemplary anti-CD248 antibody (alpha-CD248 1) LCDR2
KASNLHT

SEQ ID NO: 248 - protein, 9 aa, Artificial Sequence
  Exemplary anti-CD248 antibody (alpha-CD248 1) LCDR3
QQGQSYPWT

SEQ ID NO: 249 - protein, 124 aa, Artificial Sequence
  Exemplary anti-CD248 antibody (alpha-CD248 2) VH
QVQLQESGPGLVRPSQTLSLTCTASGYTFTDYVIHWVKQPPGRGLEWIGYINPYDDDTTYNQKFKGRVTMLVDTSSNTAYLRLSSVTAEDTAVYYCARRGNSYDGYFDYSMDYWGSGTPVTVSS

SEQ ID NO: 250 - protein, 108 aa, Artificial Sequence
  Exemplary anti-CD248 antibody (alpha-CD248 2) VL
DIQMTQSPSSLSASVGDRVTITCRASQNVGTAVAWLQQTPGKAPKLLIYSASNRYTGVPSRFSGSGSGTDYTFTISSLQPEDIATYYCQQYTNYPMYTFGQGTKVQIK

SEQ ID NO: 251 - protein, 5 aa, Artificial Sequence
  Exemplary anti-CD248 antibody (alpha-CD248 2) HCDR1
DYVIH

SEQ ID NO: 252 - protein, 17 aa, Artificial Sequence
  Exemplary anti-CD248 antibody (alpha-CD248 2) HCDR2
YINPYDDDTTYNQKFKG

SEQ ID NO: 253 - protein, 15 aa, Artificial Sequence
  Exemplary anti-CD248 antibody (alpha-CD248 2) HCDR3
RGNSYDGYFDYSMDY

SEQ ID NO: 254 - protein, 11 aa, Artificial Sequence
  Exemplary anti-CD248 antibody (alpha-CD248 2) LCDR1
RASQNVGTAVA

SEQ ID NO: 255 - protein, 7 aa, Artificial Sequence
  Exemplary anti-CD248 antibody (alpha-CD248 2) LCDR2
SASNRYT

SEQ ID NO: 256 - protein, 10 aa, Artificial Sequence
  Exemplary anti-CD248 antibody (alpha-CD248 2) LCDR3
QQYTNYPMYT

SEQ ID NO: 257 - protein, 115 aa, Artificial Sequence
  Exemplary anti-GPC3 antibody (alpha-GPC3 1) VH
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSA

SEQ ID NO: 258 - protein, 112 aa, Artificial Sequence
  Exemplary anti-GPC3 antibody (alpha-GPC3 1) VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 259 - protein, 5 aa, Artificial Sequence
  Exemplary anti-GPC3 antibody (alpha-GPC3 1) HCDR1
DYEMH

SEQ ID NO: 260 - protein, 17 aa, Artificial Sequence
  Exemplary anti-GPC3 antibody (alpha-GPC3 1) HCDR2
ALDPKTGDTAYSQKFKG

SEQ ID NO: 261 - protein, 6 aa, Artificial Sequence
  Exemplary anti-GPC3 antibody (alpha-GPC3 1) HCDR3
FYSYTY

SEQ ID NO: 262 - protein, 16 aa, Artificial Sequence
  Exemplary anti-GPC3 antibody (alpha-GPC3 1) LCDR1
RSSQSLVHSNGNTYLH

SEQ ID NO: 263 - protein, 7 aa, Artificial Sequence
  Exemplary anti-GPC3 antibody (alpha-GPC3 1) LCDR2
KVSNRFS

SEQ ID NO: 264 - protein, 9 aa, Artificial Sequence
  Exemplary anti-GPC3 antibody (alpha-GPC3 1) LCDR3
SQNTHVPPT

SEQ ID NO: 265 - protein, 126 aa, Artificial Sequence
  Exemplary anti-TIGIT antibody (alpha-TIGIT 1) VH
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 266 - protein, 113 aa, Artificial Sequence
  Exemplary anti-TIGIT antibody (alpha-TIGIT 1) VL
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 267 - protein, 7 aa, Artificial Sequence
  Exemplary anti-TIGIT antibody (alpha-TIGIT 1) HCDR1
SNSAAWN

SEQ ID NO: 268 - protein, 18 aa, Artificial Sequence
  Exemplary anti-TIGIT antibody (alpha-TIGIT 1) HCDR2
KTYYRFKWYSDYAVSVKG

SEQ ID NO: 269 - protein, 14 aa, Artificial Sequence
  Exemplary anti-TIGIT antibody (alpha-TIGIT 1) HCDR3
ESTTYDLLAGPFDY

SEQ ID NO: 270 - protein, 17 aa, Artificial Sequence
  Exemplary anti-TIGIT antibody (alpha-TIGIT 1) LCDR1
KSSQTVLYSSNNKKYLA

SEQ ID NO: 271 - protein, 7 aa, Artificial Sequence
  Exemplary anti-TIGIT antibody (alpha-TIGIT 1) LCDR2
WASTRES

SEQ ID NO: 272 - protein, 9 aa, Artificial Sequence
  Exemplary anti-TIGIT antibody (alpha-TIGIT 1) LCDR3
QQYYSTPFT

SEQ ID NO: 273 - protein, 120 aa, Artificial Sequence
  Exemplary anti-PVRIG antibody (alpha-PVRIG 1) VH
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSS

SEQ ID NO: 274 - protein, 107 aa, Artificial Sequence
  Exemplary anti-PVRIG antibody (alpha-PVRIG 1) VL
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIK

SEQ ID NO: 275 - protein, 5 aa, Artificial Sequence
  Exemplary anti-PVRIG antibody (alpha-PVRIG 1) HCDR1
DYNIN

SEQ ID NO: 276 - protein, 17 aa, Artificial Sequence
  Exemplary anti-PVRIG antibody (alpha-PVRIG 1) HCDR2
YIYPYIGGSGYAQKFQG

SEQ ID NO: 277 - protein, 11 aa, Artificial Sequence
  Exemplary anti-PVRIG antibody (alpha-PVRIG 1) HCDR3
EDKTARNAMDY

SEQ ID NO: 278 - protein, 11 aa, Artificial Sequence
  Exemplary anti-PVRIG antibody (alpha-PVRIG 1) LCDR1
RVSENIYSNLA

SEQ ID NO: 279 - protein, 7 aa, Artificial Sequence
  Exemplary anti-PVRIG antibody (alpha-PVRIG 1) LCDR2
EATNLAE

SEQ ID NO: 280 - protein, 9 aa, Artificial Sequence
  Exemplary anti-PVRIG antibody (alpha-PVRIG 1) LCDR3
QHFWGTPYT

SEQ ID NO: 281 - protein, 447 aa, Artificial Sequence
  alpha-ANTXR1 1 mIgG2a S239D/I332E Chain 1
QVQLVQSGAEVKKPGTSVKVSCKVPGYTFSSYAISWVRQAPGQGLEWMGGIIPIFGTTNYAQKFQGRVTITGEESTSTVYMELSSLRSEDTAVYYCARDTDYMFDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 282 - protein, 213 aa, Artificial Sequence
  alpha-ANTXR1 1 mIgG2a S239D/I332E Chain 2
SSELTQDPVVSVALGETVSITCQGDNLRDFYASWYQQKPGQAPLLVMYGKNRRPSGIPDRFSGSTSGNTLSLTITGAQAEDEADYYCSSRDNSKHVVFGGGTKVTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 283 - protein, 447 aa, Artificial Sequence
  alpha-ANTXR1 2 mIgG2a S239D/I332E Chain 1
QVQLKESPGALVKPTQTLTLTCTFSGFSLSTSGGGVSWIRQPPGKALEWLAHIYSNDDKSYSTSLKTRLTISKDTSKNQVVLTMTNMDPVDTATYYCARGGYFLDYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 284 - protein, 212 aa, Artificial Sequence
  alpha-ANTXR1 2 mIgG2a S239D/I332E Chain 2
DIELTQPPSVSVAPGQTARISCSGDNIGGIYVHWYQQKPGQAPVLVIYADSKRPSGIPERFSGSNSGNTATLTISGTQAEDEADYYCQSYDITSLVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 285 - protein, 446 aa, Artificial Sequence
  alpha-CD248 1 mIgG2a S239D/I332E Chain 1
QVQLQESGPGLVAPSQSLSITCTVSGFSLTGYGVNWVRQPPEKGLEWLGMIWVDGSTDYNSALKSRLNISKDKSKSQVFLKMNSLQTDDTARYYCARGGYGAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 286 - protein, 214 aa, Artificial Sequence
  alpha-CD248 1 mIgG2a S239D/I332E Chain 2
DIQMNQSPSSLSASLGDTITITCHASQNINVWLTWYQQKPGNIPKLLIYKASNLHTGVPSRFSGSGSGTGFTLTISSLQPEDIATYYCQQGQSYPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 287 - protein, 454 aa, Artificial Sequence
  alpha-CD248 2 mIgG2a S239D/I332E Chain 1
QVQLQESGPGLVRPSQTLSLTCTASGYTFTDYVIHWVKQPPGRGLEWIGYINPYDDDTTYNQKFKGRVTMLVDTSSNTAYLRLSSVTAEDTAVYYCARRGNSYDGYFDYSMDYWGSGTPVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 288 - protein, 215 aa, Artificial Sequence
  alpha-CD248 2 mIgG2a S239D/I332E Chain 2
DIQMTQSPSSLSASVGDRVTITCRASQNVGTAVAWLQQTPGKAPKLLIYSASNRYTGVPSRFSGSGSGTDYTFTISSLQPEDIATYYCQQYTNYPMYTFGQGTKVQIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 289 - protein, 445 aa, Artificial Sequence
  alpha-GPC3 1 mIgG2a S239D/I332E Chain 1
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 290 - protein, 219 aa, Artificial Sequence
  alpha-GPC3 1 mIgG2a S239D/I332E Chain 2
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 291 - protein, 444 aa, Artificial Sequence
  alpha-SIRPA 2 mIgG2a S239D/I332E Chain 1
EVQLQQSGAELVKPGASVKVSCTGSGFNFKDMYLHWVKQRPEQGLEWIGRIDPENDSTKYDPKFQDKATLTADTASNTAYLQISSLTSEDTAVYYCARGNYVHWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPDVFIFPPKIKDVLMISLSPIVTCVVVDVSEDDPDVQISWFVNNVEVHTAQTQTHREDYNSTLRVVSALPIQHQDWMSGKEFKCKVNNKDLPAPEERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 292 - protein, 217 aa, Artificial Sequence
  alpha-SIRPA 2 mIgG2a S239D/I332E Chain 2
DIVLTQSPASLAVSLGQRATISCRASESVQYYGTSLMQWYQQKPGQPPKLLIYAASNVGSGVPARFSGSGSGTDFSLNIHPVEEDDIAMYFCQQTRRVWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 293 - protein, 456 aa, Artificial Sequence
  alpha-TIGIT 1 IgG1 Chain 1
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 294 - protein, 220 aa, Artificial Sequence
  alpha-TIGIT 1 IgG1 Chain 2
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 446 aa, Artificial Sequence
  alpha-PVRIG 1 IgG4 Chain 1
QVQLVQSGAEVKKPGASVKVSCKASGYTFTDYNINWVRQAPGQGLEWMGYIYPYIGGSGYAQKFQGRVTMTRDTSTSTVYMELSSLRSEDTAVYYCAREDKTARNAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 296 - protein, 214 aa, Artificial Sequence
  alpha-PVRIG 1 IgG4 Chain 2
DIQMTQSPSSLSASVGDRVTITCRVSENIYSNLAWYQQKPGKAPKLLIYEATNLAEGVPSRFSGSGSGTDFTLTISSLQPEDFATYYCQHFWGTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 297 - protein, 448 aa, Artificial Sequence
  alpha-PDGFRb 1-NKG2D KIH S239D/I332E Chain 1
QVQLQESGPGLVKPSETLSLTCTVSGFSLTNYAINWVRQPPGKGLEWLGIIWTGGGTSYNSALKSRLTISKDTSKNQVSLKLSSVTAADTAVYYCARTGTRGYFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 298 - protein, 537 aa, Artificial Sequence
  alpha-PDGFRb 1-NKG2D KIH S239D/I332E Chain 2
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGSGGGSGFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVGGGGSGGGGSGGGGSGGGGSPKSSDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 214 aa, Artificial Sequence; identical to SEQ ID NO: 107
  alpha-PDGFRb 1-NKG2D KIH S239D/I332E Chain 3